SEQUENCE LISTING (147 sequences)

SEQ ID NO: 1 - DNA, 1773 bp, Arabidopsis thaliana
atgccgaagcattacagaccaacggggaaaaagaaggaaggaaatgcggctaggtatatgaccaggtcgcaagctcttaaacatcttcaagttaacttgaatctattcaggagactatgtattgtcaaaggtatatttccccgagaaccaaagaagaagattaagggaaaccatcacacttactaccatgtcaaggacattgctttcctcatgcacgagcctcttcttgagaagtttagggaaatcaaaacataccaaaagaaggtcaaaaaagccaaggccaagaaaaacgaggagcttgcacgccttctgctcacccgccaacctacttacaagcttgatagattgatccgtgagaggtatccaacatttattgatgcactgcgagacttggatgactgtctcactatggttcatctttttgcggtgttacctgcatcagacagggaaaatcttgaagttaagcgagtccacaactgtcgaagattgacccatgaatggcaagcttacatttcacgttctcatgcgttacgtaaagtgtttgtgtctgtcaagggtatttactatcaggctgaaatagaaggtcaaaagatcacttggttgacccctcatgcaatccaacaagtttttacaaatgatgttgactttggtgtcctgcttaccttcttggaattttacgagactcttcttgcctttattaacttcaagctttaccattctctgaatgttaaatacccgccaatccttgactctcggttggaggctttggctgcagatctctatgcactgtctagatacatagatgccagctccagaggcatggcggtggaacccaaagttgatgcttcatttagctcacagtcaaatgaccgtgaagagtctgaacttagacttgcacagcttcagcaccagctgccttcaagtgagcctggagcattgatgcatctggttgcagataacaataaagaggttgaagaggatgaagaaacaagagtgtgcaagtcactcttcaaggatctgaagtttttcttgagccgtgaggttccaagagagtccctgcaattggtgattactgcttttggtgggatggtgtcttgggaaggagaaggtgcacctttcaaggaggatgatgagagtattacacatcatatcatcgataagccaagcgctggccatttgtacctttctagggtatatgtgcaaccacagtggatctatgactgtgtgaatgctcgcataatcttgccaactgaaaagtacttggtcggaagaattccaccgccacacttgtcaccatttgtggacaatgaagcagaaggatacgttcctgattatgccgaaaccatcaaaagactacaggcagcagcaagaaacgaagtgcttccattgccaggtgttgggaaagaggatcttgaagatcctcaaaatttattgtacgctggtgttatgagtcgtgcggaggaagccgaagctgcaaagaacaagaagaagatggcggcgcaggagaagcaataccatgaggaactgaagatggaaataaatggaagtaaggatgttgtagcgcctgtgttggctgaaggtgagggtgaagaatcagttccggatgctatgcaaatagctcaagaggacgctgatatgcccaaagtgttgatgtcccgtaagaagaggaagctctacgatgccatgaagatttcgcagtcaaggaagagatcaggtgttgaaataatcgagcagcgcaagaaaaggttgaatgatactcaaccatcatcatga

SEQ ID NO: 2 - DNA, 1299 bp, Arabidopsis thaliana
atggatcctcaagctttcattcgtctttcggttggctctcttgctttgagaattcccaaggtccttataaactctacttcaaaatctaatgagaagaagaacttttcttctcaatgctcttgcgaaataaaactacgaggctttcctgttcaaacaacatctatccctttgatgccgtcccttgatgcagctcctgaccatcacagtatttccactagcttttatcttgaagaatctgatttaagagctcttttgacacctggatgcttctatagtcctcatgctcacttggaaatctcggttttcacgggtaaaaagagtttgaattgcggtgttggtggcaaaagacagcagattgggatgtttaagttggaggtaggtcctgaatggggagaaggaaaaccaatgattcttttcaatggttggatcagtattggaaagaccaagcgggatggtgctgcagagcttcatttgaaagtgaaacttgatcctgatcctcgatatgtttttcagtttgaggatgttactaccttgagccctcagatagttcagctccgtggctcggtcaagcaacctatcttcagttgcaagtttagcagagacagggtgtcacaggtggatccgttgaatgggtactggtcaagttcaggcgatggaactgagcttgagagtgagagacgtgaaagaaaaggatggaaggtgaagatacatgatctctctggctctgcagttgctgctgctttcataacaactccttttgttccatccactggatgtgattgggtcgcaaagtccaacccgggtgcttggcttgtggtccggcctgacccatctcgaccaaacagctggcagccatggggaaagctcgaagcttggcgggaacgcgggatcagagactccgtgtgttgcagattccatcttctatcaaacggtctagaagttggagatgttttaatgtctgaaatcctcatcagcgctgagaaaggtggggaatttttaatcgacacggataaacagatgctaacagttgcagctacaccaattccaagcccgcagagtagtggagacttctcagggttgggacagtgtgtctctggaggtgggtttgtaatgagctcgagagtgcaaggggaagggaaaagcagcaagcccgttgtacaattagctatgagacatgtaacttgtgtggaagatgcagccattttcatggcacttgctgcagctgttgatcttagcattcttgcttgtaaaccttttaggagaacgagtcggagaaggttccggcattactcctggtag

SEQ ID NO: 3 - DNA, 699 bp, Zea mays
atggtgcggggcaagacgcagatgaagcggatagagaacccgaccagccgccaggtcaccttctccaagcgccgcaacggcctgctcaagaaggcgttcgagctctccgtcctctgcgacgccgaggtcgccctcgtcgtcttctccccgcgcggcaagctctacgaattcgccagcggaagtgcgcagaaaacgattgaacgttatagaacatacacaaaggataatgttagcaacaagacagtgcagcaggatattgagcgagtaaaagctgatgcggatggcctgtcaaagagactcgaagcacttgaagcttacaaaaggaaacttttgggtgagaggttggaagactgctccattgaagagctgcacagtttggaagtcaagcttgagaagagcctgcattgcatcaggggaagaaagactgagctgctggaggagcaagtccgtaagctgaagcagaaggagatgagtctgcgcaagagcaacgaagatttgcgtgaaaagtgcaagaagcagccgcctgtgccgatggcttcggcgccgcctcgtgcgccggcagtcgacaacgtggaggacggtcaccgggagccgaaggacgacgggatggacgtggagacggagctgtacataggattgcccggcagagactaccgctcaagcaaagacaaggctgcagtggcggtcaggtcaggctag

SEQ ID NO: 4 - DNA, 1134 bp, Arabidopsis thaliana
atggccgtaagtttcgtaacaacatctcctgaggaagaagacaaaccgaagctaggccttggaaatattcaaactccgttaatcttcaacccttcaatgcttaaccttcaagccaatatcccaaaccaattcatctggcctgacgacgaaaaaccttccatcaacgttctcgagcttgatgttcctctcatcgaccttcaaaaccttctctctgatccatcctccactttagatgcttcgagactgatctctgaggcctgtaagaagcacggtttcttcctcgtggtcaatcacggcatcagcgaggagcttatttcagacgctcatgaatacacgagccgcttctttgatatgcctctctccgaaaaacagagggttcttagaaaatccggtgagagtgttggctacgcaagcagtttcaccggacgcttctccaccaagcttccatggaaggagaccctttctttccggttttgcgacgacatgagccgctcaaaatccgttcaagattacttctgcgatgcgttgggacatgggtttcagccatttgggaaggtgtatcaagagtattgtgaagcaatgagttctctatcactgaagatcatggagcttctggggctaagtttaggcgtaaaacgggactactttagagagtttttcgaagaaaacgattcaataatgagactgaattactaccctccatgtataaaaccagatctcacactaggaacaggacctcattgtgatccaacatctcttaccatccttcaccaagaccatgttaatggccttcaagtctttgtggaaaatcaatggcgctccattcgtcccaaccccaaggcctttgtggtcaatatcggcgatactttcatggctctatcgaacgatagatacaagagctgcttgcaccgggcggtggtgaacagcgagagcgagaggaaatcacttgcattcttcttgtgtccgaaaaaagacagagtagtgacgccaccgagagagcttttggacagcatcacatcaagaagataccctgacttcacatggtctatgttccttgagttcactcagaaacattatagagcagacatgaacactctccaagccttttcagattggctcaccaaacccatctag

SEQ ID NO: 5 - DNA, 2133 bp, Oryza sativa
atgtcagcgtcgccgtcgtcgatgagcggcgccggcgccggcgaggcgggggtgcggacggtggtgtggttcaggcgggacctgcgcgtggaggacaacccggcgctggcggcggcggcgcgggcggccggggaggtggtgccggtgtacgtgtgggcgccggaggaggacgggccgtactacccggggcgggtgtcccggtggtggctcagccagagcctcaagcacctggacgcctcgctccggcggctcggcgccagcaggctcgtcacccgccgctccgccgacgccgtcgtcgcgctcatcgagctcgtccgcagcatcggcgccacgcatctcttcttcaaccacctctacgacccgctgtcgctggtgagggaccaccgggtgaaggcgctgctgacggccgagggcatcgccgtgcagtcgttcaacgccgacctgctgtacgagccatgggaggtggtcgacgacgacggctgcccgttcaccatgttcgcgccgttctgggacaggtgcctgtgcatgcccgacccggcggcgccgctgctgccgcccaagaggatcgcgcccggcgagctgccggcgaggaggtgcccctccgacgagctggtgttcgaggacgagtccgagcgggggagcaacgcgctgctggcgagggcgtggtcgcccgggtggcagaacgccgacaaggcgctggccgcgttcctcaacgggccactcatggactactcggtgaaccggaagaaggccgacagcgccagcacgtcactgctgtcgccgtacctgcacttcggcgagctcagcgtacgcaaggtgttccaccaggtgaggatgaagcagctcatgtggagcaacgaggggaaccacgccggcgacgagagctgcgtcctcttcctccggtccatcggcctcagggagtactcgaggtacctcacgttcaaccacccgtgcagcctggagaagccactcctggcgcacctcaggttcttcccctgggtggtcgacgaggtgtacttcaaggtgtggaggcaggggaggacagggtaccctctcgtcgacgccgggatgcgcgagctctgggccaccggctggctgcacgaccggatacgcgtcgtcgtctccagcttcttcgtcaaggtgctccagcttccatggcgctgggggatgaagtacttctgggacaccctgctcgacgccgacctcgagagcgacgcgctcggctggcagtacatctccggctctctccccgatggccgtgagctcgaccgcatcgacaaccctcagcttgaaggatacaagtttgatccgcacggggagtatgtccggcgatggctgccggagctggcaaggctgccgacggagtggatacaccacccatgggacgcaccggagtcggtgctccaggctgcagggattgagctaggctccaactatcctctccccatcgtggagctggacgcggcgaagaccaggctgcaggatgcactgtcagagatgtgggagctcgaggccgcgtcacgcgcagcgatggagaacggaatggaggagggcctcggcgactcctccgacgtgccgccgatcgccttcccaccggagctgcagatggaagttgaccgagcaccggcccagcctactgttcacggaccgacaacggctggccggcgacgagaggatcagatggttcccagcatgacctcctcgctggtcagagctgaaacagaactttcagcggattttgacaacagcatggacagtaggccggaggtgccgtcgcaggtgctcttccagcctcggatggaaagggaagaaacagtggacggcggcggtggcggcggaatggtcggcaggagcaacggcggcggccaccaaggccaacaccagcagcaacagcacaactttcagactacaattcaccgggcacggggcgttgcgccgtctacgtcagaggcatcaagcaactggactgggagagaaggcggcgtggtgcccgtctggtcgcctccggcagcgtcaggcccctcagatcactacgctgccgatgaagctgacattaccagtagaagttatttggacaggcatccacagtcgcatacgttgatgaactggagtcagctctcgcagtcattgtag

SEQ ID NO: 6 - DNA, 1044 bp, Synechocystis sp. PCC 6803
atgaccgttagtgagattcatattcctaactctttactagaccgggattgcaccaccctttcacgccacgtactccaacaactgaatagctttggggccgatgcccaggatttgagtgccatcatgaaccgcattgccctagcgggaaaactgattgcccgtcgcctgagtcgagctgggttaatggccgatgtgttgggcttcactggggaaaccaacgtccagggggaatcggtgaaaaaaatggacgtatttgccaatgatgtttttatttctgtctttaagcaaagtggcttggtttgtcgtctggcttcggaggagatggaaaaaccctactatattcctgaaaattgccccattggtcgctatactttgctgtacgaccccattgatggttcctccaacgtggacattaacctcaacgtgggttccatttttgccattcggcaacaggaaggggacgatctagacggcagtgcgtcagatttattggctaacggagacaagcaaattgctgctggttatatcctctacggcccctccaccatcctggtttattccctcggctccggagtgcatagctttatcctcgatcccagtttgggggaatttattttagcccaggaaaatatccgcattcccaaccacggccccatttacagcaccaatgaaggtaacttttggcaatgggatgaagccctgagggattacacccgttacgtccatcgccacgaaggttacactgcccgttatagcggtgctctggtgggggatattcaccggattttgatgcaagggggagtgtttctttatcctggtacggaaaaaaatcccgacggcaaattgcgtttgctctatgaaactgcgccgctggcctttttggtggaacaggctgggggaagggctagtgacggccaaaaacgtttactggacttaattccttctaaattacatcagcgtacccccgccattattggcagcgcagaagatgtgaaattggtggaatctttcatcagcgaccacaaacaacggcagggtaattag

SEQ ID NO: 7 - DNA, 1050 bp, Zea mays
atgggcggcctcattatggaccaggccttcgtgcaggcccccgagcaccgccccaagcccatcgtcaccgaggccaccggcatccctctcatcgacctctcgcctctgtccgccagcggcggcgccgtggacgcgctggccgctgaggtgggcgcggcgagccgggactggggcttcttcgtggtcgtgggccacggcgtgcccgcagagaccgtggcgcgcgcgacggaggcgcagcgcgcgttcttcgcgctgccggcagagcggaaggcctccgtgcggaggaacgaggcggagccgctcgggtactacgagtcggagcacaccaagaacgtgagggactggaaggaggtgtacgacctcgcgccgcgcgagccgccgccgccggcagccgtggccgacggcgagctcgtgttcgagaacaagtggccccaggatctgccgggcttcagagaggcgctggaggagtacgcgaaagcgatggaagagctggcgttcaagctgctggagctgatcgcccggagcctgaagctgaggcccgaccggctgcacggcttcttcaaggaccagacgaccttcatccggctgaaccactaccctccatgcccgagccccgacctggccctcggcgtgggacggcacaaggacgccggcgccctgaccatcctgtaccaggacgacgtcggcgggctcgacgtccggcggcgctccgacggcgagtgggtccgcgtcaggcccgtgcccgactctttcatcatcaacgtcggcgacctcatccaggtgtggagcaacgacaggtacgagagcgcggagcaccgggtgtcagtgaactcggcgagagagaggttctccatgccctacttcttcaacccggcgacctacaccatggtggagccggtggaggagctggtgagcgaggacgatccgcccaggtacgacgcctacaactggggcgacttcttcagcaccaggaagaacagcaacttcaagaagctcaacgtggagaacattcagatcgcgcatttcaagaagagcctcgtcctcgcctag

SEQ ID NO: 8 - DNA, 1161 bp, Zea mays
atggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccagtggggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaa

SEQ ID NO: 9 - DNA, 468 bp, Arabidopsis thaliana
atggaaactttacaatgtcgtcatcagcatgtcttcattttgcttcttgtcctatttcattcctctctgtttgttttagcttcaaagatcgatgtttctgacgatgcacgaggcatcagaatcgacggtggccagaaacgttttctaactaattctcctcaacatggcaaggaacatgcagcgtgtacgaacgaagaacctgatctcggtccgctgacgcgtatttcttgcaatgaacctgaatatgttattacaaagatcaatttcgctgattatggcaatcccactggtacatgtggacactttagacgtgacaattgcggtgcacgagctaccatgaggatcgtcaaaaagaattgtcttggaaaagagaagtgtcaccttttggttacggatgagatgtttggtccgagcaagtgcaaaggagctcctatgctcgctgttgaaaccacttgtacaatagcttag

SEQ ID NO: 10 - DNA, 1119 bp, Zea mays
atggtgctggctgcgcacgatccccctccccttgtgttcgacgctgcccgcctgagcggcctctccgacatcccgcagcagttcatctggccggcggacgagagccccaccccggacgccgccgaggagctggccgtgccgctcatcgacctctccggggacgccgccgaggtggtccggcaggtccggcgcgcctgcgacctgcacggcttcttccaggtggtggggcacggcatcgacgcggcgctgacggcggaggcccaccgctgcatggacgccttcttcacgctgccgctcccggacaagcagcgcgcgcagcgccgccagggggacagctgcggctacgccagcagcttcacgggccggttcgcgtccaagctgccctggaaggagacgctgtcgttccgctacaccgacaacgacgacgacggcgacaagtccaaggacgtcgtggcgtcctacttcgtggacaagctgggcgaggggttccggcaccacggggaggtgtacgggcgctactgctctgagatgagccgtctgtcgctggagctcatggaggtgctaggcgagagcctgggcgtgggccggcgccacttccggcgcttcttccaggggaacgactccatcatgcgcctcaactactacccgccgtgccagcggccctacgacacgctgggcacggggccgcattgcgaccccacgtcgctcaccatcctgcaccaggacgacgtgggcggactccaggtgttcgacgccgccacgctcgcgtggcgctccatcaggccccgcccgggcgccttcgtcgtcaacatcggcgacaccttcatggcgctctccaacgggcgctacaggagctgcctccaccgcgccgtcgtcaacagccgggtggcacgccgctcgctcgccttcttcctgtgcccggagatggacaaggtggtcaggccgcccaaggagctggtggacgacgccaacccgagggcgtacccggacttcacgtggaggacgctgctggacttcaccatgaggcactacaggtcggacatgaggacgctcgaggccttctccaactggctcagcaccagtagcaatggcggacagcacctgctggagaagaagtag

SEQ ID NO: 11 - DNA, 1341 bp, Zea mays
atgcaggagcaggacgtggacgatggcggcggcaggacgacccagcagcaggagaagtcgatcgacgactggctccctatcaactcctccaggaaggccaagtggtggtactccgccttccacaatgtcaccgccatggtcggcgccggcgtgctcggcctcccctacgccatgtccgagcttggctggggccctggcatcgcggtgatgatcctgtcgtggataatcaccctatacacgctatggcagatggtggagatgcacgagatggtgcctgggaagcggttcgaccggtaccacgagctcgggcagcacgtcttcggcgacaggctgggcctctggatcgtggtgccgcagcagctggcagtagaggtgagcctgaacatcatctacatggtcaccggcggccagtccctcaagaagttccacgacgtcatctgcgacggcggcaggtgcggcggcgacttgaagctctcctacttcatcatgatcttcgcctccgtccacctggtcctctcccagctccccaacttcaactccatctccgccgtgtcgctcgccgccgccgtcatgtcgctcagttactccaccattgcgtggggcgcgtcgctgcacagagggaggagagaggacgtggactaccacctgcgcgccacgaccaccccagggaaggtgttcggcttcctgggaggcctaggggacgtggcgttcgcctactcggggcacaacgtggtgctggagatccaggccaccatcccgtccacgccggacaagccgtccaagaaggccatgtggaagggcgcctttgtcgcctacgtcgtcgtcgccatctgctacttccccgtcacgttcgtcgggtactgggccttcggcagcggcgtcgacgagaacatcctcatcacgctctccaagcccaagtggctcattgccctcgccaacatgatggtcgtcgtccatgtcattggcagttaccaggtttatgccatgccggtgtttgacatgatagagacggtgctggtcaagaaaatgaggttcgctccgagcctcacgctccgtcttattgcccggagcgtctatgttgcgttcacaatgtttctaggcatcactttccccttcttcggtggattgctcagtttcttcggcggattagccttcgcaccgacaacttattttcttccctgcatcatgtggctcaaggtttacaagcccaaacggttcggcctttcatggttcatcaactggatctgcatcgttattggagtgctgctgttgattctgggtccgataggagggctccggcagatcattttgtcagccaccacatacaaattctaccagtaa

SEQ ID NO: 12 - DNA, 405 bp, Arabidopsis thaliana
atggattttcagacaattcaagtgatgccatgggaatatgttctagcttctcaatctcttaataactatcaagagaatcatgttcgttggtctcagtctccagattctcacactttctctgttgatcttcctgggttaaggaaagaagaaataaaagttgagatcgaagattcgatatacttaatcatacgaacggaggcaacccctatgtcgcctccggatcagcctttgaagacttttaagaggaaattccggttgccggaatcaatagatatgatcggaatatcagctggttacgaagatggtgtgttgactgtgattgtacccaagaggattatgacaaggaggctcattgatccttctgatgttcctgaaagtcttcaacttcttgctagagctgcttaa

SEQ ID NO: 13 - DNA, 726 bp, Zea mays
atgaaccgggcgccgtcgctgtccgcggccggtgccgccgcggaggaggacgaggagcaggacgaggcgggggcggccgcggcggcggcatcgtcgtcgcccaacaacagcgcgagctccttcccgacggacttctccgcgcacggccaggtggcgcccggcgccgaccgcgcgtgctcccgcgccagcgacgaggacgacggcggctccgcgcgcaagaagctgcgcctctccaaggagcagtccgcgttcctggaggacagcttcaaggagcacgccacgctgaacccgaagcagaagctcgcgctggcgaagcagctcaacctccggccgcgccaggtggaggtgtggttccagaaccgcagagccaggacgaagctgaagcagacggaggtggactgcgagtacctcaagcgatgctgcgagacgctgacggaggagaaccggcggctgcagaaggagctatccgagctccgcgcgctcaagacggtgcaccccttctacatgcacctcccggccaccaccctttccatgtgcccctcctgcgagcgcgtcgcctccaactccgcgccggcgcccgcgtcatcgccgtcccccgctactggcattgcggccccggcaccggagcagaggccctcgtcgttcgcggctctgttctcgtcccctctgaaccgcccgctggccgcccaggcgcaaccgcaaccgcaggcgccggccaactcgtga

SEQ ID NO: 14 - DNA, 846 bp, Arabidopsis thaliana
atgtcaacctccgccgcttccttgtgttgttcatcaacccaggtcaatgggtttggtcttaggcctgaaaggtcgcttctttaccaacccacttccttttctttctccagaaggagaactcatggaattgtcaaggcctcatctcgggttgataggttttcgaaaagtgatatcattgtttctccctctattctctcggctaatttcgccaaattaggcgagcaggtaaaagcagtggagttggcaggttgtgattggattcatgttgatgtcatggacggtcgttttgttcccaacattactatcggacctctcgtggttgatgctttgcgccctgtgacagatcttcctttggatgttcatctgatgatagtggaacccgagcagagagtaccggatttcatcaaagcaggtgcagatattgtcagtgtacattgtgaacagcaatccaccatccatttgcatcgtaccgtcaatcaaataaaaagcttaggggctaaagctggagttgttctaaaccctggaaccccattgagtgcaatagaatatgtcttggatatggtggatctggtcttgatcatgtcggtcaaccctggttttggtggacagagctttattgaaagccaagtaaagaaaatctcggacttgaggaaaatgtgtgcagagaagggagtaaacccatggattgaagttgatggtggtgtcactccagcgaatgcgtacaaggttattgaggctggagcaaatgctctagtggctggatcagctgtatttggagctaaggactacgcagaagctataaaaggaattaaggccagcaaacgaccagcagctgtagctgtgtaa

SEQ ID NO: 15 - DNA, 1365 bp, Saccharomyces cerevisiae
atgtcagagccagaatttcaacaagcttacgaagaagttgtctcctctttggaagactctactcttttcgaacaacacccggaatacagaaaggttttgccaattgtttctgttccagaaagaatcatacaattcagagtcacctgggaaaatgacaagggtgaacaagaagttgctcaaggttacagagtgcaatataactccgccaagggtccatacaagggtggtctacgtttccatccttccgtgaacttgtctatcttgaaattcttgggtttcgaacaaatcttcaagaactccttgaccggcctagacatgggtggtggtaaaggtggtctatgtgtggacttgaagggaagatctaataacgaaatcagaagaatctgttatgctttcatgagagaattgagcagacacattggtcaagacactgacgtgccagctggtgatatcggtgttggtggtcgtgaaattggttacctgttcggtgcttacagatcatacaagaactcctgggaaggtgtcttaaccggtaagggtttgaactggggtggttctttgatcagaccagaagccactggttacggtttagtttactatactcaagctatgatcgactatgccacaaacggtaaggaatctttcgaaggtaagcgcgtcaccatctctggtagtggtaacgttgctcaatacgctgccttgaaggttattgagctaggtggtactgtcgtttccctatctgactccaagggttgtatcatctctgaaactggtatcacctccgaacaagtcgctgatatttccagtgctaaggtcaacttcaagtccttggaacaaatcgtcaacgaatactctactttctccgaaaacaaagtgcaatacattgctggtgctcgtccatggacccacgtccaaaaggtcgacattgctttgccatgtgccacccaaaatgaagtcagcggtgaagaagccaaggccttggttgctcaaggtgtcaagtttattgccgaaggttccaacatgggttccactccagaagctattgccgtctttgaaactgctcgttccaccgccactggaccaagcgaagctgtttggtacggtccaccaaaggctgctaacttgggtggtgttgctgtttctggtttagaaatggcacaaaactctcaaagaatcacatggactagcgaaagagttgaccaagagttgaagagaattatgatcaactgtttcaatgaatgtatcgactatgccaagaagtacactaaggacggtaaggtcttgccatctttggtcaaaggtgctaatatcgcaagtttcatcaaggtctctgatgctatgtttgaccaaggtgatgtattttaa

SEQ ID NO: 16 - DNA, 1740 bp, Zea mays
atggcgtccctcttcggggctcgccgccggcgatcgccggagtacgacggcgaggacgatagatccggcggagggagggccaagcgccggcgcctgtcgccggaggaggcggcggcgtcgccggcggagccgggcgcggcgacggggactagccacggctggctctccggcttcgtctccggagcgaagagggccatttcttccgttttgctgtcctcttcgcccgaggagaccggctcgggggaggacggggaggtggaggaggaggacgacgacgtatacgaagagggcatcgacttgaatgaaaatgaagatattcatgatattcacggggaaatagttccttatagcgagtcaaaacttgctattgagcaaatggttatgaaggaaacattctcaagggatgaatgtgataggatggtagagctaataaaatcaagagttagagattctactcctgaaacccatgagtatggaaagcaagaagaaatcccaagtaggaatgcaggcattgcacatgacttcacaggaacatgccgctccttgagccgtgataggaatttcactgaatcggtcccattctctagtatgagaatgagacctggtcattcttctccaggctttccactccaagcatcacctcagctatgcactgcagcagttagggaagcaaaaaaatggttggaagagaaaaggcagggactgggcgtaaaacctgaagacaatggatcatgcacattaaatacagatatatttagttctcgtgatgactctgacaagggttctccagttgatttggcgaaatcatacatgcggtcattacctccttggcaatccccattcttaggccatcaaaagtttgacacatcaccctccaaatactctatctcgtcaacaaaggtaactacaaaggaggactacctttccagcttttggacaaaattggaggaatcacgaatagctcgcattggatcatctggagattctgctgttgcttctaaattatggaattatggttccaattccagattatttgagaatgacacttccatattctcattgggcaccgatgagaaagttggagatcctaccaaaactcataatggctctgagaaagttgcagcaacagaaccactcggtagatgctccttacttattacaccaactgaagatagaactgatggtattactgagcctgtggaccttgcaaagaataatgagaatgcaccccaagaataccaagctgcatctgaaattatccctgataaagttgcagagggtaatgatgtgtcttccacaggaattactaaggatactactggccatagtgcagatggtaaagctctcacttcagaaccgcatataggggaaacacatgtcaactcagcttcagaatccataccaaatgacgcagctcccccaacccagagcaaaatgaatgggtcgaccaagaaatcattagtcaacggtgttttggatcaaccaaatgccaactcagggctagaatcttcgggaaatgattatcctagctacactaactcgagcagtgctatgccacccgcgagtaccgagttaattgggtctgcagctgctgttatagatgttgattctgctgagaatggcccaggtacgaaaccagaacaaccggctaagggagcctcgagagcatcgaaatccaaggttgttccacgaggacagaagagggtgttgcgaagcgcaacaagaggaagagcgacgtag

SEQ ID NO: 17 - DNA, 426 bp, Eutrema halophilum
atggctgcttcagtgatgctttcatcggtgacattgaaaccggcgggtttcacggtggagaagatgtcggcgagaggattgccgtcgctcacaagagcttctccttcctccttcagaattgtcgccagcggcgtcaagaagatcaagaccgacaagccctttggagttaacggcagcatggacttgagggacggcgtcgacgcctccggcagaaagggcaagggatacggtgtttacaaattcgtcgacaagtacggtgctaacgtcgatggatacagtcctatttacaacgaggaggagtgggcaccgggtggtgacacgtacaagggaggagtcaccggattggcaatttgggcggtgacgctcgccggaattctcgccggaggagctcttcttgtgtacaacaccagtgctttggctcagtaa

SEQ ID NO: 18 - DNA, 789 bp, Sorghum bicolor
atggagctgggagacgccaccgccggccagggagcgcaaggggacgccgcctccggggcccttgtcaggaagaagaggatgaggaggaagagcactggccctgactccattgccgagacgatcaagcggtggaaggagcagaaccagaagctccaggatgagagcggttccaggaaggcgccggccaagggttccaagaaagggtgcatgacgggcaaaggagggcctgagaacgtcaactccatgtaccgcggtgtgaggcagcggacgtggggcaagtgggtggcggagatccgcgagcccaaccgtggccggaggctatggctgggctccttccctaatgccgtggaagctgcccatgcatacgatgaggcggcaaaggccatgtatggccccaaggcacgtgtcaacttctcggataactctgctgacgccaactctggctgcacgtcggcgctttcgttgctggcatctagtgtaccggttgccacgttgcaacggtctgatgagaaagtggagactgaggtggaatctgtggagactgaagtccatgaggtgaaaactgaagggaatgatgacttgggaagtgtccacgttgcctgcaagaccgtggacgtcattcaatctgagaagagtgtgttacacaaagcaggggaagtaagttatgattacttcaacgttgaagaggtggttgagatgataattatagaattgaatgctgataaaaaaattgaagcacatgaagaataccatgatggagatgacgggtttagcctttttgcatattag

SEQ ID NO: 19 - DNA, 909 bp, Arabidopsis thaliana
atgcaggagatagatcttagtgttcacactataaagtcccatggaggaagagtcgcttctaaacacaagcacgattggatcatactcgtcatcttgattgccatcgagataggcttgaacctcatctctcctttctaccgctacgtgggaaaagacatgatgactgacctcaagtaccctttcaaggacaacaccgtacctatctggtctgtccctgtgtacgctgtgcttcttcccatcatagtgttcgtctgcttctacctgaagaggacatgtgtgtacgatctgcaccacagcatcctcgggctgctcttcgccgtcttgataactggtgtcatcactgactccatcaaggtagccaccggacgccctcgtcctaacttctactggcgctgcttccccgacggcaaagagctgtatgatgcgttgggaggtgtggtatgccacggcaaggcagctgaggtcaaggaaggccacaagagcttcccgagcggacacacttcctggtcctttgcggggcttacattcctttccctttacctctctggcaaaatcaaggccttcaacaatgaaggacatgtggcgaaactctgcctcgtgatcttccctctgcttgccgcttgtcttgtggggatatctcgtgtggatgactactggcaccactggcaagatgtcttcgcaggagctctcattggcacccttgtagccgccttctgctaccgtcagttctaccccaacccttaccacgaagaaggatggggtccctacgcctatttcaaggcagctcaagaacgaggagtccctgtgacctcctcccaaaacggagatgccttgagggctatgtctctgcagatggattcaacatctctcgaaaacatggaatctggcacttccaccgctcccagatga

SEQ ID NO: 20 - DNA, 339 bp, Eutrema halophilum
atgatggttgcgatcgatgagagcgattcgagcttctacgctcttcaatgggtcattgaccatttctctagcctcttaatgaccactgaggcggctgtggcggaaggtgtcatgctcacggtggttcatgtgcagtctccgttccatcactttgctgcttttccggctggacccggcggcgccacagctgtctacgcatcttcgacgatgatagagtctgtgaaaaaaaagcacaacaggagacctctgcagcgcttctctcgcgtgcactccaaatgtgccgagccaaacagatacgtactgaaactctggtgcttgaaggcgaggccaaggacatga

SEQ ID NO: 21 - DNA, 1272 bp, Arabidopsis thaliana
atgcccgaaccaatcgtccgagcttttggtgtcttgaagaaatgtgctgccaaggttaacatggagtatggtcttgatccaatgattggggaagccataatggaagctgcacaagaagtagcagaaggaaagctcaatgatcatttccctcttgttgtatggcaaactggtagtgggacgcagagtaatatgaatgctaatgaggtcattgccaatagagcagctgagattcttggtcacaaacgtggtgaaaaaattgtgcacccaaatgaccatgtgaacagatcacaatcttctaatgacacttttccaactgtcatgcacattgcagctgcaaccgagattacttcgaggctaatccctagtttgaaaaatttgcatagctctttggaatctaagtccttcgagtttaaagatatagtgaaaatcggaagaactcatactcaagatgctacacctttgacattaggacaagaatttggtggctatgctactcaagttgagtatggacttaatagagtcgcatgtactctaccccgcatctatcagcttgcacaaggtggaactgctgttgggaccggattaaacactaagaaagggtttgatgtaaagatcgctgctgcagtagctgaagaaacaaacttgccattcgtcaccgcagaaaacaagtttgaagctctggctgcacacgatgcttgtgttgaaacaagtggatctcttaacacaatcgccacatcattgatgaagattgccaatgatatacgttttcttggaagtggtccaagatgtggtcttggtgaactttctctgcctgagaatgaaccaggaagcagtattatgcctggaaaggtaaatcctacacagtgtgaggccttgactatggtttgtgctcaagttatgggaaaccatgtagccgtgacaattggtgggtcgaatggtcattttgaattgaatgtattcaagccggttatcgcaagcgctctcttacattccattagactaatagcagatgcttcagcttcatttgagaaaaactgtgttagaggcattgaggccaacagagaaaggatctcaaagctattgcacgagtctcttatgcttgtgacatcattgaatcctaaaattggctatgacaatgctgcagcagtagccaaaagagctcacaaagaaggatgcacattaaagcacgcagctatgaagttaggtgttcttacttcggaagagtttgatactcttgttgttcccgagaagatgattggtccatctgattaa

SEQ ID NO: 22 - DNA, 687 bp, Zea mays
atggcgagggagcgacgggagataaagaggatagagagcgcggcggcgcggcaggtcacgttctccaagcgccgccgcggcctcttcaagaaggctgaggagctctccgtgctgtgcgatgccgacgtcgcgctcatcgtcttctcctccacgggaaagctctcccagttcgccagctccagtatgaatgagatcattgacaagtacagcacacattctaaaaacctggggaaagcagaacagccttcacttgacttgaacttagaacatagcaaatatgcaaatttgaatgagcaacttgtggaagcaagccttcgactcaggcagatgagaggtgaagaacttgagggattgagtgttgaagaactccagcaattggagaagaatctggaatctggtctgcatagggtgcttcaaacaaaggatcaacaattcttggaacagatcagcgacctcgaaaaaaagagtacacaactggcagaggagaacaggcaactgaggaatcaagtatcccacatacccccagttggcaagcaatcagttgctgatactgaaaatgttatcgctgaagatgggcaatcctctgaatcagtcatgactgcgttgcattctgggagttcacaggataatgatgatggttcggatgtctctctaaaattagggctgccttgtgttgcatggaagtga

SEQ ID NO: 23 - DNA, 573 bp, Glycine max
atgtcaactccagaacaaaaatatctgggaaatatcttacaaataccacattcaattgaacaagttttcattgcacaaaaaatggagttctacacaaggccaaataggagtgacatccacctctcagcagaggaagaagccaccatagaggcaaagaccagagactactttgatggggttgcaccacaacgccacacaaagcctcaacgaagtgagtattcagctcaatatgtggatgctttctccaatgcccatcactcttcttcttcttcttctataccagaattcatgcaattccaacgcctcgagaatgatccccaagagaagaaattggagtacaatggaagtcaagtaccggaagaatttgtggaaacagagtattaccaagatctcaacagcgtggacaaacaccaccatacgacgggaacaggatttatcaaagtagagaaaaatggaaatgactttcacatagaaccagataatgacactggttgccatcactcttgcaagtgcaatccagcaaccaatgattgggttccttctccttccaacgaggtaccataccatatatag

SEQ ID NO: 24 - DNA, 3129 bp, Arabidopsis thaliana
atgatttcttattttcttaaccaagacttttcaaggaagaagcaaggaagaatggctgcttcaggaccaaaaagctcaggtcccagaggttttgggcgacgaacaacagtaggaagtgctcagaaaagaactcagaagaagaatggtgaaaaagatagtaatgccacttctacagcaacaaacgaggtttcagggattagtaagttgcccgcagctaaagtggatgtacagaagcaaagctctgttgttttgaatgagagaaatgtgttagataggtcggatattgaggatggaagtgatcgtttggacaagaaaacaaccgatgatgatgatttgttagaacaaaagttaaaacttgaaagagagaatcttcgtaggaaggaaatagaaacgcttgcagcggaaaatttggcgagaggtgatagaatgtttgtgtatcccgttattgtgaaacctgatgaagacatagaagtgtttctcaacaggaatctgtcgactctgaataacgaacccgatgttttgatcatgggggcgtttaacgaatggagatggaagtctttcacaaggagattggaaaagacctggatccatgaagattggttgtcatgtctccttcatatccccaaagaagcgtataagatggacttcgtgtttttcaatgggcaaagtgtatatgacaacaatgactcaaaagatttttgtgtagagataaaaggtgggatggataaagttgactttgagaattttcttctagaagagaaactgcgagagcaagagaagttagccaaggaagaagctgagagggagaggcaaaaagaagagaagagaagaatcgaagctcaaaaggctgcaattgaagctgatagagcacaagcaaaggcggagactcagaagagacgtgaattgcttcaaccggctattaagaaagctgtagtctcggctgagaatgtttggtacattgagccgagtgatttcaaggctgaagatacagtgaagctatattacaataaaaggtcaggtcctctgactaattccaaagaactgtggttacatggagggtttaataattgggttgatggattatctatcgttgtaaagcttgttaatgctgagttaaaggatgttgatccaaagagcggaaattggtggttcgctgaagttgtagtgcctggcggtgcactagtcattgactgggtctttgctgatggaccacctaaaggagcgtttctgtatgacaataatggttaccaagacttccacgcacttgttcctcaaaaacttcctgaagaactttactggttagaggaagaaaatatgatttttagaaaacttcaggaggataggcggttaaaagaggaagttatgcgtgccaagatggaaaaaacagctcgcttgaaagctgaaactaaggaaagaacactgaaaaagtttctgctatcccagaaagacgtggtttacaccgagcctctagagattcaagcaggaaaccctgtgactgtattgtacaatcctgcaaacacggttttgaatggaaaacctgaagtttggtttagaggctcttttaatcgttggactcaccgcttgggccctttgccacctcagaaaatggaagcaacagatgatgaaagctcacatgtgaagactacggctaaggtcccattggatgcttacatgatggactttgtgttctctgagaaagaggatggcggaatatttgataacaaaaatggtctggattaccatttaccagtcgtgggaggtatttcaaaggaaccaccattgcacattgttcatattgctgttgaaatggcacccatcgcaaaggttggtggcctaggtgatgttgtcactagtctatctcgcgctgttcaagaattaaaccataatgtggatatagtttttccaaagtatgattgcataaagcacaattttgtgaaggacttgcaatttaacagaagctatcactggggaggaactgaaataaaagtttggcatggaaaagtagaaggcctttcggtttacttcttagatccacaaaatggattgtttcagcgaggatgtgtttacggttgtgcagatgatgcaggaagattcggtttcttctgtcatgcggctcttgaatttcttctccaaggaggtttccatccagacattcttcactgtcatgactggtctagtgctccggtttcatggttattcaaggatcattacacacagtacggtttaattaaaacccgtattgtcttcacaattcataatttggaatttggagcgaatgccattggtaaagcaatgacatttgcagacaaagccacaacggtttcaccaacttatgctaaggaagttgctggaaactctgtaatctctgcacatttatacaaatttcacggaattataaacgggattgacccagatatatgggatccatataacgataactttattcccgtaccttatacttcagagaacgttgtagaaggcaaaagagcagccaaggaagaattgcaaaacaggcttggactaaagagtgccgattttccagtagtaggaattattacgcgcttaacacaccagaagggaatacatttgatcaagcacgctatttggcgtaccttggaacggaatggacaggttgtcttattaggttcagctccagatcctcggatccaaaatgattttgtaaacttggcaaaccaattacattcttctcatggtgaccgggctcggcttgttctaacctacgatgaacctctttcccatttgatttatgctggggctgactttattcttgtaccgtcgatatttgagccatgtggactgacacagctcatagccatgagatacggcgctgttcctgttgttagaaaaactggaggactctttgatacggtttttgatgttgaccacgataaagaaagggcacaagctcaagttctagaacctaatggtttcagcttcgacggagctgatgctcctggtgttgattatgctctcaatagggcgatatcggcgtggtacgatggtagagagtggtttaactcgctgtgcaagacggtgatggagcaagactggtcatggaaccgtcctgcacttgagtatcttgagctctatcactctgcacgcaagtaa

SEQ ID NO: 25 - DNA, 1023 bp, Oryza sativa
atgggcggcgtggcggcgggcaccaggtggatccaccacgtccggcggctcagcgccgccaaggtgtcggcggacgccctggagcgcggccagagccgggtcatcgacgcctccctcaccctcatccgcgagcgcgccaagctcaaggcagagttgctgcgcgctcttggtggtgtgaaagcttcagcatgcctcttaggtgttcctcttggtcacaactcatcgttcttacagggacctgcatttgctcctccccggataagggaagccatttggtgtggaagtaccaactctagcacagaagaaggcaaagaactcaatgatcctcgagtgctaacagatgttggtgatgtccccatacaagagattcgtgactgtggtgttgaagatgacagattgatgaatgttgtaagcgagtctgtcaaaacagtgatggaggaagatcctcttcggccattggtcctgggaggcgatcactcaatatcttatccagttgttagggctgtgtctgaaaagcttggtggacctgttgacattcttcaccttgacgcacatccagatatctacgatgcttttgaaggaaacatctattcgcatgcttcttcatttgcaagaataatggaaggaggttatgctaggaggcttctacaggttggaatcagatcaattaccaaagaagggcgtgagcaggggaagagatttggtgtggaacagtatgagatgcgcactttttcaaaagatagggagaagcttgaaagtctgaaacttggggaaggtgtgaagggagtgtacatctcagttgacgtggactgcctcgatcccgctttcgcgccaggtgtctctcacattgagccaggaggcctctccttccgcgacgtgctcaacatcctccataacctgcaaggagatgttgtcgccggagatgtggtggagttcaacccgcagcgtgacacggtggacgggatgacggctatggttgcagccaagctggtccgggagctcacagccaagatctccaagtga

SEQ ID NO: 26 - DNA, 726 bp, Medicago truncatula
atggaatatagccaatatagtagttattcagcagaagcaggagaagaagaaacatacacaactagtagcatatcttccatgagaaagaagaaaaacaagaatacaaagaggtttactgatgaacaaatcaaatcattggaaactatgtttgaaactgagacaagacttgaaccaagaaagaagttgcagttagctagagagcttggattgcagccaagacaagttgctatatggtttcaaaacaaaagagctagatggaaatcaaagcaacttgaaagagaatacaacaaacttcaaaatagttacaataatttggcttcaaagtttgaatctatgaagaaggaaagacaaacattactaatacagttgcagaagctgaatgatctaatacaaaagccaatagagcaaagtcagagtagttcacaagttaaagaagcaaagagcatggaaagtgcatcagaaaatggaggaagaaacaaatgtgaggctgaggtgaaaccaagtccttcaatggaaagatcagaacatgtacttgatgttctatcagatgatgacacaagcataaaggttgaatactttggtttagaagatgaaactggtcttatgaattttgctgaacatgctgatggttctttaacatcaccagaagattggagtgcttttgaatcaaatgatttattaggccaatcaagttgtgattatcaatggtgggacttttggtcttga

SEQ ID NO: 27 - DNA, 558 bp, Zea mays
atggcgaggatactcgtcgaagcgcccgcaggctcgggctcgccggaggactccatcaactcggacatgatcctcatcctcgccggcctgctctgcgcgctggtctgcgtcctcggcctgggcctcgtcgcccgctgcgcgtgctcgtggcgctgggccaccgagtccggccgggcgcagccgggcgccgccaaagccgcgaacaggggcgtcaagaaggaggtgctgcgctcgctcccgaccgtcacgtacgtctccgacagcggcaaggcggagggaggggccgacgagtgcgccatctgcctcgccgagttcgagggaggccaggccgtgcgcgtgctgccgcagtgcggccacgcgttccacgccgcctgcgtcgacacgtggctgcgcgcgcactcctcctgcccgtcctgccgccgggtgctggccgtcgacctgccccccgccgagcggtgccgccgctgcggcgcgcgccccggtgccggtgccggcatcagcgcgctctggaaggcgcccacgcgctgcagcgccgaggggccgacgttcttggcgtag

SEQ ID NO: 28 - DNA, 1791 bp, Zea mays
atggatgaggtccctgccaccgccgccgtcctcgacttccgacccggctcttctgtaccacgcgtctccgccgtcccgcgccgtgccgtgcagtgccccccggacaccggcggtgcagaggcggcgacgggaggtcgtccaggtatcgggaacactgccgccgtttccgcgaagttgacggggtcaagttcggccggtcccgatatccagtctgtggattgcgatacatcgggaggccttgccggcggtgacgctggagacgtgggggttttgtgcttagagaacgctgccgagaccgaatcggtggaaccaggtgtttcggacgtgaggttgggagctccagtggaggaacgccatggcaggacgctggatagtacgggattgggctctggtaaagctggtgagaccaatgagatttcactggttgaggtgtcccaatcaggtgccacttcaagcctagatgccactgcgtcgattggtggtggctactccctcgtggaggggtctctgccagaggcgagtggtgcccgcagatgtaaacccgaggtccatgaagtaccaacagggacccctgcaactgtgggattcccgattgaggacggtggctatggatttggaattcagcctaatgacgatgtggacggtagaaacgaccctgccggaggggaatgggaaccgcctactgatggcaatgatgccgaggacgtcacagatatgggtggaatcttgtgtgacgaaagagtggagaggatggagacgaattcggtagaacgtgaggcctcgaatggttccaccgtttcttctgaggaaggagtggacaggatggggacgagcttggatgactctgaggcttccgatggctcaaccacacaagattctgatacagacgtcgagaccgagtcaagtgtttctagtatagaggagcaagaagcaggatacggagcccacatccctcaaccggatccagcagtctgcaaggtggccaaagaaaacaacacggcaggagtgaagatctctgataggatgacttcagtgtccgaattaacacttgtgctggcttcaggtgcatcaatgttaccgcatccttcgaaggtacggacaggtggtgaagatgcatattttatagcttgtgatggctggtttggtgtagcagatggagttggtcagtggtcatttgaagggatcaatgccggactttatgccagagaactaatggatggctgcaaaaagatcgtggaagagacccaaggagctccagggatgagaaccgaggaagttcttgccaaggctgcagatgaagcacggtcccctggttcttccactgttttagttgctcactttgatggaaaggttcttcacgcatcaaacatcggagattctggattcctcgtgattaggaatggagaggtccataagaaatcaaacccaatgacttacggtttcaatttcccgttgcagattgagaagggtgacgaccctctaaaacttgtacagaaatacgccatctgtctacaagaaggcgacgtcgttgtaacagcgtcagacggtcttttcgacaacgtctacgaggaagaagtggcaggcatcgtctcgaaatcattagaagctgatctgaagcccacggaaatcgccgatttactggttgcccgagcgaaggaggtggggcggtgtgggtttggaaggagcccgttctcggactccgctctcgcggctgggtacctgggctactccggcggcaagctggatgatgtaacggttgttgtatcgattgttcggaaatctgaagtttaa

SEQ ID NO: 29 - DNA, 3432 bp, Glycine max
atggcatcaaaattgtttagggaaagccgatcatctatatcatcatcgtctgatgcacccgatggccagaagcctcctttacctccaagtgtacaatttggccggagaacttcctcgggtcgctatgtcagttactctagagatgatcttgacagtgagctagggagtactgacttcatgaattatacagtgcatataccacctacccctgataaccaacctatggatccatcaatctcacagaaagttgaggaacaatatgtgtcaaattcacttttcacaggtggattcaacagtgtcactcgagcccatctcatggataaggtgattgaatctgaagcaaatcatccacagatggctggtgcaaaaggatcttcatgtgcaattcctggttgtgattctaaggtgatgagcgatgaacgtggtgctgatattcttccatgtgagtgtgattttaagatatgcagagattgttatatagatgcagtaaaaacaggaggtgggatatgcccaggatgcaaggagccatataagaacacagaactagatgaagtggctgtagataatgggcgtccccttccacttcctccaccaagtggaatgtctaaaatggagaggagattgtccatgatgaagtcaacgaagtcagcactggtgaggagccaaactggagattttgatcataataggtggctctttgaaacaaagggaacctatggctatggcaatgctatatggccaaaggaaggtggttttggaaatgaaaaagaggatgattttgttcagccaactgaattgatgaacagaccctggagaccacttactcggaaactgaagatacctgctgccgttttgagcccatatcgtcttatcattttcattcgtttggttgtcttggcactgttcttggcgtggaggatcaaacaccaaaatactgatgcagtctggctatggggcatgtctgttgtttgtgagatatggtttgccttttcctggctgctggatcaactgcccaaactatgcccagtgaatcgttccactgatcttaatgttctgaaagagaaatttgaaacaccaacccctaacaatcctactggaaaatctgatcttccaggcatagatatctttgtttctactgctgatcctgagaaagaacctcctcttgtcactgcaaacactatcttgtctattttagctgctgattaccctgttgagaagctttcttgttatgtttctgatgatggaggagcacttctaacttttgaggcaatggctgaagctgccagctttgctaatgtgtgggttcccttctgtcgtaaacatgatatagagcccaggaatcctgaatcatacttcaacttaaaaagagatccttacaaaaacaaagtgaagcctgattttgtcaaggatcgtagacgggtaaagcgtgagtatgatgaattcaaggttaggatcaatagtctgcctgactctatccgtcgccggtctgatgcctatcatgcaagagaggaaatcaaggccatgaaagttcagagacaaaacagggaagatgaacctttagaagctgtaaagattccaaaagcaacatggatggctgatggaactcattggccaggaacttggttgagtcctacatctgagcattctaagggtgaccatgccggcataattcaggtgatgctgaaacctccgagcgatgaacctcttttaggaagttctgatgatacaaggctcattgacctgactgatattgatatccgtcttccccttcttgtttatgtttctcgagagaagcgcccaggctatgatcacaacaaaaaagccggggccatgaatgcattggttcgagcctcagctataatgtctaatggcccttttatactcaatcttgactgtgatcactatatctacaactcaaaggcaatgagggaaggcatgtgctttatgatggatcgtggaggtgaccgcctttgttatgtccagttcccccagaggtttgaaggaattgatccctctgatagatatgctaaccataatactgttttctttgatgtcaatatgcgagcccttgatggactccaaggaccagtctatgtgggaactgggtgccttttcagacgggttgcactttatggttttgacccaccacgttctaaagagcaccacacaggttgctgtaattgttgctttggtcgtcagaagaagcatgcatcactggcaagcaccccagaagagaaccgttcactgaggatgggtgattctgatgatgaggaaatgaatctatcgttgtttcctaagaagtttggaaactctactttcctcattgactcaattccagtggcagagttccaaggtcgtccgctagctgatcaccctgctgtgaaaaatggacgtccacctggtgctctcaccataccccgcgatcttcttgatgcatcaactgtcgcagaagccatcagtgtcatctcctgttggtacgaggacaagactgagtggggaaatcgtgttggatggatctatggatctgtgacagaggatgtggtcactggatataggatgcacaataggggatggaaatcagtttactgcgtgaccaagcgtgatgccttccgtggaactgctcccatcaatctcactgacaggctgcatcaggtccttagatgggctactggctcagttgaaatattcttctctcgaaacaatgcgctgctggctagcccaagaatgaaaattcttcaaagaattgcatacctaaatgttggaatctaccccttcacgtccatattcctaattgtctactgcttccttcctgcactatctctcttctctggccagttcattgtccaaaccctcaatgtcacttttctttcttacctcttgggcatcactgtgactctgtgcatgcttgctgtgcttgaaattaaatggtctggcattgagctggaagaatggtggagaaatgagcagttttggctgattggagggaccagtgcccatctagctgcggtgcttcaaggtttgctcaaagtcatagcagggattgaaatatcattcaccttgacttcaaaatctggtggtgatgatgtagatgatgagtttgctgatctctatattgtgaaatggacatcccttatgataccacctatcacaattatgatggttaacttaatagctatagcagttggagttagcagaaccatatacagtgtcataccacagtggagccgtctacttggtggtgttttcttcagtttttgggtcttggctcatctctatccctttgcaaaaggtttgatgggaagaagagggaggacacctaccatagtttttgtgtggtcaggcctcatagcaatcacaatttctctcctctgggtggctatcaacccccctgctggtactgaccaaattgggggttcattccagttcccttga

SEQ ID NO: 30 - DNA, 483 bp, Hordeum vulgare
atggcgaactaccggctcggcggcggtggcaacgggcactacgagatggcggcagcagcgtggagggagccggagagcccgcagctgagcctcatgagtgggtgcagctctctcttctccatctccggcctgcgggacgacgataccgacctccacctcctcgccggagcgcgctctctgccgtccacgccggtctcatttggcgggttcgccggcggtgacgaagtcgacatggagctgccgcagggcggcagtggcggcgacgaccggaggacggtccgcatgatgcggaatagggagtccgccctgcgctccagggccaggaagagggcatatgtggaggaactggagaaggaggttcgccgtctggtggatgacaacctcaagctgaagaagcagtgcaaagagctgaagcgggaagtggcggcgctggtcctgcccaccaagagctctctccggcgaacctcgtccacgcagttctga

SEQ ID NO: 31 - DNA, 519 bp, Glycine max
atgtctaggctcatggaaccacttgttgtgggaagagtgataggagaagtggtcgacattttcagcccaagtgtaaaaatgaatgtgacatattccaccaagcaagttgccaatggtcatgagttaatgccttctactattatggccaagccacgcgttgagattggtggtgatgacatgaggactgcttataccttgatcatgactgacccagatgctccaagtcctagtgacccatgtctaagggaacatctccactggatggttacagatatccctggcaccacagatgtctcttttggaaaagagattgtaggctatgagagtccaaagccagtaataggaatccacaggtatgtgttcatcttgttcaagcagagaggaagacaaacagtgaggcctccatcttcaagagaccacttcaacacaaggaggttctcagaagagaatggccttggcctaccagttgctgcagtttacttcaatgctcaaagagagactgctgcaagaaggaggtga

SEQ ID NO: 32 - protein, 590 aa, Arabidopsis thaliana
MPKHYRPTGKKKEGNAARYMTRSQALKHLQVNLNLFRRLCIVKGIFPREPKKKIKGNHHTYYHVKDIAFLMHEPLLEKFREIKTYQKKVKKAKAKKNEELARLLLTRQPTYKLDRLIRERYPTFIDALRDLDDCLTMVHLFAVLPASDRENLEVKRVHNCRRLTHEWQAYISRSHALRKVFVSVKGIYYQAEIEGQKITWLTPHAIQQVFTNDVDFGVLLTFLEFYETLLAFINFKLYHSLNVKYPPILDSRLEALAADLYALSRYIDASSRGMAVEPKVDASFSSQSNDREESELRLAQLQHQLPSSEPGALMHLVADNNKEVEEDEETRVCKSLFKDLKFFLSREVPRESLQLVITAFGGMVSWEGEGAPFKEDDESITHHIIDKPSAGHLYLSRVYVQPQWIYDCVNARIILPTEKYLVGRIPPPHLSPFVDNEAEGYVPDYAETIKRLQAAARNEVLPLPGVGKEDLEDPQNLLYAGVMSRAEEAEAAKNKKKMAAQEKQYHEELKMEINGSKDVVAPVLAEGEGEESVPDAMQIAQEDADMPKVLMSRKKRKLYDAMKISQSRKRSGVEIIEQRKKRLNDTQPSS

SEQ ID NO: 33 - protein, 432 aa, Arabidopsis thaliana
MDPQAFIRLSVGSLALRIPKVLINSTSKSNEKKNFSSQCSCEIKLRGFPVQTTSIPLMPSLDAAPDHHSISTSFYLEESDLRALLTPGCFYSPHAHLEISVFTGKKSLNCGVGGKRQQIGMFKLEVGPEWGEGKPMILFNGWISIGKTKRDGAAELHLKVKLDPDPRYVFQFEDVTTLSPQIVQLRGSVKQPIFSCKFSRDRVSQVDPLNGYWSSSGDGTELESERRERKGWKVKIHDLSGSAVAAAFITTPFVPSTGCDWVAKSNPGAWLVVRPDPSRPNSWQPWGKLEAWRERGIRDSVCCRFHLLSNGLEVGDVLMSEILISAEKGGEFLIDTDKQMLTVAATPIPSPQSSGDFSGLGQCVSGGGFVMSSRVQGEGKSSKPVVQLAMRHVTCVEDAAIFMALAAAVDLSILACKPFRRTSRRRFRHYSW

SEQ ID NO: 34 - protein, 232 aa, Zea mays
MVRGKTQMKRIENPTSRQVTFSKRRNGLLKKAFELSVLCDAEVALVVFSPRGKLYEFASGSAQKTIERYRTYTKDNVSNKTVQQDIERVKADADGLSKRLEALEAYKRKLLGERLEDCSIEELHSLEVKLEKSLHCIRGRKTELLEEQVRKLKQKEMSLRKSNEDLREKCKKQPPVPMASAPPRAPAVDNVEDGHREPKDDGMDVETELYIGLPGRDYRSSKDKAAVAVRSG

SEQ ID NO: 35 - protein, 377 aa, Arabidopsis thaliana
MAVSFVTTSPEEEDKPKLGLGNIQTPLIFNPSMLNLQANIPNQFIWPDDEKPSINVLELDVPLIDLQNLLSDPSSTLDASRLISEACKKHGFFLVVNHGISEELISDAHEYTSRFFDMPLSEKQRVLRKSGESVGYASSFTGRFSTKLPWKETLSFRFCDDMSRSKSVQDYFCDALGHGFQPFGKVYQEYCEAMSSLSLKIMELLGLSLGVKRDYFREFFEENDSIMRLNYYPPCIKPDLTLGTGPHCDPTSLTILHQDHVNGLQVFVENQWRSIRPNPKAFVVNIGDTFMALSNDRYKSCLHRAVVNSESERKSLAFFLCPKKDRVVTPPRELLDSITSRRYPDFTWSMFLEFTQKHYRADMNTLQAFSDWLTKPI

SEQ ID NO: 36 - protein, 710 aa, Oryza sativa
MSASPSSMSGAGAGEAGVRTVVWFRRDLRVEDNPALAAAARAAGEVVPVYVWAPEEDGPYYPGRVSRWWLSQSLKHLDASLRRLGASRLVTRRSADAVVALIELVRSIGATHLFFNHLYDPLSLVRDHRVKALLTAEGIAVQSFNADLLYEPWEVVDDDGCPFTMFAPFWDRCLCMPDPAAPLLPPKRIAPGELPARRCPSDELVFEDESERGSNALLARAWSPGWQNADKALAAFLNGPLMDYSVNRKKADSASTSLLSPYLHFGELSVRKVFHQVRMKQLMWSNEGNHAGDESCVLFLRSIGLREYSRYLTFNHPCSLEKPLLAHLRFFPWVVDEVYFKVWRQGRTGYPLVDAGMRELWATGWLHDRIRVVVSSFFVKVLQLPWRWGMKYFWDTLLDADLESDALGWQYISGSLPDGRELDRIDNPQLEGYKFDPHGEYVRRWLPELARLPTEWIHHPWDAPESVLQAAGIELGSNYPLPIVELDAAKTRLQDALSEMWELEAASRAAMENGMEEGLGDSSDVPPIAFPPELQMEVDRAPAQPTVHGPTTAGRRREDQMVPSMTSSLVRAETELSADFDNSMDSRPEVPSQVLFQPRMEREETVDGGGGGGMVGRSNGGGHQGQHQQQQHNFQTTIHRARGVAPSTSEASSNWTGREGGVVPVWSPPAASGPSDHYAADEADITSRSYLDRHPQSHTLMNWSQLSQSL

SEQ ID NO: 37 - protein, 347 aa, Synechocystis sp. PCC 6803
MTVSEIHIPNSLLDRDCTTLSRHVLQQLNSFGADAQDLSAIMNRIALAGKLIARRLSRAGLMADVLGFTGETNVQGESVKKMDVFANDVFISVFKQSGLVCRLASEEMEKPYYIPENCPIGRYTLLYDPIDGSSNVDINLNVGSIFAIRQQEGDDLDGSASDLLANGDKQIAAGYILYGPSTILVYSLGSGVHSFILDPSLGEFILAQENIRIPNHGPIYSTNEGNFWQWDEALRDYTRYVHRHEGYTARYSGALVGDIHRILMQGGVFLYPGTEKNPDGKLRLLYETAPLAFLVEQAGGRASDGQKRLLDLIPSKLHQRTPAIIGSAEDVKLVESFISDHKQRQGN

SEQ ID NO: 38 - protein, 349 aa, Zea mays
MGGLIMDQAFVQAPEHRPKPIVTEATGIPLIDLSPLSASGGAVDALAAEVGAASRDWGFFVVVGHGVPAETVARATEAQRAFFALPAERKASVRRNEAEPLGYYESEHTKNVRDWKEVYDLAPREPPPPAAVADGELVFENKWPQDLPGFREALEEYAKAMEELAFKLLELIARSLKLRPDRLHGFFKDQTTFIRLNHYPPCPSPDLALGVGRHKDAGALTILYQDDVGGLDVRRRSDGEWVRVRPVPDSFIINVGDLIQVWSNDRYESAEHRVSVNSARERFSMPYFFNPATYTMVEPVEELVSEDDPPRYDAYNWGDFFSTRKNSNFKKLNVENIQIAHFKKSLVLA

SEQ ID NO: 39 - protein, 386 aa, Zea mays
MDASPTPPLPLRAPTPSIDLPAGKDRADAAANKAAAVFDLRREPKIPEPFLWPHEEARPTSAAELEVPVVDVGVLRNGDGAGLRRAAAQVAAACATHGFFQVCGHGVDAALGRAALDGASDFFRLPLAEKQRARRVPGTVSGYTSAHADRFASKLPWKETLSFGFHDGAAAPVVVDYFTGTLGQDFEPVGRVYQRYCEEMKELSLTIMELLELSLGVERGYYREFFEDSRSIMRCNYYPPCPVPERTLGTGPHCDPTALTILLQDDVGGLEVLVDGEWRPVRPVPGAMVINIGDTFMALSNGRYKSCLHRAVVNRRQERQSLAFFLCPREDRVVRPPASAAPRQYPDFTWADLMRFTQRHYRADTRTLDAFTRWLSHGPAAAAPCT

SEQ ID NO: 40 - protein, 155 aa, Arabidopsis thaliana
METLQCRHQHVFILLLVLFHSSLFVLASKIDVSDDARGIRIDGGQKRFLTNSPQHGKEHAACTNEEPDLGPLTRISCNEPEYVITKINFADYGNPTGTCGHFRRDNCGARATMRIVKKNCLGKEKCHLLVTDEMFGPSKCKGAPMLAVETTCTIA

SEQ ID NO: 41 - protein, 372 aa, Zea mays
MVLAAHDPPPLVFDAARLSGLSDIPQQFIWPADESPTPDAAEELAVPLIDLSGDAAEVVRQVRRACDLHGFFQVVGHGIDAALTAEAHRCMDAFFTLPLPDKQRAQRRQGDSCGYASSFTGRFASKLPWKETLSFRYTDNDDDGDKSKDVVASYFVDKLGEGFRHHGEVYGRYCSEMSRLSLELMEVLGESLGVGRRHFRRFFQGNDSIMRLNYYPPCQRPYDTLGTGPHCDPTSLTILHQDDVGGLQVFDAATLAWRSIRPRPGAFVVNIGDTFMALSNGRYRSCLHRAVVNSRVARRSLAFFLCPEMDKVVRPPKELVDDANPRAYPDFTWRTLLDFTMRHYRSDMRTLEAFSNWLSTSSNGGQHLLEKK

SEQ ID NO: 42 - protein, 446 aa, Zea mays
MQEQDVDDGGGRTTQQQEKSIDDWLPINSSRKAKWWYSAFHNVTAMVGAGVLGLPYAMSELGWGPGIAVMILSWIITLYTLWQMVEMHEMVPGKRFDRYHELGQHVFGDRLGLWIVVPQQLAVEVSLNIIYMVTGGQSLKKFHDVICDGGRCGGDLKLSYFIMIFASVHLVLSQLPNFNSISAVSLAAAVMSLSYSTIAWGASLHRGRREDVDYHLRATTTPGKVFGFLGGLGDVAFAYSGHNVVLEIQATIPSTPDKPSKKAMWKGAFVAYVVVAICYFPVTFVGYWAFGSGVDENILITLSKPKWLIALANMMVVVHVIGSYQVYAMPVFDMIETVLVKKMRFAPSLTLRLIARSVYVAFTMFLGITFPFFGGLLSFFGGLAFAPTTYFLPCIMWLKVYKPKRFGLSWFINWICIVIGVLLLILGPIGGLRQIILSATTYKFYQ

SEQ ID NO: 43 - protein, 134 aa, Arabidopsis thaliana
MDFQTIQVMPWEYVLASQSLNNYQENHVRWSQSPDSHTFSVDLPGLRKEEIKVEIEDSIYLIIRTEATPMSPPDQPLKTFKRKFRLPESIDMIGISAGYEDGVLTVIVPKRIMTRRLIDPSDVPESLQLLARAA

SEQ ID NO: 44 - protein, 241 aa, Zea mays
MNRAPSLSAAGAAAEEDEEQDEAGAAAAAASSSPNNSASSFPTDFSAHGQVAPGADRACSRASDEDDGGSARKKLRLSKEQSAFLEDSFKEHATLNPKQKLALAKQLNLRPRQVEVWFQNRRARTKLKQTEVDCEYLKRCCETLTEENRRLQKELSELRALKTVHPFYMHLPATTLSMCPSCERVASNSAPAPASSPSPATGIAAPAPEQRPSSFAALFSSPLNRPLAAQAQPQPQAPANS

SEQ ID NO: 45 - protein, 281 aa, Arabidopsis thaliana
MSTSAASLCCSSTQVNGFGLRPERSLLYQPTSFSFSRRRTHGIVKASSRVDRFSKSDIIVSPSILSANFAKLGEQVKAVELAGCDWIHVDVMDGRFVPNITIGPLVVDALRPVTDLPLDVHLMIVEPEQRVPDFIKAGADIVSVHCEQQSTIHLHRTVNQIKSLGAKAGVVLNPGTPLSAIEYVLDMVDLVLIMSVNPGFGGQSFIESQVKKISDLRKMCAEKGVNPWIEVDGGVTPANAYKVIEAGANALVAGSAVFGAKDYAEAIKGIKASKRPAAVAV

SEQ ID NO: 46 - protein, 454 aa, Saccharomyces cerevisiae
MSEPEFQQAYEEVVSSLEDSTLFEQHPEYRKVLPIVSVPERIIQFRVTWENDKGEQEVAQGYRVQYNSAKGPYKGGLRFHPSVNLSILKFLGFEQIFKNSLTGLDMGGGKGGLCVDLKGRSNNEIRRICYAFMRELSRHIGQDTDVPAGDIGVGGREIGYLFGAYRSYKNSWEGVLTGKGLNWGGSLIRPEATGYGLVYYTQAMIDYATNGKESFEGKRVTISGSGNVAQYAALKVIELGGTVVSLSDSKGCIISETGITSEQVADISSAKVNFKSLEQIVNEYSTFSENKVQYIAGARPWTHVQKVDIALPCATQNEVSGEEAKALVAQGVKFIAEGSNMGSTPEAIAVFETARSTATGPSEAVWYGPPKAANLGGVAVSGLEMAQNSQRITWTSERVDQELKRIMINCFNECIDYAKKYTKDGKVLPSLVKGANIASFIKVSDAMFDQGDVF

SEQ ID NO: 47 - protein, 579 aa, Zea mays
MASLFGARRRRSPEYDGEDDRSGGGRAKRRRLSPEEAAASPAEPGAATGTSHGWLSGFVSGAKRAISSVLLSSSPEETGSGEDGEVEEEDDDVYEEGIDLNENEDIHDIHGEIVPYSESKLAIEQMVMKETFSRDECDRMVELIKSRVRDSTPETHEYGKQEEIPSRNAGIAHDFTGTCRSLSRDRNFTESVPFSSMRMRPGHSSPGFPLQASPQLCTAAVREAKKWLEEKRQGLGVKPEDNGSCTLNTDIFSSRDDSDKGSPVDLAKSYMRSLPPWQSPFLGHQKFDTSPSKYSISSTKVTTKEDYLSSFWTKLEESRIARIGSSGDSAVASKLWNYGSNSRLFENDTSIFSLGTDEKVGDPTKTHNGSEKVAATEPLGRCSLLITPTEDRTDGITEPVDLAKNNENAPQEYQAASEIIPDKVAEGNDVSSTGITKDTTGHSADGKALTSEPHIGETHVNSASESIPNDAAPPTQSKMNGSTKKSLVNGVLDQPNANSGLESSGNDYPSYTNSSSAMPPASTELIGSAAAVIDVDSAENGPGTKPEQPAKGASRASKSKVVPRGQKRVLRSATRGRAT

SEQ ID NO: 48 - protein, 141 aa, Eutrema halophilum
MAASVMLSSVTLKPAGFTVEKMSARGLPSLTRASPSSFRIVASGVKKIKTDKPFGVNGSMDLRDGVDASGRKGKGYGVYKFVDKYGANVDGYSPIYNEEEWAPGGDTYKGGVTGLAIWAVTLAGILAGGALLVYNTSALAQ

SEQ ID NO: 49 - protein, 262 aa, Sorghum bicolor
MELGDATAGQGAQGDAASGALVRKKRMRRKSTGPDSIAETIKRWKEQNQKLQDESGSRKAPAKGSKKGCMTGKGGPENVNSMYRGVRQRTWGKWVAEIREPNRGRRLWLGSFPNAVEAAHAYDEAAKAMYGPKARVNFSDNSADANSGCTSALSLLASSVPVATLQRSDEKVETEVESVETEVHEVKTEGNDDLGSVHVACKTVDVIQSEKSVLHKAGEVSYDYFNVEEVVEMIIIELNADKKIEAHEEYHDGDDGFSLFAY

SEQ ID NO: 50 - protein, 302 aa, Arabidopsis thaliana
MQEIDLSVHTIKSHGGRVASKHKHDWIILVILIAIEIGLNLISPFYRYVGKDMMTDLKYPFKDNTVPIWSVPVYAVLLPIIVFVCFYLKRTCVYDLHHSILGLLFAVLITGVITDSIKVATGRPRPNFYWRCFPDGKELYDALGGVVCHGKAAEVKEGHKSFPSGHTSWSFAGLTFLSLYLSGKIKAFNNEGHVAKLCLVIFPLLAACLVGISRVDDYWHHWQDVFAGALIGTLVAAFCYRQFYPNPYHEEGWGPYAYFKAAQERGVPVTSSQNGDALRAMSLQMDSTSLENMESGTSTAPR

SEQ ID NO: 51 - protein, 112 aa, Eutrema halophilum
MMVAIDESDSSFYALQWVIDHFSSLLMTTEAAVAEGVMLTVVHVQSPFHHFAAFPAGPGGATAVYASSTMIESVKKKHNRRPLQRFSRVHSKCAEPNRYVLKLWCLKARPRT

SEQ ID NO: 52 - protein, 423 aa, Arabidopsis thaliana
MPEPIVRAFGVLKKCAAKVNMEYGLDPMIGEAIMEAAQEVAEGKLNDHFPLVVWQTGSGTQSNMNANEVIANRAAEILGHKRGEKIVHPNDHVNRSQSSNDTFPTVMHIAAATEITSRLIPSLKNLHSSLESKSFEFKDIVKIGRTHTQDATPLTLGQEFGGYATQVEYGLNRVACTLPRIYQLAQGGTAVGTGLNTKKGFDVKIAAAVAEETNLPFVTAENKFEALAAHDACVETSGSLNTIATSLMKIANDIRFLGSGPRCGLGELSLPENEPGSSIMPGKVNPTQCEALTMVCAQVMGNHVAVTIGGSNGHFELNVFKPVIASALLHSIRLIADASASFEKNCVRGIEANRERISKLLHESLMLVTSLNPKIGYDNAAAVAKRAHKEGCTLKHAAMKLGVLTSEEFDTLVVPEKMIGPSD

SEQ ID NO: 53 - protein, 228 aa, Zea mays
MARERREIKRIESAAARQVTFSKRRRGLFKKAEELSVLCDADVALIVFSSTGKLSQFASSSMNEIIDKYSTHSKNLGKAEQPSLDLNLEHSKYANLNEQLVEASLRLRQMRGEELEGLSVEELQQLEKNLESGLHRVLQTKDQQFLEQISDLEKKSTQLAEENRQLRNQVSHIPPVGKQSVADTENVIAEDGQSSESVMTALHSGSSQDNDDGSDVSLKLGLPCVAWK

SEQ ID NO: 54 - protein, 190 aa, Glycine max
MSTPEQKYLGNILQIPHSIEQVFIAQKMEFYTRPNRSDIHLSAEEEATIEAKTRDYFDGVAPQRHTKPQRSEYSAQYVDAFSNAHHSSSSSSIPEFMQFQRLENDPQEKKLEYNGSQVPEEFVETEYYQDLNSVDKHHHTTGTGFIKVEKNGNDFHIEPDNDTGCHHSCKCNPATNDWVPSPSNEVPYHI

SEQ ID NO: 55 - protein, 1042 aa, Arabidopsis thaliana
MISYFLNQDFSRKKQGRMAASGPKSSGPRGFGRRTTVGSAQKRTQKKNGEKDSNATSTATNEVSGISKLPAAKVDVQKQSSVVLNERNVLDRSDIEDGSDRLDKKTTDDDDLLEQKLKLERENLRRKEIETLAAENLARGDRMFVYPVIVKPDEDIEVFLNRNLSTLNNEPDVLIMGAFNEWRWKSFTRRLEKTWIHEDWLSCLLHIPKEAYKMDFVFFNGQSVYDNNDSKDFCVEIKGGMDKVDFENFLLEEKLREQEKLAKEEAERERQKEEKRRIEAQKAAIEADRAQAKAETQKRRELLQPAIKKAVVSAENVWYIEPSDFKAEDTVKLYYNKRSGPLTNSKELWLHGGFNNWVDGLSIVVKLVNAELKDVDPKSGNWWFAEVVVPGGALVIDWVFADGPPKGAFLYDNNGYQDFHALVPQKLPEELYWLEEENMIFRKLQEDRRLKEEVMRAKMEKTARLKAETKERTLKKFLLSQKDVVYTEPLEIQAGNPVTVLYNPANTVLNGKPEVWFRGSFNRWTHRLGPLPPQKMEATDDESSHVKTTAKVPLDAYMMDFVFSEKEDGGIFDNKNGLDYHLPVVGGISKEPPLHIVHIAVEMAPIAKVGGLGDVVTSLSRAVQELNHNVDIVFPKYDCIKHNFVKDLQFNRSYHWGGTEIKVWHGKVEGLSVYFLDPQNGLFQRGCVYGCADDAGRFGFFCHAALEFLLQGGFHPDILHCHDWSSAPVSWLFKDHYTQYGLIKTRIVFTIHNLEFGANAIGKAMTFADKATTVSPTYAKEVAGNSVISAHLYKFHGIINGIDPDIWDPYNDNFIPVPYTSENVVEGKRAAKEELQNRLGLKSADFPVVGIITRLTHQKGIHLIKHAIWRTLERNGQVVLLGSAPDPRIQNDFVNLANQLHSSHGDRARLVLTYDEPLSHLIYAGADFILVPSIFEPCGLTQLIAMRYGAVPVVRKTGGLFDTVFDVDHDKERAQAQVLEPNGFSFDGADAPGVDYALNRAISAWYDGREWFNSLCKTVMEQDWSWNRPALEYLELYHSARK

SEQ ID NO: 56 - protein, 340 aa, Oryza sativa
MGGVAAGTRWIHHVRRLSAAKVSADALERGQSRVIDASLTLIRERAKLKAELLRALGGVKASACLLGVPLGHNSSFLQGPAFAPPRIREAIWCGSTNSSTEEGKELNDPRVLTDVGDVPIQEIRDCGVEDDRLMNVVSESVKTVMEEDPLRPLVLGGDHSISYPVVRAVSEKLGGPVDILHLDAHPDIYDAFEGNIYSHASSFARIMEGGYARRLLQVGIRSITKEGREQGKRFGVEQYEMRTFSKDREKLESLKLGEGVKGVYISVDVDCLDPAFAPGVSHIEPGGLSFRDVLNILHNLQGDVVAGDVVEFNPQRDTVDGMTAMVAAKLVRELTAKISK

SEQ ID NO: 57 - protein, 241 aa, Medicago truncatula
MEYSQYSSYSAEAGEEETYTTSSISSMRKKKNKNTKRFTDEQIKSLETMFETETRLEPRKKLQLARELGLQPRQVAIWFQNKRARWKSKQLEREYNKLQNSYNNLASKFESMKKERQTLLIQLQKLNDLIQKPIEQSQSSSQVKEAKSMESASENGGRNKCEAEVKPSPSMERSEHVLDVLSDDDTSIKVEYFGLEDETGLMNFAEHADGSLTSPEDWSAFESNDLLGQSSCDYQWWDFWS

SEQ ID NO: 58 - protein, 185 aa, Zea mays
MARILVEAPAGSGSPEDSINSDMILILAGLLCALVCVLGLGLVARCACSWRWATESGRAQPGAAKAANRGVKKEVLRSLPTVTYVSDSGKAEGGADECAICLAEFEGGQAVRVLPQCGHAFHAACVDTWLRAHSSCPSCRRVLAVDLPPAERCRRCGARPGAGAGISALWKAPTRCSAEGPTFLA

SEQ ID NO: 59 - protein, 596 aa, Zea mays
MDEVPATAAVLDFRPGSSVPRVSAVPRRAVQCPPDTGGAEAATGGRPGIGNTAAVSAKLTGSSSAGPDIQSVDCDTSGGLAGGDAGDVGVLCLENAAETESVEPGVSDVRLGAPVEERHGRTLDSTGLGSGKAGETNEISLVEVSQSGATSSLDATASIGGGYSLVEGSLPEASGARRCKPEVHEVPTGTPATVGFPIEDGGYGFGIQPNDDVDGRNDPAGGEWEPPTDGNDAEDVTDMGGILCDERVERMETNSVEREASNGSTVSSEEGVDRMGTSLDDSEASDGSTTQDSDTDVETESSVSSIEEQEAGYGAHIPQPDPAVCKVAKENNTAGVKISDRMTSVSELTLVLASGASMLPHPSKVRTGGEDAYFIACDGWFGVADGVGQWSFEGINAGLYARELMDGCKKIVEETQGAPGMRTEEVLAKAADEARSPGSSTVLVAHFDGKVLHASNIGDSGFLVIRNGEVHKKSNPMTYGFNFPLQIEKGDDPLKLVQKYAICLQEGDVVVTASDGLFDNVYEEEVAGIVSKSLEADLKPTEIADLLVARAKEVGRCGFGRSPFSDSALAAGYLGYSGGKLDDVTVVVSIVRKSEV

SEQ ID NO: 60 - protein, 1143 aa, Glycine max
MASKLFRESRSSISSSSDAPDGQKPPLPPSVQFGRRTSSGRYVSYSRDDLDSELGSTDFMNYTVHIPPTPDNQPMDPSISQKVEEQYVSNSLFTGGFNSVTRAHLMDKVIESEANHPQMAGAKGSSCAIPGCDSKVMSDERGADILPCECDFKICRDCYIDAVKTGGGICPGCKEPYKNTELDEVAVDNGRPLPLPPPSGMSKMERRLSMMKSTKSALVRSQTGDFDHNRWLFETKGTYGYGNAIWPKEGGFGNEKEDDFVQPTELMNRPWRPLTRKLKIPAAVLSPYRLIIFIRLVVLALFLAWRIKHQNTDAVWLWGMSVVCEIWFAFSWLLDQLPKLCPVNRSTDLNVLKEKFETPTPNNPTGKSDLPGIDIFVSTADPEKEPPLVTANTILSILAADYPVEKLSCYVSDDGGALLTFEAMAEAASFANVWVPFCRKHDIEPRNPESYFNLKRDPYKNKVKPDFVKDRRRVKREYDEFKVRINSLPDSIRRRSDAYHAREEIKAMKVQRQNREDEPLEAVKIPKATWMADGTHWPGTWLSPTSEHSKGDHAGIIQVMLKPPSDEPLLGSSDDTRLIDLTDIDIRLPLLVYVSREKRPGYDHNKKAGAMNALVRASAIMSNGPFILNLDCDHYIYNSKAMREGMCFMMDRGGDRLCYVQFPQRFEGIDPSDRYANHNTVFFDVNMRALDGLQGPVYVGTGCLFRRVALYGFDPPRSKEHHTGCCNCCFGRQKKHASLASTPEENRSLRMGDSDDEEMNLSLFPKKFGNSTFLIDSIPVAEFQGRPLADHPAVKNGRPPGALTIPRDLLDASTVAEAISVISCWYEDKTEWGNRVGWIYGSVTEDVVTGYRMHNRGWKSVYCVTKRDAFRGTAPINLTDRLHQVLRWATGSVEIFFSRNNALLASPRMKILQRIAYLNVGIYPFTSIFLIVYCFLPALSLFSGQFIVQTLNVTFLSYLLGITVTLCMLAVLEIKWSGIELEEWWRNEQFWLIGGTSAHLAAVLQGLLKVIAGIEISFTLTSKSGGDDVDDEFADLYIVKWTSLMIPPITIMMVNLIAIAVGVSRTIYSVIPQWSRLLGGVFFSFWVLAHLYPFAKGLMGRRGRTPTIVFVWSGLIAITISLLWVAINPPAGTDQIGGSFQFP

SEQ ID NO: 61 - protein, 160 aa, Hordeum vulgare
MANYRLGGGGNGHYEMAAAAWREPESPQLSLMSGCSSLFSISGLRDDDTDLHLLAGARSLPSTPVSFGGFAGGDEVDMELPQGGSGGDDRRTVRMMRNRESALRSRARKRAYVEELEKEVRRLVDDNLKLKKQCKELKREVAALVLPTKSSLRRTSSTQF

SEQ ID NO: 62 - protein, 172 aa, Glycine max
MSRLMEPLVVGRVIGEVVDIFSPSVKMNVTYSTKQVANGHELMPSTIMAKPRVEIGGDDMRTAYTLIMTDPDAPSPSDPCLREHLHWMVTDIPGTTDVSFGKEIVGYESPKPVIGIHRYVFILFKQRGRQTVRPPSSRDHFNTRRFSEENGLGLPVAAVYFNAQRETAARRR

SEQ ID NO: 63 - DNA, 1356 bp, Zea mays
atggcgggcgcgggcgcgggcgggtggaagaagcgggtgggccgctacgaggtgggccggaccatcggccggggcaccttcgccaaggttaagttcgccgtcgacgccgacaccggcgcggctttcgccattaaggtgctcgacaaggagaccatcttcacccaccgcatgctccaccagatcaaaagggaaatatctatcatgaagatcgtaagacatcccaacatagttaggcttaatgaggtgttggccggcaggacaaagatatacatagtcttggaacttgtcactggaggtgaactgtttgatagaatagtccgccatgggaagctacgtgagaatgaagctaggaagtatttccagcagcttattgatgccattgattattgccacagcaaaggagtttatcatagagatttgaagcctcaaaacttgcttcttgactctcgtggaaacttgaaactttctgattttggacttagcacattgtctcaaaatggagtaggccttgtacacacgacatgtggaacaccaaattatgttgcacctgaggtgctaagtagcaatggatatgacggatctgcagcagacatttggtcgtgtggtgtcattctctatgttttaatggctggttaccttccctttgaggagaacgaccttccacatttgtatgaaaagataactgcagctcagtactcatgcccatattggttctctccaggagccaagtcattgatccagagaatacttgatccaaatccaagaactcgtatcactattgaagaaataagagaagacccatggtttaagaagaactacgtaactattagatgtggtgaagatgaaaatgtcagcctagacgatgttcaagctatttttgacaatattgaggacaagtatgtatcagacgaagtaacgcacaaggatggtggtcctcttatgatgaatgcctttgagatgattgcactatctcaaggtttggatctctcagcattgtttgataggcaacaggagtttgtcaagcgccaaacacgtttcgtctcaagaaagccagccaagactgtagtagctacaattgaggttgttgctgagtcaatgggtctcaaggtccactcccggaactacaaggtgaggcttgaaggtccagcgtcaaacagagcgagccaatttgctgttgttctagaggtctttgaagttgctccttctctgttcatggtcgatgttcgaaaggttgccggtgacactccggaataccacaggttttacgagaacctatgcagcaaactttgcagcataatctggaggccaaccgaagtttctgccaaatctacgccgctgaggacgaccacctgctag

SEQ ID NO: 64 - DNA, 1026 bp, Zea mays
  misc_feature (648)..(648): n is a, c, g, or t
atggggaagggagcgcaagggagcgatgcggcggcggcgggcggcgaggtggaggagaacatggcggcgtggctggttgccaagaacaccctcaagatcatgcccttcaagctcccgcccgtcggcccttatgatgtccgcgtgcgcatgaaggcagtggggatttgcggcagcgatgtgcactacctcagggagatgcgcatcgcgcacttcgtggtgaaggagccgatggtgatcgggcacgagtgcgcgggcgtggtcgaggaggtgggcgccggcgtgacgcacctgtccgtgggcgaccgcgtggcgctggagccgggcgtcagctgctggcgctgccgccactgcaagggcgggcggtacaacctgtgcgaggacatgaagttcttcgccaccccgccggtgcacggctcgctggcgaaccaggtggtgcacccggccgacctgtgcttcaagctccccgacggggtgagcctggaggagggcgccatgtgcgagccgctgagcgtgggcgtgcacgcgtgccgccgcgcgggggtggggcccgagacgggcgtgctcgtggtgggcgccggccccatcggcctggtgtcgctgctggcggcgcgggccttcggcgcgccgcgcgtggtggtcgtggatgtnggacgaccaccgcctggccgtggccaggtcgctgggccgcggacgcggccgtgccgggtgtcgccccgcgcggaggacctggcggacgaggtggagcgcatccgcgcggccatgggctcggacatcgacgtcagcctggactgcgccgggttcagcaagaccatgtcgacggcgctggaggcgacgcggcccggcgggaaggtgtgcctggtcgggatgggccacaacgagatgacgctgccgctgacggcggcggcggcgcgggaggtcgcagcggcaaggtggacgtcaagccgctcatcacccaccgcttcggcttctcgcagcgggacgtggaggaggccttcgaggtcagcgcccgcggccgcgatgccataa

SEQ ID NO: 65 - DNA, 738 bp, Glycine max
atggaatatagtcaatatactacttattcagcagaaggtgttgaggcagaaacttacacaagtagctgcaccaccccatcaagatcaaagaagagaaacaacaacaacacaagaaggttcagtgatgaacaaatcaaatcattggagaccatgtttgagtcagagacaaggcttgagcctagaaagaagttgcagcttgcaagagagcttggattgcagccaaggcaagttgctatatggtttcagaacaagagggctagatggaagtcaaagcaacttgagagagactatggcatactccaatccaattataacactttggcttcacgttttgaagctctgaagaaggaaaaacaaacattactaattcagttgcagaagctgaatcatctaatgcagaagccaatggagccaagtcagagatgcacacaagttgaagcagcaaacagcatggacagtgaatcagaaaatggaggcaccatgaaatgtaaagctgagggaaagccaagcccatcatcattggaaagatcagaacatgtacttggtgttctgtctgatgatgacactagcataaaggtggaagactttaacctagaagatgaacatggccttctgaattttgttgagcatgctgatggttccttgacttcaccagaagattggagtgcttttgaatccaatgatctatttggccaatcaaccactgatgattaccaatggtgggacttctggtcctga

SEQ ID NO: 66 - DNA, 1671 bp, Zea mays
atgatcagtgaagaaagagaaagcataagaccgggctggtggcaatataatgacgcggtgcctcatgttcatgccgccgctgttcctcgtgtcctccctcatctccaccgtggggctgccggtggagccgcccgcggagctcctgcagctcggaggcgacgtcagcggcgggcgcctcagcgtggacgcgtccgacatcgcggaggcgtcgcgggacttcgggggcctctcccgcgccgagcccatggcggtgttccagccgcgcgcggccggcgacgtggcgggcctggtccgcgccgcgttcgggtcggcgcgcgggttccgcgtgtcggcgcggggccacggccactccatcagcggccaggcgcaggcgcccggcggcgtggtcgtggacatgggccacggcggcgccgtggcgcgggcgcttcccgtgcactcgccggcgctgggcgggcactacgtggacgtctggggcggcgagctgtgggtggacgtgctcaactggacgctgtcgcacggcgggctcgcgccgcggtcgtggacggactacctgtacctgtccgtgggcggcaccctctccaacgccggcatcagcgggcaggcgttccaccacgggccccagatcagcaatgtctacgagctcgacgtcgtcacagggaagggagaggtggtgacctgctcggagacggagaacccggacctattcttcggcgtgctgggcgggctgggccagttcggcatcatcacaagggcgcgcatcgccctggaacgtgctccccaaagggttcggtggatccgggcgctctactccaacttcaccgagttcacggcggaccaggagcgcctcatctccctgggcagccgccggttcgactacgtggagggcttcgtcgtcgccgccgagggcctcatcaacaactggaggtcctccttcttctcgccgcagaacccggtgaagctcagctcgctcaagcaccactccggcgtcctctactgcctcgaggtcaccaagaactacgacgacgccaccgccgggtcggtcgagcaggatgtggatgcgctgttgggcgagctgaacttcatcccaggcacggtgttcacgacggacctgccgtacgtggacttcctggaccgcgtgcacaaggcggagctgaagctgcgcgccaaggggatgtgggaggtgccgcacccgtggctgaacctcttcgtgccggcgtcccgcatcgccgacttcgaccgcggcgtcttccgtggcgtgctggggggcggcaccgccggcgccggcggtcccatcctcatctaccccatgaacaagcacaggtgggacccgaggagctcggtggtgaccccggacgaggacgtgttctacctggtggcgttcctgcggtcggcgctgccgggcgcgccggagagcctggaggcgctggcgcggcagaaccggcgggtcctcgacttctgcgcggaggccggcatcggcgccaagcagtacctgcccaaccacaaggcgccgggcgagtgggcggagcacttcggcgccgcgcggtgggagcggttcgccaggctcaaggcccagttcgacccgcgggccatcctggccgccgggcagggcatcttccggccgccgggctcgccgccgctcgtcgccgactcgtgatcggtactactgactga

SEQ ID NO: 67 - DNA, 1578 bp, Zea mays
atgatgctcgcgtacatggaccgcgcgacggcggccgccgagccagaggacgccggccgcgagcccgccaccacggcgggcgggtgcgcggcggcggcggcgacggatttcggcgggctggcgagcgccatgcccgcggccgtggtccgcccggcgagcgcggacgacgtggccagcgccatccgcgcggcggcgctgacgccgcacctcaccgtggccgcccgcgggaacgggcactcggtggccggccaggccatggccgagggcgggctggtcctcgacatgcgctcgctcgcggcgccgtcccggcgcgcgcagatgcagctcgtcgtgcagtgccccgacggcggcggcggccgccgctgcttcgccgacgtccccggcggcgcgctctgggaggaggtgctccactgggccgtcgacaaccacgggctcgccccggcgtcctggacggactacctccgcctcaccgtgggcggcacgctctccaatggcggcgtcagcggccagtccttccgctacgggccccaggtgtccaacgtggccgagctcgaggtggtcaccggcgacggcgagcgccgcgtctgctcgccctcctcccacccggacctcttcttcgccgtgctcggcgggctcggccagttcggcgtcatcacgcgcgcccgcatcccgctccacagggcgccccaggcggtgcggtggacgcgcgtggtgtacgcgagcatcgcggactacacggcggacgcggagtggctggtgacgcggccccccgacgcggcgttcgactacgtggagggcttcgcgttcgtgaacagcgacgaccccgtgaacggctggccgtccgtgcccatccccggcggcgcccgcttcgacccgtccctcctccccgccggcgccggccccgtcctctactgcctggaggtggccctgtaccagtacgcgcaccggcccgacgacgtcgacgacgacgatgaggaggaccaggcggcggtgaccgtgagccggatgatggcgccgctcaagcacgtgcggggcctggagttcgcggcggacgtcgggtacgtggacttcctgtcccgcgtgaaccgggtggaggaggaggcccggcgcaacggcagctgggacgcgccgcacccgtggctcaacctcttcgtctccgcgcgcgacatcgccgacttcgaccgcgccgtcatcaagggcatgctcgccgacggcatcgacgggcccatgctcgtctaccctatgctcaagagcaagtgggaccccaacacgtcggtggcgctgccggagggcgaggtcttctacctggtggcgctgctgcggttctgccggagcggcgggccggcggtggacgagctggtggcgcagaacggcgccatcctccgcgcctgccgcgccaacggctacgactacaaggcctacttcccgagctaccgcggcgaggccgactgggcgcgccacttcggcgccgccaggtggaggcgcttcgtggaccgcaaggcccggtacgacccgctggcgatcctcgcgccgggccagaagatcttccctcgggtcccggcgtccgtcgccgtgtag

SEQ ID NO: 68 - DNA, 834 bp, Glycine max
atggatccttttgttaaaaagagtgaccagatccaaagaaaaagacctggcaagagagacaggcacagcaagatcaacaccgcaagagggttgagggatcggagaatgagactttcccttgaagttgcaaagaggtttttcggccttcaagatatgctgaactttgacaaagcaagcaagaccgtggagtggttattgaaccaagcaaaagtagaaatcaaccgtttagtgaaagagaagaagaagaatgatcatcatcatcaaagttgtagcagtgctagttcggaatgtgaagaaggtgtgtctagtcttgatgaggttgtagtaagtcgagatcaagaacaacaacaacaacaacaacaagagaaggtggaaaaagttgtaaagagaagggtcaaaaactctagaaagatcagtgcatttgaccctcttgcaaaagagtgtagggaaagggcaagggaaagagcaagagagaggacaagagaaaagatgagaagccgtggagttctagctgaagaatcaaagcaatgtggagaggaaacaaatcaggatctgatccaattgggttcttcgaacccctttgaaaccggagatcaagaatctggtgccaagacaagtcacagtgttgatgtgcatccttcttccttggacgtgattgctactgaggctaaagaacaaagctaccgtgcagtaaaggagcataatgatgatgatgatgattctttggttgttttgagcaaatggagcccctccttgattttcaataactctggattctctcaagatcaccaatttgcagaatttcagtccttaggaaagccgtgggagacctaa

SEQ ID NO: 69 - DNA, 746 bp, Glycine max
atgggaaggggtagggttcagctgaaacggatcgagaacaaaactagccagcaagtgacgttttccaagcgtagatcgggacttctcaagaaagccaacgaaatctctgtgctatgtgatgctcaagttgctttgattatgttctctaccaaaggaaaactttttgagtattcctctgaacgcagcatggaagacgtcctggaacgttacgagagatatacacatacagcacttactggagctaataacaatgaatcacagggaaattggtctttcgaatatatcaagctcaccgccaaagttgaagtcttggacaggaacgtaaggaatttcttgggaaatgatctggatcccttgagtttgaaagagcttcagagtttggagcagcagcttgacacagctctgaagcgcatccgaacaagaaagaatcaagttatgaatgaatccatctcagacctgcataaaagggcaaggacattacaagagcaaaacagcaagctagcaaagatgaaggagaaagcgaaaacagtgactgaaggtccacatactggcccagaaactctaggcccaaattcatcgacccttaacttaacttctccacagctaccaccaccaccacaaagactggttccttctctaactctctgtgagacattccaaggaagagcattggtggaagaaacgggaaaggctcaaacagtccctagtggcaattctctcatcccaccatggatgcttcatatctg

SEQ ID NO: 70 - protein, 451 aa, Zea mays
MAGAGAGGWKKRVGRYEVGRTIGRGTFAKVKFAVDADTGAAFAIKVLDKETIFTHRMLHQIKREISIMKIVRHPNIVRLNEVLAGRTKIYIVLELVTGGELFDRIVRHGKLRENEARKYFQQLIDAIDYCHSKGVYHRDLKPQNLLLDSRGNLKLSDFGLSTLSQNGVGLVHTTCGTPNYVAPEVLSSNGYDGSAADIWSCGVILYVLMAGYLPFEENDLPHLYEKITAAQYSCPYWFSPGAKSLIQRILDPNPRTRITIEEIREDPWFKKNYVTIRCGEDENVSLDDVQAIFDNIEDKYVSDEVTHKDGGPLMMNAFEMIALSQGLDLSALFDRQQEFVKRQTRFVSRKPAKTVVATIEVVAESMGLKVHSRNYKVRLEGPASNRASQFAVVLEVFEVAPSLFMVDVRKVAGDTPEYHRFYENLCSKLCSIIWRPTEVSAKSTPLRTTTC

SEQ ID NO: 71 - protein, 341 aa, Zea mays
  misc_feature (216)..(216): Xaa can be any naturally occurring amino acid
MGKGAQGSDAAAAGGEVEENMAAWLVAKNTLKIMPFKLPPVGPYDVRVRMKAVGICGSDVHYLREMRIAHFVVKEPMVIGHECAGVVEEVGAGVTHLSVGDRVALEPGVSCWRCRHCKGGRYNLCEDMKFFATPPVHGSLANQVVHPADLCFKLPDGVSLEEGAMCEPLSVGVHACRRAGVGPETGVLVVGAGPIGLVSLLAARAFGAPRVVVVDXGRPPPGRGQVAGPRTRPCRVSPRAEDLADEVERIRAAMGSDIDVSLDCAGFSKTMSTALEATRPGGKVCLVGMGHNEMTLPLTAAAAREVAAARWTSSRSSPTASASRSGTWRRPSRSAPAAAMP

SEQ ID NO: 72 - protein, 245 aa, Glycine max
MEYSQYTTYSAEGVEAETYTSSCTTPSRSKKRNNNNTRRFSDEQIKSLETMFESETRLEPRKKLQLARELGLQPRQVAIWFQNKRARWKSKQLERDYGILQSNYNTLASRFEALKKEKQTLLIQLQKLNHLMQKPMEPSQRCTQVEAANSMDSESENGGTMKCKAEGKPSPSSLERSEHVLGVLSDDDTSIKVEDFNLEDEHGLLNFVEHADGSLTSPEDWSAFESNDLFGQSTTDDYQWWDFWS

SEQ ID NO: 73 - protein, 556 aa, Zea mays
MISEERESIRPGWWQYNDAVPHVHAAAVPRVLPHLHRGAAGGAARGAPAARRRRQRRAPQRGRVRHRGGVAGLRGPLPRRAHGGVPAARGRRRGGPGPRRVRVGARVPRVGAGPRPLHQRPGAGARRRGRGHGPRRRRGAGASRALAGAGRALRGRLGRRAVGGRAQLDAVARRARAAVVDGLPVPVRGRHPLQRRHQRAGVPPRAPDQQCLRARRRHREGRGGDLLGDGEPGPILRRAGRAGPVRHHHKGAHRPGTCSPKGSVDPGALLQLHRVHGGPGAPHLPGQPPVRLRGGLRRRRRGPHQQLEVLLLLAAEPGEAQLAQAPLRRPLLPRGHQELRRRHRRVGRAGCGCAVGRAELHPRHGVHDGPAVRGLPGPRAQGGAEAARQGDVGGAAPVAEPLRAGVPHRRLRPRRLPWRAGGRHRRRRRSHPHLPHEQAQVGPEELGGDPGRGRVLPGGVPAVGAAGRAGEPGGAGAAEPAGPRLLRGGRHRRQAVPAQPQGAGRVGGALRRRAVGAVRQAQGPVRPAGHPGRRAGHLPAAGLAAARRRLVIGTTD

SEQ ID NO: 74 - protein, 525 aa, Zea mays
MMLAYMDRATAAAEPEDAGREPATTAGGCAAAAATDFGGLASAMPAAVVRPASADDVASAIRAAALTPHLTVAARGNGHSVAGQAMAEGGLVLDMRSLAAPSRRAQMQLVVQCPDGGGGRRCFADVPGGALWEEVLHWAVDNHGLAPASWTDYLRLTVGGTLSNGGVSGQSFRYGPQVSNVAELEVVTGDGERRVCSPSSHPDLFFAVLGGLGQFGVITRARIPLHRAPQAVRWTRVVYASIADYTADAEWLVTRPPDAAFDYVEGFAFVNSDDPVNGWPSVPIPGGARFDPSLLPAGAGPVLYCLEVALYQYAHRPDDVDDDDEEDQAAVTVSRMMAPLKHVRGLEFAADVGYVDFLSRVNRVEEEARRNGSWDAPHPWLNLFVSARDIADFDRAVIKGMLADGIDGPMLVYPMLKSKWDPNTSVALPEGEVFYLVALLRFCRSGGPAVDELVAQNGAILRACRANGYDYKAYFPSYRGEADWARHFGAARWRRFVDRKARYDPLAILAPGQKIFPRVPASVAV

SEQ ID NO: 75 - protein, 277 aa, Glycine max
MDPFVKKSDQIQRKRPGKRDRHSKINTARGLRDRRMRLSLEVAKRFFGLQDMLNFDKASKTVEWLLNQAKVEINRLVKEKKKNDHHHQSCSSASSECEEGVSSLDEVVVSRDQEQQQQQQQEKVEKVVKRRVKNSRKISAFDPLAKECRERARERARERTREKMRSRGVLAEESKQCGEETNQDLIQLGSSNPFETGDQESGAKTSHSVDVHPSSLDVIATEAKEQSYRAVKEHNDDDDDSLVVLSKWSPSLIFNNSGFSQDHQFAEFQSLGKPWET

SEQ ID NO: 76 - protein, 248 aa, Glycine max
MGRGRVQLKRIENKTSQQVTFSKRRSGLLKKANEISVLCDAQVALIMFSTKGKLFEYSSERSMEDVLERYERYTHTALTGANNNESQGNWSFEYIKLTAKVEVLDRNVRNFLGNDLDPLSLKELQSLEQQLDTALKRIRTRKNQVMNESISDLHKRARTLQEQNSKLAKMKEKAKTVTEGPHTGPETLGPNSSTLNLTSPQLPPPPQRLVPSLTLCETFQGRALVEETGKAQTVPSGNSLIPPWMLHI

SEQ ID NO: 77 - DNA, 325 bp, Artificial Sequence
  suppression miRNA precursor sequence
ggcagagccgtgcccgtctcatcccctgcccgtgcaagcagctaggtaggacgatttgagcgtggtgttaggccgaaccgctgaaggaagattgctccactgttgactgcattatcaaacagttcacctccaaaaatgtattgcttatattcagcaatataatgttcttggaggtgccatgtttgataatatagtcgatagtggaagaacggtaacatatgtggtttgcagcaggtgagcaggatgggtgtggatgattgaatatctctgttcagtgttttcatcatctgactgaacactgaatcagcttgctgacgttagaggt

SEQ ID NO: 78 - DNA, 328 bp, Artificial Sequence
  suppression miRNA precursor sequence
ggcagagccgtgcccgtctcatcccctgcccgtgcaagcagctaggtaggacgatttgagcgtggtgttaggccgaaccgctgaaggaagattgctccactgttgactgcatttgaagggcatgatcttgaggaaatgtattgcttatattcagcaatataatgttccctcaagatacggcccttcaaatatagtcgatagtggaagaacggtaacatatgtggtttgcagcaggtgagcaggatgggtgtggatgattgaatatctctgttcagtgttttcatcatctgactgaacactgaatcagcttgctgacgttagaggttag

SEQ ID NO: 79 - DNA, 328 bp, Artificial Sequence
  suppression miRNA precursor sequence
ggcagagccgtgcccgtctcatcccctgcccgtgcaagcagctaggtaggacgatttgagcgtggtgttaggccgaaccgctgaaggaagattgctccactgttgactgcatttgacttccatctagccctcaaaatgtattgcttatattcagcaatataatgttctgagggctatcgggaagtcaaatatagtcgatagtggaagaacggtaacatatgtggtttgcagcaggtgagcaggatgggtgtggatgattgaatatctctgttcagtgttttcatcatctgactgaacactgaatcagcttgctgacgttagaggttag

SEQ ID NO: 80 - DNA, 328 bp, Artificial Sequence
  suppression miRNA precursor sequence
ggcagagccgtgcccgtctcatcccctgcccgtgcaagcagctaggtaggacgatttgagcgtggtgttaggccgaaccgctgaaggaagattgctccactgttgactgcattgtgatgatgccgaactggcaaaatgtattgcttatattcagcaatataatgttctgccagttcttaatcatcacaatatagtcgatagtggaagaacggtaacatatgtggtttgcagcaggtgagcaggatgggtgtggatgattgaatatctctgttcagtgttttcatcatctgactgaacactgaatcagcttgctgacgttagaggttag

SEQ ID NO: 81 - DNA, 328 bp, Artificial Sequence
  suppression miRNA precursor sequence
ggcagagccgtgcccgtctcatcccctgcccgtgcaagcagctaggtaggacgatttgagcgtggtgttaggccgaaccgctgaaggaagattgctccactgttgactgcatttgagcatagggtagacgagaaaatgtattgcttatattcagcaatataatgttctctcgtctaaaatatgctcaaatatagtcgatagtggaagaacggtaacatatgtggtttgcagcaggtgagcaggatgggtgtggatgattgaatatctctgttcagtgttttcatcatctgactgaacactgaatcagcttgctgacgttagaggttag

SEQ ID NO: 82 - DNA, 328 bp, Artificial Sequence
  suppression miRNA precursor sequence
ggcagagccgtgcccgtctcatcccctgcccgtgcaagcagctaggtaggacgatttgagcgtggtgttaggccgaaccgctgaaggaagattgctccactgttgactgcattagtaatcacgtccaaggaaaaaatgtattgcttatattcagcaatataatgttctttccttggcattgattactaatatagtcgatagtggaagaacggtaacatatgtggtttgcagcaggtgagcaggatgggtgtggatgattgaatatctctgttcagtgttttcatcatctgactgaacactgaatcagcttgctgacgttagaggttag

SEQ ID NO: 83 - DNA, 328 bp, Artificial Sequence
  suppression miRNA precursor sequence
ggcagagccgtgcccgtctcatcccctgcccgtgcaagcagctaggtaggacgatttgagcgtggtgttaggccgaaccgctgaaggaagattgctccactgttgactgcattttatgcaggtctgagacggcaaatgtattgcttatattcagcaatataatgttcgccgtctcatcactgcataaaatatagtcgatagtggaagaacggtaacatatgtggtttgcagcaggtgagcaggatgggtgtggatgattgaatatctctgttcagtgttttcatcatctgactgaacactgaatcagcttgctgacgttagaggttag

SEQ ID NO: 84 - DNA, 20 bp, Artificial Sequence
  suppresison target recognition sequence
tatcaaacagttcacctcca

SEQ ID NO: 85 - DNA, 21 bp, Artificial Sequence
  suppresison target recognition sequence
ttgaagggcatgatcttgagg

SEQ ID NO: 86 - DNA, 21 bp, Artificial Sequence
  suppresison target recognition sequence
tttgacttccatctagccctc

SEQ ID NO: 87 - DNA, 21 bp, Artificial Sequence
  suppresison target recognition sequence
ttgtgatgatgccgaactggc

SEQ ID NO: 88 - DNA, 20 bp, Artificial Sequence
  suppresison target recognition sequence
ttgagcatagggtagacgag

SEQ ID NO: 89 - DNA, 16 bp, Artificial Sequence
  suppresison target recognition sequence
aatcacgtccaaggaa

SEQ ID NO: 90 - DNA, 18 bp, Artificial Sequence
  suppresison target recognition sequence
ttttatgcaggtctgaga

SEQ ID NO: 91 - DNA, 408 bp, Oryza sativa
ggcagagccgtgcccgtctcatcccctgcccgtgcaagcagctaggtaggacgatttgagcgtggtgttaggccgaaccgctgaaggaagattgctccactgttgactgcattaggattcaatccttgctgctaaatgtattgcttatattcagcaatataatgttcagcagcaagaactggatcttaatatagtcgatagtggaagaacggtaacatatgtggtttgcagcaggtgagcaggatgggtgtggatgattgaatatctctgttcagtgttttcatcatctgactgaacactgaatcagcttgctgacgttagaggtttcagtttacctaatttatggtctgtacccatgaaaagtgggaaaaggctgaagaattcgatttctttctttctttcaatgtt

SEQ ID NO: 92 - DNA, 325 bp, Oryza sativa
ggcagagccgtgcccgtctcatcccctgcccgtgcaagcagctaggtaggacgatttgagcgtggtgttaggccgaaccgctgaaggaagattgctccactgttgactgcattaggattcaatccttgctgctaaatgtattgcttatattcagcaatataatgttcagcagcaagaactggatcttaatatagtcgatagtggaagaacggtaacatatgtggtttgcagcaggtgagcaggatgggtgtggatgattgaatatctctgttcagtgttttcatcatctgactgaacactgaatcagcttgctgacgttagaggt

SEQ ID NO: 93 - DNA, 280 bp, Oryza sativa
ggcagagccgtgcccgtctcatcccctgcccgtgcaagcagctaggtaggacgatttgagcgtggtgttaggccgaaccgctgaaggaagattgctccactgttgactgcattaggattcaatccttgctgctaaatgtattgcttatattcagcaatataatgttcagcagcaagaactggatcttaatatagtcgatagtggaagaacggtaacatatgtggtttgcagcaggtgagcaggatgggtgtggatgattgaatatctctgttcagtgttt

SEQ ID NO: 94 - DNA, 249 bp, Oryza sativa
ggcagagccgtgcccgtctcatcccctgcccgtgcaagcagctaggtaggacgatttgagcgtggtgttaggccgaaccgctgaaggaagattgctccactgttgactgcattaggattcaatccttgctgctaaatgtattgcttatattcagcaatataatgttcagcagcaagaactggatcttaatatagtcgatagtggaagaacggtaacatatgtggtttgcagcaggtgagcaggatgggt

SEQ ID NO: 95 - DNA, 1528 bp, Artificial Sequence
  Root Preferred Promoter Seqeunce
ggaagctaactagtcacggcgaatacatgacgacatcggcctacaacgcacaacttcttggcataaaagcttcaatttcaatgcccctatctggaagccctaggcgccgcgcaaatgtaaaacattcgcttcgcttggcttgttatccaaaatagagtatggacctccgacagattggcaacccgtgggtaatcgaaaatggctccatctgcccctttgtcgaaggaatcaggaaacggccctcacctcctggcggagtgtagatatgtgaaagaatctaggcgacacttgcagactggacaacatgtgaacaaataagaccaacgttatggcaacaagcctcgacgctactcaagtggtgggaggccaccgcatgttccaacgaagcgccaaagaaagccttgcagactctaatgctattagtcgcctaggatatttggaatgaaaggaaccgcagagtttttcagcaccaagagcttccggtggctagtctgatagccaaaattaaggaggatgccaaaacatgggtcttggcgggcgcgaaacaccttgataggtggcttaccttttaacatgttcgggccaaaggccttgagacggtaaagttttctatttgcgcttgcgcatgtacaattttattcctctattcaatgaaattggtggctcactggttcattaaaaaaaaaagaatctagcctgttcgggaagaagaggattttattcgtgagagagagagagagagagagagagagagggagagagaaggaggaggaggattttcaggcttcgcattgcccaacctctgcttctgttggcccaagaagaatcccaggcgcccatgggctggcagtttaccacggacctacctagcctaccttagctatctaagcgggccgacctagtagctacgtgcctagtgtagattaaagttggcgggccagcaggaagccacgctgcaatggcatcttcccctgtccttcgcgtacgtgaaaacaaacccaggtaagcttagaatcttcttgcccgttggactgggacacccaccaatcccaccatgccccgatattcctccggtctcggttcatgtgatgtcctctcttgtgtgatcacggagcaagcattcttaaacggcaaaagaaaatcaccaacttgctcacgcagtcacgctgcaccgcgcgaagcgacgcccgataggccaagatcgcgagataaaataacaaccaatgatcataaggaaacaagcccgcgatgtgtcgtgtgcagcaatcttggtcatttgcgggatcgagtgcttcacggctaaccaaatattcggccgatgatttaacacattatcagcgtagatgtacgtacgatttgttaattaatctacgagccttgctagggcaggtgttctgccagccaatccagatcgccctcgtatgcacgctcacatgatggcagggcagggttcacatgagctctaacggtcgattaattaatcccggggctcgactataaatacctccctaatcccatgatcaaaacc

SEQ ID NO: 96 - DNA, 997 bp, Artificial Sequence
  Seed Preferred Promoter Sequence
gctgcttccggtagcctgaagcagaaaaaaactgaaagaaacatgacagataattccctcggagaaacttggcatgtttcccgttggtcatgtaggacgacgataatgataaattggtaagcaaagaaaaaggctactaagctcgagcagtagaagctacctagctcgtcgtaacgaagaaacttctcgtccttcaggtagacccttgcttgtttgcagtactttagttagggttcggtctttaattcttttgctgggcagcagtaaacggagatgagaagcgcgagctgatcattgttgccattctgtgcaacgaagctaggggaccaatgctgactcgcacgagggcatagttgctgatggtcatagacgacgcgttcacttaaaataataaagaattataaattgttgtcataagtcgtgcagcctaatataggagagtgcggcattgctgtagctaattaagagagtattccggtcatgcttgagcttggagaatttttgagggtccgttcgcttggagagtcggagatttttgagggcccgttcgcttgcacaataataaacaaagatttgttctagctcatccaaatctatataaattaaagaagtaattcggttaggaatcaatccagagctctaattcttaaaaaccgaacagggcctgagttgtttgtctagacgacattatctgattaagttattttcatcttcaatttcaaatgtgatctagcggcataaaacttgttgtctgacagatatttgacttccacacgggccacagctcaattacaaacatacttcaaacatcaggcagaggcagagcactagcagcattcgctacgtggcggtgggcagcagtggccagcacattcgacaactgccacggatcccgtactacttcaaacacgtatcgcttccagaatccagagtcacacgtgtgcagctgcatgaacccagctcactcccttaagaacagctcgacgctcacctgtct

SEQ ID NO: 97 - DNA, 1456 bp, Artificial Sequence
  Endosperm Preferred Promoter Sequence
tgtttggactccagaaaatttacgggagttggtggagcaggtcattaagtactataaaaaatcatgtagctgaagctgcaagtatttagaagacatttagataagttattttatttatcatttagattaagaaaatttaaaactatttaaattgatattataaactacagctccacactggagctagatcctggagtcattacaaacacccccttaatgggaaaagagaagataatgtatatctaattattgtttctgtgtcacctatagctattagttcaaaacttcataatcactggtacaaataagctctagagaggcggttcggaacccatttttattgttgtttttcaaaaccactagtgttagggaccgccagtggaaactgaaacgccattggaaattgattttcactgatggtgagctaagaaaaccgccattggtaatcctttgcagaaaacataaactaggttttaaaaatagtaaacaaatatttttattaggagaggccccacatagtcgcaccatttttcgcgcattattcacgcgctacgcaaccaatggtaattgaacctcagagacttcactcttgtgtagcctcctttgccactccactaaacacttacttgtgtcttgattgcattttgttgcccacatattagaacaaacagagtgtaaattgattgtttgaggctgtaaacaaattcaaatgaaaaagtagtcaactactaaattgaataattgtttatgttctaccacttttattttggtacttttcccatcggaggcggtttgtaaaatttgcattttaagttttacaaatttcaatgaaattttgagagcccaaatgatttcaaataaaaaagttgtcaactacaatgttttataacttttaatttggtggttttttaaacaagctcatttgaaaaactaaaatgatcgattctacatgatttttaggtcgattttttaaggaatcgcctgtacaaatatttctactgacagtttttaagaaaccacctgtggaaatcatagatttgtactagcggtttttctcaagtaactgctagtagaaatatggtggttttcttaagaaaactgtttgtaggaatgcacgatttatataaatggatttgttaagaaaaccgctagtggaatgttctttcaactaacggttattgagtcgtgacagccaatttaatttccttgataactaaaagcggctgtaaaaattagaccatgatgtaggcacggagctgttttgtactgaatgcgcccactgttttgttggaaaagtgcatgtacttattattcattctgtttatttctagctggcattcagttcttacagccacagattatgcaaaacgcctatttctgccagcaaatttacaggaaaagtcatggacttttccgggttattttcctataagtacagccattcctttcacttaca

SEQ ID NO: 98 - DNA, 363 bp, Artificial Sequence
  Meristem Preferred Promoter Sequence
catacaaattatatatatatattttaaatatcaaatctttataagaatgatgatccactgtccactgctgcccacttcccacgcccaaaacaagttcacctccgtggcgcgtgttccgaaaagtcctcttgttgtgggcgggagaatggaggcgtaatatttcggcgtccccgaaatttgcttgcaccttattggccgagccacccctcccacggatcgtgccctgctggcaacattgcagccatcggtgcccctctagatccaaccatccactgtcctcgcacgcggatccacgggcccaccagcctcggcagccgagttgtttaaactttataaatacccgtcgccgcctgctactttccc

SEQ ID NO: 99 - DNA, 1160 bp, Artificial Sequence
  Leaf Bundle Sheath Preferred Promoter Sequence
atgtgctggtgccccataaggtaggcacctaggtctgtgtttgaagcatcgacagatttgtaaacatgttcctatgaacctatttctgattgataatttgtcaaaactcatcatttgtcttcatccttgcctgcttgcgttcacgtgacaaagtacgtgtatgtcttcggcctttgctgtgtatgtttcgcattgcttagatgtggtgaaagaacatcagaagatgcattgatggcgtgcttaaaccagtgatgtgctccaggtgttcctgcagtctgcagagatatttactcttgtagtcttgttgacagcacagttgtatgtgatttcttggatgtaatgtaaaccaaatgaaagataggaacagttcgtcctcttccgtatacgaaggtcactgtatcatttgtcgtggcacaagatgatctgcaggcaggactgcaacatggtttcttggactgtcctgaatgcccgttcttgttctttagttgagccagagcagcagcctggtgtcggtgcctgagacctgacgaagcacacggcaaacaaacaagtcgcagcagctagcaggggcgttgccatcgccacaagcccccaagagacccgccgaggaaaagaaaaaaaaactacggccgccgttgccaagccgagcgtgcgaaccgatccacggatgggagatcagagatcacccaccgcaggcgggcggcagtggctggcgaggtgcgtccacagaacctgctgcaggtccctgtccgtcccggcgaccccttttctaggcgagcaactccccatggcagagctgcacgcagcagggcccgtcgttggttgcagctttaaccctttttgttttaaccatacaatgcagagtcgcagaggtgaaacaggacggaaattacagaaaagatggtggtgtgccagcagccccagcatgaagaagatcaggacaaaagaaaagcttgtgattggtgacagcaacaggattggattggagccaagctaggcagtgagaggcaggcagcaagacgcgtcagccactgaaatccagagggcaacctcggcctcacaactcatatccccttgtgctgttgcgcgccgtggttagccaggtgtgctgcaggcctcctccttgtttatatatgggagatgctctcaccctctaaggt

SEQ ID NO: 100 - DNA, 1696 bp, Artificial Sequence
  Above Ground Preferred Promoter Sequence
caaatttattatgtgttttttttccgtggtcgagattgtgtattattctttagttattacaagacttttagctaaaatttgaaagaatttactttaagaaaatcttaacatctgagataatttcagcaatagattatatttttcattactctagcagtatttttgcagatcaatcgcaacatatatggttgttagaaaaaatgcactatatatatatatattattttttcaattaaaagtgcatgatatataatatatatatatatatatatgtgtgtgtgtatatggtcaaagaaattcttatacaaatatacacgaacacatatatttgacaaaatcaaagtattacactaaacaatgagttggtgcatggccaaaacaaatatgtagattaaaaattccagcctccaaaaaaaaatccaagtgttgtaaagcattatatatatatagtagatcccaaatttttgtacaattccacactgatcgaatttttaaagttgaatatctgacgtaggatttttttaatgtcttacctgaccatttactaataacattcatacgttttcatttgaaatatcctctataattatattgaatttggcacataataagaaacctaattggtgatttattttactagtaaatttctggtgatgggctttctactagaaagctctcggaaaatcttggaccaaatccatattccatgacttcgattgttaaccctattagttttcacaaacatactatcaatatcattgcaacggaaaaggtacaagtaaaacattcaatccgatagggaagtgatgtaggaggttgggaagacaggcccagaaagagatttatctgacttgttttgtgtatagttttcaatgttcataaaggaagatggagacttgagaagttttttttggactttgtttagctttgttgggcgtttttttttttgatcaataactttgttgggcttatgatttgtaatattttcgtggactctttagtttatttagacgtgctaactttgttgggcttatgacttgttgtaacatattgtaacagatgacttgatgtgcgactaatctttacacattaaacatagttctgttttttgaaagttcttattttcatttttatttgaatgttatatatttttctatatttataattctagtaaaaggcaaattttgcttttaaatgaaaaaaatatatattccacagtttcacctaatcttatgcatttagcagtacaaattcaaaaatttcccatttttattcatgaatcataccattatatattaactaaatccaaggtaaaaaaaaggtatgaaagctctatagtaagtaaaatataaattccccataaggaaagggccaagtccaccaggcaagtaaaatgagcaagcaccactccaccatcacacaatttcactcatagataacgataagattcatggaattatcttccacgtggcattattccagcggttcaagccgataagggtctcaacacctctccttaggcctttgtggccgttaccaagtaaaattaacctcacacatatccacactcaaaatccaacggtgtagatcctagtccacttgaatctcatgtatcctagaccctccgatcactccaaagcttgttctcattgttgttatcattatatatagatgaccaaagcactagaccaaacct

SEQ ID NO: 101 - DNA, 786 bp, Artificial Sequence
  Leaf Mesophyll Preferred Promoter Sequence
gacatggaggtggaaggcctgacgtagatagagaagatgctcttagctttcattgtctttcttttgtagtcatctgatttacctctctcgtttatacaactggttttttaaacactccttaacttttcaaattgtctctttctttaccctagactagataattttaatggtgattttgctaatgtggcgccatgttagatagaggtaaaatgaactagttaaaagctcagagtgataaatcaggctctcaaaaattcataaactgttttttaaatatccaaatatttttacatggaaaataataaaatttagtttagtattaaaaaattcagttgaatatagttttgtcttcaaaaattatgaaactgatcttaattatttttccttaaaaccgtgctctatctttgatgtctagtttgagacgattatataattttttttgtgcttaactacgacgagctgaagtacgtagaaatactagtggagtcgtgccgcgtgtgcctgtagccactcgtacgctacagcccaagcgctagagcccaagaggccggaggtggaaggcgtcgcggcactatagccactcgccgcaagagcccaagagaccggagctggaaggatgagggtctgggtgttcacgaattgcctggaggcaggaggctcgtcgtccggagccacaggcgtggagacgtccgggataaggtgagcagccgctgcgataggggcgcgtgtgaaccccgtcgcgccccacggatggtataagaataaaggcattccgcgtgcaggatt

SEQ ID NO: 102 - DNA, 1588 bp, Artificial Sequence
  Leaf Preferred Promoter Sequence
tggtggagatgtattgtggtgaacatgaaatcagttgatgtgttgtttgtctgaatttctatgtacctggcacagtgaactggaaaatatcttaggtggaaagcacaagtcactagaccatttgcgctggattcacctgtgtgagaaaactgcaacattttcactttgttagctaggaagcaaggccatgttccattggctgcctggctgggcatttttaggactgctccgcttaaaaaaaaaataacttcactccaccaacttaaagctccactccaactaagaaaatagggagctacgaggttaatagtgtaaacttcagctccaaaaattataggaattggcgagaatggtctctcttgcccttggttgggattgatgtttttttttaccagctacgttaaccgactgctacagtaccgatcgctacggtaacactttgctacatcgcttcgcctactttgcagacttcattgtgcgcagctgaccgttggaatttttctggaataaaactccgcgtcattgtaataaaggatcacttttgaaaaccagcacgatcgtcatgtgttcgtatgaggtcattcggtagtccatgatactgaaagtgatttgtatccttttccaaaaagaatccgcagaaatcttcacaaagtaagttgatttggactaccataagaaatccagtgctctcaaatgttgcagaatagtttacgtcatacgcgttgaaaattatgccacccgaagattgcgaccatcttaaagcattgaaaattgatgtggcctgcgacattgggcccaggagatgaatcatccagcatttcagcaaaccgggctcaccaaggatgcagatgaccaaattggggtgaaatcggacgccagaggaaggggagatggcggcgacgacggaatagaagagtgtgagagggagggagaggcggcagcgatggaagatgaacaagcacggggaggtgtcaatcttttttccccctaaatcggtatgtgtaattggtgacattagtgagtaattttcaactaacttctattggaatgaaacttgtattttcgaagcaccctttgagaagctccagaaaaaatcttgaagttagctcctagatgcatattttttccttgaagttagtattattagagctagaaatgtttggcaagataattttaaagtggagttagaaaatctaaagcgaagcggtccaaacacgcctttgagatccgttaccgtcaacaacagaagcagagagcttcacgcccattttgagttccaaaaccacgtgatctccaaaatcattttggccgatatttcccagccccaggcactccgaagaccacaaatctcaggcgctccagacgctctcctcccaatcacggccctccacgtgtcccgaggccctccgccagcagcccatccattggaccatgcagataagacccggataaggcagagcccatcgctgccgtcaaatagatcacatccgtcccagggccgtccgatctgccacgcaggagatagcaccggaactctttcttatttgtaccccgggacgagccctctccacgccacacaccaccacca

SEQ ID NO: 103 - DNA, 710 bp, Artificial Sequence
  Endosperm Preferred Promoter Sequence
ttcagcgttatttgaacaccgtaaagcctctccagcagattgtgaatacacagttgtggagaacgctatttataacgcagacactatttataatgcagatgtgtaaaagtgaaatttaaaatagtagatgagataggagagatagaatgagtaaactgctggagagcaaatcgtgcatatgatcgtgcaaaacaccgtttttcgtagagtgaagtttaaaatagcaggtgagagagtagataggatgagtaagctgatggagagcaaatattgtatatacgtggtcggtgcaatagagtgaaatttgaaataactgacacagttttggtgcgtggaaatagacgaggataattctagtgcaatccgcactgccagtggaccccgcccgacgataattctacgcacgggcggcgcactgcactactagttcatcgatcggatgcgttagcgtgcccctcctcatattgtttccttgtacgtactagtgcaatccgtcagccgcacggctccagtccactccagtccagcaacagcgtcacctccagctccgaaaggcttatccttgcaacaaacatcgtacgaaaaaggcgcaggaaaaagaaaagtgtcgaaatacgacataaaaaaagcatcaaaatacgctgcgagtgagcgagacattggcctccccatcccatatatatatagctatagctatccctcggttcttc

SEQ ID NO: 104 - protein, 377 aa, Arabidopsis thaliana
MAVSFVRTSPEEEDKPKLGLGNIQTPLIFNPSMLDLQANMANQFHWPDDEKPSTLQLELDVPLIDLQNLLSDPSSTLDASRLISEACKKHGFFLVVNHGISEELISDAHEYTSRFFDMPLSEKQRVLRKSGESVGYASSFTGRFSTKLPWKETLSFRFCDDMSRSKSVQDYFCDALGHGFQPFGKVYQEYCEAMSSLSLKIMELLGLSLGVKRDYFREFFEENDSIMRLNYYPPCIKPDLTLGTGPHCDPTSLTILHQDHVNGLQVFVENQWRSIRPNPKAFVVNIGDTFMALSNDRYKSCLHRAVVNSERMRKSLAFFLCPKKDRVVTPPRELLDSITSRRYPDFTWSMFLEFTQKHYRADMNTLQAFSDWLTKPI

SEQ ID NO: 105 - protein, 262 aa, Sorghum bicolor
MELGDATAGQGAQGDAASGALVRKKRMRKKSTGPDSIAETIKWWKEQNQKLQDESGSRKAPAKGSKKGCMTGKGGPENVNCVYRGVRQRTWGKWVAEIREPNRGRRLWLGSFPTAVEAAHAYDEAAKAMYGPKARVNFSDNSADANSGCTSALSLLASSVPVATLQRSDEKVETEVESVETEVHEVKTEGNDDLGSVHVACKTVDVIQSEKSVLHKAGEVSYDYFNVEEVVEMIIIELNADKKIEANEEYHDGDDGFSLFAY

SEQ ID NO: 106 - protein, 262 aa, Sorghum bicolor
MELGDATAGQGAQGDAASGALVRKKRMRRKSTGPDSIAETIKWWKEQNQKLQDESGSRKAPAKGSKKGCMTGKGGPENVNCVYRGVRQRTWGKWVAEIREPNRGRRLWLGSFPTAVEAAHAYDEAAKAMYGPKARVNFSDNSADANSGCTSALSLLASSVPVATLQRSDEKVETEVESVETEVHEVKTEGNDDLGSVHVACKTVDVIQSEKSVLHKAGEVSYDYFNVEEVVEMIIIELNADKKIEANEEYHDGDDGFSLFAY

SEQ ID NO: 107 - protein, 228 aa, Zea mays
MARERREIKRIESAAARQVTFSKRRRGLFKKAEELSVLCDADVALIVFSSTGKLSQFASSSMNEIIDKYSTHSKNLGKAEQPSLDLNLEHSKYANLNEQLVEASLRLRQMRGEELEGLSVEELQQLEKNLESGLHRVLQTKDQQFLEQISDLEQKSTQLAEENRQLRNQVSHIPPVGKQSVADTENVIAEDGQSSESVMTALHSGSSQDNDDGSDVSLKLGLPCVAWK

SEQ ID NO: 108 - protein, 155 aa, Arabidopsis thaliana
METLQCRHQHVFILLLVLFHSSLFGLASKIDVSDDARGIRIDGGQKRFLTNSPQHGKEHAACTNEEPDLGPLTRISCNEPGYVITKINFADYGNPTGTCGHFRRGNCGARATMRIVKKNCLGKEKCHLLVTDEMFGPSKCKGAPMLAVETTCTIA

SEQ ID NO: 109 - protein, 454 aa, Saccharomyces bayanus
MSEPEFQQAYDEVVSSLEDSTLFEQHPKYRKVLPIVSVPERIIQFRVTWENDKGEQEVAQGYRVQYNSAKGPYKGGLRFHPSVNLSILKFLGFEQIFKNSLTGLDMGGGKGGLCVDLKGRSNNEIRRICYAFMRELSRHIGQDTDVPAGDIGVGGREIGYLFGAYRTYKNSWEGVLTGKGLNWGGSLIRPEATGYGLVYYTQAMIDYATNGKESFEGKRVTISGSGNVAQFAALKVIELGGTVVSLSDSKGCIISETGITSEQVADISSAKVNFKSLEQIVGEYSTFTENKVQYISGARPWTHVQKVDIALPCATQNEVSGDEAKALVAQGVKFVAEGSNMGSTPEAIAVFETARATASTLKESVWYGPPKAANLGGVAVSGLEMAQNSQRITWSSERVDQELKKIMVNCFNECIDSAKKYTKEGNALPSLVKGANIASFIKVSDAMFDQGDVF

SEQ ID NO: 110 - protein, 1025 aa, Arabidopsis thaliana
MAASGPKSSGPRGFGRRTTVGSAQKRTQKKNGEKDSNATSTATNEVSGISKLPAAKVDVQKQSSVVLNERNVLDRSDIEDGSDRLDKKTTDDDDLLEQKLKLERENLRRKEIETLAAENLARGDRMFVYPVIVKPDEDIEVFLNRNLSTLNNEPDVLIMGAFNEWRWKSFTRRLEKTWIHEDWLSCLLHIPKEAYKMDFVFFNGQSVYDNNDSKDFCVEIKGGMDKVDFENFLLEEKLREQEKLAKEEAERERQKEEKRRIEAQKAAIEADRAQAKAETQKRRELLQPAIKKAVVSAENVWYIEPSDFKAEDTVKLYYNKRSGPLTNSKELWLHGGFNNWVDGLSIVVKLVNAELKDVDPKSGNWWFAEVVVPGGALVIDWVFADGPPKGAFLYDNNGYQDFHALVPQKLPEELYWLEEENMIFRKLQEDRRLKEEVMRAKMEKTARLKAETKERTLKKFLLSQKDVVYTEPLEIQAGNPVTVLYNPANTVLNGKPEVWFRGSFNRWTHRLGPLPPQKMEATDDESSHVKTTAKVPLDAYMMDFVFSEKEDGGIFDNKNGLDYHLPVVGGISKEPPLHIVHIAVEMAPIAKVGGLGDVVTSLSRAVQELNHNVDIVFPKYDCIKHNFVKDLQFNRSYHWGGTEIKVWHGKVEGLSVYFLDPQNGLFQRGCVYGCADDAGRFGFFCHAALEFLLQGGFHPDILHCHDWSSAPVSWLFKDHYTQYDLIKTRIVFTIHNLEFGANAIGKAMTFADKATTVSPTYAKEVAGNSVISAHLYKFHGIINGIDPDIWDPYNDNFIPVPYTSENVVEGKRAAKEELQNRLGLKSADFPVVGIITRLTHQKGIHLIKHAIWRTLERNGQVVLLGSAPDPRIQNDFVNLANQLHSSHGDRARLVLTYDEPLSHLIYAGADFILVPSIFEPCGLTQLIAMRYGAVPVVRKTGGLFDTVFDVDHDKERAQAQVLEPNGFSFDGADAPGVDYALNRAISAWYDGREWFNSLCKTVMEQDWSWNRPALEYLELYHSACK

SEQ ID NO: 111 - protein, 228 aa, Zea mays
MARERREIKRIESAAARQVTFSKRRRGLFKKAEELSVLCDADVALIVFSSTGKLSQFASSSMNEIIDKYSTHSKNLGKAEQPSLDLNLEHSKYANLNEQLVEASLRLRQMRGEELEGLSVEELQQLEKNLESGLHRVLQTKDQQFLEQISDLEQKSTQLAEENRQLRNQVSHIPPVGKQSVADAENVIAEDGQSSESVMTALHSGSSQDNDDGSDVSLKLGLPCVAWK

SEQ ID NO: 112 - protein, 377 aa, Arabidopsis thaliana
MAVSFVTTSPEEEDKPKLGLGNIQTPLIFNPSMLNLQANIPNQFIWPDDEKPSINVLELDVPLIDLQNLLSDPSSTLDASRLISEACKKHGFFLVVNHGISEELISDAHEYTSRFFDMPLSEKQRVLRKSGESVGYASSFTGRFSTKLPWKETLSFRFCDDMSRSKSVQDYFCDALGHGFQPFGKVYQEYCEAMSSLSLKIMELLGLSLGVKRDYFREFFEENDSIMRLNYYPPCMKPDLTLGTGPHCDPTSLTILHQDHVNGLQVFVENQWRSIRPNPKAFVVNIGDTFMALSNDRYKSCLHRAVVNSKSERKSLAFFLCPKKDRVVTPPRELLDSITSRRYPDFTWSMFLEFTQKHYRADMNTLQAFSDWLTKPI

SEQ ID NO: 113 - protein, 377 aa, Arabidopsis thaliana
MAVSFVTTSPEEEDKPKLGLGNIQTPLIFNPSMLNLQANIPNQFIWPDDEKPSINVLELDVPLIDLQNLLSDPSSTLDASRLISEACKKHGFFLVVNHGISEELISDAHEYTSRFFDMPLSEKQRVLRKSGESVGYASSFTGRFSTKLPWKETLSFRFCDDMSRSKSVQDYFCDALGHGFQPFGKVYQEYCEAMSSLSLKIMELLGLSLGVKRDYFREFFEENDSIMRLNYYPPCIKPDLTLGTGPHCDPTSLTILHQDHVNGLQVFVENQWRSIRPNPKAFVVNIGDTFMALSNDRYKSCLHRAVVNSERMRKSLAFFLCPKKDRVVTPPRELLDSITSRRYPDFTWSMFLEFTQKHYRADMNTLQAFSDWLTKPI

SEQ ID NO: 114 - protein, 454 aa, Saccharomyces cerevisiae
MSEPEFQQAYEEVVSSLEDSTLFEQHPEYRKVLPIVSVPERIIQFRVTWENDKGEQEVAQGYRVQYNSAKGPYKGGLRFHPSGNLSILKFLGFEQIFKNSLTGLDMGGGKGGLCVDLKGRSNNEIRRICYAFMRELSRHIGQDTDVPAGDIGVGGREIGYLFGAYRSYKNSWEGVLTGKGLNWGGSLIRPEATGYGLLYYTQAMIDYATNGKESFEGKRVTISGSGNVAQYAALKVIELGGTVVSLSDSKGCIILETGITSEQVAVISSAKVNFKSLEQIVNEYSTFSENKVQYIAGARPWTHVQKVDIALPCATQNEVSGEEAKALVAQGVKFIAEGSNMGSTPEAIAVFETARSTATGPSEAVWYGPPKAANLGGVAVSGLEMAQNSQRITWTSERVDQELKRIMINCFNECIDYAKKYTKDGKVLPSLVKGANIASFIKVSDAMFDQGDVF

SEQ ID NO: 115 - protein, 349 aa, Zea mays
MGGLTMDQAFVQAPEHRPKPIVTEATGIPLIDLSPLAASGGAVDALAAEVGAASRDWGFFVVVGHGVPAETVARATEAQRAFFALPAERKAAVRRNEAEPLGYYESEHTKNVRDWKEVYDLVPREPPPPAAVADGELVFDNKWPQDLPGFREALEEYAKAMEELAFKLLELIARSLKLRPDRLHGFFKDQTTFIRLNHYPPCPSPDLALGVGRHKDAGALTILYQDDVGGLDVRRRSDGEWVRVRPVPDSFIINVGDLIQVWSNDRYESAEHRVSVNSARERFSMPYFFNPATYTMVEPVEELVSKDDPPRYDAYNWGDFFSTRKNSNFKKLNVENIQIAHFKKSLVLA

SEQ ID NO: 116 - protein, 377 aa, Arabidopsis thaliana
MAVSFVTTSPEEEDKPKLGLGNIQTPLIFNPSMLNLQANIPNQFIWPDDEKPSINVLELDVPLIDLQNLLSDPSSTLDASRLISEACKKHGFFLVVNHGISEELISDAHEYTSRFFDMPLSEKQRVLRKSGESVGYASSFTGRFSTKLPWKETLSFRFCDDMSRSKSVQDYFCDALGHGFQPFGKVYQEYCEAMSSLSLKIMELLGLSLGVKRDYFREFFGENDSIMRLNYYPPCIKPDLTLGTGPHCDPTSLTILHQDHVNGLQVFVENQWRSIRPNPKAFVVNIGDTFMALSNDRYKSCLHRAVVNSESERKSLAFFLCPKKDRVVTPPRELLDSITSRRYPDFTWSMFLEFTQKHYRADMNTLQAFSDWLTKPI

SEQ ID NO: 117 - protein, 262 aa, Sorghum bicolor
MELGDATAGQGAQGDAASGALVRKKRMRRKSTGPDSIAETIKWWKEQNQKLQDESGSRKAPAKGSKKGCMTGKGGPENVNCVYRGVRQRTWGKWVAEIREPNRGRRLWLGSFPTAVEAAHAYDEAAKAMYGPKARVNFSDNSADANSGCTSALSLLASSVPVATLQRSDEKVESEVESVETEVHEVKTEGNDDLGSVHVACKTVDVIQSEKSVLHKAGEVSYDYFNVEEVVEMIIIELNADKKIEANEEYHDGDDGFSLFAY

SEQ ID NO: 118 - protein, 262 aa, Sorghum bicolor
MELGDATAGQGAQGDAASGALVRKKRMRRKSTGPDSIAETIKWWMEQNQKLQDESGSRKAPAKGSKKGCMTGKGGPENINCVYRGVRQRTWGKWVAEIREPNRGRRLWLGSFPTAVEAAHAYDEAAKAMYGPKARVNFSDNSADANSGCTSALSLLASSVPVATLQRSDEKVETEVESVETEVHEVKTEGNDDLGSVHVACKTVDVIQSEKSVLHKAGEVSYDYFNVEEVVEMIIIELNADKKIEANEEYHDGDDGFSLFAY

SEQ ID NO: 119 - protein, 141 aa, Brassica rapa
MAASVMLSSVTLKPAGFTVEKMSARGLPSLTRASPSSFRIVASGVKKIKTDKPFGVNGSMDLRDGVDASGRKGKGYGVYKFVDKYGANVDGYSPIYNEDEWSASGDVYKGGVTGLAIWAVTLAGILAGGALLVYNTSALAQ

SEQ ID NO: 120 - protein, 371 aa, Zea mays
MVLAAHDPPPLVFDAARLSGLSDIPQQFIWPADESPTPDSAEELAVPLIDLSGDAAEVVRQVRRACDLHGFFQVVGHGIDAALTAEAHRCMDAFFTLPLPDKQRAQRRQGDSCGYASSFTGRFASKLPWKETLSFRYTDDDDGDKSKDVVASYFVDKLGEGYRHHGEVYGRYCSEMSRLSLELMEVLGESLGVGRRHFRRFFQGNDSIMRLNYYPPCQRPYDTLGTGPHCDPTSLTILHQDDVGGLQVFDAATLAWRSVRPRPGAFVVNIGDTFMALSNGRYRSCLHRAVVNSRVARRSLAFFLCPEMDKVVRPPKELVDDANPRAYPDFTWRTLLDFTMRHYRSDMRTLEAFSNWLSTRSNGGQHLLEKK

SEQ ID NO: 121 - protein, 262 aa, Sorghum bicolor
MELGDATAGQGAQGDAASGALVRKKRMRRKSTGPDSIAETIKWWKEQNQKLQDESGSMKAPAKGSKKGCMTGKGGPENVNCVYRGVRQRTWGKWVAEIREPNRGRRLWLGSFPTAVEAAHAYDEAAKAMYGPKARVNFSDNSADANSGCTSALSLLASSVPVATLQRSDEKVETEVESVETEVHEVKTEGNDDLGSVHVACKTVDVIQSEKSVLHKAGEVSYDYFNVEEVVEMIIIELNADKKIEANEEYHDGDDGFSLFAY

SEQ ID NO: 122 - protein, 156 aa, Arabidopsis thaliana
METLQCRHQHVFILLLVLFHSSLFGLASKIDVSDDARGIRIDGGQKRFLTNSPQHGKEHAACTNEEPDLGPLTRISCNEPGYVITKINFADYGNPTGTCGHFRRGNCGARATMRIVKKNCLGKEKCHLLVTDEMFGPSKCKGAPMLAVETTCTIAG

SEQ ID NO: 123 - protein, 349 aa, Zea mays
MGGLTMDQAFVQAPEHRPKPLVTEATGIPLIDLSPLSASGGAVDALAAEVGAASRDWGFFVVVGHGVPAETMARATEAQRAFFALPAERKAAVRRNEAEPLGYYESEHTKNVRDWKEVYDLVPREPPPPAAVADGELVFENKWPQDLPGFREALEEYAKAMEELAFKLLELIARSLKLRPDRLHGFFKDQTTFIRLNHYPPCPSPDLALGVGRHKDAGALTILYQDDVGGLDVRRRSDGEWVRVRPVPDSFIINVGDLIQVWSNDRYESAEHRVSVNSARERFSMPYFFNPATYTMVEPVEELVSKDDPPRYDAYNWGDFFSTRKNSNFKKLNVENIQIAHFKKSLVLA

SEQ ID NO: 124 - protein, 1025 aa, Arabidopsis thaliana
MAASGPKSSGPRGFGRRTTVGSAQKRTQKKNGEKDSNATSTATNEVSGISKLPAAKVDVQKQSSVVLNERNVLDRSDIEDGSDRLDKKTTDDDDLLEQKLKLERENLRRKEIETLAAENLARGDRMFVYPVIVKPDEDIEVFLNRNLSTLNNEPDVLIMGAFNEWRWKSFTRRLEKTWIHEDWLSCLLHIPKEAYKMDFVFFNGQSVYDNNDSKDFCVEIKGGMDKVDFENFLLEEKLREQEKLAKEEAERERQKEEKRRIEAQKAAIEADRAQAKAETQKRRELLQPAIKKAVVSAENVWYIEPSDFKAEDTVKLYYNKRSGPLTNSKELWLHGGFNNWVDGLSIVVKLVNAELKDVDPKSGNWWFAEVVVPGGALVIDWVFADGPPKGAFLYDNNGYQDFHALVPQKLPEELYWLEEENMIFRKLQEDRRLKEEVMRAKMEKTARLKAETKERTLKKFLLSQKDVVYTEPLEIQAGNPVTVLYNPANTVLNGKPEVWFRGSFNRWTHRLGPLPPQKMEATDDESSHVKTTAKVPLDAYMMDFVFSEKEDGGIFDNKNGLDYHLPVVGGISKEPPLHIVHIAVEMAPIAKVGGLGDVVTSLSRAVQELNHNVDIVFPKYDCIKHNFVKDLQFNRSYHWGGTEIKVWHGKVEGLSVYFLDPQNGLFQRGCVYGCADDAGRFGFFCHAALEFLLQGGFHPDILHCHDWSSAPVSWLFKDHYTQYGLIKTRIVFTIHNLEFGANAIGKAMTFADKATTVSPTYAKEVAGNSVISAHLYKFHGIINGIDPDIWDPYNDNFIPVPYTSENVVEGKRAAKEELQNRLGLKSADFPVVGIITRLTHQKGIHLIKHAIWRTLERNGQVVLLGSAPDPRIQNDFVNLANQLHSSHGDRARLVLTYDEPLSHLIYAGADFILVPSIFEPCGLTQLIAMRYGAVPVVRKTGGLFDTVFDVDHDKERAQAQVLEPNGFSFDGADAPGVDYALNRAISAWYDGREWFNSLCKTVMEQDWSWNRPALEYLELYHSARK

SEQ ID NO: 125 - protein, 719 aa, Oryza sativa
MSASPSSMSGAGAGEAGVRTVVWFRRDLRVEDNPALAAAARAAGEVVPVYVWAPEEDGPYYPGRVSRWWLSQSLKHLDASLRRLGASRLVTRRSADAVVALIELVRSIGATHLFFNHLYGSIDPEFQIDPLSLVRDHRVKALLTAEGIAVQSFNADLLYEPWEVVDDDGCPFTMFAPFWDRCLCMPDPAAPLLPPKRIAPGELPARRCPSDELVFEDESERGSNALLARAWSPGWQNADKALAAFLNGPLMDYSVNRKKADSASTSLLSPYLHFGELSVRKVFHQVRMKQLMWSNEGNHAGDESCVLFLRSIGLREYSRYLTFNHPCSLEKPLLAHLRFFPWVVDEVYFKVWRQGRTGYPLVDAGMRELWATGWLHDRIRVVVSSFFVKVLQLPWRWGMKYFWDTLLDADLESDALGWQYISGSLPDGRELDRIDNPQLEGYKFDPHGEYVRRWLPELARLPTEWIHHPWDAPESVLQAAGIELGSNYPLPIVELDAAKTRLQDALSEMWELEAASRAAMENGMEEGLGDSSDVPPIAFPPELQMEVDRAPAQPTVHGPTTAGRRREDQMVPSMTSSLVRAETELSADFDNSMDSRPEVPSQVLFQPRMEREETVDGGGGGGMVGRSNGGGHQGQHQQQQHNFQTTIHRARGVAPSTSEASSNWTGREGGVVPVWSPPAASGPSDHYAADEADITSRSYLDRHPQSHTLMNWSQLSQSL

SEQ ID NO: 126 - protein, 580 aa, Zea mays
MASLFGARRRRSPEYDGEDDRSGGGRAKHRRLSPEEAAASPADPGAATGTSHGWLSGFVSGAKRAISSVLLSSSPEETGSGEDGEVEEEEEDDEYEEGIDLNENEDIHDIHGEIVPYSESKLAIEQMVMKETFSRDECDRMVELIKSRVRDSTPETHEYGKQEEIPSRNAGIAHDFTGTWRSLSRDRNFTESVPFSSMRMRPGHSSPGFPLQASPQLCTAAVREAKKWLEEKRQGLGVKPEDNGSCTLNTDIFSSRDDSDKGSPVDLAKSYMRSLPPWQSPFLGHQKFDTSPSKYSISSTKVTTKEDYLSSFWTKLEESRIARIGSSGDSAVASKLWNYGSNSRLFENDTSIFSLGTDEKVGDPTKTHNGSEKVAATEPLGRCSLLITPTEDRTDGITEPVDLAKNNENAPQEYQAASEIIPDKVAEGNDVSSTGITKDTTGHSADGKALTSEPHIGETHVNSASESIPNDAAPPTQSKMNGSTKKSLVNGVLDQPNANSGLESSGNDYPSYTNSSSAMPPASTELIGSAAAVIDVDSAENGPGTKPEQPAKGASRASKSKVVPRGQKRVLRSATRGRAT

SEQ ID NO: 127 - protein, 340 aa, Oryza sativa
MGGVAAGTRWIHHVRRLSAAKVSTDALERGQSRVIDASLTLIRERAKLKAELLRALGGVKASACLLGVPLGHNSSFLQGPAFAPPRIREAIWCGSTNSSTEEGKELNDPRVLTDVGDVPIQEIRDCGVEDDRLMNVVSESVKTVMEEDPLRPLVLGGDHSISYPVVRAVSEKLGGPVDILHLDAHPDIYDAFEGNIYSHASSFARIMEGGYARRLLQVGIRSITKEGREQGKRFGVEQYEMRTFSKDREKLESLKLGEGVKGVYISVDVDCLDPAFAPGVSHIEPGGLSFRDVLNILHNLQGDVVAGDVVEFNPQRDTVDGMTAMVAAKLVRELTAKISK

SEQ ID NO: 128 - protein, 327 aa, Arabidopsis thaliana
MTIGSFFSSLLFWRNSQDQEAQRGRMQEIDLSVHTIKSHGGRVASKHKHDWIILVILIAIEIGLNLISPFYRYVGKDMMTDLKYPFKDNTVPIWSVPVYAVLVPIIVFVCFYLKRTCVYDLHHSILGLLFAVLITGVITDSIKVATGRPRPNFYWRCFPDGKELYDALGGVVCHGKAAEVKEGHKSFPSGHTSWSFAGLTFLSLYLSGKIKAFNNEGHVAKLCLVIFPLLAACLVGISRVDDYWHHWQDVFAGALIGTLVAAFCYRQFYPNPYQEEGWGPYAYFKAAQERGVPVTSSQNGDALRAMSLQMDSTSLENMESGTSTGPR

SEQ ID NO: 129 - protein, 262 aa, Sorghum bicolor
MELGDATAGQGAQGDAASGALVRKKRMRKKSTGPDSIAETIKWWKEQNQKLQDESGSRKAPAKGSKKGCMTGKGGPENVNCVYRGVRQRTWVKWVAEIREPNRGRRLWLGSFPTAVEAAHAYDEAAKAMYGPKARVNFSDNSADANSGCTSALSLLASSVPVATLQRSDEKVETEVESVETEVHEVKTEGNDDLGSVHVACKTVDVIQSEKSVLHKAGEVSYDYFNVEEVVEMIIIELNADKKIEANEEYHDGDDGFSLFAY

SEQ ID NO: 130 - protein, 262 aa, Sorghum bicolor
MELGDATAGQGAQGDAASGALVRKKRMRRKSTGPDSIAETIKWWKEQNQKLQDESGSRKAPAKGSKKGCMTGKGGPENVNCVYRGVRQRTWGKWVAEIREPNRGRRLWLGSFPTAVEAAHAYDEAAKAMYGPKARVNFSDNSADANSGCTSALSLLASSVPVATLQRSDEKVETEVESVETEVHEVKTEGNDDLGSVHVACKTVDVIQSEKSVLHKAGEVSYDYFNVEEVVEMIIIELNADKKIEAHEEYHDGDDGFSLFAY

SEQ ID NO: 131 - protein, 262 aa, Sorghum bicolor
MELGDATAGQGAQGDAASGALVRKKRMRRKSTGPDSIAETIKWWKEQNQKLQDESGSRKAPAKGSKKGCMTGKGGPENVNCVYRGVRQRTWGKWVAEIREPNRGRRLWLGSFPTAVEAAHAYDEAAKAMYGPKARVNFSDNSADANSGCTSALSLLASSVPVATLQRSDEKVETEVESVESEVHEVKTEGNDDLGSVHVACKTVDVIQSEKSVLHKAGEVSYDYFNVEEVVEMIIIELNADKKIEANEEYHDGDDGFSLFAY

SEQ ID NO: 132 - protein, 453 aa, Saccharomyces cerevisiae
MSEPEFQQAYEEVVSSLEDSTLFEQHPEYRKVLPIVSVPERIIQFRVTWENDKGEQEVAQGYRVQYNSAKGPYKGGLRFHPSVNLSILKFLGFEQIFKNSLTGLDMGGGKGGLCVDLKGRSNNEIRRICYAFMRELSRHIGQDTDVPAGDIGVGGREIGYLFGAYRSYKNSWEGVLTGKGLNWGGSLIRPEATGYGLVYYTQAMIDYATNGKESFEGKRVTISGSGNVAQYAALKVIELGGTVVSLSDSKGCIISETGITSEQVADISSAKVNFKSLEQIVNEYSTFSENKVQYIAGARPWTHVQKVDIALPCATQNEVSGEEAKALVAQGVKFIAEGSNMGSTPEAIAVFETARSTPLDQATVWYGPPKAANLGGVAVSGLEMAQNSQRITWTSERVDQELKRIMINCFNECIDYAKKYTKDGKVLPSLVKGANIASFIKVSDAMFDQGDVF

SEQ ID NO: 133 - protein, 454 aa, Saccharomyces bayanus
MSEPEFQQAYDEVVSSLEDSTLFEQHPKYRKVLPIVSVPERIIQFRVTWENDKGEQEVAQGYRVQYNSAKGPYKGGLRFHPSVNLSILKFLGFEQIFKNSLTGLDMGGGKGGLCVDLKGRSNNEIRRICYAFMRELSRHIGQDTDVPAGDIGVGGREIGYLFGAYRSYKNSWEGVLTGKGLNWGGSLIRPEATGYGLVYYTQAMIDYATNGKESFEGKRVTISGSGNVAQFAALKVIELGGTVVSLSDSKGCIISETGITSEQIADISSAKVNFKSLEQIVGEYSTFTENKVQYISGARPWTHVQKVDIALPCATQNEVSGDEAKALVAQGVKFVAEGSNMGSTPEAIAVFETARATASTLKESVWYGPPKAANLGGVAVSGLEMAQNSQRITWSSERVDQELKKIMVNCFNECIDSAKKYTKEGNALPSLVKGANIASFIKVSDAMFDQGDVF

SEQ ID NO: 134 - protein, 718 aa, Oryza sativa
MSASPSSMSGAGAGEAGVRTVVWFRRDLRVEDNPALAAAARAAGEVVPVYVWAPEEDGPYYPGRVSRWWLSQSLKHLDASLRRLGASRLVTRRSADAVVALIELVRSIGATHLFFNHLYDPLSLVRDHRVKALLTAEGIAVQSFNADLLYEPWEVVDDDGCPFTMFAPFWDRCLCMPDPAAPLLPPKRIAPGELPARRCPSDELVFEDESERGSNALLARAWSPGWQNADKALAAFLNGPLMDYSVNRKKADSASTSLLSPYLHFGELSVRKVFHQVRMKQLMWSNEGNHAGDESCVLFLRSIGLREYSRYLTFNHPCSLEKPLLAHLRFFPWVVDEVYFKVWRQGRTGYPLVDAGMRELWATGWLHDRIRVVVSSFFVKVLQLPWRWGMKYFWDTLLDADLESDALGWQYISGSLPDGRELDRIDNPQLEGYKFDPHGEYVRRWLPELARLPTEWIHHPWDAPESVLQAAGIELGSNYPLPIVELDAAKTRLQDALSEMWELEAASRAAMENGMEEGLGDSSDVPPIAFPPELQMEVDRAPAQPTVHGPTTAGRRREDQMVPSMTSSLVRAETELSADFDNSMDSRPEVPSQVLFQPRMEREETVDGGGGGGMVGRSNGGGHQGQHQQQQHNFQTTIHRARGVAPSTSEASSNWTGREGGVVPVWSPPAASGPSDHYAADEADITSRSYLDRHPQSHTLMNWSQLSQSLTTGWEVEN

SEQ ID NO: 135 - protein, 377 aa, Arabidopsis thaliana
MAVSFVTTSPEEEDKPKLGLGNIQTPLIFNPSMLNLQANIPNQFIWPDDEKPSINVLELDVPLIDLQNLLSDPSSTLDASRLISEACKKHGFFLVVNHGISEELISDAHEYTSRFFDMPLSEKQRVLRKSGESVGYASSFTGRFSTKLPWKETLSFRFCDDMSRSKSVQDYFCDALGHGFQPFGKVYQEYCEAMSSLSLKIMELLGLSLGVKRDYFREFFEENDSIMRLNYYPPCIKPDLTLGTGPHCDPTSLTILHQDHVNGLQVFVENQWRSIRPNPKAFVVNIGDTFMALSNDRYKSCLHRAVVNSKSERKSLAFFLCPKKDRVVTPPRELLDSITSRRYPDFTWSMFLEFTQKHYRADMNTLQAFSDWLTKPI

SEQ ID NO: 136 - protein, 371 aa, Zea mays
MVLAAHDPPPLVFDAARLSGLSDIPQQFIWPADESPTPDSAEELAVPLIDLSGDAAEVVRQVRRACDLHGFFQVVGHGIDAALTAEAHRCMDAFFTLPLPDKQRAQRRQGDSCGYASSFTGRFASKLPWKETLSFRYTDDDDGDKSKDVVASYFVDKLGEGYRHHGEVYGRYCSEMSRLSLELMEVLGESLGVGRRHFRRFFQGNDSIMRLNYYPPCQRPYDTLGTGPHCDPTSLTILHQDDVGGLQVFDAATLAWRSIRPRPGAFVVNIGDTFMALSNGRYRSCLHRAVVNSRVARRSLAFFLCPEMDKVVRPPKELVDDANPRAYPDFTWRTLLDFTMRHYRSDMRTLEAFSNWLSTSSNGGQHLLEKK

SEQ ID NO: 137 - protein, 141 aa, Brassica juncea
MAASVMLSPVTLKPAGFTVEKMSARGLPSLTRASPSSFRIVASGVKKIKTDKPFGVNGSMDLRDGVDASGRKGKGYGVYKFVDEYGANVDGYSPIYNEEEWSASGDVYKGGVTGLAIWAVTLAGILAGGALLVYNTSALAQ

SEQ ID NO: 138 - protein, 327 aa, Arabidopsis lyrata
MTIGSFFSSLLFWRNSQDQEAQRGRIQEIDLGVHTIKTHGGRVASKHKHDWIILVILIAIEIGLNLISPFYRYVGKDMMTDLKYPFKDNTVPIWSVPVYAVLLPIILFVCFYLKRRCVYDLHHSILGLLFAVLITGVITDSIKVATGRPRPNFYWRCFPDGKELYDALGGVICHGKAAEVKEGHKSFPSGHTSWSFAGLTFLSLYLSGKIKAFNGEGHVAKLCLVIFPLLAACLVGISRVDDYWHHWQDVFAGALIGILVAAFCYRQFYPNPYHEEGWGPYAYFKAAQERGVPVASSQNGDALRAMSLQMDSTSLENMESGTSTAPR

SEQ ID NO: 139 - protein, 596 aa, Zea mays
MDEVPATAAVLDFRPGSSVPRVSAVPRRAVQCPPDTGGAEAATGGRPGIGNTAAVSAKLTGSSSAGPDIQSVDCDTSGGLAGGDAGDVGVLCLENAAETESVEPGVSDVRLGAPVEERHGRTLDSTGLGSGKAGETNEISLVEVSQSGATSSLDATASIGGGYSLVEGSLPEASGARRCKPEVHEVPTGTPATVGFPIEDGGYGFGIGPNDDVDGRNDPAGGEWEPPTDGNDAEDVTDMGGILCDERVERMETNSVEREASNGSTVSSEEGVDRMGTSLDDSEASDGSTTQDSDTDVETESSVSSIEEQEAGYGAHIPQPDPAVCKVAKENNTAGVKISDRMTSVSELTLVLASGASMLPHPSKVRTGGEDAYFIACDGWFGVADGVGQWSFEGINAGLYARELMDGCKKIVEETQGAPGMRTEEVLAKAADEARSPGSSTVLVAHFDGKVLHASNIGDSGFLVIRNGEVHKKSNPMTYGFNFPLQIEKGDDPLKLVQKYAICLQEGDVVVTASDGLFDNVYEEEVAGIVSKSLEADLKPTEIADLLVARAKEVGRCGFGRSPFSDSALAAGYLGYSGGKLDDVTVVVSIVRKSEV

SEQ ID NO: 140 - protein, 187 aa, Zea mays
MARILVEAPAGSGSPEDSINSDMILILAGLLCALVCVLGLGLVARCACSWRWATESGRAQPDAAKAANRGVKKEVLRSLPTVTYVSDSGKAAAAAEGGADECAICLAEFEGGQAVRVLPQCGHAFHAACVDTWLRAHSSCPSCRRVLAVDLPPAERCRRCGARPGAGISALWKAPTRCSAEGPTFLA

SEQ ID NO: 141 - DNA, 14852 bp, Zea mays
taaaatttagggtaaaatatatatctaatttaggtatttatatatggacaataaatcatagataacgttgctagagaagaatgaaatataaaagagaaaatcttttaaacagactataaggaaggatatagagtataaataaatatagagaatgttcttgagacaggtagagatggcaacagataaatacccatcagatagttctattacatacccgtatctgccaataaaatttataccctctataatactcatacccactcatgggtatgaaatagtacccatacccgatttgatacccactacatatattaaatagaacaaatctaataaatacttcccccatcccattgattaagaagcgcatatatttgaaaaagtgaatcttttaatttttttacttataatttagcccatataaatatatttttagtgtatacatactacatatttagattcattaaaatacttcaagatgagggtattttttatttttcaaaacatatcatcaacatcatcagttgatgatgatatattttgaaagatatgaatgattaaagttttgttttaaagagcgtgctaaaaatttatacgccataatcaatgagacggagggagtatatgtcacaattaaaataataaatcatcaattatgagtaaaaaaaggcatgccaataatgaataatattgtaacatgcaagttgagcttatttttgccaatatatttagaacatttgtattagttatgtaaacagcttcctacgtgtgaatttaataagtttgcaagaaaaactctccccaacggttatgcaaagaagtccctaaatttataaagtccctacaaaacttaattttgtttctacatttatcaaccttattgagaccctctaaatccatgttggcgtcaagtccatcacgaaaagcctgcttctgccactgtttcatggcatctcaacaccacattgaccataaatccatgtccaccgctctagaagccttgacgccactgttgtcagacaacctcttgtgtcgatgtgccgccataacacctattgtgctgcttccacgtcgtcgccgtgaggacctccatggacggtgtgtgcctgtcgccgtagtcgtctagccttccatcgacaactccgaccttctaagacgttgctggtatttttttatggaagtcgttggaaaagttcacctcgttaagatatgtagtagatcctatgccgccaccgcttaaaagtaagggaagattgttgacatagatttctgtcgtcagtccaattaaaaatagaaaacataccaatttttttgttgacgtggacaacttaaaaaaatagcgaccaataaatggagaactcttggaccacaactttttttaaatctttaaagtattttagtagctttttaaatctataatttagggagctaaatttacataactattgtagatgcttttaatatgtgtgcttaattaaatagggtgtgtatatgtttgggacggttgtaaattacctgtgagatatggcatatgggtagtctatatccttacccacacccatctactcgacgaatatacgattaattgctttcattaacataaacacgtaaagaagttatcttataccatcgttatatccggtaaaacttatcaaatactcaggtttcgagtataatttgtcatatctagagacaggcttatcgctactgctactgagccttccgctacgcagctcaaggtaaaaacagctatcagcgggccacagaagctgctgccggacgcggcaacggccgtcgtccctgtacagttatatatacagaatatacagcgaattctgccgctctgccctactcagtaggcagttgtcgccgcgcataatcatactagtgacacaatcacagctagccacctccccgtccccagcatcgccaaaggtcacatcggtcggccacgggcgcggggcaagaaggaccaggtgacggacgagcaggagggaggaataatggcgggcgcgggcgcgggcgggtggaagaagcgggtgggccgctacgaggtgggccggaccatcggccggggcaccttcgccaaggttaagttcgccgtcgacgccgacaccggcgcggctttcgccattaaggtgctcgacaaggagaccatcttcacccaccgcatgctccaccaggtacctcttccagcgctctccgttttcatgcaggcgctggaacatcaccatgtcagattggatgaatccctatgctctaagcttcagcttgattttttttaaaattatcttttccgagtaagtaaacgatgtttcccgtcgaaagaagtcgccgaattggggtgcgggaccgagccggaatgacctggcgagcaacgccagccacacagcgtaatcccagcaagttgtcttgtgagcagcaaacccatatgccaccgtcacttgtttttttccctttcgtgaacggaataaacacttcccacactcgatttcagctacatatgatgttattttccattcatatttaaatagcttcttccaaaagattctcccattgttcctttttctctgcgactattcagcttctctatatactatagttatatttatctaatatttttatatcagtttttaagtttaaaaacatacaatacttatagtactataaacacatcaatattaggtattaaaacttgagaaaaaagataattttatagaaaaacataatgattaacgaaaggataagactagagcttccatgcaagagagaggagcctctctctctcttccccataatcgaccatcgaggaggcgtcgttggagcaccacaaggagctcgagctgccgtacaccaaaagccaacgctcggagcgcaggagtaaggtcggttctgtttggaagcaattttttaaggtagttcgaaaacacatgtttctaagggatttctattttttaagagaaattagtttatttttctttaaaaaaatagaaattttttagaaaaataaaatttctaaactagccctaagaaactgatttttatcttttagtttagagaggtcagttttttggtttttaagaaactggaaactcaatttctataaactaatatgtttagaactacatcaatttatataaatcagtctcttaaaaactgaatgcttcgaaatagtcactaagccgttaaacatgtcaccttcaaattggctccacggacacacgtacgattgtatccgcgcttggtccctggtccggcaagtgggttgcattcagcctcgcctgaccaccgccaccaaaaatgggatggaggtattttgtttctatgtcttgaagtgaatctatcctagtatcttgtgcgattagcattgaccaggttgattaacccattttggcgtttcgtacaggttctgtttatagagtcaagacattccatatttccaagggccctgttcgtttcttttctattctaacttggaatcgttacttgtgatcaaagcttatacaaattacacacgaggtccggctaggaatcgtttgtacccacggttcctaaccgcattattggttggcctgcctattcgcatcgggctcttgccgcctagacgggctagcgaagctcccacacccatttccccacacatgcagtcgtggaccaccaatcagctgctcgtgcgtagtcaatgcatacagcatgggcccagcatggacgcgatgggcaggccaaccaatcacgccctaagcaaccgaacagaccctaaatgactagtagaaagagaaaagtttgtccagactgatcaccatcgctttcttttcccacaacgttaaacctaaatttgagtggcagcagcagagatctgtatttttaggtgtaatatgttgtggtggtacactgataccaacaattacgcatagagtgtagaaacactagcaatggaaagcaccatttcttggacagaccggtggcggagtgacctgctggtgactccatatcggggagtaatggattattacctcttaagggctcgttcggttatccaatccagaaaaggattagaggggtttaaatcccctactagtcattttttattaagggctaatttggtgaccggggatcccgaggggaagaaatcctcttgctattcaaaattgaattgcaagggaattcctcccccatggatcctctagggatccctaatcaccaaatcagtcataaaaggggattcaatccccttcgggattggtgtaaccgaccctaacagttaaaccaaatatcttttgccagtttattaaaaaggacgaagtttccttgccccagtgagcttactacttgtttgctgccagatcaaaagggaaatatctatcatgaagatcgtaagacatcccaacatagttaggcttaatgaggtacgtggtgtgccacaacggtcggtgcagtgcttacagttctcgatctgttttgcctatctaatcgtattattatactggaactgtattccgacaaattctagcccctctatatatcaccagctggttttctttcttcactagcaatatttcattactttgtaggtgttggccggcaggacaaagatatacatagtcttggaacttgtcactggaggtgaactgtttgatagaatagtaagaatctcttgttccatgacatctccaattaagtagcttaaatatctattcctgcattatgtacatcacatatttttagcagatatgtatggtatatatcaaaatctataattttcatataaaatgtatcttcatttgactccgtacaaaaagaaatatgattttttttaaagcaacaacctttgcacgagataaatatgccgctcaaatctggaagaaatgggtactccatgaacctagaaggttataggtgacttcaacgttatgaagaaataagcaccgaaattcgcaatagagaatatctaaacctgggtgttgatgggtacttgaactgttatgacaaatctaaaattttaaaatagatatgtacttcctccttttttatttgtcatattttagttcaaaaataaactagcggaccacaaggtagtattaaatttgatagtaatcttttcttgtgttatcaagcacattattacggataagaataaaattttggataaattgttttctacattaccggttctctacaacaaaaaagtgaaaaatatctaaatccaaattttattcctatcatttaagaagatataagataaatttgaagtttattttttatgaaactttacaagcttaatgctaaaaacaagaatatttacatagcagattttatatcctatttattcataatcaaagaaaaaagaccaaaaattgatgtccgaataagtatctgtttgcatccctagtcagagctcacttggttccagtttcctaaatacatcaacaaagcctcctaagcaagttggatagacttgagttaaaaaacctaatagaacccacaattaaggttcaagcacatagatatttaagggtaaatctttagatatatcctatcatttcaagtctccctttattgtttttccttatatgtcaactatgatctccctctttatctgttctcattgctatcgcgcgttaggatcctactacacactaaaaggtctcagttggacatatctaaaccatttcagccgatgttggacaaacttttattcaattggtgttggtgctactcctaacctatcatgtatatcatcattttggactcgatcttttcttgtatggccacaaatcaacgcaacatatatatttttgcaacacttatctgttgaacatgtcgtaattttgtaggccaatagtttgcactatacaacatagtaagtctaatctttgtcctataaatttgccttttagctttgtggaaccctcttatcatatagaacaccagatgcttgacaccacttcttctatcctgctttgattctatggctaacaccttcatcaatgtccccagcactctgtagcgttgatcctaaatcctaaaggtgttcattcctagccactacttgaccttatggactaacatatcatttctcatgtgtagtgccaaaatcacgtctcatatattttgttttagttataccgagtctaaaacctttagattctagtgtttcctgccacaactctagtttcttatttacttctgactgacttccatcaacttggaaagtcattggtgtcctcatcacttattcgaacactagttataacattgttgtacatgtccttaatggatctaacatacttctttgaaactttttgttcgtccaaagtcccaccacataacattcattggttttttctcataaatcttcttcaagtcaatgaaactaacaatctgggcatgaaaccaaatgggttcaaagagatcttcattattcctcgctgacgatgctcgataactctctcttatagctttatagtgtggatcatcaacttaatcccttagtaattagtacaactttgaatatctctcttattcttgtagatcggtactaatatactatttatgcactcgtcagacatcttgttcgaccgaaagatatgattggacaacttggttaaccatactatagatatgtcgctcaagcatccccatgcttcaattgttataccgttagggcccatcgccttacccctttcatccttttaatgcctccataacctcagtttttttggatcctacgcataaaatgtttattgatgcaatcaaaagataatccaacgattttgtctccattttcaaatataaatctcacagataacatcaaatatttgtaggtccgccatgggaagctacgtgagaatgaagctaggaagtatttccagcagcttattgatgccattgattattgccacagcaaaggagtttatcatagagatttgaaggtatccctttgataattgatgtaagtttaattttagctattcattaattacatatcttacactgatagtttgggcattgttctgtagcctcaaaacttgcttcttgactctcgtggaaacttgaaactttctgattttggacttagcacattgtctcaaaatgtaagcttgttgatttctatcgctgtgctgccgacatatattaatgctttaatagtctaatatggagcttcccccagggagtaggccttgtacacacgacatgtggaacaccaaattatgttgcacctgaggtatggattgctgcttaaaagtactaaatggtgtaaacatgcatggtaggtgaaatgttctctattacaacaagatcaaacaaaaaacaaattcatttgccctacataatgcataaatggtcttttaagattttcttttgaacaaagcatgaactttctttcaagaaatcctgtaacaaaatatacacaacaagttagagggttccttgacaatggatgtgagagtcagattcgagaagagttatgacggatgatgtttctttataatgaaaaactgaatattccttttgccatgtaaatttttaagatgttgctagttgcatgttcttcactagtatgagttgtgtccgtcaagtcgttatctttgacatcattttgagaaaaaatcatgagactctaacttggattactattttaggtgctaagtagcaatggatatgacggatctgcagcagacatttggtcgtgtggtgtcattctctatgttttaatggctggttaccttccctttgaggagaacgaccttccacatttgtatgaaaaggtgtcagattcttttcaagtgtgaaattttgaatatatcaatgtctagaattgttgatttgcacaattacttgggtgttggctcatctaacactgaaacccctctcctcatcttttgtatttcaagataactgcagctcagtactcatgcccatattggttctctccaggagccaagtcattgatccagagaatacttgatccaaatccaagaactgtaagtaaaagcacaagcttgtttcatttcatcttcgagtatttggactctaaacaatgtttttcgtgtaactttaagctttcagaagattttgatcaccagttactttttttgttattttcagcctatagtgtgtgtgaaactctctacatgtattttgtcatatatttgtatatattgatgttctgatttgtgtttgggaaataccatatacaagatcaatgtaaggttatctctagtaatttactcatcatatcttttatcttatcacctattttaaactacactatgtaaacagtgcaaaacgtgttttgtacgaccatatgtacgatttgctggaggcaacctaaggactaaccacaataaattatttgcatttattgcgaggcatcttttacctaatatgtagaatttcagccatttgtattgacaaataaattgatcgtatcagtttgtcttgtctctatgcattatttccagcgtatcactattgaagaaataagagaagacccatggtttaagaagaactacgtaactattagatgtggtgaagatgaaaatgtcagcctagacgatgttcaagctatttttgacaatattgaggtttgtgagaaattggtcaatccaagcatgctctgcaaactcaattatctcattccgagtcaacagaaagatccctatggttctattgtccatttgaagatgttgttccttctaatgatgattttatattatgtttccttttcttaggacaagtatgtatcagacgaagtaacgcacaaggatggtggtcctcttatgatgaatgcctttgagatgattgcactatctcaaggtttggatctctcagcattgtttgataggcaacaggtacttcacatctccttttactcagcatagtggaccattgcatggacatacttaggtgaccctgaacccaagaagcacaaccatgctgatgtctggcatccacatcacaagggaaaggacataggagattctagaaaccttggtaaacaaatcagcaagctgtaactttgaaggcacatattgaagggaaacatgtagactaaacatagacagaattgatgccaatgtgtttagtgagctgatacttcactggatcctgagcaatactaatggcgtcagtactatcgcagtgcacatgcatagaagtagtaagaggattcctaaagttatctaacagccattgaagccatgtcacctcaaccgtcacacaagccaaatcatgcaactcagccgcaacactggaacgagcaacaacagtctgcttcttggtcttccatgaaaccaaggaggacccaaggaagacacaatagatagagatgaacttaaaattttaaggatcgatacaccaagccgcatcagaatatgcatagagctgtagagagctggtgctagaaaagaacatatgacagtcaatgatacctcgaaggtattgaaggacacgaaaaaatgtccatagtggatactagtgggagtaggcatgaattgactcaaaatgtgaacaacatagaatatatcaggacaagtgatgccaatgtagacaagactgctaacaagatggtgatagcgagtgggatcctcaacaggaacaccatctatggcgcgaagacgaaggtgaagatacatgggagtgtccatagaacggtgatcagtgagaccaacatgatcaaagatgtcatgaatgtatatgcgctgagaaaggtaatagccatcatgtgtggaggtgacctcaatcccaagaaagaagctaagaggacccaagtcaaacatgtggaattgcacactgagtcacgccttgacgaaggcaatgtaatcaagatcatcacaagtggtcaacatatagaagaatgatggtgcgatcttgagagcaagtgtgaataaagagagcagggtcatgttggatggtaacgaaactagcaacaacgataacagaggtgaagcgctcaaaccaggtgtgaggagactgcttgaggctatagagataacgacgaagccgatagacatgaccatcaagaaaaaggtaaccaggtggaggatgtgtgtagacttcctcacgcaactcaccatgaaggaaagcattcttcacatcaagttgtgagatagcccaccggcgaacagaggcaacaacaacaaaagtgtgaatggtggtctgatgggcaacatgaacaaaggtctcctcatagtcacgcccatactcttattgaaaaccacatgcaatcgtgctttatagcgttcaatggagccatcaaagcgggtcttaatcttatagacccacttgcacgtgataggagaagcaaatgatggaagtgggacaagatcccaagtgctagtgcgctcaagagcagcaatctctgccatagctaattgccactcgggatgggaaagtgcgtcatggaaggaggtagtctcagagggaatggaaacagaagcagcaaaaccaagacgagtaggacgacaaagcgtacgtcgatcgccaaggtcatagtgcggaggaggcacaccaagcacagatggagacatgctagatggtgctggaggggatggtggaggacgcgaacaccaagcatagtgaaaaagaatgggaggacatgagggtatggtggaggtggaggtggtggaggtagaggaggcgatggtggaggtggagaagacaatagtggtggaggtggtggtgatagtgatgaaagaggaataggggaaggtagcaaagacaaggtgataggaagaggtggaaccaaactaaggaaatcaacagactcagggagaggaagaagagcacaaatcagtagttaaaaaaggacaagactcatcaaaggtgacatcctgagacacccgaataatgcagcgagcaataaggtcccaacaacgatatcccttacgctcagagtcatactcaaggaagacacattgaacagactgtgcagtgagcttggtcttgaaaaggaaaaaggcaacataggcaaaagcatgaaggtgactatactgcagtggccgaccaaagagacgctcaagaggagtgacaccatgtagagcaatagagggttcaatgttcaaaaggtaaacagttgtggacacagcttcggcccagaaccaaggaggaatagaggatgtcaggagtaatgcgtgagaagtcacaagaagatgatagtgcttacgctcatcaacaccgttttgagcgtgagcaccagtacaagaatactaaggaagggtgccttgattagcgagaaactgacggagatcgtgagacaagtacttgcctctagagttggcctagaaaacacgaatgtgagaatcaaattgggtgcgaaccatagtagcaaaattttgataggcagtgagcagttgagctggggattccatgaaatacacccctgtgaaacgagaaaaaccatcaatgaagatcacataataatgacgtccccctttcgaaacgaagggaacagggccccatacattagaagtgaacgagatcaaaaaggtcgagtagacacaatttgactactaggataaggaaactgtagccgcttgccaagattacaacccatacaagggatgtaggtatcaccaaaaaccttctcaataacactacgaaccagggtctccgaaagacgatcttgaatagagaaagagtcagactcaagaatgatatgacaatcatgatcagtgatctgactagctgacaacaattgcaaattaagattaggaacatgagaagcaaaaggaaccctaaaacgcgaagtataaagggtgacatgactaagaacaggtgggaaatgccattagcagtgttatcaaaccgggaagcagatgatggactcaaggaacaaagagaaggagaagcatgggtcatatgaaaaaagctcctgagtcaagaatctaggacactacaatacctaacgacgaggcacctggctatggccctggaaccgaggagtccatggaggaagctgatgaagtagcacaaaggtgctccatgtgctccatgtgccgagtcaggtcgagggcccggtcaagatgttactaaggcgaggcaggagcagtagtagcagcaggggcaccgccccagcggcatggaggacactgcctggacttctcgatggtatgtgtcgttgtcttgtagtagcggtagaagatagcagcaacagcgcccccttgggcaagaggaaccgctgctattgccgaagcagaagtaggagctggagagcggcaaggactgctgaggaagacaacaacataccacgaagacgagtctcctcaactatggctacggtgacaacctcgactagggacgggagaggagaatgagagaggagctgagcccgaagctgctcgaactccaaatggagatggtgaaggaactcgtggaggctaagaacatcagagagttgatgccacacgtcagccaactcgtggtagaagacatcgacggaggagtcctgttgtcgaagagagctggagccgagctccaggatagagatgtagagagtaatgcagcctgtgcccgctcccacatcgcctgagtagagggtagagcagagagatccatggcaaattccaccttcatactaccaagaagaatatgagtcaccatactgggagaagagaagaaaccttggccctgcttctgatgaaaacaaaatactatgtgacagactgggaattccatcttacaatattttagaaccaagctagcactgtcacacatttcagtgacgcaacaatagacttgaacaagatttgagtgagcaaccagtcaataggcatagttcagtagcaaaaccgaaccaaacaattcaatatctcattatgaacaagatttaagcaactgtttcgtatttaaaatattaaatcaacttgtccagcatcgaaaagtatttacctgctgcaattgacatctctttacaggaatcgcgattaattcgagtatggtaggagacattttatctatatatagacatccatccttttgtgttctcttcatttgtttcgtcatctcgttaatacgcggtcggaatttgctttacacatatctctcgatgcttatcgatgacagcttatcatttgttctatctaatgcctcctgcattgcgtcacatggtctttattgtaggagtttgtcaagcgccaaacacgtttcgtctcaagaaagccagccaagactgtagtagctacaattgaggttgttgctgagtcaatgggtctcaaggtccactcccggaactacaaggtacacttgaaacatcgattcaaaaacatctgctggataatcattgagcttttgtcttgtctccatctgctgaattgctgatacgtattttactaactttgcaggtgaggcttgaaggtccagcgtcaaacagagcgagccaatttgctgttgttctagaggtggtgaagccatgcatgcagcgcatcagttggtcgcttgtcctttacacagtgttatttccatgctctgaagtgcacttgattgaatcaggtctttgaagttgctccttctctgttcatggtcgatgttcgaaaggttgccggtgacactccggaataccacagggtctgttctgtttgcatctcacaccctcttactgatcatgttgttggacattgactaagttccactctggaatatgttgctggcattaataactaagtgctagatcagacgtttctctctctctccgttatgccttgtgaatgtgatttcagtcttgtaatgactgctaacctgcgtcttaaaccgattgcagttttacgagaacctatgcagcaaactttgcagcataatctggaggccaaccgaagtttctgccaaatctacgccgctgaggacgaccacctgctagccgcctagcccagcactgagctactgtagtggcatcaccaagagacgtgattcgtagccgtcagaaagggagagcttctgttgaatctagcattgctcgtggagctgggattcatgttgaccaccaggtgtttgtatattgtagtaacgctattgtagatagtctgtatagctccatttgtgcagttgtacgtagcaggaatatgttaaaaaacaacttttaatgtcaggtataatcggtttgcatccaggaagcatctccaacagtgtcttaaatcagtactctatttttaaatatagggttcaacttataaaaacagttttaacaatgccctattttatatttttttgtcgaaaaaagtataggacactatcaagtgacctaagtatactgcatcctatactagtgctctagccttgttttctatatcatttttaatatttcttttctaacaatgtaatttatttcaaaaggtacatgatttaggacctaattgttggagcacaatttatttttaatacctaaacattttacttttttaggatactataatgagtcgctttaaacaagcttgctcatgtggcctctacgtgactacatctgaggcaaatttctgccccgtttctatggcatttttatatttggatccatggcaaaaggctatgcaaaaagggaccctcggtaccgtgcatcggacttggtgagcccccaacattgtggtgcgcaacatgctcaatgtagaaatcagtaatggcctctggacactcacgtctctatgttctacgagtctttatctagaaaccatttttcacaatttattttttatgcctcctctctgcctaatcatctgcccccgctttcttcatctccacccatcactattgcaggaataaaatgtggttagttctagaaacaccaatagctagctttacccagactcactcatgaaggcaggagacgatgatcaattgcggcttgcgtgtgcgtagtgctgaatatc

SEQ ID NO: 142 - DNA, 5578 bp, Zea mays
ttagaaattgcagcaggccatgcatccatcgtgcagctactctatctacaaaatctacaaaaaggacgcgtttccttggtcgtcatcgcgctgctatctatcgcaggtcgccatcttccggacggaatatcaaaatgctgtggacgaaacatgagggcaccttcggtcgagtcacccaccgtttcgtgtgtcacatacataccagcgtctcgcgcgcagggatagaaatagatgtctggatgggaactcgaattattcgatccgctaaaacttgtaatatagctatctgtattcattttttttctaatataagtactaaataaatgtactagaatttgttttttatagccggtccaaaaccgtattggataaacatataaagtagtcgatattcatctcgagaacaagatataataaacctatttgaaagtttttcttttcgtaactagtgactaattatttgactagtttagttgtccatgtatcactacggtttttatatatgatatatggttttctttgtataacgcaaatcaatataagctgcaattgagacggtggtttgcatcctaactgctgcattttttttgatatattcactgacgcgcgataaatgcttacggaggggaatgatgcatgacgaaactccgacttgatattagtaaggatttaaatagtactaagaataaattgaaactatttacgatatctttcaatattgatttcatactattagtatacatgaatttaaaataaatttagtatttttctaatttaatttgaacctaacgtatctgttgttgtcaattattttaatagtctattttttgggaatataatattgttttagttcaatggtaaatattacacaaataataattgattatttggtatgtctaaatattaaatatttatgagtaactagcttattttatttaagtttattcagtttatctattgttttattaaatatccgtatctaatataatttagtttatttcaatgttagagaccattataaggctattttgatttttatttcatgttaattatcgatgaacttagtcatgaaattctatttatctatgttaaatttagcaataatacacgcgctggtctctgagttaaattaagtgaacattcgaatagaaacctgaatccagtatatttattttgaattcgtattcaaaaggatttgtactgaatctagattaaaatatgataagaagatggtgtccagatctgattccatacgcgtttcggtttgtttcggtttcgattactgctctccagacagataccgtgcccgacatgcatgttctaatcacacgcctccccgcccactgcatttcgcatcaatccagaagatttcgcagccaaagcagtatccaacggatgaatggtggtcaccagcccagcagccctcgactcgacgacgactctgtgagcgcgaccacaggtcacaggtgcttgcactgcactcatcctggtggtggagtgatggttcagttcatcagttgtggcttgtggcgccgcggcgagtggctgcgcgcgtgactgtttgtttggttcactacctcagttgccacactttgcctaacttttctgtctaatgttagttattcaattcgaacgactaaccttaggcaaagtgtggtatatttagccacaaaccaaacatgccaggcgggaagcgagccaaggccaggtccaggccaaaaaatctcacgcttcgcctgacgcctgctcctggtcgttacagatagagacggataagcatgacagcgcaggccgcgcggcgtggactccatgcctgcaaggggacaatagagatgccgtctgcgtccgcggcggcatcggcgccggcgtcaccccccgctataaatccgtcgcacccgcccacccacctgccgtgccagtgctctcatctgcgtacacggtctccctcttcctgtcagtagtagagtgagagtgaggcagcgagtaggagacaaggggaaatggggaagggagcgcaagggagcgatgcggcggcggcgggcggcgaggtggaggagaacatggcggcgtggctggttgccaagaacaccctcaagatcatgcccttcaagctcccgcccgtcggtacgtgcctcggttccatccttctttctcgctgcttattgtttgctctgttctaggaatcagagagatcgataagttttttttttatcaaatcgatcggtaccctgcacctgcagtacagccttgcaagttgccaagttcccatctttttttgcattgtcttatcgtgtttgcacgtgctgaacacgatggcgaatatgtagtcaggaatctatatcgaagatttggatcagcgtcagcgttttcctcccttttgggatggaattagtagggcatgttcttcttcgttttttaggaaacggtatgttctttttattaaaaaaatctatggtcaaaagaagggggatattgaactatttattgcacaaggtagttacagtatcatcagcactggatcgcgtgtacaggaaacatcagtgcatcttttccaagatcctaactactccagcacaagggaacccactcattcaataggctagcagaatattaaccgtgctaatgctacggttccgaagagcggggaaggagggaggacccggtggtggcggtgaggggaggtgtggcagggtcgcggggagagaggagaattgtactatatgtgtgaggcatgagaaaagcacgagtaaagaagaatgatataataatatcggttattacgtttgaataagaaagagaagggttatactttaaccgtttgttttgaaagtgtgttatgtatatgtgcaatttgtttgatgaatttagaggaggtcatgagttggaggtgatttggttagaagtgtttctcaaagtttagtctggtgggttgtatatggggtatagatatagataaaaggacagaatttgcagtaacttcaaagttcagatctgaattagataaaatcagtagtgcgccatacagactttgctgttcgcaatttcttttcgtttactggagagaattgcatctgtaaggtgtacgtgatattaaaaatgagcatttgagacatgccactatgaactcagcgatgcacagcacccctgagtgcagccactcaggaagccgtcgtttcgagctgcaggaatagcttctatagttattaacacggtaacacccttgctgttgcacagcgcatatctcagttcagaagaactgaacttatgtgtaatgctactgaggtgcagtttatcaacagctttcatttaggacttaggtgtggtggatgtagctgttccaagtagcaatcaaatacggcctgaagtgctaaaacaaaatagaatatcagaacttttgtaggctggtcgcataccatgtgaggaaattctttaggtcggaagattagtacttattacaactgaataataagtatgctgacagtgaattttggctggcattttcaggcccttatgatgtccgcgtgcgcatgaaggcagtggggatttgcggcagcgatgtgcactacctcagggtgcgcgatcctatccgatgtctctgtaattctacggcgcgggaattgttgcacggctaatggatttcgaccctttacgcatcatcgattctcgcaggagatgcgcatcgcgcacttcgtggtgaaggagccgatggtgatcgggcacgagtgcgcgggcgtggtcgaggaggtgggcgccggcgtgatgcacctgtccgtgggcgaccgcgtggcgctggagccgggcgtcagctgctggcgctgccgccactgcaagggcgggcggtacaacctgtgcgaggacatgaagttcttcgccaccccgccggtgcacggctcgctggcgaaccaggtggtgcacccggccgacctgtgcttcaagctccccgacggtgtgagcctggaggagggcgccatgtgcgagccgctgagcgtgggcgtgcacgcgtgccgccgcgcgggggtggggcccgagacgggcgtgctcgtggtgggcgccggccccatcggcctggtgtcgctgctggcggcgcgggccttcggcgcgccgcgcgtggtggtcgtggacgtggacgaccaccgcctggccgtggccaggtcgctgggcgcggacgcggcggtgcgggtgtcgccccgcgtggaggacctggcggacgaggtggagcgcatccgcgcggccatgggctcggacatcgacgtcagcctggactgcgccgggttcagcaagaccatgtcgacggcgctggagtcgacgcggcccggcgggaaggtgtgcctggtcgggatgggccacaacgagatgacgctgcccctgacggcggcggcggcgcgggaggtggacgtggtgggcgtgttccggtacaaggacacctggccgctgtgcatcgacttcctgcgcagcggcaaggtggacgtcaagccgctcatcacccaccgcttcggcttctcgcagcgggacgtggaggaggccttcgaggtcagcgcccgcggccgcgatgccatcaaagtcatgttcaacctctaggcgggcaagccgcctccctctcggtccagccgtctaggcgccgtcgccgtatgcccctgcccccacccaggccacaactcctggaataaaaatgacagaaaagaaactttatagttcgatgaatgggcagtttgccgtggttttggaataatttggacttcgtcttttttcgcttctgttgtcgtaccattgtcttaatcgatttgtggattttgaagtctccttttattgccaaaagttcgcgagaactaagtactcactccgttttaaaatcgtattagttttagttttcaatttttatgtctaaatttaaatgtataatgataaacctagatgcatttataaaacacaaatcaagtattatataaatctattactttttctaaaataaatttaaatttaaggcggtgggtattaaatagtgaaagaaaagggagatagccgtttttgttgccaaaagttactctgcaaaggcctaaaactacatgtgcgggcatcaaatctctgtaacacgcgggattacaagagtgtctccaatagcgttctctatatacattttctatatcatttttaaagattacaatagaaagtttctcgtcggacaatagctttccagccccgtcttcatccccacgaacgctcacggacaatagaaaatgtattgtttggacacaaatttaaattgatcatatcaaatcaataaaaattgaataagtaagatttaaaattacaagagccgtcaactttagagtttagtttgttgtttttataatagactatatattcaggttttttgtagaatttgaacatgccaattgactagtttagatcaagagacgtttgcggggatcagggataatttcccataccaaccatccccttctctgcgggccctatgtggactatttttttttttttttttttgctgtttggaccctatagcgaccaaattactcatatccatgtctctaatagagaaatgagatatggccccgtagaa

SEQ ID NO: 143 - DNA, 3950 bp, Glycine max
gccatccttaaataagttactccagtataatttttaattttttgtaaatcccctccttaaataagttatctgagttgaattaatgaagttccgaaaactgaattctaaatgcaatcagtctcaaataaaaaatagtttttttgcagtagactaaaaccaattaacaattttttatatgtacacacttaagaaaaactttgctgataaaaaaaaaacttgagcattaactttaatgtcgtaaacacacaatgatcttctctatgatgccccaaatttcttacttagattttgttaatgtttagacacgcatttttaactgatgtctttgggaattttcatgtgaaatgttaaaaagaggctaaagcaactattgaaagtagctttaactagcctcagatccattttttccaaatgcatatccaaacatgttttgttagtggtactaaattgcttcctaactacaactttttatttatttgctaccaaaagtccaaaacacaatcttcttttgttgttgttgttgttgttttttttttttttatcaaacatgatgttttaagtatactcttttaagagctttataggataagtcatttttaaaaaagtgtttttaaagtatgcaagtgtgaattcatcgctaatttaaaataaagtgctttataattttaagacttacatatatataagatccttgatatatatttgatttttgttttgagtctcagttaattgttttttcattgttttgagtcattgatatattaaaagatttgttctgagtatctgtcatgaatgagtcaactaatgatgtgacactatttcaataattatttcaatatataatagtcgttgagatgatattatatcatcatttaactaatgacaaagattcaaaccaacagaaaaaattattaaagacttaaaacaaaaatcaaatatatattatagaccttgaaattatttaaacctaattttaatgtacaaaattagtacacattcaacaataccataatttaaaattttatttcttccaatctctttttttttaaatgatttacacacaagatcagaaacacagacaagattaccctgtggcctcttgttgaggtgctttaaaattaagtgaacaaaaatgacccacgtgaatcatgttaagtgtagagacttctctgcagtaataaagaaaacttgactaaacgtctaaacgcagaagatttgttaaagtaatactattagtctaagatatacgtagtaaagaaaaaatggtgtcaataataataataatactaaattaatcattaagctgccacagtaattattattaaactagcataaattagagtttaatgaagaaacgtgattcatgtaaccgacacaactctagaggacaaaaccttgaagttaaaaaaatgatgacagtgataacgaccatatgaatatcaaatgcagcattctcactcacataaatcattaaaattgtgattaaacaagacaaataatgaaagattgaagactacgcacgctggaatgtgatcacacattaattaagatatatcattatattatttattattaaattataaattgaaccaccaagcaaagcaagccacacaagccaagccaacatttaagaggctcagaagtaagaaccaaaagtagtgcacaaagtttatctcaaaaaagctagatgaaaaatacatataagcataaacgtcactaagataagaaaatagggtaatattcacagtgtacaggtggaagaacattgtatgttgaggtgtcaacaggaccaatgagttttggatataaagccaacaattggggacttcactctgaccaccatgataggaactccaataagcaaaagccaattgatacgtgcatatcacaaaaactactcttacacaccaaaaaaatcctctatcaccaaactcacctataccctcaaactcaaggccctatagtccaagcaagtgagtgagtgttacaacttacacagcatggaatatagtcaatatactacttattcagcagaaggtgttgaggcagaaacttacacaagtagctgcaccaccccatcaagatcaaagaagagaaacaacaacaacacaagaaggttcagtgatgaacaaatcaaatcattggagaccatgtttgagtcagagacaaggcttgagcctagaaagaagttgcagcttgcaagagagcttggattgcagccaaggcaagttgctatatggtttcagaacaagagggctagatggaagtcaaagcaacttgagagagactatggcatactccaatccaattataacactttggcttcacgttttgaagctctgaagaaggaaaaacaaacattactaattcaggtactttatacaatacaaattattgatatattcttgagaaatgagtagggttaatacaattcctttatgtattgttattgttgttcttcaatcagaattgaagtttcgctttgataatccgcgtcataaagatatgtattgaaagattacgtggattaacgaagtgaaattttatattacatggttataacttaggtatctattttcaacagttgcagaagctgaatcatctaatgcagaagccaatggagccaagtcagagatgcacacaagttgaagcagcaaacagcatggacagtgaatcagaaaatggaggcaccatgaaatgtaaagctgagggaaagccaagcccatcatcattggaaagatcagaacatgtacttggtgttctgtctgatgatgacactagcataaaggtggaagactttaacctagaagatgaacatggccttctgaattttgttgagcatgctgatggttccttgacttcaccagaagattggagtgcttttgaatccaatgatctatttggccaatcaaccactgatgattaccaatggtgggacttctggtcctgaatgaaacagaataaccaaaattttgtggaggaaatatatatatatatatatatatatatatatatatatatatatatatatatatatatatatatatgatgaatgcttcatgtttggttccactttggcgacttgattcgataattgggtagcaattagttatagcttccatgtttttaagcaaaaagcgatgcaattttttttattaataacgagtttccaaacacctaaatcaataattaagagaacatcattaagcaccacctaagataaacgcagaattaaaaaatatttcttacatgaaagatgtgtgttatgtctggctattagttatcatcaattccacaaatttctgtggaaaaccaaactgtacttacactttaaaaggaaaaaaaaaatctcaaatctgaaagaagagattttattaataacaagttcacagaggttcattgctacatctttataattaaagctgtatgtttggctttcttatggcaaccgaaacataaaaagttcacactaacaggtatccaacatgaagggccaagacgtgccccactccacattattatggtggtgacacaaactataaaataagcatttgaattaatgaaaagcagctaccacaagtatggtgttcattgcctatgaaaattgatgaccaaattggagccaaatatggctattccaaaatttaaataatattcaaaataagacctagactgtttaatttcatgcaaatataaattccattcgtcctgtgaaatagaagcaagttgttcaatgattatatcaaactaggaaatcctcttcacactttcagagcataaccgttccaagaaatagaattaacaaaatctcatgcctaagaacttcggtgacttttggtcaaatatttatatgcaattgcaatatgacactgcctaatatattcataaaaataataatgttttcccgtgtatatcgttatgggagcagataaagatagaaaggaaaagaa

SEQ ID NO: 144 - DNA, 6854 bp, Zea mays
tgactgtcatgtttgtcgtgcgctgtctactgtgataatgtgcaaattgtttattgattgataaagctagttcaatatcaatgtaccaagtacattgattcattttattcgagacaaggttgctagtggagttgttcaagctcttagcgtccttgcatcattttactggttatcttatcgaaatagaacataactcctttttggaacaagagtttcgtcacaactagccagcggtgaagttttatactatgtacgtttttataaaaagctgttagactctctttaacgtctcatgaagctacccctcacgcctgctccgcgtatgaccggtcactggcacgctccaacagctccatctcctcctcactctcttctcaatgagaccatctccaaccaggggcggtcccaggatttaaagtacgggtattcgaaattttggtagaacatattttttttgacaatctaaaatcatattattatattgtatgatctgttgcagggtagagaaatgaagagggctagtgaaaaccaaagtttagacaaaatagctgtaaaatagtatgattttatgccttctataatattttagcttatatacataacatatacatgtatatataattgttttgccaaaaataatgggtattcaattgaatacccttgcttgcatgtaggcccgccactgtctccaaccatccccacaatcccccctatatgtactttctattatatttactacattaccctaaaagatacccccacatcgaggacgtctccaatcattaaccctatccaactcttcatatacactataatccagttacttgacctctttcatcagtttttaaagttctagaaatcatagactattcatactgccataatatttattgttttcgggttatgataattaaaggtgttaatatcataaagaaatagactaaataaaaatgtggtggaagattataactcacccatatgtagggggaggtgcctatctccctcatgtggggtgcgctgtagggggaccgttggaggtgtttgtcgccccataagccactggacaggggtgggggggaaggttacgaggtatggcccgagagaagatttcttcaccccgtcgagagggatagctctatcctcccccttttcgctaatcaacgataataattacaaatttaaatatttttgggtttaattagcagataacaatatgaataacgatactacgaatattgtacattttttaaaactccaaaaaatatgtataaaagttaaatagcttagttaaaggttaaatgagagccaatgaaggaaatggttggagagtagatgaaatagaggaaagaatggttgagtgagagatatttaaatacgaatagaggatttggaactgggagtggctggagtcagccttatacaaaatttgtactgtccgaagcgatcctcatcaggaagggcgcaggattcgtcccagctaagcgcaccggccaagtactacaccagtgcagtcaatagctaaagaaattgggcggcaagtgaaagtctctgcgggacggaatgtgcatgagtcatgacgcgcctcctccggagttgtttattcgcggcccgccggtcccgtccgtgttctctgttccctcccctcggcacatcgtcaccctacccattttctttgtctctctctcctatcttcctcggtgtcccccctaatcccttgcctactttaattccccgctactaccaggccgccaccatcaccaccctcctcctatctcctgcaggctgcagcctataaataggccagcttcgccaccaccggccacacaccacttcattcatcccaccttccattcctcttcctctctctctctctctcccgtgctccggtagctatagctcttggacctccaagaagcacacggccggagtgatcagtgaagaaacggccggagtgatcagtgaagaaagagaaagcataagaccgggctggtggcaatataatgacgcggtgcctcatgttcatgccgccgctgttcctcgtgtcctccctcatctccaccgtggggctgccggtggagccgcccgcggagctcctgcagctcggaggcgacgtcagcggcgggcgcctcagcgtggacgcgtccgacatcgcggaggcgtcgcgggacttcgggggcctctcccgcgccgagcccatggcggtgttccagccgcgcgcggccggcgacgtggcgggcctggtccgcgccgcgttcgggtcggcgcgcgggttccgcgtgtcggcgcggggccacggccactccatcagcggccaggcgcaggcgcccggcggcgtggtcgtggacatgggccacggcggcgccgtggcgcgggcgcttcccgtgcactcgccggcgctgggcgggcactacgtggacgtctggggcggcgagctgtgggtggacgtgctcaactggacgctgtcgcacggcgggctcgcgccgcggtcgtggacggactacctgtacctgtccgtgggcggcaccctctccaacgccggcatcagcgggcaggcgttccaccacgggccccagatcagcaatgtctacgagctcgacgtcgtcacaggtagctagctagctagctcgcccagccagccgttttttagctgctccatggccatccaaattcccaaggctcctactgattgtcccgacgagtacatgcaaaggcgctgtggaccgctgaatgaaccgactgacacacgtgcgctgtgcgtctgaattggcagggaagggagaggtggtgacctgctcggagacggagaacccggacctattcttcggcgtgctgggcgggctgggccagttcggcatcatcacaagggcgcgcatcgccctggaacgtgctccccaaagggtaacagccacaacaacatccttctctctccctcctgtctctccctctctctctcgctcgctctcgacgtgttctctccatctcgctcccttcagccgcgcgcgcgcgcactgatggatcacctgcatcggtcggttctttcgtttccctcccgcgtgacggcgtgagtcatgagtggcactggtgtggtgtcccgatcgtgtggcagtggcacccggaaagaggccggtggcgcgctgggcagtgtaccacgctcgccgtcgcatgtgtcgccggcctcgctttccaacgagtgaccggccgagcagggatctcaaccacactccacaccggccagccatgcaagtggcggtcgccgagcgcgctgctgatagcggatagccctctgctaggtgcaaacgactagctagcacatgtccgggaaggcgccccacgcgtacgtacgtcttgctaatcaggcagctagctacctgaacagtcttgtcgttacactgttcgtcagagtttgccggaatgatatgggtactgtacatgactactcgctagcagtagcaagtacgccaccccggtcgatcagccgtcgacgtcgtcgtcgtcgtcatcgtccgcgccggtgtttgtttagccccgtgcgggggatctctcgcaagattttattcctccctctaattaatgcatggatcggtctgtgtgatccaaggagaagaggcagctaattaacggccggaattgaacgcatggctgctgctggtcgctgcaggttcggtggatccgggcgctctactccaacttcaccgagttcacggcggaccaggagcgcctcatctccctgggcagccgccggttcgactacgtggagggcttcgtcgtcgccgccgagggcctcatcaacaactggaggtcctccttcttctcgccgcagaacccggtgaagctcagctcgctcaagcaccactccggcgtcctctactgcctcgaggtcaccaagaactacgacgacgccaccgccgggtcggtcgagcaggtccgccctcgccgccgcctttctttgtttatgtggccggcgtgctctgatatactaagttgtttgggaaaccgccccatttcggggtggcgagcagcgagagctaatcattggagccgtgcccagacgagtatggctggctgctactagatcttggtccatcggtagagcaagtattttattttattttgcaggggcggcggcggcggcggcggcggcaaacgacagcacgggggaagggaaacatatactaacgaaatttggaattcttttttcgttcaggatgtggatgcgctgttgggcgagctgaacttcatcccaggcacggtgttcacgacggacctgccgtacgtggacttcctggaccgcgtgcacaaggcggagctgaagctgcgcgccaaggggatgtgggaggtgccgcacccgtggctgaacctcttcgtgccggcgtcccgcatcgccgacttcgaccgcggcgtcttccgtggcgtgctggggggcggcaccgccggcgccggcggtcccatcctcatctaccccatgaacaagcacaggtaacaagtcaacccacaaattaaaacacgggtcctcacaagccaagaaagtgctccgtactagcaggcgcaggcgcatcccatcaagcctgccgcgcgcctggctggctggctgcgtgcgccacgcatccatctgccgccacgcacgcggggggcggcgcgtcgccgtcgccgcccgcgcttccaaccgaaccaaccaaccaaccaaccttccatcccatcccatcccacatgcggtgcacgggacacgtgaacagcgcatgagtcgccacgtttccgcgccgtccgacggcgccgcccgcctcgctccctcacatgcggcgcacgcacgcgcacgcgcgtcgcgcggacggcaaccgcacgcgcagcgcagctgtcgtctcgcctcgcctcgcctcgcccagggccgtcatgcgtcttgatggtgtttgcctttgccatgattggcgccacgcacacaaatcaccatcccatcagcagcagcgggtttttataataattgactaattaattgccgaactgcgtgagccgaatctggcgccccgattgaatggccgcggcggcaggggcctttttcgattctgttgccatggacgccacgcaacgcccgtaaccccggcgcgcggctggtgtggtggcactggcgccggccagccattttttttaaccggcgccgcgagagctttccagtgcgcagagagcccggcgcggtcagaactggaatttcttacccgagacgtgatcgcgctttttttactttgttttgtttggggtcagtgcctcagtggccaggtagatgcaccgattgttttgttgctttcaagcttcggcgcatcgcatacacaagctggcaaaacctaaaaaggcgtgtcagaatcgtcaggccatactgctaagaaccgccgcgtgctttattgcattgcatactctattgtctttgtctgtctatcggccctccaaaccgttcgagccggttaggtaataatctagcaagcaatcgcacagttccgaaagctcaacttgtttgtttcttggtgcgcgcaggtgggacccgaggagctcggtggtgaccccggacgaggacgtgttctacctggtggcgttcctgcggtcggcgctgccgggcgcgccggagagcctggaggcgctggcgcggcagaaccggcgggtcctcgacttctgcgcggaggccggcatcggcgccaagcagtacctgcccaaccacaaggcgccgggcgagtgggcggagcacttcggcgccgcgcggtgggagcggttcgccaggctcaaggcccagttcgacccgcgggccatcctggccgccgggcagggcatcttccggccgccgggctcgccgccgctcgtcgccgactcgtgatcggtactactgactgattattaggcgcgttttagtgtaggtagtagctacagcggtaaccgtacaggattatttagtttgttgttattattattattattatttagctccggttgatgtacaaatgtgggtcacgtgattctgtacatgtacatgggagtcaaatatgaatgtcgccaagtgatgctcttctcttctaatggttaaatacaggtgcctgactctctctgattactgttgttggtgttttgattagtgctgggaattggaccggtgttggaacttgctatcaaccgcaaggaggccaacaagggtgaacagtggtgacagaagggttacgcgtgggatggcaaatagctccgttaaccaggcctctcacgggcactgtgcgggggtatttataggtacctgaacgcccaacgccttgtgttaaggacgcatgtgccctcagctacctagtttatccccagaatattcccataaagcagggttacagactgtaattacagggatgcctttacgaattaggcccgtaacacgcggcggctacgcagggcccgttacaatggaccggatcgcacgtgggcctctgagctggacgaggccgcactgtgggatgccttcgtcgccggtcttcgtctggtgccgatcaagcgaaggatgcccctgcctgctttgtctctcagagcagcggctaagcagcggagacttcgagcgaagggtggcgtttctgccttcgctccaacagccgggtcagcccggtctaagcccattgtgcttcgtgtcgaacgggtttgggccaacaaaactcaaaagatttttgggccgtgtcgtgccggcccgaagtgtaaaaacagtggtccagcccggccctaaaccacgtcgtgccttctttaggtcgtgccggcccaagcccgacccgtatatttgaccatataaaatctaaatattacaatcatataaagttcatagcttagtaaattaaagatattaaacaatt

SEQ ID NO: 145 - DNA, 5775 bp, Zea mays
gttctcaactagggcctaaaattctcaaaatatctgttggggaccattatcgtcgacgatcctcagaatatgttattaccaaattaaaaggtgtgtttcaggtactgtgcaaagcagcagcgaagctatccttcgtcaaaagtggctcaatgaaccaggtggagaagctatggagcttcgtctgcgtagagcgtgccgaaggaggaagctttggctctgaatgcatcgacttacgaagcatgggagaagaagactcagaaggcttgtccagcgtgggaataaaaaggagaaaatacaattttgcccttgtgggatttgtaaatcatgtgcaaggctcatggatatgtttgtaattttatatgatatgtttgtaaatcatggatatgttttgtaaatcaggtggactagaggagagggagggtggacatagtgacttgcatcttgatcatggtagagtggtcatggtagagggaaaggggtaggtcaattctggagtgcggccacggtggcttgagtgtcggccatggtaggggaaaggggtagcccaattctagggccggcatcagggaaggccgacatgtgcacgtcaggaggtagtgttagaggtatgaacggaaaaaattgaacatgttagtatgatgagttgtgtaattgctgggaattgtggataatttccacttaactacggccctgtttatttacccctagattataaaatccagcttaaaaaagttgagatgtaaacaaacaacatatattattaggtggattatgttatctagaaatctggatgataataatttataagtcggttaataggtgtttacataatcgataagctggattatataatcctggaacacggcttttgcgagagcgtattaaaacaggattccgtgaagcacactatctgaggagctccaccaaaagctgaatctagcccgcactcttttttggaggattcaaatttggtgtcactggagcattcggcattttgtttcatggcgtgaagctatttttactaattacagaagctgtttcaaatagacctttaaatgatggctgagtataaaaggaggcaatttttttatctcgccgatggagccaggtcgcgtcgcgccgcggccgtgctgcgctctcgacgcgatctagcggcgatgtgcacagtacagttttgccatgccattggttaagcctgcatacaacacaccagcgtactgccctgcacaagatctcctcggctcggcctctcctgatggaacgttcagcttgaacagcggagcgtgggggcatcccggggatgggcgccgcggccgagaaattttgcaacctggcgaatctgccctgtcgcatactaccatccacccccaggcgccaagaacgcctccgagtttcaggcttgcagctcagctctgtgttgaattggaacgggcggagtttctgggttccagacttccagtacaaggcgatcaattggtagggcgaattacttgcaggcccagatgcatggcccatctatctggttctctatcggttgcttttacttgcacaatagtggcagacaaactacaagtcagatccgatcctatccatccatccatctcgcagcgcgatgcaaatatgcaatcgtctgtggaactcgaaaaaaaacagaggtccggcctcgcacgaggttaagggaaaaaaaacgaagcgtttggaactttggttggcattcgcagcatgctgtgctgccaccgtatgtttttatttttgctttgtttgtcttctttgagaaacgtgagggagccgcgtgtccgctcgttataaaaccccccggcgacccaaactaccacgagctcaagcctcaagcaagcagagcgccgtgacatcacgaaacatatagagctagctgctctgcctctgcttcaccaatcacctcgcggaggggaaggtttccccctttgacacagccgagctcccctccatcagcagccagctcctcgtcgcaaagcaagaagatgatgctcgcgtacatggaccgcgcgacggcggccgccgagccagaggacgccggccgcgagcccgccaccacggcgggcgggtgcgcggcggcggcggcgacggatttcggcgggctggcgagcgccatgcccgcggccgtggtccgcccggcgagcgcggacgacgtggccagcgccatccgcgcggcggcgctgacgccgcacctcaccgtggccgcccgcgggaacgggcactcggtggccggccaggccatggccgagggcgggctggtcctcgacatgcgctcgctcgcggcgccgtcccggcgcgcgcagatgcagctcgtcgtgcagtgccccgacggcggcggcggccgccgctgcttcgccgacgtccccggcggcgcgctctgggaggaggtgctccactgggccgtcgacaaccacgggctcgccccggcgtcctggacggactacctccgcctcaccgtgggcggcacgctctccaatggcggcgtcagcggccagtccttccgctacgggccccaggtgtccaacgtggccgagctcgaggtggtcaccggcgacggcgagcgccgcgtctgctcgccctcctcccacccggacctcttcttcgccgtgctcggcgggctcggccagttcggcgtcatcacgcgcgcccgcatcccgctccacagggcgccccaggcggtgagcgcgcggacatcgggtcggggcgaaagctaaagcttgctttttgcttgggcactactaactgactgacgttgccattcaggtgcggtggacgcgcgtggtgtacgcgagcatcgcggactacacggcggacgcggagtggctggtgacgcggccccccgacgcggcgttcgactacgtggagggcttcgcgttcgtgaacagcgacgaccccgtgaacggctggccgtccgtgcccatccccggcggcgcccgcttcgacccgtccctcctccccgccggcgccggccccgtcctctactgcctggaggtggccctgtaccagtacgcgcaccggcccgacgacgtcgacgacgacgatgaggaggaccaggtaggtagcagtaattgccaacctctccccccgctgagacttggcgcattcccgtacttgaccccctcgcccgctctggcgtgtacttttccgcgggcagggcatgtctgactcgcctcgtcgtgtatctcccgctggattcggtgacgggggggctgcgtcctgccaaaccaaaccaccctagactagacagacccccaggggcaggggtcgcgccattggccgcacgcggggaccggcgccagtgagtgcgccgcgccgcacggccgcgccccgatctcgctcgctcgctcgctggtgatcgaatcggcgcgtacaatgcggcatggccccgagccccacacccgcagtggccgtgacgcgattgcgctgcctccggtccggcccatgacccagcggatcgcgtcgcgtcttttggcaacgcccgcgtcatcatatcgcgctctttgtcgtccccacggagcacagcgcagcgcagcgcagcgcagccaaccttttctccgccacgcacgcttcggcggcattcattatttggattttgttcctaccggtcgatccgcgtccgtccgtgcactgcaggcgctaccgtcatgctgaccaacccattgccattggttttgtttcttctctctctctctcgctctcgttggttatggttcgtgcgtgcctgcaggcggcggtgaccgtgagccggatgatggcgccgctcaagcacgtgcggggcctggagttcgcggcggacgtcgggtacgtggacttcctgtcccgcgtgaaccgggtggaggaggaggcccggcgcaacggcagctgggacgcgccgcacccgtggctcaacctcttcgtctccgcgcgcgacatcgccgacttcgaccgcgccgtcatcaagggcatgctcgccgacggcatcgacgggcccatgctcgtctaccctatgctcaagagcaagtgagttgccctccgctccgctccgctccttcgccctgcgtgcagtagtacagtacaggagtggctgagtggtggtactgccattcagtgtgcagttgccgtttgcggcccgccaagctagctaggggccgggacgcatgtgagccgccctgccttctctctgctcgtcgtgtcactgacgcctggtcctccgggacagttgctgagccggcccgtacgtacctgtaagacgacggtcccgagcctccaccgccgcttctgttttggatttagccgtgtcacacagatcttacggaggaggaggagtactatgattgacaaattattgcttcgcccgacccgaggctagcgcacagtccatgtcatgtgggcctggctgtgtggtttccgtcctgatgctgatgcctgaagggacctgcgtgcgtgtgcgtgcgtgcaggtgggaccccaacacgtcggtggcgctgccggagggcgaggtcttctacctggtggcgctgctgcggttctgccggagcggcgggccggcggtggacgagctggtggcgcagaacggcgccatcctccgcgcctgccgcgccaacggctacgactacaaggcctacttcccgagctaccgcggcgaggccgactgggcgcgccacttcggcgccgccaggtggaggcgcttcgtggaccgcaaggcccggtacgacccgctggcgatcctcgcgccgggccagaagatcttccctcgggtcccggcgtccgtcgccgtgtagagcaaggggggaggaccagccagctgccagccaagacaggaggaggaggaggaggggaggctgatggatcgccgctgctgttgccggtaatgatggcgattacgctgctgatcctggtgatgatgatggacgatcgaggaagccgcagggccgggcaatgatggcgatagggccaccgttaggtgtgcatccgggggcgcaaattaaagggattgctgtgtggagatctgcacgagtttttgctccatgcatgcttgccgttcgtgtccgcgtgtccctctcccccttgttattattccttcgcccgccgaggccgagcgagcgggtggtggcgacgctggatttgtctgctctgctttgctccgccgccgtggccaccccggtggcgtgcgcccgcaagctgttccttccgcgcgcttctgttccgtttcgttccgttcctccgtggtagcttccccccctcgccgtcctggtcccccccgcccggcaccccacgtggcacaccagcccgatccaaacgccgcgaccgcgacgcgcggggccgttggttcgcgttcccgttccgtagtagcttggccgcagtacacgacgaccgcgaacaaagcgcggccaaaaccgacgggtctcgccgccgccgccgcggacgcgcccacgggacaggaggaatatcactctggggccatccgcgcgggaccagagaactggtcgggtcgatcgatatcggcactgtgctggctggcgacggggaccgagcggcagggacgtgacggttgttgccgcccgagcgcgacggcgaccgtcgttcgtctctgggccggggcggcgcgggcggcgttttcgtttggaaattttgtggacttctacttgtatatataaaaaaaacgatcggtacgtatacaaccagtcttcctttccctgtcgtgcccagtcgcattccgtgatgcgagccggatcgcgacggaagcggctcgacgagcgtcgtccctgctgcaccctgcta

SEQ ID NO: 146 - DNA, 4779 bp, Glycine max
gaaaaaatgatgtgataagaagagaaaataaaaagaataaaagatagtaatgagatgtttaaaataatgagatcttcatatatcattattttataaacaaagagtaataatacacaaacacatttcattattttcaacacctctttaatacttctcatttatttttatctctctttttctattacatcataaatcttatcgtacctatatttttctcttttttctccctctctctaggtattaaataacataacgggtgttcatgtaatatttttcataaaacaatatgaatttttttaaatatataatgggaattgtttcttgaacaccccatatactttctttgatcataattataaaactctttttgaaaatttgtttgtccttttttgtaagttttttttttaatttatagatgcattaattcttttttccctacatactcttaattattctttctttaaacattaatgagaaacaattaagtggatagagagataataaagggtaattttggaatgataatacacataattaattgatagatttaatataattaactatttttcttaaaaaacgtgaattaattgaaagaatcttaaaattagggacgaatagaatactgatatacaccaagagagagaaaaaataatagagaaaaatggtgttcaagaaacatttcctatcatgagtaatgatatacaaatacctacttttctaacaccactcatttctttccatctctcttttcttgtgacatcataaattttatcataccaatattttctttttttttctctcgttctctaggtgttaaataacacattgagtattcatataacatttttcatgaataaatatgatttttttaaaatatataatgagaattatttcttgaacaccccatatactaatatacacctagaaaaaaaaatcatgtaataagtactctcccaatttttattataagatctaattaattaatttatacatgttaatatctaaactattagagtgttgatgtatcacggttaaaggtgtactgaaattgtgtgaatcttaaaaaaaaaaaaaaagtgagatggcatgtgaaaccccttctccatcggcactattctgtggggacaaagcatgaaaatttagtcatgcaatgtcaataggaggctttggtagagtatatagcaaatgaaacctgatgaggggtagtataagggacgtgatgagtgatgtgatgaggggtagtataagggacgggcgtggaggtttgttagttggctttagttccctgtcaactcgcacctgcgcaccacgtgaagtgctacaactttaaacttcttctatccactcatttcttacattgcatatcatatgctaaataccttcactttttttttctccaaaaaaagcaaagattacgaaaaaataaaaaaaagtaaaaagatgctttttccttctttgtctacaataaattgcccttctcggattctttatctcttacaccacaccacatcacatctagctatctttctttactgctcactcaggttcccttttcactctctttctctccctgtaatgaatttactgtttgtgctgtctttctcaatctgtagtactagcgtgatgaattgccaattgtacaattacactatggtcactctcactctctctcacacacacactcgaacacaaacacacacatgttgcatcattagttaaaccctgtctacctcaatcactctctactaaacacaaagtatatctcaaattaattgttggattttgtttgcagggagaaagaaagaaagtaaaatctaaaacgcaatgaaaaggctgaaatagatcgatgatatatgctgttgttcaatagtagagggagggtgaataagtgaagtactacacatcgttctcaatctttttcttaatagtcctataatatctataactctgcacctccaacgagggtagtagtaaagtagggttatatttggtgcaataatatgcaatctcaaagttcagatacgtttccctctcacaatggcaatgacccaatttcctgttatgacccagcattttactactacaatagcaggcccttttctcccattgactctgaaagcaacccaacatcaaaacaagacaacaacaacaactgtgacctcgttccttctcctactcctttgtcctttttccactttccttcttctccctttgaagacactcaaaatcaaattttgctcgaacaacaccacgattttctccttcagtttcaccatcaatctctccccaaagaccccgtgcctcaaccggcagttatcaccaccatggatccttttgttaaaaagagtgaccagatccaaagaaaaagacctggcaagagagacaggcacagcaagatcaacaccgcaagagggttgagggatcggagaatgagactttcccttgaagttgcaaagaggtttttcggccttcaagatatgctgaactttgacaaagcaagcaagaccgtggagtggttattgaaccaagcaaaagtagaaatcaaccgtttagtgaaagagaagaagaagaatgatcatcatcatcaaagttgtagcagtgctagttcggaatgtgaagaaggtgtgtctagtcttgatgaggttgtagtaagtcgagatcaagaacaacaacaacaacaacaacaagagaaggtggaaaaagttgtaaagagaagggtcaaaaactctagaaagatcagtgcatttgaccctcttgcaaaagagtgtagggaaagggcaagggaaagagcaagagagaggacaagagaaaagatgagaagccgtggagttctagctgaagaatcaaagcaatgtggagaggaaacaaatcaggatctgatccaattgggttcttcgaacccctttgaaaccggagatcaagaatctggtgccaagacaagtcacagtgttgatgtgcatccttcttccttggacgtgattgctactgaggctaaagaacaaagctaccgtgcagtaaaggagcataatgatgatgatgatgattctttggttgttttgagcaaatggagcccctccttgattttcaataactctggattctctcaagatgtaagtttctattagtgtctggtagattataacttccttcaaattattaaataaatgtaatgctgctgcattcacgataatgtttagtcattttttaaaattgagggaaaggaatattattattattattatattaagaatataagaagtaatcaagcccatacacataaatattattaacagaatcaatatctaaacttttttttgttgaataatcaatatctaaactaaaggatacacaaaccagaaaatacaagtgaagggattaacccaccacatccagtcaaacaaacaagtagccttattttttaaccaagaaccatctctaaacaattcatcttaatcccattattcccatataatatttaacaaatcatacctcttatagttctttgaattagattcaattttttttatataatttattccaatttcatgttaaaagtcaaaataataaatatctgttgccactttttaaatattttttaccatgattttcagaatcaaaatcaattttgataactatcttaaaacattgttaatattttatatatgcaaagtgaatgctcaaattgcatcaatatataacattctgctgactagtcaatgaaatttttgcagcaccaatttgcagaatttcagtccttaggaaagccgtgggagacctaaaacaatcacatctttgatgtgacaacagaaatgcgcgagcctgatatgtatttgtttactttcctttcagaaatttaacggagtaaggtactttccacttctacgaatctggtaaaaaatcaatatataaaacggttctattttcatctctataccacgtttccttttccatgtttctgctctgatcctggctacgtacttactactcagtgacatggtacaaattcaatagtatctataacggccttattcatgcttttaattaagaaaaataactaattacatttttttttatctcaacctgggttaatgcattcattataccacttggaacattttagtgaagatgtttcaaatgcatttacatatttttttgtcttctttgtgatggtattgacacatttcattattttaaaataagtccaataggaatcggataatattgggtcacttctcagatgtatattttagattgtctgtgggtagcatccccagtgtattccaatgtgttatacttatctttatagacgtgactgaatagacacttgggtgatccacacatttccttcaaattcaagcttaaaattgacttcgtcatttgaatattcaacatagtattatctgctcgttagaaaatattaaggaatataaggatgtaactaaacatgttaaaggaaattacttaaaactaaaatatatttaatgaataatgttataggatattcttgaacacactttttatatacattttctattggttaaatttattgaaagttacataatgatgaatatatataactcattaaacaaagtgtgagcccttattacttttaataaaatttcactcatcaataaatattgttatcatttctgaaaatataaaatagtgtatgaatatcaagtttctaaattgaaacgtcagaattggagactaataagacaaagaaagaaaaaaagtgtaagtgtaatatatataaagagggagagagagggagagaga

SEQ ID NO: 147 - DNA, 14765 bp, Glycine max
tatgacgcggaaaaggcataagagaaatcataaagttgataaatgaaaagacgcatgatgacaaattcacaatcatggcagcagacttgcttcttccacattacagtcgtggggaaattcatttagggcagcgaatagttaatggacaaagcatttttatttcttttctttttgccaactgaaacagagaaaagaaaaataacggaatgttaaattatcagcgagaactaaaaatatttgtttatataaaagtgagcaccaaataaaataaatatggaaatcaaaatctgatatttttttttgaagaaaacatgcttaatcttaaaaatttgaaaaactttgggttaattatctaaatttttgcaaaattcacatttagtctataaacaaaaaacttctagtctttgttcttgtacttttcctaaattaatatttttactctaggtcattaaatgtcaccgttaaataattatgtgtacaatcatattaacattcatttttcacaacatcaactagtctttgttcttacatgagaaacacatgtattttttttatagacattatatttttaatcccttatattatcatgtcaaactagtagatgatatattgagtcaagtgttcaccagcgtggctaatgactatcgtgccatcagtacttaagaatgatacttgataataaagacctattacagaagaagaataggaactattgcacttcatatagaaaattgtcgttaggatatcgttacccaattaatgcagttagaggtatacaacctagggactcaacgcaaggaatgacatattacacttcatactaaatgtttttgggatttggtctcttgcttcgttagacatgatccagttacaaaaaccagaataaaaataaaagaataaataagttcaaaaaaataaatagtacacaaatactttttcaaatattttttaaaaaaaagcacacacgcacccacaaaagacaaaacacacgtaaatgcaatattgaatttaataaaagttaaataaataacacactaacacaaacacatagtacaattttgtgtttgttatctttttgtcctttttttagatttctgttttcactcctttaaaaaaaattgttccgatttgttggtccaaatgtattttttttgagagattaaaaaagttaataaacctaaaagaatatataaaacgaagatgggtctgagttgatcagcccgaaatactgcatgcacctcacaaattgacaagtgcttttttccttttaatattctcttgatttgttctatttgggaagaagaagaagaagaaaaagaaaggcctatactataacactacactacagtgagggggcgccgtcaaccaatgagaagcgaaggctgagtgccacaaagacaacagaacaaacccgtttcccgtttcccgtttcccgtttcccgttttctcacttatccatgggacccacatcatcgtacggtctgtctccaaaacgttacgccctgtcaacacgacacatcaatactctcacactcttgttcaattctagctcgacaacgcattgtaccttaaccctttaaccaatcacaactcgacaacgcatcgtaccttaattccccctcctttccccaatttttgttcttattcttctttattttttcgttcacaattaattaaagtagccattatttccgaagcacaaaaatagaaaaaggaaattcccatgctatcctattattagacatcctccacttgttttgctttcatgttccttttttatttttctctttctcagtcttcgtttgctcaggtgaataccactcactctctctacttcctcttctagctagggtttccttctttatacaaaatacaacctaacaagtaacaaccttctttatatgtacatattccttaacccttgtgtttctcttttgagctaattctatttgtgcttttcatagaattgctagttacgttggaaaattaagcaaaagaagatgggaaggggtagggttcagctgaaacggatcgagaacaaaactagccagcaagtgacgttttccaagcgtagatcgggacttctcaagaaagccaacgaaatctctgtgctatgtgatgctcaagttgctttgattatgttctctaccaaaggaaaactttttgagtattcctctgaacgcaggttcctactcattcatcatttctcactttctttttcttctcttattccttttcggtttttatcatttctatctacacatgcttttttcttgttttggatccaccttttatttttgctttatatatgatcaatctggttatggaaatcaatatttatatattatcatatatcataactactaatttaattatttcgagttttcttaatttgtttccttgttttttctatctgatttgtgctcatcttcttcttcttaatttggtaattatctgtttatatatgtgtgtttttgtttcaccttaccctgaaatatagtgctaaagtttcgaccaatacaatagtagtattcacttcagttctacaacttgctggagtttatttttaatctttattacatatacacttttttttcaacgctaagtttttttgtttttaaaaaggtctatttttggtctggtcacgagtatttcactatcctatgtacttttctctcatcacatatagttatctcataagtcataaactcgaaatggaagacttcttttagttattacttttttaaaagtctctttaaatgttttttttttgcattcgtaggcatgagagctttatatgtttgcgtcagcttgtggattttttttttatatatatatatatgtgtgtgtgagaatatgtttactataaatatcaagtcaatcaccttattattttctattaatatacgatgaaatctaactaacaaattaataacaccctagagctttgtgtcaccttttggaagggagtgacattatgccctatactttgtaacctagtaatgtgcctactttggtccattgggtaataagagctctaatccatgactacttgtaatacaatttgtatgcaactgttggaagagggtgacgcaagttaaacggtatcttttcttttaggattttacaaaagtagtcacatagtaactgaaaaaggcttctggagtctggaagacttattggataaaatgtgagttattttaaattttttactactttcgtttaaggataaaaaaatacattataatctttcattgatataactcatttgtaagtaattatgggattaagtctatttgaagaaaatgtttgtattcaaattctaaaagatatatatttacatttaatgtttcatagaatcttgaacataattactctgataaaaaaaatcatttgcaagtagtgtaacaataggtttttgttgtgatcagtgacatgtcttaatcatagaatgaaacaattcttccctttaatatttttctaaaattaaaaactcatgttattaattatgatattttaatacttaaattttattttaaagattaatatccttattttgattacccaagacatatcataagaaattaagattaccatgtttcataaatacattttgtgaaattaacttaacaagtttccttaaactattaatgcgttcatgaatattattatccagcgggttaaaactcagctgctaaagaacatgcatgcaaaaaaaactgtagaggatttgataggagcttaaaattgtaatatctgatttggtggttgcatggtatacgagcttgatttggttgcaaagtatatttgatttgatgacttaaggttagggtcatcaaatgatggtggtctggtggaaaatataattgggtatatataaacaatatatgacaaaaaaaaaaagttatggaaatacaacagttgtacgtgtctatgatagacaactctaagctctatacttgcattgtttttattttgtttggaagataatgacatgagagatacaatggatcctactcgagagttcaataatcacttagtagggaataaaactaccacttatcctttcatcctttaaatactatatataacaaactgaaaacagcagatattgctttaccctacttcttacttctcaaatccccccaaaaaaaaaaaaaacactgaaatggaatggatatgcaagctttcactgttttatatatataatacattgcattatgataaatggggatattagactattcgaaaatagtacaacttgcataatatattgtattaaatgcaattattttaattttgttgcaattcttccgtggaagacattcttaaaaaaaggttaattgcgttttctcttccatgtttaatcattgctactttctataaatacactaaaaggtttattttcccaaccactacattcgatcttttctctcacgattaattattagaacctaataacagtttattaatgcaccggatacacttatatatatatatatatatatatagcttctgtgtacataggggtgttcgtgagctatttatttgtgattcttttgaatttcactcaataaatttccaaagtttgtttgttgaaataaacaaactcaataattattagataattgtaaatttatttatcatatttatttaaaattttatttcttattaattgtgtatctactataaaccacacattgctctgtctatatatatatatatataagtgaggtaaagtaatattgtataataactatgctaatcaaatcctgatgatatacatatgtgaaattaattcaaacataggttacatgtgttatattaaacatcatcttaaattagagcgcacaaatcatatatgctaagtttcctacaacctgatgagacctttgagggatttggtgaacaagccaccgtgtagccaactaaaattgatagcggttgttgttattactcctacaaaaaccttttcttgatcatgaagtgataattgatctatatatatgtttggttctctagtacgaaagactgattagaagcaatgcggagaacattctggttatcacaaatcaactacatttccttgagatttcctaattaattgacttaaatgaagtaacagtcaaagctacgttagttcacatgatgtggctgctacagtagacttgataattatattttgtttgttgcttctccatagaatgaagtattccctagaaaggaacacaatagcctaatgtaaatctccaacgagatggaggtcatgcctaattgttatggggagcatgtctgacttaaaaccttaagtcaatattttttgtatccaataataacattgacttggaagcaattgttaggtaaaaagtgttattttacgttagttatatttctaatcgatgtaaaatggttttaaaaaacttgagccttacaaactcaccaccaaaagctctctccctccaaacaatacccaaaccctatagtgcattgacaactcaaaggtggttatgggaatgaaggatgcaaacaatgcataaacgatgcactttagttgtagaagccacccgaggaagtggagggacttcagggtcatagtggtgcgaagcttgttaagggtgcgttccaagtagaagtctcacatcgtacgatgttggatagtgttcaattttcacggcggttcttgtatcatcttagcctattcgtttctaattgaaaaatcgtctttgaagctgtcgttgtggaaagtgttcaattttcacgacggttcttgtaccgccttaatctgtttgttaagggtgcgctccaagtagaagtcccacatcgtacgatgttggatacacatgattttagggttttgggtgagacaataaggtttgaaaccttagctctatccacacctcggtatatgcttgaaatcttgatctcacactactagaaaattgtatttcaacgacggttatttagtgctttcaatgacgattgacaaatcgtctttgaagctgtcattgtggaaagtgttcaattttcacgatggttcttgtattgtcttagtttattcgtttctaagtcggttttgcaaaccgtcttagataggtttacttattgtttaaaaatgtaaaaaacatcaatatttgacgacagtttttatctaaaaaccatcttataattgtaatattctaagatgattttactccaaaatcgtcttagaatgttacagttttaagacgattttaatttataaccgatattgattttttttaaaatatttgttttgtttttatatcaaaccaaattaaacctacaacttcggtcatgcagacccaaacagatcaattattataatatattacaaaattaattttggtaatgaatttcgactaagcaaaaccaattagtaactaaagaaaagttgtgaattaaatttataggtttcttattgaacaagcgtaagcattcacaagtgcattatatttacatgtagaaacaaatcataaacaaatgaacaagtattatttattgaattcaccaataacttacaatgtgaaagttgtgaattaagcacaaagctctaacattcactaacccgaagctagataaacatgagttacattgacaatattgtagacatacatgaagcatatatgataaaatttccacttacatagaaagcagtgacaatttcgatagagtcaccctccacaagcttgagctggatggtcactttcccaaacatatatctactcttagatccaaatctagcaattgcaatgttcaaaaaaaggtcacatgtttagctaaaaaataacaaattttgcaaggctattaaatgaactaatataagaatcaaatcttacaaaaaaaagtgtaaaattagacataacgggctattaacaaataacattactaaaaatgccacaccaaaatagaacctttcaatgttctatcgtacataatggaagtaaaaccatatttaggtcctatttagataaatttttctataaatacaattttcaaggcaaaaatacatcacaagagtccagtttcatcaaaatcttgaattacgcaataaccacctctgtaatcttatcgttttcctcaaacataaaaaaactaaatagtatcataaattgaaatatcaaaagcttagatcctctaagtctattctagagaaccaacaagggtcaacgacaaatgacccttatatcacactgatagattaggattgaagcagagtataactatatctattttttgcacaaaacttaccatagtatgatcatgataaacataagagaaaacaatctatgcactggattttacattgttgtccaatttcaaacatgtatgtatggtaagtttgttgattttaacaataattaccttaaaagccataaatatcatgatttttatttaatccttttccactataagtgcatagaaattacattctaaacataagcctatattgaaatagacacttaaattgccaaataaactcaactagcaaataacatttgaaaacttacaactaaagatttaaggaagcccaaaacaactttgctttccgcagtcaatatacgttcagcttcttccaccgtagtaatgttggacacattgggtcctatcttcttaatctatgtcactatagtgtctctgcattgcaatgcaacaaaaaaattcaaacaatcaactccaattgaaaattctaaaagcaattacacttaattgagagcaaaattcaaagatccaaaacgtgcattgaattttacttggttctttgggtagtgtagggcttgtggaccctatgaacgaagaagaagacgatggggaaatccttaacattgtacttgtttgccaattcgttttcgacggtggcgttgaccttggcaagaatgatgtcatcgggcttgagcacgatggccgtagtggcatacttcgacgcgagggcctggcagtggccgcaccagggtgcgtagaacttgaccatgaaaaagcgattgttcttgatgacgatggtgaagttgcgctccttcaaaacaacgacatccttgttgtccacctcgggctccttgacacctcctctgacgagtggtcgaagccggtgaagtcgtcgcagtcgcactcgttgtcagcgtcctcctcgtcgaattggtggggatctagaaaatggtcgtgtggagaagtggtggcggcatcattgggttccttgaggaagctaagatcttcgttgttgttgtttttgttgtcgttttgtggggggggggggggctttatcgcagagagtgagggagaggaatgagaagagagtggcgagagagagagaaacaaatagcttggtcaaggagggggcgaggaaggcaaggaagcacgggagggaggagaaggcaaagatagtgagtgagcggggtgagatgacacaactaaaacatataaaaaattaaaccctaatttaaagacgattctcgcgaaactctctttgaatattacaatatgacgatgatttttctaaaaccatcgttgttttgaggtttcaaagacgttttcagtgaaactatctttgaaatgttggcaataattacaagattacctccgttatccaaacgacaacaatttttcaacaatcatcattgaccttgtgtcataaaaacatgtatttttagtagtgttatcatctttcctctctccattattgactttttgatctagtgctttctattggttctccaatatgcgttgtgaaatttagggtttagttcttttgatgctgatattcgttactttgattctcttatgaggaatttaagggcataattctttgatttgggttcacacatcctttgattctttggtattcttcgtcaattgcaaattaatagcacaaggaagatgagagtctagaaagctaatatggaaaactggcattggaatttgggaattggaaatgaaaaattctatgtcaattctaataaaactgacgtagaaaaataaacaagctttttacattataacatctacaaaatgtcaaaacaattggtgtgaaatatcccttttaactaatgtaaaagatctaaatctaaagtgttatagcgttgtaacttgtaagttattttgactaactttttaaattattttaaagtttatttgttacacaagtttatatatttagtagtttttagtgttttgcgtaaactactttaactattaactaacttatgaaaaaaagttaacggatatgttagttttttctttcttttacttgtatcctctctaatttatttgaattatttttattgtgtaactaaatgtattcttttatatttattaattaattaacttattagctaatcttacaaaatatttttagaaatttacaaattttcaactcttggccagtttatgtgtttcaaagatgcttataatcgtttttgttttgatctagggatcaagattttcagcttggtcattactttatctagggattagtttgctttagtgtagaggtctagaaaactagaaatacctcaaattaatttaaaaggaaaaaaaatctacaataaacatctttagaatcttcacaggtttgtagctagctataagctgaactacaggagtagctcgttgaacatacaaatacagttagttggcatgcatggttgtccgtgcactctgactatcgggagttctgtaggggagtgtaggtgccaactaaaattaaagtttagcagcaaatattatgatgactatcttcaagaacacataggtcccctgccctctaatgttttaaaattaaatatgcaaaatattaactctttgtttggttagagggaaaaagagaggaaatgaaggaaaaattttaaaaattaatctggatttcatatttttattttaatttaaatgttttttctttttcattctcttttctcaaacagaccctaaaaattatattattcaatttttttataattagtataatattattttttatgcatcatattacttgtaggttgtatgttaattggtagtaggttttgtgtgatgttttttgttagaagcaaatttccgcaatcaaaagttatatattagattaatcttcaagtggtagagattattagagtgagtttgatttcttctaataaaatctttttttaggtatgacgagataatcaaatccttttcattatgtttaaaaattcaatcatttaccaatagtgttggaccttatttgtattttaggtgtaatgttgatgtaataagttttaacttctggaaattatattaaaaaaatactctgaacttagaaacctatataatcaacaactatttttgtcacaacaaagtcatattgaaaaaaaggtgagaaatgaaatatgaaaaagaaagaaaaaaaatgaaaagtaataaaagaaaaataagttaacttataagttattttttatgaaattatttaaaatttatttttatcattaataattaaatatttcttttacgttattttatatttatcaactaacttattagttaattttatcaaatattttcttagtttttttctaaaagaaattacttttttaaatctttttataacagattcgtattagttaaatttgcattcattcctatattaatccagcaagccttacagtccaagcttaagcaaatgctatatataggccatcaaaatcttcttgtgatgaatttggtgacaggtggtgaatctaaataatttatcggggagtaacatgttaatcaatgtttttcattaacacccaatggtaaatttactatggtttgtagttgaaattttgtatgtattacttcgatcgaagagctacttattgaaagtacaaaagtatatatattcagctaagcatagactgattaactaattaactatttttctaaattacatgcctgtttgatgtaattttttatgaatgtgtgtaattctcctctccaatgtccccaacattttagtgtacttcatagagaaaaataaaattacgagttttcaggtttttctattggttatatagtttttcgttttgacttgtgtaacagcatggaagacgtcctggaacgttacgagagatatacacatacagcacttactggagctaataacaatgaatcacaggtaattcatgatcttatatatatatatatatatatataggtaagtggtatccaaggaaagaaaataaaataggttctgaaaatatcccaattggtctaatagccatgtgtcgttgtcattatacacatgaatggaagtgaatatgaatatacttctttccatgtaagtcacttcagtagtaactgaagatcatttgacgttgtagggaaattggtctttcgaatatatcaagctcaccgccaaagttgaagtcttggacaggaacgtaaggtacgtactttatttagatttcatgggtaacatgtacacttggcaccaccccacacactaacaattagtttggccatccttgctttcttctctttcatgaaattggtagttaataagaacaccatttgaaagtaatatacattttccaaaggtcaattcagttttcatagcccaacaaatatagtgctaacataaaaataatatggaaggaagagggctagtaatacactttgttttttcttttataggccaatgtttatgattagttttttttattagcggatagaattcaaatctataatttttttttcaataagtcaatcttacaccttcattaataattatatatgtttgtaacacattttttttaatatactttctattattagttaaaatttattaaaaattataattttttgttaaattttaactaataataaaaaaatatgtttaaaatagtgtgtacatatcacttctctcaaagcccagatctatttactaaatagagatttcataaactgttgggtcagacacttgacaaacctagtaacgttacgtgattatatgcttcaaaattaacaatatactggaaaagaatgatgtagtttatataatcttcaatacaggaatttcttgggaaatgatctggatcccttgagtttgaaagagcttcagagtttggagcagcagcttgacacagctctgaagcgcatccgaacaagaaaggtgaagtgtgctaacttcttcattagtttacaagaaataattaagaagattgaaacaacagaagagaataaatttaatcttaaaattaaagaattaacattaccacatatgatcactcttgtttttttttcttccaattttgaactgactgatcaatttgatttttgcagaatcaagttatgaatgaatccatctcagacctgcataaaagggtaagatgtctgtgttgtaattgtgggttaaggccttaagggtgatatattgaatattggctagactgcatggcttgtaaaatgatatgttcttagggacattgaatatatcacagaaaaaatctctaataacagaatgtctaatgatttacatgtttgtaactgaatttcatgagcagaaacaaggacatgataaagaaaattcataaacaatttactataatttgaattgtcccccaagaaggaagcatatggctagtatgaagctacccgaagtaatcttagatataattgttagtagagttttttgaaattatatttctcttcttaaatgacattaccgaaagcaatgttaaatgtttttacttggtttcttctttgactttatggttggtaggttgtgttaaattaattttctgcttctgaagggaaattaattagttttctactgactgctagaaggtttgtacataaaaacatttgggatttataaagataaatttttgttgtgcatatgacatgacaggcaaggacattacaagagcaaaacagcaagctagcaaaggttgctagaagccatatattactcttgtaacatttcaatctaaagcttctagacaaaaagtacaaagaaaaagtaattaattttttcattacattttatgactagatgaaggagaaagcgaaaacagtgactgaaggtccacatactggcccagaaactctaggcccaaattcatcgacccttaacttaacttctccacagctaccaccaccaccacaaagactggttccttctctaactctctggtacttttcttgcctctctcttctaagcagatactacagcgaaggagcatctttcattgtcaaaccttatgttttacattctagctacaagggcaggggaattttttttattttttttttcacaggcataatacctttgggcaaatattagtattgcaagtatgcatgtaagtttaaactttatacaagtaccaagttaaaatgataatataataaacaagtttgattttatgatattttatggtccaaatgtcaattatagattcctaattttctaatacctggatttcttccatgttgtcataagcttcgaaaatgggatttcagtaacaaatttaataaattaacatggaaaaaaatgcatacaatatatgatattagttcatttgattcattaagtatatttggtttctgatcgtatcccgaacaagtttttcttgaaaaaatattagccaaattgatttcattaagtacattattgtttgagatgataaaagtgtctaaatgatgcagtgagacattccaaggaagagcattggtggaagaaacgggaaaggctcaaacagtccctagtggcaattctctcatcccaccatggatgcttcatatctgactagttaggctggcatgattctgccaagtatgtgacatgtgatattatgacaaaggctcaactatatatatatgttagaacttagaatagccaattatatgccgatcgagcttaataactgctcaaacttctcgcatgaactgtgcccttcactttgaaatcaatggtattaagcagttattttgacaaatggcacgtaatggatggtgctagttaaattggaagtcatcaaagaaaacatttagcccacaaaaaagacactacgtgatgtgtaccaccactaccaaaagagtttttccctacaaagctcacactttttttcctccgggttaggcttcatttattaaacctagttggataaagatgaatgaaaaagattgaaatgaaatgtaacttttacctagtgattacgtagattataatttgaaaaggttttaacaggctcgaagccgccgcatgtatgatattttggattgtggcctcttcgtggctacgttttttctggttgctgctggcacaacttgtgcggcagcaatcgtgttaatgaaatgatgttttgtttgccttccaaaaaaaaaggatttcattcgtattcatttttctttcttgtaaaaaaaatagttaaatacaagaggtaaactaaaaaaaaagttttggaaacccaaaacaataaaagaattgcacctatgaaaaatctaattgaccatattacaattttttcctcattaatgttaagatttaccaatctctccattaaaattgataattcaaactattatattaaattaataatgagaaaaaaggtaaatcccgccagaaagacagatcaaaggcaacccgaaattttgcaaggatgtaatgcttttgaggctgaattgtaggagagccaagaagggttgcaagttgcaattggcttggaaacttggttactgaaagcttaaagttgataatggttagtgctctaaaaagcagtgtacatcatgaatgtgcggataacctt

(19) United States
(12) Patent Application Publication
(10) Pub. No.: US 2020/0362360 A1
(43) Pub. Date: Nov. 19, 2020

(54) MODIFIED PLANTS WITH ENHANCED TRAITS

(71) Applicant: Monsanto Technology LLC, St. Louis, MO (US)

(72) Inventors: Robert M. Alba, St. Louis, MO (US); Edwards M. Allen, O'Fallon, MO (US); Brent Brower-Toland, St. Louis, MO (US); Molian Deng, Glencoe, MO (US); Todd DeZwaan, Apex, NC (US); Charles Dietrich, Chesterfield, MO (US); Alexander Goldshmidt, Davis, CA (US); Cara L. Griffith, Catawissa, MO (US); Miya D. Howell, Ballwin, MO (US); Niranjani J. Iyer, Middleton, WI (US); Hongwu Jia, Apex, NC (US); Saritha V. Kuriakose, Kottayam (IN); Hong Li, Chesterfield, MO (US); Linda L. Lutfiyya, St. Louis, MO (US); Anil Neelam, Wildwood, MO (US); Shengzhi Pang, Chesterfield, MO (US); Mingsheng Peng, Wildwood, MO (US); Monnanda Somaiah Rajani, Chesterfield, MO (US); Daniel Ruzicka, St. Louis, MO (US); Daniel P. Schachtman, Lincoln, NE (US); Vijay K. Sharma, Wildwood, MO (US); Tyamagondlu V. Venkatesh, St. Louis, MO (US); Huai Wang, Chesterfield, MO (US); Xiaoyun Wu, Chesterfield, MO (US); Nanfei Xu, Cary, NC (US)

(21) Appl. No.: 16/765,867

(22) PCT Filed: Nov. 21, 2018
(86) PCT No.: PCT/US2018/062238

§ 371 Date: May 20, 2020

Related U.S. Application Data
(60) Provisional application No. 62/589,171, filed on Nov. 21, 2017.

(51) Int. Cl. C12N 15/82

(52) U.S. Cl. CPC C12N 15/8218; C12N 15/8261; C12N 15/8213

(57) ABSTRACT

This disclosure provides recombinant DNA constructs and modified or transgenic plants having enhanced traits such as increased yield, increased nitrogen use efficiency, and enhanced drought tolerance or water use efficiency. Modified or transgenic plants may include field crops as well as plant propagules, plant parts and progeny of such modified or transgenic plants. Methods of making and using such modified or transgenic plants are also provided, as are methods of producing seed from such modified or transgenic plants, growing such seed, and selecting progeny plants with enhanced traits. Further disclosed are modified or transgenic plants with altered phenotypes or traits which are useful for screening and selecting transgenic events, edits or mutations with a desired enhanced trait.

### CROSS-REFERENCE TO RELATED APPLICATIONS

This application claims the benefit of U.S. Provisional Application No. 62/589,171, filed Nov. 21, 2017, herein incorporated by reference in its entirety.

### INCORPORATION OF SEQUENCE LISTING

The sequence listing file named “MONS454WO_ST25.txt”, which is 395 kilobytes (measured in MS-WINDOWS) and was created on Nov. 20, 2018, is filed herewith and incorporated herein by reference in its entirety.

### FIELD OF THE INVENTION

Disclosed herein are recombinant DNA constructs, plants having altered phenotypes, enhanced traits, increased yield, increased nitrogen use efficiency and increased water use efficiency; propagules, progenies and field crops of such plants; and methods of making and using such plants. Also disclosed are methods of producing seed from such plants, growing such seed and/or selecting progeny plants with altered phenotypes, enhanced traits, increased yield, increased nitrogen use efficiency and increased water use efficiency.

### SUMMARY

In one aspect, the present disclosure provides recombinant DNA constructs each comprising: (a) a polynucleotide sequence with at least 90%, at least 91%, at least 92%, at least 93%, at least 94%, at least 95%, at least 96%, at least 97%, at least 98%, at least 99%, or 100% identity to a sequence selected from the group consisting of SEQ ID NOs: 1-31; (b) a polynucleotide sequence that encodes a polypeptide comprising an amino acid sequence with at least 90%, at least 91%, at least 92%, at least 93%, at least 94%, at least 95%, at least 96%, at least 97%, at least 98%, at least 99%, or 100% identity to a sequence selected from the group consisting of SEQ ID NOs: 32-62 and 104-140; (c) a polynucleotide sequence that encodes a RNA molecule for suppressing the expression of an endogenous gene, wherein the endogenous gene encodes a mRNA molecule comprising a polynucleotide sequence with at least 90%, at least 91%, at least 92%, at least 93%, at least 94%, at least 95%, at least 96%, at least 97%, at least 98%, at least 99%, or 100% identity to a sequence selected from the group consisting of SEQ ID NOs: 63-69; or (d) a polynucleotide sequence that encodes a RNA molecule for suppressing the expression of an endogenous gene, wherein the endogenous gene encodes a protein comprising an amino acid sequence with at least 90%, at least 91%, at least 92%, at least 93%, at least 94%, at least 95%, at least 96%, at least 97%, at least 98%, at least 99%, or 100% identity to a sequence selected from the group consisting of SEQ ID NOs: 70-76.

Recombinant DNA constructs of the present disclosure may comprise a polynucleotide sequence encoding a RNA molecule for suppressing the expression of an endogenous gene, and wherein the RNA comprises a polynucleotide sequence that is at least 80%, at least 85%, at least 90%, at least 91%, at least 92%, at least 93%, at least 94%, at least 95%, at least 96%, at least 97%, at least 98%, at least 99% identity, or 100% complementary to at least 15, at least 16, at least 17, at least 18, at least 19, at least 20, at least 21, at least 22, at least 23, at least 24, at least 25, at least 26, or at least 27 consecutive nucleotides of a sequence selected from the group consisting of SEQ ID NOs: 63-69, or to at least 15, at least 16, at least 17, at least 18, at least 19, at least 20, at least 21, at least 22, at least 23, at least 24, at least 25, at least 26, or at least 27 consecutive nucleotides of a mRNA sequence transcribed from the endogenous gene encoding a protein that is at least 90%, at least 91%, at least 92%, at least 93%, at least 94%, at least 95%, at least 96%, at least 97%, at least 98%, at least 99%, or 100% identical to an amino acid sequence selected from the group consisting of SEQ ID NOs: 70-76. According to some embodiments, recombinant DNA constructs of the present disclosure may comprise a polynucleotide sequence encoding a RNA molecule for suppressing the expression of an endogenous gene, wherein the RNA comprises a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to a sequence selected from the group consisting of SEQ ID NOs: 84-90. Recombinant DNA constructs of the present disclosure may comprise a polynucleotide sequence selected from the group consisting of SEQ ID NOs: 77-83.

The recombinant DNA construct may comprise a heterologous promoter functional in a plant cell and operably linked to the polynucleotide sequence. Vectors, plasmids, plants, propagules and plant cells are further provided comprising such a recombinant DNA construct. The suppression RNA encoded by the recombinant DNA construct may be selected from the group consisting of a double-stranded RNA, an antisense RNA, a miRNA and a ta-siRNA.

Plants comprising a recombinant DNA construct may be a field crop plant, such as corn, soybean, cotton, canola, rice, barley, oat, wheat, turf grass, alfalfa, sugar beet, sunflower, quinoa and sugarcane. A plant comprising a recombinant DNA construct may have an altered phenotype or an enhanced trait as compared to a control plant. The enhanced trait may be, for example, decreased days from planting to maturity, increased stalk size, increased number of leaves, increased plant height growth rate in vegetative stage, increased ear size, increased ear dry weight per plant, increased number of kernels per ear, increased weight per kernel, increased number of kernels per plant, decreased ear void, extended grain fill period, reduced plant height, increased number of root branches, increased total root length, increased yield, increased nitrogen use efficiency, and increased water use efficiency as compared to a control plant. The altered phenotype may be, for example, plant height, biomass, canopy area, anthocyanin content, chlorophyll content, water applied, water content, and water use efficiency.

According to another aspect, the present disclosure provides methods for altering a phenotype, enhancing a trait, increasing yield, increasing nitrogen use efficiency, or increasing water use efficiency in a plant comprising producing a transgenic plant comprising a recombinant DNA construct of the present disclosure. The step of producing a transgenic plant may further comprise transforming a plant cell or tissue with the recombinant DNA construct, and regenerating or developing the transgenic plant from the plant cell or tissue comprising the recombinant DNA construct. The transgenic plant may then be crossed to (a) itself; (b) a second plant from the same plant line; (c) a wild type plant; or (d) a second plant from a different plant line, to produce one or more progeny plants; and a plant may be selected from the progeny plants having increased yield, increased nitrogen use efficiency, or increased water use efficiency, or other altered phenotype or enhanced trait as compared to a control plant. Plants produced by this method are further provided.

According to another aspect, the present disclosure provides recombinant DNA molecules for use as a donor template in site-directed integration, wherein a recombinant DNA molecule comprises an insertion sequence comprising: (a) a polynucleotide sequence with at least 90%, at least 91%, at least 92%, at least 93%, at least 94%, at least 95%, at least 96%, at least 97%, at least 98%, at least 99%, or 100% identity to a sequence selected from the group consisting of SEQ ID NOs: 1-31; (b) a polynucleotide sequence that encodes a polypeptide comprising an amino acid sequence with at least 90%, at least 91%, at least 92%, at least 93%, at least 94%, at least 95%, at least 96%, at least 97%, at least 98%, at least 99%, or 100% identity to a sequence selected from the group consisting of SEQ ID NOs: 32-62 and 104-140; (c) a polynucleotide sequence that encodes a RNA molecule for suppressing the expression of an endogenous gene, wherein the endogenous gene encodes a mRNA molecule comprising a polynucleotide sequence with at least 90%, at least 91%, at least 92%, at least 93%, at least 94%, at least 95%, at least 96%, at least 97%, at least 98%, at least 99%, or 100% identity to a sequence selected from the group consisting of SEQ ID NOs: 63-69; or (d) a polynucleotide sequence that encodes a RNA molecule for suppressing the expression of an endogenous gene, wherein the endogenous gene encodes a protein comprising an amino acid sequence with at least 90%, at least 91%, at least 92%, at least 93%, at least 94%, at least 95%, at least 96%, at least 97%, at least 98%, at least 99%, or 100% identity to a sequence selected from the group consisting of SEQ ID NOs: 70-76.

The insertion sequence of a recombinant DNA molecule may comprise a heterologous promoter functional in a plant cell and operably linked to the polynucleotide sequence. The recombinant DNA molecule may further comprise at least one homology arm flanking the insertion sequence to direct the integration of the insertion sequence into a desired genomic locus. Plants, propagules and plant cells are further provided comprising the insertion sequence. According to some embodiments, the recombinant DNA molecule may further comprise an expression cassette encoding a site-specific nuclease and/or one or more guide RNAs.

According to another aspect, the present disclosure provides recombinant DNA molecules for use as a donor template in site-directed integration, wherein a recombinant DNA molecule comprises an insertion sequence for modulation of expression of an endogenous gene, wherein the endogenous gene comprises: (a) a polynucleotide sequence encoding a mRNA molecule with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 1-31; or (b) a polynucleotide sequence that encodes a polypeptide having an amino acid sequence with at least 90%, at least 91%, at least 92%, at least 93%, at least 94%, at least 95%, at least 96%, at least 97%, at least 98%, at least 99% identity, or 100% identity to a sequence selected from the group consisting of SEQ ID NOs: 32-62 and 104-140.

The insertion sequence may comprise a promoter, an enhancer, an intron, or a terminator region, which may correspond to a promoter, an enhancer, an intron, or a terminator region of an endogenous gene. Plants, propagules and plant cells are further provided comprising the insertion sequence. The recombinant DNA molecule may further comprise at least one homology arm flanking the insertion sequence. According to some embodiments, the recombinant DNA molecule may further comprise an expression cassette encoding a site-specific nuclease and/or one or more guide RNAs.

According to another aspect, the present disclosure provides methods for altering a phenotype, enhancing a trait, increasing yield, increasing nitrogen use efficiency, or increasing water use efficiency in a plant comprising: (a) modifying the genome of a plant cell by: (i) identifying an endogenous gene of the plant corresponding to a gene selected from the list of genes in Tables 1 and 17 herein, and their homologs, and (ii) modifying a sequence of the endogenous gene in the plant cell via site-directed integration to modify the expression level of the endogenous gene; and (b) regenerating or developing a plant from the plant cell.

According to another aspect, the present disclosure provides a modified corn plant or plant part comprising at least one cell having a mutation or edit in an endogenous gene introduced by a mutagenesis or genome editing technique that reduces the expression level or activity of the endogenous gene in the at least one corn cell, relative to a wild type allele of the endogenous gene not having the mutation or edit, wherein the endogenous gene is a calcineurin B-like (CBL) interacting protein kinase 8 (Zm.CIPK8) gene, a sorbitol dehydrogenase (Zm.SDH) gene, a cytokinin dehydrogenase/oxidase 4b (CKX4b) gene, or a cytokinin dehydrogenase/oxidase 10 (CKX10) gene. The modified corn plant may have an altered phenotype or enhanced trait relative to a control plant.

According to another aspect, the present disclosure provides a modified soybean plant or plant part comprising at least one cell having a mutation or edit in an endogenous gene introduced by a mutagenesis or genome editing technique that reduces the expression level or activity of the endogenous gene in the at least one soybean cell, relative to a wild type allele of the endogenous gene not having the mutation or edit, wherein the endogenous gene is a homeobox transcription factor 1 (Gm.HB1) gene, a branched 1 (Gm.BRC1) gene, or a fruitful c (Gm.FULc) gene. The modified soybean plant may have an altered phenotype or enhanced trait relative to a control plant.

According to another aspect, the present disclosure provides a composition comprising a guide RNA molecule, wherein the guide RNA molecule comprises a guide sequence that is at least 95%, at least 96%, at least 97%, at least 99%, or 100% identical or complementary to at least 10, at least 11, at least 12, at least 13, at least 14, at least 15, at least 16, at least 17, at least 18, at least 19, at least 20, at least 21, at least 22, at least 23, at least 24, or at least 25 consecutive nucleotides of a target DNA sequence at or near the genomic locus of an endogenous target gene of a corn plant, wherein the endogenous target gene is a calcineurin B-like (CBL) interacting protein kinase 8 (Zm.CIPK8) gene, a sorbitol dehydrogenase (Zm.SDH) gene, a cytokinin dehydrogenase/oxidase 4b (CKX4b) gene, or a cytokinin dehydrogenase/oxidase 10 (CKX10) gene. According to some aspects, the guide RNA molecule may comprise a guide sequence that is at least 95%, at least 96%, at least 97%, at least 99% or 100% complementary to at least 10, at least 11, at least 12, at least 13, at least 14, at least 15, at least 16, at least 17, at least 18, at least 19, at least 20, at least 21, at least 22, at least 23, at least 24, or at least 25 consecutive nucleotides of SEQ ID NO: 141, 142, 144, or 145, or a sequence complementary thereto. According to some aspects, the endogenous target gene may comprise a sequence that is at least 80%, at least 85%, at least 90%, at least 95%, at least 96%, at least 97%, at least 98%, at least 99%, at least 99.5%, or 100% identical to SEQ ID NO: 63, 64, 66, or 67, and/or wherein the endogenous target gene encodes a protein that is at least 80%, at least 85%, at least 90%, at least 95%, at least 96%, at least 97%, at least 98%, at least 99%, at least 99.5%, or 100% identical to SEQ ID NO: 70, 71, 73, or 74. According to some aspects, the composition may comprise a recombinant DNA donor template comprising at least one homology sequence or homology arm, wherein the at least one homology sequence or homology arm is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a homology arm target DNA sequence, wherein the homology arm target DNA sequence is a genomic sequence at or near the genomic locus of the endogenous target gene of a corn plant, wherein the endogenous target gene is a calcineurin B-like (CBL) interacting protein kinase 8 (Zm.CIPK8) gene, a sorbitol dehydrogenase (Zm.SDH) gene, a cytokinin dehydrogenase/oxidase 4b (CKX4b) gene, or a cytokinin dehydrogenase/oxidase 10 (CKX10) gene.

According to another aspect, the present disclosure provides a composition comprising a guide RNA molecule, wherein the guide RNA molecule comprises a guide sequence that is at least 95%, at least 96%, at least 97%, at least 99%, or 100% identical or complementary to at least 10, at least 11, at least 12, at least 13, at least 14, at least 15, at least 16, at least 17, at least 18, at least 19, at least 20, at least 21, at least 22, at least 23, at least 24, or at least 25 consecutive nucleotides of a target DNA sequence at or near the genomic locus of an endogenous target gene of a soybean plant, wherein the endogenous target gene is a homeobox transcription factor 1 (Gm.HB1) gene, a branched 1 (Gm.BRC1) gene, or a fruitful c (Gm.FULc) gene. According to some aspects, the guide RNA molecule may comprise a guide sequence that is at least 95%, at least 96%, at least 97%, at least 99% or 100% complementary to at least 10, at least 11, at least 12, at least 13, at least 14, at least 15, at least 16, at least 17, at least 18, at least 19, at least 20, at least 21, at least 22, at least 23, at least 24, or at least 25 consecutive nucleotides of SEQ ID NO: 143, 146, or 147, or a sequence complementary thereto. According to some aspects, the endogenous target gene may comprise a sequence that is at least 80%, at least 85%, at least 90%, at least 95%, at least 96%, at least 97%, at least 98%, at least 99%, at least 99.5%, or 100% identical to SEQ ID NO: 65, 68, or 69, and/or wherein the endogenous target gene encodes a protein that is at least 80%, at least 85%, at least 90%, at least 95%, at least 96%, at least 97%, at least 98%, at least 99%, at least 99.5%, or 100% identical to SEQ ID NO: 72, 75, or 76. According to some aspects, the composition may further comprise a recombinant DNA donor template comprising at least one homology sequence or homology arm, wherein the at least one homology sequence or homology arm is at least 70%, at least 75%, at least 80%, at least 85%, at least 90%, at least 95%, at least 96%, at least 97%, at least 99% or 100% complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of a homology arm target DNA sequence, wherein the homology arm target DNA sequence is a genomic sequence at or near the genomic locus of the endogenous target gene of a corn plant, wherein the endogenous target gene is a homeobox transcription factor 1 (Gm.HB1) gene, a branched 1 (Gm.BRC1) gene, or a fruitful c (Gm.FULc) gene.

According to another aspect, the present disclosure provides a recombinant DNA construct comprising a transcribable DNA sequence encoding a non-coding guide RNA molecule, wherein the guide RNA molecule comprises a guide sequence that is at least 95%, at least 96%, at least 97%, at least 99% or 100% complementary to at least 10, at least 11, at least 12, at least 13, at least 14, at least 15, at least 16, at least 17, at least 18, at least 19, at least 20, at least 21, at least 22, at least 23, at least 24, or at least 25 consecutive nucleotides of a target DNA sequence at or near the genomic locus of (i) an endogenous target gene of a corn plant, wherein the endogenous target gene is a calcineurin B-like (CBL) interacting protein kinase 8 (Zm.CIPK8) gene, a sorbitol dehydrogenase (Zm.SDH) gene, a cytokinin dehydrogenase/oxidase 4b (CKX4b) gene, or a cytokinin dehydrogenase/oxidase 10 (CKX10) gene, or (ii) an endogenous target gene of a soybean plant, wherein the endogenous target gene is a homeobox transcription factor 1 (Gm.HB1) gene, a branched 1 (Gm.BRC1) gene, or a fruitful c (Gm.FULc) gene. According to some aspects, the guide RNA molecule may comprise a guide sequence that is at least 95%, at least 96%, at least 97%, at least 99% or 100% complementary to at least 10, at least 11, at least 12, at least 13, at least 14, at least 15, at least 16, at least 17, at least 18, at least 19, at least 20, at least 21, at least 22, at least 23, at least 24, or at least 25 consecutive nucleotides of SEQ ID NO: 141, 142, 143, 144, 145, 146, or 147, or a sequence complementary thereto. The transcribable DNA sequence may be operably linked to a plant-expressible promoter. According to some aspects, the endogenous target gene may comprise a sequence that is at least 80%, at least 85%, at least 90%, at least 95%, at least 96%, at least 97%, at least 98%, at least 99%, at least 99.5%, or 100% identical to SEQ ID NO: 63, 64, 65, 66, 67, 68, or 69, and/or wherein the endogenous target gene encodes a protein that is at least 80%, at least 85%, at least 90%, at least 95%, at least 96%, at least 97%, at least 98%, at least 99%, at least 99.5%, or 100% identical to SEQ ID NO: 70, 71, 72, 73, 74, 75, 76 or 77. Further provided are a DNA molecules, vectors, bacteria and host cells that may comprise the recombinant DNA construct. According to some aspects, a composition comprising the recombinant DNA construct is provided, which may further comprise a RNA-guided endonuclease.

According to another aspect, the present disclosure provides a composition comprising a first DNA molecule or vector and a second DNA molecule or vector, wherein the first DNA molecule or vector comprises a recombinant DNA construct encoding a guide RNA molecule that is complementary to a DNA target site at or near an endogenous target gene of a corn or soybean plant, and the second DNA molecule or vector comprises a second recombinant DNA construct encoding a RNA-guided endonuclease. According to some aspects, the composition may further comprise a recombinant DNA donor template comprising at least one homology sequence or homology arm, wherein the at least one homology sequence or homology arm is complementary to a target DNA sequence at or near the genomic locus of an endogenous target gene of a corn or soybean plant.

According to another aspect, the present disclosure provides an engineered site-specific nuclease that binds to a target site at or near the genomic locus of an endogenous target gene of a corn or soybean plant and causes a double-strand break or nick at the target site. According to some aspects, the site-specific nuclease may be a meganuclease, homing endonuclease, a zinc finger nuclease (ZFN), or a transcription activator-like effector nuclease (TALEN). According to some aspects, the endogenous target gene may be a calcineurin B-like (CBL) interacting protein kinase 8 (Zm.CIPK8) gene, a sorbitol dehydrogenase (Zm.SDH) gene, a cytokinin dehydrogenase/oxidase 4b (CKX4b) gene, or a cytokinin dehydrogenase/oxidase 10 (CKX10) gene in corn, or a homeobox transcription factor 1 (Gm.HB1) gene, a branched 1 (Gm.BRC1) gene, or a fruitful c (Gm.FULc) gene in soybean. According to some aspects, the target site bound by the site-specific nuclease may be at least 80%, at least 85%, at least 90%, at least 95%, at least 96%, at least 97%, at least 99% or 100% identical or complementary to at least 20, at least 25, at least 30, at least 35, at least 40, at least 45, at least 50, at least 60, at least 70, at least 80, at least 90, at least 100, at least 150, at least 200, at least 250, at least 500, at least 1000, at least 2500, or at least 5000 consecutive nucleotides of SEQ ID NO: 141, 142, 143, 144, 145, 146, or 147, or a sequence complementary thereto.

According to another aspect, the present disclosure provides a recombinant DNA construct comprising a transgene encoding a site-specific nuclease, wherein the site-specific nuclease binds to a target site at or near the genomic locus of an endogenous target gene of a corn or soybean plant and causes a double-strand break or nick at the target site, wherein the transgene is operably linked to a plant-expressible promoter. According to some aspects, the endogenous target gene may be a calcineurin B-like (CBL) interacting protein kinase 8 (Zm.CIPK8) gene, a sorbitol dehydrogenase (Zm.SDH) gene, a cytokinin dehydrogenase/oxidase 4b (CKX4b) gene, or a cytokinin dehydrogenase/oxidase 10 (CKX10) gene in corn, or a homeobox transcription factor 1 (Gm.HB1) gene, a branched 1 (Gm.BRC1) gene, or a fruitful c (Gm.FULc) gene in soybean.

According to another aspect, the present disclosure provides a method for producing a corn or soybean plant having a genomic edit at or near an endogenous target gene, comprising: (a) introducing into at least one cell of an explant of the corn or soybean plant a site-specific nuclease or a recombinant DNA molecule comprising a transgene encoding a site-specific nuclease, wherein the site-specific nuclease binds to a target site at or near the genomic locus of the endogenous target gene and causes a double-strand break or nick at the target site, and (b) regenerating or developing an edited corn or soybean plant from the at least one explant cell comprising the genomic edit at or near the endogenous target gene of the edited corn or soybean plant. According to some aspects, the method may further comprise (c) selecting the edited corn or soybean plant based on a plant phenotype or trait or a molecular assay.

### DETAILED DESCRIPTION

In the attached sequence listing:

SEQ ID NOs 1 to 31 are nucleotide or DNA coding sequences or strands that may be used in recombinant DNA constructs to impart an enhanced trait in plants, each representing a coding sequence for a protein.

SEQ ID NOs 32 to 62 are amino acid sequences encoded by the nucleotide or DNA sequences of SEQ ID NOs 1 to 31, respectively in the same order.

SEQ ID NOs: 63 to 69 are nucleotide or DNA sequences, each representing a coding sequence of a suppression target gene.

SEQ ID NOs 70 to 76 are amino acid sequences encoded by the nucleotide or DNA sequences of SEQ ID NOs 63 to 69, respectively in the same order.

SEQ ID NOs 77 to 83 are nucleotide or DNA sequences that may be used in recombinant DNA constructs to impart an enhanced trait or altered phenotype in plants, each encoding an engineered miRNA precursor sequence.

SEQ ID NOs: 84 to 90 are nucleotide or DNA targeting sequences of engineered miRNA precursors represented by the nucleotide sequences of SEQ ID NOs 77 to 83, respectively in the same order.

SEQ ID NOs 91 to 94 are nucleotide or DNA sequences of variants of a rice MIR gene.

SEQ ID NOs 95 to 103 are nucleotide or DNA sequences that may be used in recombinant DNA constructs to impart an enhanced trait or altered phenotype in plants, each representing a promoter with a specific type of expression pattern.

SEQ ID NOs 104 to 140 are amino acid sequences of proteins homologous to the proteins with amino acid sequences of SEQ ID NOs 32 to 62 and 70 to 76, respectively.

SEQ ID NOs 141 to 147 are genomic DNA sequences for the corn and soybean target genes for suppression identified in Table 2 below that may also be targeted for genome editing. In addition to the gene sequence comprising exon and intron sequences, both upstream and downstream sequences are included.

Unless otherwise stated, nucleic acid sequences in the text of this specification are given, when read from left to right, in the 5′ to 3′ direction. One of skill in the art would be aware that a given DNA sequence is understood to define a corresponding RNA sequence which is identical to the DNA sequence except for replacement of the thymine (T) nucleotide of the DNA with uracil (U) nucleotide. Thus, providing a specific DNA sequence is understood to define the exact RNA equivalent. A given first polynucleotide sequence, whether DNA or RNA, further defines the sequence of its exact complement (which can be DNA or RNA), i.e., a second polynucleotide that hybridizes perfectly to the first polynucleotide by forming Watson-Crick base-pairs. By “essentially identical” or “essentially complementary” to a target gene or a fragment of a target gene is meant that a polynucleotide strand (or at least one strand of a double-stranded polynucleotide) is designed to hybridize (generally under physiological conditions such as those found in a living plant or animal cell) to a target gene or to a fragment of a target gene or to the transcript of the target gene or the fragment of a target gene; one of skill in the art would understand that such hybridization does not necessarily require 100% sequence identity or complementarity. As used herein “operably linked” means the association of two or more DNA fragments in a recombinant DNA construct so that the expression or function of one (for example, protein-encoding DNA), is controlled or influenced by the other (for example, a promoter). A first nucleic acid sequence is “operably” connected or “linked” with a second nucleic acid sequence when the first nucleic acid sequence is placed in a functional relationship with the second nucleic acid sequence. For example, a promoter sequence is “operably linked” to DNA if the promoter provides for transcription or expression of the DNA. Generally, operably linked DNA sequences are contiguous.

As used herein, the terms “percent identity” and “percent identical” (including any numerical percentage identity) in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. For percent identity, two or more polynucleotide or protein sequences are optimally aligned if the maximum number of ordered nucleotides or amino acids of the two or more sequences are linearly aligned or matched (i.e., identical) with allowance for gap(s) in their alignment. For purposes of calculating “percent identity” between DNA and RNA sequences, a uracil (U) of a RNA sequence is considered identical to a thymine (T) of a DNA sequence. If the window of comparison is defined as a region of alignment between two or more sequences (i.e., excluding nucleotides at the 5′ and 3′ ends of aligned polynucleotide sequences, or amino acids at the N-terminus and C-terminus of aligned protein sequences, that are not identical between the compared sequences), then the “percent identity” may also be referred to as a “percent alignment identity”. If the “percent identity” is being calculated in relation to a reference sequence without a particular comparison window being specified, then the percent identity is determined by dividing the number of matched positions over the region of alignment by the total length of the reference sequence. Accordingly, for purposes of the present disclosure, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%.

As used herein, the terms “percent complementarity” or “percent complementary” (including any numerical percentage complementarity) in reference to two nucleotide sequences is similar to the concept of percent identity, but refers to the percentage of nucleotides of a query sequence that optimally base-pair or hybridize to nucleotides of a subject sequence when the query and subject sequences are linearly arranged and optimally base paired. Such a percent complementarity may be between two DNA strands, two RNA strands, or a DNA strand and a RNA strand. The “percent complementarity” is calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences may be determined based on the known pairings of nucleotide bases, such as G-C, A-T, and A-U, through hydrogen bonding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present disclosure, when two sequences (query and subject) are optimally base-paired (with allowance for mismatches or non-base-paired nucleotides but without folding or secondary structures), the “percent complementarity” for the query sequence is equal to the number of base-paired positions between the two sequences divided by the total number of positions in the query sequence over its length (or by the number of positions in the query sequence over a comparison window), which is then multiplied by 100%.

As used herein, the term “expression” refers to the production of a polynucleotide or a protein by a plant, plant cell or plant tissue which can give rise to an altered phenotype or enhanced trait. Expression can also refer to the process by which information from a gene is used in the synthesis of functional gene products, which may include but are not limited to other polynucleotides or proteins which may serve, e.g., an enzymatic, structural or regulatory function. Gene products having a regulatory function include but are not limited to elements that affect the occurrence or level of transcription or translation of a target protein. In some cases, the expression product is a non-coding functional RNA.

“Modulation” of expression refers to the process of effecting either overexpression or suppression of a polynucleotide or a protein.

The term “suppression” as used herein refers to a lower expression level of a target polynucleotide or target protein in a plant, plant cell or plant tissue, as compared to the expression in a wild-type or control plant, cell or tissue, at any developmental or temporal stage for the gene. The term “target protein” as used in the context of suppression refers to a protein which is suppressed; similarly, “target mRNA” refers to a polynucleotide which can be suppressed or, once expressed, degraded so as to result in suppression of the target protein it encodes. The term “target gene” as used in the context of suppression refers to a “target protein” and/or “target mRNA”. In alternative non-limiting embodiments, suppression of a target protein and/or target polynucleotide can give rise to an enhanced trait or altered phenotype directly or indirectly. In one exemplary embodiment, the target protein is one which can indirectly increase or decrease the expression of one or more other proteins, the increased or decreased expression, respectively, of which is associated with an enhanced trait or an altered phenotype. In another exemplary embodiment, the target protein can bind to one or more other proteins associated with an altered phenotype or enhanced trait to enhance or inhibit their function and thereby affect the altered phenotype or enhanced trait indirectly.

Suppression can be applied using numerous approaches. Non-limiting examples include: suppressing an endogenous gene(s) or a subset of genes in a pathway, suppressing one or more mutation(s) that has/have resulted in decreased activity of a protein, suppressing the production of an inhibitory agent, to elevate, reduce or eliminate the level of substrate that an enzyme requires for activity, producing a new protein, activating a normally silent gene; or accumulating a product that does not normally increase under natural conditions.

Conversely, the term “overexpression” as used herein refers to a greater expression level of a polynucleotide or a protein in a plant, plant cell or plant tissue, compared to expression in a wild-type plant, cell or tissue, at any developmental or temporal stage for the gene. Overexpression can take place in plant cells normally lacking expression of polypeptides functionally equivalent or identical to the present polypeptides. Overexpression can also occur in plant cells where endogenous expression of the present polypeptides or functionally equivalent molecules normally occurs, but such normal expression is at a lower level. Overexpression thus results in a greater than normal production, or “overproduction” of the polypeptide in the plant, cell or tissue.

The term “target protein” as used herein in the context of overexpression refers to a protein which is overexpressed; “target mRNA” refers to an mRNA which encodes and is translated to produce the target protein, which can also be overexpressed. The term “target gene” as used in the context of overexpression refers to a “target protein” and/or “target mRNA”. In alternative embodiments, the target protein can effect an enhanced trait or altered phenotype directly or indirectly. In the latter case it may do so, for example, by affecting the expression, function or substrate available to one or more other proteins. In an exemplary embodiment, the target protein can bind to one or more other proteins associated with an altered phenotype or enhanced trait to enhance or inhibit their function.

Overexpression can be achieved using numerous approaches. In one embodiment, overexpression can be achieved by placing the DNA sequence encoding one or more polynucleotides and/or polypeptides under the control of a promoter, examples of which include but are not limited to endogenous promoters, heterologous promoters, inducible promoters and tissue specific promoters. In one exemplary embodiment, the promoter is a constitutive promoter, for example, the cauliflower mosaic virus 35S transcription initiation region. Thus, depending on the promoter used, overexpression can occur throughout a plant, in specific tissues of the plant, or in the presence or absence of different inducing or inducible agents, such as hormones or environmental signals.

Gene Suppression Elements: The gene suppression element can be transcribable DNA of any suitable length, and generally includes at least about 19 to about 27 nucleotides (for example 19, 20, 21, 22, 23, or 24 nucleotides) for every target gene that the recombinant DNA construct is intended to suppress. In many embodiments, the gene suppression element includes more than 23 nucleotides (for example, more than about 30, about 50, about 100, about 200, about 300, about 500, about 1000, about 1500, about 2000, about 3000, about 4000, or about 5000 nucleotides) for every target gene that the recombinant DNA construct is intended to suppress.

Suitable gene suppression elements useful in the recombinant DNA constructs of the invention include at least one element (and, in some embodiments, multiple elements) selected from the group consisting of: (a) DNA that includes at least one anti-sense DNA segment that is anti-sense to at least one segment of the at least one first target gene; (b) DNA that includes multiple copies of at least one anti-sense DNA segment that is anti-sense to at least one segment of the at least one first target gene; (c) DNA that includes at least one sense DNA segment that is at least one segment of the at least one first target gene; (d) DNA that includes multiple copies of at least one sense DNA segment that is at least one segment of the at least one first target gene; (e) DNA that transcribes to RNA for suppressing the at least one first target gene by forming double-stranded RNA and includes at least one anti-sense DNA segment that is anti-sense to at least one segment of the at least one target gene and at least one sense DNA segment that is at least one segment of the at least one first target gene; (f) DNA that transcribes to RNA for suppressing the at least one first target gene by forming a single double-stranded RNA and includes multiple serial anti-sense DNA segments that are anti-sense to at least one segment of the at least one first target gene and multiple serial sense DNA segments that are at least one segment of the at least one first target gene; (g) DNA that transcribes to RNA for suppressing the at least one first target gene by forming multiple double strands of RNA and includes multiple anti-sense DNA segments that are anti-sense to at least one segment of the at least one first target gene and multiple sense DNA segments that are at least one segment of the at least one first target gene, and wherein the multiple anti-sense DNA segments and the multiple sense DNA segments are arranged in a series of inverted repeats; (h) DNA that includes nucleotides derived from a miRNA, preferably a plant miRNA; (i) DNA that includes nucleotides of a siRNA; (j) DNA that transcribes to an RNA aptamer capable of binding to a ligand; and (k) DNA that transcribes to an RNA aptamer capable of binding to a ligand, and DNA that transcribes to regulatory RNA capable of regulating expression of the first target gene, wherein the regulation is dependent on the conformation of the regulatory RNA, and the conformation of the regulatory RNA is allosterically affected by the binding state of the RNA aptamer.

Any of these gene suppression elements, whether transcribing to a single double-stranded RNA or to multiple double-stranded RNAs, can be designed to suppress more than one target gene, including, for example, more than one allele of a target gene, multiple target genes (or multiple segments of at least one target gene) from a single species, or target genes from different species.

Anti-Sense DNA Segments: In one embodiment, the at least one anti-sense DNA segment that is anti-sense to at least one segment of the at least one first target gene includes DNA sequence that is anti-sense or complementary to at least a segment of the at least one first target gene, and can include multiple anti-sense DNA segments, that is, multiple copies of at least one anti-sense DNA segment that is anti-sense to at least one segment of the at least one first target gene. Multiple anti-sense DNA segments can include DNA sequence that is anti-sense or complementary to multiple segments of the at least one first target gene, or to multiple copies of a segment of the at least one first target gene, or to segments of multiple first target genes, or to any combination of these. Multiple anti-sense DNA segments can be fused into a chimera, e.g., including DNA sequences that are anti-sense to multiple segments of one or more first target genes and fused together.

The anti-sense DNA sequence that is anti-sense or complementary to (that is, can form Watson-Crick base-pairs with) at least a segment of the at least one first target gene has at least about 80%, or at least about 85%, or at least about 90%, or at least about 95% complementarity to at least a segment of the at least one first target gene. In one embodiment, the DNA sequence that is anti-sense or complementary to at least a segment of the at least one first target gene has between about 95% to about 100% complementarity to at least a segment of the at least one first target gene. Where the at least one anti-sense DNA segment includes multiple anti-sense DNA segments, the degree of complementarity can be, but need not be, identical for all of the multiple anti-sense DNA segments.

Sense DNA Segments: In another embodiment, the at least one sense DNA segment that is at least one segment of the at least one first target gene includes DNA sequence that corresponds to (that is, has a sequence that is identical or substantially identical to) at least a segment of the at least one first target gene, and can include multiple sense DNA segments, that is, multiple copies of at least one sense DNA segment that corresponds to (that is, has the nucleotide sequence of) at least one segment of the at least one first target gene. Multiple sense DNA segments can include DNA sequence that is or that corresponds to multiple segments of the at least one first target gene, or to multiple copies of a segment of the at least one first target gene, or to segments of multiple first target genes, or to any combination of these. Multiple sense DNA segments can be fused into a chimera, that is, can include DNA sequences corresponding to multiple segments of one or more first target genes and fused together.

The sense DNA sequence that corresponds to at least a segment of the target gene has at least about 80%, or at least about 85%, or at least about 90%, or at least about 95% sequence identity to at least a segment of the target gene. In one embodiment, the DNA sequence that corresponds to at least a segment of the target gene has between about 95% to about 100% sequence identity to at least a segment of the target gene. Where the at least one sense DNA segment includes multiple sense DNA segments, the degree of sequence identity can be, but need not be, identical for all of the multiple sense DNA segments.

Multiple Copies: Where the gene suppression element includes multiple copies of anti-sense or multiple copies of sense DNA sequence, these multiple copies can be arranged serially in tandem repeats. In some embodiments, these multiple copies can be arranged serially end-to-end, that is, in directly connected tandem repeats. In some embodiments, these multiple copies can be arranged serially in interrupted tandem repeats, where one or more spacer DNA segment can be located adjacent to one or more of the multiple copies. Tandem repeats, whether directly connected or interrupted or a combination of both, can include multiple copies of a single anti-sense or multiple copies of a single sense DNA sequence in a serial arrangement or can include multiple copies of more than one anti-sense DNA sequence or of more than one sense DNA sequence in a serial arrangement.

Double-stranded RNA: In those embodiments wherein the gene suppression element includes either at least one anti-sense DNA segment that is anti-sense to at least one segment of the at least one target gene or at least one sense DNA segment that is at least one segment of the at least one target gene, RNA transcribed from either the at least one anti-sense or at least one sense DNA may become double-stranded by the action of an RNA-dependent RNA polymerase. See, for example, U.S. Pat. No. 5,283,184, which is incorporated by reference herein.

In yet other embodiments, the gene suppression element can include DNA that transcribes to RNA for suppressing the at least one first target gene by forming double-stranded RNA and includes at least one anti-sense DNA segment that is anti-sense to at least one segment of the at least one target gene (as described above under the heading “Anti-sense DNA Segments”) and at least one sense DNA segment that is at least one segment of the at least one first target gene (as described above under the heading “Sense DNA Segments”). Such a gene suppression element can further include spacer DNA segments. Each at least one anti-sense DNA segment is complementary to at least part of a sense DNA segment in order to permit formation of double-stranded RNA by intramolecular hybridization of the at least one anti-sense DNA segment and the at least one sense DNA segment. Such complementarity between an anti-sense DNA segment and a sense DNA segment can be, but need not be, 100% complementary; in some embodiments, this complementarity can be preferably at least about 80%, or at least about 85%, or at least about 90%, or at least about 95% complementary.

The double-stranded RNA can be in the form of a single dsRNA “stem” (region of base-pairing between sense and anti-sense strands), or can have multiple dsRNA “stems.” In one embodiment, the gene suppression element can include DNA that transcribes to RNA for suppressing the at least one first target gene by forming essentially a single double-stranded RNA and includes multiple serial anti-sense DNA segments that are anti-sense to at least one segment of the at least one first target gene and multiple serial sense DNA segments that are at least one segment of the at least one first target gene; the multiple serial anti-sense and multiple serial sense segments can form a single double-stranded RNA “stem” or multiple “stems” in a serial arrangement (with or without non-base paired spacer DNA separating the multiple “stems”). In another embodiment, the gene suppression element includes DNA that transcribes to RNA for suppressing the at least one first target gene by forming multiple dsRNA “stems” of RNA and includes multiple anti-sense DNA segments that are anti-sense to at least one segment of the at least one first target gene and multiple sense DNA segments that are at least one segment of the at least one first target gene, and wherein the multiple anti-sense DNA segments and the multiple sense DNA segments are arranged in a series of dsRNA “stems” (such as, but not limited to “inverted repeats”). Such multiple dsRNA “stems” can further be arranged in series or clusters to form tandem inverted repeats, or structures resembling “hammerhead” or “cloverleaf” shapes. Any of these gene suppression elements can further include spacer DNA segments found within a dsRNA “stem” (for example, as a spacer between multiple anti-sense or sense DNA segments or as a spacer between a base-pairing anti-sense DNA segment and a sense DNA segment) or outside of a double-stranded RNA “stem” (for example, as a loop region separating a pair of inverted repeats). In cases where base-pairing anti-sense and sense DNA segments are of unequal length, the longer segment can act as a spacer.

miRNAs: In a further embodiment, the gene suppression element can include DNA that includes nucleotides derived from a miRNA (microRNA), that is, a DNA sequence that corresponds to a miRNA native to a virus or a eukaryote (including plants and animals, especially invertebrates), or a DNA sequence derived from such a native miRNA but modified to include nucleotide sequences that do not correspond to the native miRNA. While miRNAs have not been reported in fungi, fungal miRNAs, should they exist, are also suitable for use in the invention. An embodiment includes a gene suppression element containing DNA that includes nucleotides derived from a viral or plant miRNA.

In a non-limiting example, the nucleotides derived from a miRNA can include DNA that includes nucleotides corresponding to the loop region of a native miRNA and nucleotides that are selected from a target gene sequence. In another non-limiting example, the nucleotides derived from a miRNA can include DNA derived from a miRNA precursor sequence, such as a native pri-miRNA or pre-miRNA sequence, or nucleotides corresponding to the regions of a native miRNA, and nucleotides that are selected from a target gene sequence such that the overall structure (e.g., the placement of mismatches in the stem structure of the pre-miRNA) is preserved to permit the pre-miRNA to be processed into a mature miRNA. In yet another embodiment, the gene suppression element can include DNA that includes nucleotides derived from a miRNA and capable of inducing or guiding in-phase cleavage of an endogenous transcript into trans-acting siRNAs, as described by Allen et al. (2005) Cell, 121:207-221. Thus, the DNA that includes nucleotides derived from a miRNA can include sequence naturally occurring in a miRNA or a miRNA precursor molecule, synthetic sequence, or both.

siRNAs: In yet another embodiment, the gene suppression element can include DNA that includes nucleotides of a small interfering RNA (siRNA). The siRNA can be one or more native siRNAs (such as siRNAs isolated from a non-transgenic eukaryote or from a transgenic eukaryote), or can be one or more DNA sequences predicted to have siRNA activity (such as by use of predictive tools known in the art, see, for example, Reynolds et al. (2004) Nature Biotechnol., 22:326-330). Multiple native or predicted siRNA sequences can be joined in a chimeric siRNA sequence for gene suppression. Such a DNA that includes nucleotides of a siRNA includes at least 19 nucleotides, and in some embodiments includes at least 20, at least 21, at least 22, at least 23, or at least 24 nucleotides. In other embodiments, the DNA that includes nucleotides of a siRNA can contain substantially more than 21 nucleotides, for example, more than about 50, about 100, about 300, about 500, about 1000, about 3000, or about 5000 nucleotides or greater.

Engineered miRNAs and trans-acting siRNAs (ta-siRNAs) are useful for gene suppression with increased specificity. The invention provides recombinant DNA constructs, each including a transcribable engineered miRNA precursor designed to suppress a target sequence, wherein the transcribable engineered miRNA precursor is derived from the fold-back structure of a MIR gene, preferably a plant MIR sequence. An engineered precursor miRNA may be designed based on all or part of a MIR gene sequence, or a derivative or variant sequence thereof, but with the targeting sequence of the MIR gene being replaced with a different sequence that targets and hybridizes to the recognition site of a target mRNA of a gene of interest. For example, a precursor miRNA may be derived from one of SEQ ID NOs: 91-94, but with the targeting sequence replaced with a different sequence that targets and hybridizes to a mRNA encoded by a target gene of interest. miRNA precursors can also be useful for directing in-phase production of siRNAs (e.g., heterologous sequence designed to be processed in a trans-acting siRNA suppression mechanism in planta). The invention further provides a method to suppress expression of a target sequence in a plant cell, including transcribing in a plant cell a recombinant DNA including a transcribable engineered miRNA precursor designed to suppress a target sequence, wherein the transcribable engineered miRNA precursor is derived from the fold-back structure of a MIR gene, preferably a plant MIR sequence, whereby expression of the target sequence is suppressed relative to its expression in the absence of transcription of the recombinant DNA construct.

The mature miRNAs produced, or predicted to be produced, from these miRNA precursors may be engineered for use in suppression of a target gene, e.g., in transcriptional suppression by the miRNA, or to direct in-phase production of siRNAs in a trans-acting siRNA suppression mechanism (see Allen et al. (2005) Cell, 121:207-221, Vaucheret (2005) Science STKE, 2005:pe43, and Yoshikawa et al. (2005) Genes Dev., 19:2164-2175). Plant miRNAs generally have near-perfect complementarity to their target sequences (see, for example, Llave et al. (2002) Science, 297:2053-2056, Rhoades et al. (2002) Cell, 110:513-520, Jones-Rhoades and Bartel (2004) Mol. Cell, 14:787-799). Thus, the mature miRNAs can be engineered to serve as sequences useful for gene suppression of a target sequence, by replacing nucleotides of the mature miRNA sequence with nucleotides of the sequence that is targeted for suppression; see, for example, methods disclosed by Parizotto et al. (2004) Genes Dev., 18:2237-2242 and especially U.S. Patent Application Publications US2004/0053411A1, US2004/0268441A1, US2005/0144669, and US2005/0037988, all of which are incorporated by reference herein. When engineering a novel miRNA to target a specific sequence, one strategy is to select within the target sequence a region with sequence that is as similar as possible to the native miRNA sequence. Alternatively, the native miRNA sequence can be replaced with a region of the target sequence, preferably a region that meets structural and thermodynamic criteria believed to be important for miRNA function (see, for example, U.S. Patent Application Publication US2005/0037988). Sequences are preferably engineered such that the number and placement of mismatches in the stem structure of the fold-back region or pre-miRNA is preserved. Thus, an engineered miRNA or engineered miRNA precursor can be derived from any of the mature miRNA sequences, or their corresponding miRNA precursors (including the fold-back portions of the corresponding MIR genes) disclosed herein. The engineered miRNA precursor can be cloned and expressed (transiently or stably) in a plant cell or tissue or intact plant.

The construction and description of recombinant DNA constructs to modulate small non-coding RNA activities are disclosed in U.S. Patent Application Publication US 2009/0070898 A1, US2011/0296555 A1, US2011/0035839 A1, all of which are incorporated herein by reference in their entirety. In particular, with respect to US2011/0035839 A1, see e.g., sections under the headings “Gene Suppression Elements” in paragraphs 122 to 135, and “Engineered Heterologous miRNA for Controlling Gene Expression in paragraphs 188 to 190.

A recombinant DNA molecule, construct or vector may comprise a transcribable DNA or polynucleotide sequence encoding a RNA or non-coding RNA molecule, wherein the RNA comprises a sequence that is at least 80%, at least 85%, at least 90%, at least 95%, at least 96%, at least 97%, at least 98%, at least 99%, at least 99.5%, or 100% complementary to at least a segment or portion of a mRNA molecule expressed from an endogenous target gene in a plant, wherein the transcribable DNA sequence is operably linked to a plant-expressible promoter. The RNA molecule may target a mature mRNA and/or intronic sequence(s) of a target gene or transcript. According to many embodiments, a RNA encoded by a recombinant DNA construct targeting a gene of interest for suppression may comprise a sequence that is at least 80%, at least 85%, at least 90%, at least 95%, at least 96%, at least 97%, at least 98%, at least 99%, at least 99.5%, or 100% complementary to at least 15, at least 16, at least 17, at least 18, at least 19, at least 20, at least 21, at least 22, at least 23, at least 24, at least 25, at least 26, or at least 27 consecutive nucleotides of any one of SEQ ID NOs: 63-69, or of an endogenous mRNA molecule encoding any one of SEQ ID NOs: 70-76. According to some embodiments, a RNA encoded by a recombinant DNA construct targeting a gene of interest for suppression may comprise a sequence that is at least 80%, at least 85%, at least 90%, at least 95%, at least 96%, at least 97%, at least 98%, at least 99%, at least 99.5%, or 100% identical to at least 15, at least 16, at least 17, at least 18, at least 19, at least 20, at least 21, at least 22, at least 23, at least 24, at least 25, at least 26, or at least 27 consecutive nucleotides of any one of SEQ ID NOs: 77-83. According to some embodiments, a RNA encoded by a recombinant DNA construct targeting a gene of interest for suppression may comprise any one of SEQ ID NOs: 77-90.

As used herein, a “plant” includes a whole plant, a modified or transgenic plant, meristematic tissue, a shoot organ/structure (for example, leaf, stem and tuber), a root, a flower, a floral organ/structure (for example, a bract, a sepal, a petal, a stamen, a carpel, an anther and an ovule), a seed (including an embryo, endosperm, and a seed coat) and a fruit (the mature ovary), plant tissue (for example, vascular tissue, ground tissue, and the like) and a cell (for example, guard cell, egg cell, pollen, mesophyll cell, and the like), and progeny of same. The classes of plants that can be used in the disclosed methods are generally as broad as the classes of higher and lower plants amenable to transformation and breeding techniques, including angiosperms (monocotyledonous and dicotyledonous plants), gymnosperms, ferns, horsetails, psilophytes, lycophytes, bryophytes, and multicellular algae.

As used herein, a “modified plant cell” means a plant cell that has been modified by the introduction of a mutation or genome edit created using a mutagenesis or genome editing technique. As used herein, a “transgenic plant cell” means a plant cell that is transformed with stably-integrated, recombinant DNA, for example, by Agrobacterium-mediated transformation, by bombardment using microparticles coated with recombinant DNA, or by other means, such as site-directed integration. A plant cell of this disclosure can be an originally transformed, edited or mutated plant cell that exists as a microorganism or as a progeny plant cell that is regenerated into differentiated tissue, for example, into a modified or transgenic plant with a stably-integrated recombinant DNA or an introduced edit or mutation, or seed or pollen derived from a modified or transgenic plant or progeny plant thereof. As used herein, a “modified plant” and a “modified plant part” mean a plant or plant part, respectively, having in the genome of at least one cell of such plant or plant part a mutation or genome edit created using a mutagenesis or genome editing technique. As used herein, a “transgenic plant” and a “transgenic plant part” mean a plant or plant part, respectively, having in the genome of at least one cell of such plant or plant part a stably-integrated, recombinant DNA construct or sequence created using a transformation method.

As used herein a “control plant” means a plant that does not contain the recombinant DNA or an edit or mutation of the present disclosure that imparts an enhanced trait or altered phenotype. A control plant is used to identify and select a modified or transgenic plant that has an enhanced trait or altered phenotype. A suitable control plant can be a non-transgenic and non-modified plant of the parental line used to generate a modified or transgenic plant, for example, a wild type plant devoid of a recombinant DNA or engineered mutation. A suitable control plant can also be a modified or transgenic plant that contains recombinant DNA, mutation or edit that imparts other traits, for example, a transgenic plant having enhanced herbicide tolerance. A suitable control plant can in some cases be a progeny of a heterozygous or hemizygous modified or transgenic plant line that does not contain the recombinant DNA, mutation or edit, known as a negative segregant, or a negative isogenic line.

As used herein a “propagule” includes all products of meiosis and mitosis, including but not limited to, plant, seed and part of a plant able to propagate a new plant. Propagules include whole plants, cells, pollen, ovules, flowers, embryos, leaves, roots, stems, shoots, meristems, grains or seeds, or any plant part that is capable of growing into an entire plant. Propagule also includes graft where one portion of a plant is grafted to another portion of a different plant (even one of a different species) to create a living organism. Propagule also includes all plants and seeds produced by cloning or by bringing together meiotic products, or allowing meiotic products to come together to form an embryo or a fertilized egg (naturally or with human intervention).

As used herein a “progeny” includes any plant, seed, plant cell, and/or regenerable plant part comprising a recombinant DNA, edit or mutation of the present disclosure derived from an ancestor plant. A progeny can be homozygous or heterozygous for the transgene, edit or mutation. Progeny can be grown from seeds produced by a modified or transgenic plant comprising a recombinant DNA, edit or mutation of the present disclosure, and/or from seeds produced by a plant fertilized with pollen or ovule from a modified or transgenic plant comprising a recombinant DNA, edit or mutation of the present disclosure.

As used herein a “trait” is a physiological, morphological, biochemical, or physical characteristic of a plant or particular plant material or cell. In some instances, this characteristic is visible to the human eye and can be measured mechanically, such as seed or plant size, weight, shape, form, length, height, growth rate and development stage, or can be measured by biochemical techniques, such as detecting the protein, starch, certain metabolites, or oil content of seed or leaves, or by observation of a metabolic or physiological process, for example, by measuring tolerance to water deprivation or particular salt or sugar concentrations, or by the measurement of the expression level of a gene or genes, for example, by employing Northern analysis, RT-PCR, microarray gene expression assays, or reporter gene expression systems, or by agricultural observations such as hyperosmotic stress tolerance or yield. Any technique can be used to measure the amount of, comparative level of, or difference in any selected chemical compound or macromolecule in the transgenic plants, however.

As used herein an “enhanced trait” means a characteristic of a modified or transgenic plant as a result of stable integration and expression of a recombinant DNA in the transgenic plant. Such traits include, but are not limited to, an enhanced agronomic trait characterized by enhanced plant morphology, physiology, growth and development, yield, nutritional enhancement, disease or pest resistance, or environmental or chemical tolerance. In some specific aspects of this disclosure an enhanced trait is selected from the group consisting of decreased days from planting to maturity, increased stalk size, increased number of leaves, increased plant height growth rate in vegetative stage, increased ear size, increased ear dry weight per plant, increased number of kernels per ear, increased weight per kernel, increased number of kernels per plant, decreased ear void, extended grain fill period, reduced plant height, increased number of root branches, increased total root length, drought tolerance, increased water use efficiency, cold tolerance, increased nitrogen use efficiency, increased yield and altered phenotypes as shown in Tables 7-9 and 11-16. In another aspect of the disclosure the trait is increased yield under non-stress conditions or increased yield under environmental stress conditions. Stress conditions can include both biotic and abiotic stress, for example, drought, shade, fungal disease, viral disease, bacterial disease, insect infestation, nematode infestation, cold temperature exposure, heat exposure, osmotic stress, reduced nitrogen nutrient availability, reduced phosphorus nutrient availability and high plant density. “Yield” can be affected by many properties including without limitation, plant height, plant biomass, pod number, pod position on the plant, number of internodes, incidence of pod shatter, grain size, ear size, ear tip filling, kernel abortion, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Yield can also be affected by efficiency of germination (including germination in stressed conditions), growth rate (including growth rate in stressed conditions), flowering time and duration, ear number, ear size, ear weight, seed number per ear or pod, seed size, composition of seed (starch, oil, protein) and characteristics of seed fill.

Also used herein, the term “trait modification” encompasses altering the naturally occurring trait by producing a detectable difference in a characteristic in a plant comprising a recombinant DNA, edit or mutation of the present disclosure relative to a plant not comprising the recombinant DNA, edit or mutation, such as a wild-type plant, or a negative segregant. In some cases, the trait modification can be evaluated quantitatively. For example, the trait modification can entail an increase or decrease, in an observed trait characteristics or phenotype as compared to a control plant. It is known that there can be natural variations in a modified trait. Therefore, the trait modification observed entails a change of the normal distribution and magnitude of the trait characteristics or phenotype in the plants as compared to a control plant.

The present disclosure relates to a plant with improved economically important characteristics, more specifically increased yield. More specifically the present disclosure relates to a modified or transgenic plant comprising a recombinant polynucleotide, edit or mutation of this disclosure, wherein the plant has increased yield as compared to a control plant. Many plants of this disclosure exhibited increased yield or improved yield trait components as compared to a control plant. In an embodiment, a modified or transgenic plant of the present disclosure exhibited an improved trait that is related to yield, including but not limited to increased nitrogen use efficiency, increased nitrogen stress tolerance, increased water use efficiency and increased drought tolerance, as defined and discussed infra.

Yield can be defined as the measurable produce of economic value from a crop. Yield can be defined in the scope of quantity and/or quality. Yield can be directly dependent on several factors, for example, the number and size of organs, plant architecture (such as the number of branches, plant biomass, etc.), flowering time and duration, grain fill period. Root architecture and development, photosynthetic efficiency, nutrient uptake, stress tolerance, early vigor, delayed senescence and functional stay green phenotypes can be important factors in determining yield. Optimizing the above mentioned factors can therefore contribute to increasing crop yield.

Reference herein to an increase in yield-related traits can also be taken to mean an increase in biomass (weight) of one or more parts of a plant, which can include above ground and/or below ground (harvestable) plant parts. In particular, such harvestable parts are seeds, and performance of the methods of the disclosure results in plants with increased yield and in particular increased seed yield relative to the seed yield of suitable control plants. The term “yield” of a plant can relate to vegetative biomass (root and/or shoot biomass), to reproductive organs, and/or to propagules (such as seeds) of that plant.

Increased yield of a plant of the present disclosure can be measured in a number of ways, including test weight, seed number per plant, seed weight, seed number per unit area (for example, seeds, or weight of seeds, per acre), bushels per acre, tons per acre, or kilo per hectare. For example, corn yield can be measured as production of shelled corn kernels per unit of production area, for example in bushels per acre or metric tons per hectare. This is often also reported on a moisture adjusted basis, for example at 15.5 percent moisture. Increased yield can result from improved utilization of key biochemical compounds, such as nitrogen, phosphorous and carbohydrate, or from improved responses to environmental stresses, such as cold, heat, drought, salt, shade, high plant density, and attack by pests or pathogens. This disclosure can also be used to provide plants with improved growth and development, and ultimately increased yield, as the result of modified expression of plant growth regulators or modification of cell cycle or photosynthesis pathways. Also of interest is the generation of plants that demonstrate increased yield with respect to a seed component that may or may not correspond to an increase in overall plant yield.

In an embodiment, “alfalfa yield” can also be measured in forage yield, the amount of above ground biomass at harvest. Factors leading contributing to increased biomass include increased vegetative growth, branches, nodes and internodes, leaf area, and leaf area index.

In another embodiment, “canola yield” can also be measured in pod number, number of pods per plant, number of pods per node, number of internodes, incidence of pod shatter, seeds per silique, seed weight per silique, improved seed, oil, or protein composition.

Additionally, “corn or maize yield” can also be measured as production of shelled corn kernels per unit of production area, ears per acre, number of kernel rows per ear and number of kernels per row, kernel number or weight per ear, weight per kernel, ear number, ear weight, fresh or dry ear biomass (weight)

In yet another embodiment, “cotton yield” can be measured as bolls per plant, size of bolls, fiber quality, seed cotton yield in g/plant, seed cotton yield in lb/acre, lint yield in lb/acre, and number of bales.

Specific embodiment for “rice yield” can also include panicles per hill, grain per hill, and filled grains per panicle.

Still further embodiment for “soybean yield” can also include pods per plant, pods per acre, seeds per plant, seeds per pod, weight per seed, weight per pod, pods per node, number of nodes, and the number of internodes per plant.

In still further embodiment, “sugarcane yield” can be measured as cane yield (tons per acre; kg/hectare), total recoverable sugar (pounds per ton), and sugar yield (tons/acre).

In yet still further embodiment, “wheat yield” can include: cereal per unit area, grain number, grain weight, grain size, grains per head, seeds per head, seeds per plant, heads per acre, number of viable tillers per plant, composition of seed (for example, carbohydrates, starch, oil, and protein) and characteristics of seed fill.

The terms “yield”, “seed yield” are defined above for a number of core crops. The terms “increased”, “improved”, “enhanced” are interchangeable and are defined herein.

In another embodiment, the present disclosure provides a method for the production of plants having altered phenotype, enhanced trait, or increased yield; performance of the method gives plants altered phenotype, enhanced trait, or increased yield.

“Increased yield” can manifest as one or more of the following: (i) increased plant biomass (weight) of one or more parts of a plant, particularly aboveground (harvestable) parts, of a plant, increased root biomass (increased number of roots, increased root thickness, increased root length) or increased biomass of any other harvestable part; or (ii) increased early vigor, defined herein as an improved seedling aboveground area approximately three weeks post-germination. “Early vigor” refers to active healthy plant growth especially during early stages of plant growth, and can result from increased plant fitness due to, for example, the plants being better adapted to their environment (for example, optimizing the use of energy resources, uptake of nutrients and partitioning carbon allocation between shoot and root). Early vigor in corn, for example, is a combination of the ability of corn seeds to germinate and emerge after planting and the ability of the young corn plants to grow and develop after emergence. Plants having early vigor also show increased seedling survival and better establishment of the crop, which often results in highly uniform fields with the majority of the plants reaching the various stages of development at substantially the same time, which often results in increased yield. Therefore early vigor can be determined by measuring various factors, such as kernel weight, percentage germination, percentage emergence, seedling growth, seedling height, root length, root and shoot biomass, canopy size and color and others.

Further, increased yield can also manifest as (iii) increased total seed yield, which may result from one or more of an increase in seed biomass (seed weight) due to an increase in the seed weight on a per plant and/or on an individual seed basis an increased number of panicles per plant; an increased number of pods; an increased number of nodes; an increased number of flowers (“florets”) per panicle/plant; increased seed fill rate; an increased number of filled seeds; increased seed size (length, width, area, perimeter), which can also influence the composition of seeds; and/or increased seed volume, which can also influence the composition of seeds. In one embodiment, increased yield can be increased seed yield, and is selected from one or more of the following: (i) increased seed weight; (ii) increased number of filled seeds; and (iii) increased harvest index.

Increased yield can also (iv) result in modified architecture, or can occur because of modified plant architecture.

Increased yield can also manifest as (v) increased harvest index, which is expressed as a ratio of the yield of harvestable parts, such as seeds, over the total biomass

Increased yield can also manifest as (vi) increased kernel weight, which is extrapolated from the number of filled seeds counted and their total weight. An increased kernel weight can result from an increased seed size and/or seed weight, an increase in embryo size, increased endosperm size, aleurone and/or scutellum, or an increase with respect to other parts of the seed that result in increased kernel weight.

Increased yield can also manifest as (vii) increased ear biomass, which is the weight of the ear and can be represented on a per ear, per plant or per plot basis.

The disclosure also extends to harvestable parts of a plant such as, but not limited to, seeds, leaves, fruits, flowers, bolls, pods, siliques, nuts, stems, rhizomes, tubers and bulbs. The disclosure furthermore relates to products derived from a harvestable part of such a plant, such as dry pellets, powders, oil, fat and fatty acids, starch or proteins.

The present disclosure provides a method for increasing “yield” of a plant or “broad acre yield” of a plant or plant part defined as the harvestable plant parts per unit area, for example seeds, or weight of seeds, per acre, pounds per acre, bushels per acre, tones per acre, tons per acre, kilo per hectare.

This disclosure further provides a method of altering phenotype, enhancing trait, or increasing yield in a plant by producing a plant comprising a polynucleic acid sequence of this disclosure where the plant can be crossed with itself, a second plant from the same plant line, a wild type plant, or a plant from a different line of plants to produce a seed. The seed of the resultant plant can be harvested from fertile plants and be used to grow progeny generations of plant(s) of this disclosure. In addition to direct transformation of a plant with a recombinant DNA construct, transgenic plants can be prepared by crossing a first plant having a stably integrated recombinant DNA construct with a second plant lacking the DNA. For example, recombinant DNA can be introduced into a first plant line that is amenable to transformation to produce a transgenic plant which can be crossed with a second plant line to introgress the recombinant DNA into the second plant line.

Selected transgenic plants transformed with a recombinant DNA construct and having the polynucleotide of this disclosure provides the altered phenotype, enhanced trait, or increased yield compared to a control plant. Use of genetic markers associated with the recombinant DNA can facilitate production of transgenic progeny that is homozygous for the desired recombinant DNA. Progeny plants carrying DNA for both parental traits can be back-crossed into a parent line multiple times, for example usually 6 to 8 generations, to produce a progeny plant with substantially the same genotype as the one reoccurring original transgenic parental line but having the recombinant DNA of the other transgenic parental line. The term “progeny” denotes the offspring of any generation of a parent plant prepared by the methods of this disclosure containing the recombinant polynucleotides as described herein.

As used herein “nitrogen use efficiency” refers to the processes which lead to an increase in the plant's yield, biomass, vigor, and growth rate per nitrogen unit applied. The processes can include the uptake, assimilation, accumulation, signaling, sensing, retranslocation (within the plant) and use of nitrogen by the plant.

As used herein “nitrogen limiting conditions” refers to growth conditions or environments that provide less than optimal amounts of nitrogen needed for adequate or successful plant metabolism, growth, reproductive success and/or viability.

As used herein the “increased nitrogen stress tolerance” refers to the ability of plants to grow, develop, or yield normally, or grow, develop, or yield faster or better when subjected to less than optimal amounts of available/applied nitrogen, or under nitrogen limiting conditions.

As used herein “increased nitrogen use efficiency” refers to the ability of plants to grow, develop, or yield faster or better than normal when subjected to the same amount of available/applied nitrogen as under normal or standard conditions; ability of plants to grow, develop, or yield normally, or grow, develop, or yield faster or better when subjected to less than optimal amounts of available/applied nitrogen, or under nitrogen limiting conditions.

Increased plant nitrogen use efficiency can be translated in the field into either harvesting similar quantities of yield, while supplying less nitrogen, or increased yield gained by supplying optimal/sufficient amounts of nitrogen. The increased nitrogen use efficiency can improve plant nitrogen stress tolerance, and can also improve crop quality and biochemical constituents of the seed such as protein yield and oil yield. The terms “increased nitrogen use efficiency”, “enhanced nitrogen use efficiency”, and “nitrogen stress tolerance” are used inter-changeably in the present disclosure to refer to plants with improved productivity under nitrogen limiting conditions.

As used herein “water use efficiency” refers to the amount of carbon dioxide assimilated by leaves per unit of water vapor transpired. It constitutes one of the most important traits controlling plant productivity in dry environments. “Drought tolerance” refers to the degree to which a plant is adapted to arid or drought conditions. The physiological responses of plants to a deficit of water include leaf wilting, a reduction in leaf area, leaf abscission, and the stimulation of root growth by directing nutrients to the underground parts of the plants. Plants are more susceptible to drought during flowering and seed development (the reproductive stages), as plant's resources are deviated to support root growth. In addition, abscisic acid (ABA), a plant stress hormone, induces the closure of leaf stomata (microscopic pores involved in gas exchange), thereby reducing water loss through transpiration, and decreasing the rate of photosynthesis. These responses improve the water-use efficiency of the plant on the short term. The terms “increased water use efficiency”, “enhanced water use efficiency”, and “increased drought tolerance” are used inter-changeably in the present disclosure to refer to plants with improved productivity under water-limiting conditions.

As used herein “increased water use efficiency” refers to the ability of plants to grow, develop, or yield faster or better than normal when subjected to the same amount of available/applied water as under normal or standard conditions; ability of plants to grow, develop, or yield normally, or grow, develop, or yield faster or better when subjected to reduced amounts of available/applied water (water input) or under conditions of water stress or water deficit stress.

As used herein “increased drought tolerance” refers to the ability of plants to grow, develop, or yield normally, or grow, develop, or yield faster or better than normal when subjected to reduced amounts of available/applied water and/or under conditions of acute or chronic drought; ability of plants to grow, develop, or yield normally when subjected to reduced amounts of available/applied water (water input) or under conditions of water deficit stress or under conditions of acute or chronic drought.

As used herein “drought stress” refers to a period of dryness (acute or chronic/prolonged) that results in water deficit and subjects plants to stress and/or damage to plant tissues and/or negatively affects grain/crop yield; a period of dryness (acute or chronic/prolonged) that results in water deficit and/or higher temperatures and subjects plants to stress and/or damage to plant tissues and/or negatively affects grain/crop yield.

As used herein “water deficit” refers to the conditions or environments that provide less than optimal amounts of water needed for adequate/successful growth and development of plants.

As used herein “water stress” refers to the conditions or environments that provide improper (either less/insufficient or more/excessive) amounts of water than that needed for adequate/successful growth and development of plants/crops thereby subjecting the plants to stress and/or damage to plant tissues and/or negatively affecting grain/crop yield.

As used herein “water deficit stress” refers to the conditions or environments that provide less/insufficient amounts of water than that needed for adequate/successful growth and development of plants/crops thereby subjecting the plants to stress and/or damage to plant tissues and/or negatively affecting grain yield.

As used herein a “polynucleotide” is a nucleic acid molecule comprising a plurality of polymerized nucleotides. A polynucleotide may be referred to as a nucleic acid, a oligonucleotide, or any fragment thereof. In many instances, a polynucleotide encodes a polypeptide (or protein) or a domain or a fragment thereof. Additionally, a polynucleotide can comprise a promoter, an intron, an enhancer region, a polyadenylation site, a translation initiation site, 5′ or 3′ untranslated regions, a reporter gene, a selectable marker, a scorable marker, or the like. A polynucleotide can be single-stranded or double-stranded DNA or RNA. A polynucleotide optionally comprises modified bases or a modified backbone. A polynucleotide can be, for example, genomic DNA or RNA, a transcript (such as an mRNA), a cDNA, a PCR product, a cloned DNA, a synthetic DNA or RNA, or the like. A polynucleotide can be combined with carbohydrate(s), lipid(s), protein(s), or other materials to perform a particular activity such as transformation or form a composition such as a peptide nucleic acid (PNA). A polynucleotide can comprise a sequence in either sense or antisense orientations. “Oligonucleotide” is substantially equivalent to the terms amplimer, primer, oligomer, element, target, and probe and is preferably single-stranded.

As used herein a “recombinant polynucleotide” or “recombinant DNA” is a polynucleotide that is not in its native state, for example, a polynucleotide comprises a series of nucleotides (represented as a nucleotide sequence) not found in nature, or a polynucleotide is in a context other than that in which it is naturally found; for example, separated from polynucleotides with which it typically is in proximity in nature, or adjacent (or contiguous with) polynucleotides with which it typically is not in proximity. The “recombinant polynucleotide” or “recombinant DNA” refers to polynucleotide or DNA which has been genetically engineered and constructed outside of a cell including DNA containing naturally occurring DNA or cDNA or synthetic DNA. For example, the polynucleotide at issue can be cloned into a vector, or otherwise recombined with one or more additional nucleic acids.

As used herein a “polypeptide” comprises a plurality of consecutive polymerized amino acid residues for example, at least about 15 consecutive polymerized amino acid residues. In many instances, a polypeptide comprises a series of polymerized amino acid residues that is a transcriptional regulator or a domain or portion or fragment thereof. Additionally, the polypeptide can comprise: (i) a localization domain; (ii) an activation domain; (iii) a repression domain; (iv) an oligomerization domain; (v) a protein-protein interaction domain; (vi) a DNA-binding domain; or the like. The polypeptide optionally comprises modified amino acid residues, naturally occurring amino acid residues not encoded by a codon, non-naturally occurring amino acid residues.

As used herein “protein” refers to a series of amino acids, oligopeptide, peptide, polypeptide or portions thereof whether naturally occurring or synthetic.

As used herein a “recombinant polypeptide” is a polypeptide produced by translation of a recombinant polynucleotide.

A “synthetic polypeptide” is a polypeptide created by consecutive polymerization of isolated amino acid residues using methods known in the art.

An “isolated polypeptide”, whether a naturally occurring or a recombinant polypeptide, is more enriched in (or out of) a cell than the polypeptide in its natural state in a wild-type cell, for example, more than about 5% enriched, more than about 10% enriched, or more than about 20%, or more than about 50%, or more, enriched, for example, alternatively denoted: 105%, 110%, 120%, 150% or more, enriched relative to wild type standardized at 100%. Such enrichment is not the result of a natural response of a wild-type plant. Alternatively, or additionally, the isolated polypeptide is separated from other cellular components, with which it is typically associated, for example, by any of the various protein purification methods.

As used herein, a “functional fragment” refers to a portion of a polypeptide provided herein which retains full or partial molecular, physiological or biochemical function of the full length polypeptide. A functional fragment often contains the domain(s), such as Pfam domains (see below), identified in the polypeptide provided in the sequence listing.

A “recombinant DNA construct” as used in the present disclosure comprises at least one expression cassette having a promoter operable in plant cells and a polynucleotide of the present disclosure. DNA constructs can be used as a means of delivering recombinant DNA constructs to a plant cell in order to effect stable integration of the recombinant molecule into the plant cell genome. In one embodiment, the polynucleotide can encode a protein or variant of a protein or fragment of a protein that is functionally defined to maintain activity in transgenic host cells including plant cells, plant parts, explants and whole plants. In another embodiment, the polynucleotide can encode a non-coding RNA that interferes with the functioning of endogenous classes of small RNAs that regulate expression, including but not limited to taRNAs, siRNAs and miRNAs. Recombinant DNA constructs are assembled using methods known to persons of ordinary skill in the art and typically comprise a promoter operably linked to DNA, the expression of which provides the enhanced agronomic trait.

Other construct components can include additional regulatory elements, such as 5′ leaders and introns for enhancing transcription, 3′ untranslated regions (such as polyadenylation signals and sites), and DNA for transit or targeting or signal peptides.

As used herein, a “homolog” or “homologues” means a protein in a group of proteins that perform the same biological function, for example, proteins that belong to the same Pfam protein family and that provide a common enhanced trait in transgenic plants of this disclosure. Homologs are expressed by homologous genes. With reference to homologous genes, homologs include orthologs, for example, genes expressed in different species that evolved from common ancestral genes by speciation and encode proteins retain the same function, but do not include paralogs, i.e., genes that are related by duplication but have evolved to encode proteins with different functions. Homologous genes include naturally occurring alleles and artificially-created variants.

Degeneracy of the genetic code provides the possibility to substitute at least one base of the protein encoding sequence of a gene with a different base without causing the amino acid sequence of the polypeptide produced from the gene to be changed. When optimally aligned, homolog proteins, or their corresponding nucleotide sequences, have typically at least about 60% identity, in some instances at least about 70%, at least about 75%, at least about 80%, at least about 85%, at least about 90%, at least about 92%, at least about 93%, at least about 94%, at least about 95%, at least about 96%, at least about 97%, at least about 98%, at least about 99%, or even at least about 99.5% identity over the full length of a protein or its corresponding nucleotide sequence identified as being associated with imparting an enhanced trait or altered phenotype when expressed in plant cells. In one aspect of the disclosure homolog proteins have at least about 80%, at least about 85%, at least about 90%, at least about 92%, at least about 93%, at least about 94%, at least about 95%, at least about 96%, at least about 97%, at least about 98%, at least about 99%, or at least about 99.5% identity to a consensus amino acid sequence of proteins and homologs that can be built from sequences disclosed herein.

Homologs are inferred from sequence similarity, by comparison of protein sequences, for example, manually or by use of a computer-based tool using known sequence comparison algorithms such as BLAST and FASTA. A sequence search and local alignment program, for example, BLAST, can be used to search query protein sequences of a base organism against a database of protein sequences of various organisms, to find similar sequences, and the summary Expectation value (E-value) can be used to measure the level of sequence similarity. Because a protein hit with the lowest E-value for a particular organism may not necessarily be an ortholog or be the only ortholog, a reciprocal query is used to filter hit sequences with significant E-values for ortholog identification. The reciprocal query entails search of the significant hits against a database of protein sequences of the base organism. A hit can be identified as an ortholog, when the reciprocal query's best hit is the query protein itself or a paralog of the query protein. With the reciprocal query process orthologs are further differentiated from paralogs among all the homologs, which allows for the inference of functional equivalence of genes. A further aspect of the homologs encoded by DNA useful in the transgenic plants of the invention are those proteins that differ from a disclosed protein as the result of deletion or insertion of one or more amino acids in a native sequence.

Other functional homolog proteins differ in one or more amino acids from those of a trait-improving protein disclosed herein as the result of one or more of known conservative amino acid substitutions, for example, valine is a conservative substitute for alanine and threonine is a conservative substitute for serine. Conservative substitutions for an amino acid within the native sequence can be selected from other members of a class to which the naturally occurring amino acid belongs. Representative amino acids within these various classes include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, tyrosine, asparagine, and glutamine; and (4) neutral nonpolar (hydrophobic) amino acids such as alanine, leucine, isoleucine, valine, proline, phenylalanine, tryptophan, and methionine. Conserved substitutes for an amino acid within a native protein or polypeptide can be selected from other members of the group to which the naturally occurring amino acid belongs. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side 30 chains is cysteine and methionine. Naturally conservative amino acids substitution groups are: valine-leucine, valine-isoleucine, phenylalanine-tyrosine, lysine-arginine, alanine-valine, aspartic acid-glutamic acid, and asparagine-glutamine. A further aspect of the disclosure includes proteins that differ in one or more amino acids from those of a described protein sequence as the result of deletion or insertion of one or more amino acids in a native sequence.

In general, the term “variant” refers to molecules with some differences, generated synthetically or naturally, in their nucleotide or amino acid sequences as compared to a reference (native) polynucleotides or polypeptides, respectively. These differences include substitutions, insertions, deletions or any desired combinations of such changes in a native polynucleotide or amino acid sequence.

With regard to polynucleotide variants, differences between presently disclosed polynucleotides and polynucleotide variants are limited so that the nucleotide sequences of the former and the latter are similar overall and, in many regions, identical. Due to the degeneracy of the genetic code, differences between the former and the latter nucleotide sequences may be silent (for example, the amino acids encoded by the polynucleotide are the same, and the variant polynucleotide sequence encodes the same amino acid sequence as the presently disclosed polynucleotide). Variant nucleotide sequences can encode different amino acid sequences, in which case such nucleotide differences will result in amino acid substitutions, additions, deletions, insertions, truncations or fusions with respect to the similarly disclosed polynucleotide sequences. These variations can result in polynucleotide variants encoding polypeptides that share at least one functional characteristic. The degeneracy of the genetic code also dictates that many different variant polynucleotides can encode identical and/or substantially similar polypeptides.

As used herein “gene” or “gene sequence” refers to the partial or complete coding sequence of a gene, its complement, and its 5′ and/or 3′ untranslated regions (UTRs) and their complements. A gene is also a functional unit of inheritance, and in physical terms is a particular segment or sequence of nucleotides along a molecule of DNA (or RNA, in the case of RNA viruses) involved in producing a polypeptide chain. The latter can be subjected to subsequent processing such as chemical modification or folding to obtain a functional protein or polypeptide. By way of example, a transcriptional regulator gene encodes a transcriptional regulator polypeptide, which can be functional or require processing to function as an initiator of transcription.

As used herein, the term “promoter” refers generally to a DNA molecule that is involved in recognition and binding of RNA polymerase II and other proteins (trans-acting transcription factors) to initiate transcription. A promoter can be initially isolated from the 5′ untranslated region (5′ UTR) of a genomic copy of a gene. Alternately, promoters can be synthetically produced or manipulated DNA molecules. Promoters can also be chimeric, that is a promoter produced through the fusion of two or more heterologous DNA molecules. Plant promoters include promoter DNA obtained from plants, plant viruses, fungi and bacteria such as Agrobacterium and Bradyrhizobium bacteria.

Promoters which initiate transcription in all or most tissues of the plant are referred to as “constitutive” promoters. Promoters which initiate transcription during certain periods or stages of development are referred to as “developmental” promoters. Promoters whose expression is enhanced in certain tissues of the plant relative to other plant tissues are referred to as “tissue enhanced” or “tissue preferred” promoters. Promoters which express within a specific tissue of the plant, with little or no expression in other plant tissues are referred to as “tissue specific” promoters. For example, a “seed enhanced” or “seed preferred” promoter drives enhanced or higher expression levels of an associated transgene or transcribable nucleotide sequence (i.e., operably linked to the promoter) in seed tissues relative to other tissues of the plant, whereas a “seed specific” promoter would drive expression of an associated transgene or transcribable nucleotide sequence (i.e., operably linked to the promoter) in seed tissues with little or no expression in other tissues of the plant. Other types of tissue specific or tissue preferred promoters for other tissue types, such as roots, meristem, leaf, etc., may also be described in this way. A promoter that expresses in a certain cell type of the plant, for example a microspore mother cell, is referred to as a “cell type specific” promoter. An “inducible” promoter is a promoter in which transcription is initiated in response to an environmental stimulus such as cold, drought or light; or other stimuli such as wounding or chemical application. Many physiological and biochemical processes in plants exhibit endogenous rhythms with a period of about 24 hours. A “diurnal promoter” is a promoter which exhibits altered expression profiles under the control of a circadian oscillator. Diurnal regulation is subject to environmental inputs such as light and temperature and coordination by the circadian clock.

Examples of seed preferred or seed specific promoters include promoters from genes expressed in seed tissues, such as napin as disclosed in U.S. Pat. No. 5,420,034, maize L3 oleosin as disclosed in U.S. Pat. No. 6,433,252, zein Z27 as disclosed by Russell et al. (1997) Transgenic Res. 6(2):157-166, globulin 1 as disclosed by Belanger et al (1991) Genetics 129:863-872, glutelin 1 as disclosed by Russell (1997) supra, and peroxiredoxin antioxidant (Per1) as disclosed by Stacy et al. (1996) Plant Mol Biol. 31(6):1205-1216. The contents and disclosures of each of the above references are incorporated herein by reference. Examples of meristem preferred or meristem specific promoters are provided, for example, in International Application No. PCT/US2017/057202, the contents and disclosure of which are incorporated herein by reference.

Many examples of constitutive promoters that may be used in plants are known in the art, such as a cauliflower mosaic virus (CaMV) 35S and 19S promoter (see, e.g., U.S. Pat. No. 5,352,605), an enhanced CaMV 35S promoter, such as a CaMV 35S promoter with Omega region (see, e.g., Holtorf, S. et al., Plant Molecular Biology, 29: 637-646 (1995) or a dual enhanced CaMV promoter (see, e.g., U.S. Pat. No. 5,322,938), a Figwort Mosaic Virus (FMV) 35S promoter (see, e.g., U.S. Pat. No. 6,372,211), a Mirabilis Mosaic Virus (MMV) promoter (see, e.g., U.S. Pat. No. 6,420,547), a Peanut Chlorotic Streak Caulimovirus promoter (see, e.g., U.S. Pat. No. 5,850,019), a nopaline or octopine promoter, a ubiquitin promoter, such as a soybean polyubiquitin promoter (see, e.g., U.S. Pat. No. 7,393,948), an Arabidopsis S-Adenosylmethionine synthetase promoter (see, e.g., U.S. Pat. No. 8,809,628), etc., or any functional portion of the foregoing promoters, the contents and disclosures of each of the above references are incorporated herein by reference.

Examples of constitutive promoters that may be used in monocot plants, such as cereal or corn plants, include, for example, various actin gene promoters, such as a rice Actin 1 promoter (see, e.g., U.S. Pat. No. 5,641,876; see also SEQ ID NO: 75 or SEQ ID NO: 76) and a rice Actin 2 promoter (see, e.g., U.S. Pat. No. 6,429,357; see also, e.g., SEQ ID NO: 77 or SEQ ID NO: 78), a CaMV 35S or 19S promoter (see, e.g., U.S. Pat. No. 5,352,605; see also, e.g., SEQ ID NO: 79 for CaMV 35S), a maize ubiquitin promoter (see, e.g., U.S. Pat. No. 5,510,474), a Coix lacryma-jobi polyubiquitin promoter (see, e.g., SEQ ID NO: 80), a rice or maize Gos2 promoter (see, e.g., Pater et al., The Plant Journal, 2(6): 837-44 1992; see also, e.g., SEQ ID NO: 81 for the rice Gos2 promoter), a FMV 35S promoter (see, e.g., U.S. Pat. No. 6,372,211), a dual enhanced CMV promoter (see, e.g., U.S. Pat. No. 5,322,938), a MMV promoter (see, e.g., U.S. Pat. No. 6,420,547; see also, e.g., SEQ ID NO: 82), a PCLSV promoter (see, e.g., U.S. Pat. No. 5,850,019; see also, e.g., SEQ ID NO: 83), an Emu promoter (see, e.g., Last et al., Theor. Appl. Genet. 81:581 (1991); and Mcelroy et al., Mol. Gen. Genet. 231:150 (1991)), a tubulin promoter from maize, rice or other species, a nopaline synthase (nos) promoter, an octopine synthase (ocs) promoter, a mannopine synthase (mas) promoter, or a plant alcohol dehydrogenase (e.g., maize Adh1) promoter, any other promoters including viral promoters known or later-identified in the art to provide constitutive expression in a cereal or corn plant, any other constitutive promoters known in the art that may be used in monocot or cereal plants, and any functional sequence portion or truncation of any of the foregoing promoters. The contents and disclosures of each of the above references are incorporated herein by reference.

As used herein, the term “leader” refers to a DNA molecule isolated from the untranslated 5′ region (5′ UTR) of a genomic copy of a gene and is defined generally as a nucleotide segment between the transcription start site (TSS) and the protein coding sequence start site. Alternately, leaders can be synthetically produced or manipulated DNA elements. A leader can be used as a 5′ regulatory element for modulating expression of an operably linked transcribable polynucleotide molecule. As used herein, the term “intron” refers to a DNA molecule that can be isolated or identified from the genomic copy of a gene and can be defined generally as a region spliced out during mRNA processing prior to translation. Alternately, an intron can be a synthetically produced or manipulated DNA element. An intron can contain enhancer elements that effect the transcription of operably linked genes. An intron can be used as a regulatory element for modulating expression of an operably linked transcribable polynucleotide molecule. A DNA construct can comprise an intron, and the intron may or may not be with respect to the transcribable polynucleotide molecule.

As used herein, the term “enhancer” or “enhancer element” refers to a cis-acting transcriptional regulatory element, a.k.a. cis-element, which confers an aspect of the overall expression pattern, but is usually insufficient alone to drive transcription, of an operably linked polynucleotide. Unlike promoters, enhancer elements do not usually include a transcription start site (TSS) or TATA box or equivalent sequence. A promoter can naturally comprise one or more enhancer elements that affect the transcription of an operably linked polynucleotide. An isolated enhancer element can also be fused to a promoter to produce a chimeric promoter cis-element, which confers an aspect of the overall modulation of gene expression. A promoter or promoter fragment can comprise one or more enhancer elements that effect the transcription of operably linked genes. Many promoter enhancer elements are believed to bind DNA-binding proteins and/or affect DNA topology, producing local conformations that selectively allow or restrict access of RNA polymerase to the DNA template or that facilitate selective opening of the double helix at the site of transcriptional initiation. An enhancer element can function to bind transcription factors that regulate transcription. Some enhancer elements bind more than one transcription factor, and transcription factors can interact with different affinities with more than one enhancer domain.

Expression cassettes of this disclosure can include a “transit peptide” or “targeting peptide” or “signal peptide” molecule located either 5′ or 3′ to or within the gene(s). These terms generally refer to peptide molecules that when linked to a protein of interest directs the protein to a particular tissue, cell, subcellular location, or cell organelle. Examples include, but are not limited to, chloroplast transit peptides (CTPs), chloroplast targeting peptides, mitochondrial targeting peptides, nuclear targeting signals, nuclear exporting signals, vacuolar targeting peptides, and vacuolar sorting peptides. For description of the use of chloroplast transit peptides see U.S. Pat. Nos. 5,188,642 and 5,728,925. For description of the transit peptide region of an Arabidopsis EPSPS gene in the present disclosure, see Klee, H. J. Et al (MGG (1987) 210:437-442. Expression cassettes of this disclosure can also include an intron or introns. Expression cassettes of this disclosure can contain a DNA near the 3′ end of the cassette that acts as a signal to terminate transcription from a heterologous nucleic acid and that directs polyadenylation of the resultant mRNA. These are commonly referred to as “3′-untranslated regions” or “3′-non-coding sequences” or “3′-UTRs”. The “3′ non-translated sequences” means DNA sequences located downstream of a structural nucleotide sequence and include sequences encoding polyadenylation and other regulatory signals capable of affecting mRNA processing or gene expression. The polyadenylation signal functions in plants to cause the addition of polyadenylate nucleotides to the 3′ end of the mRNA precursor. The polyadenylation signal can be derived from a natural gene, from a variety of plant genes, or from T-DNA. An example of a polyadenylation sequence is the nopaline synthase 3′ sequence (nos 3′; Fraley et al., Proc. Natl. Acad. Sci. USA 80: 4803-4807, 1983). The use of different 3′ non-translated sequences is exemplified by Ingelbrecht et al., Plant Cell 1:671-680, 1989.

Expression cassettes of this disclosure can also contain one or more genes that encode selectable markers and confer resistance to a selective agent such as an antibiotic or an herbicide. A number of selectable marker genes are known in the art and can be used in the present disclosure: selectable marker genes conferring tolerance to antibiotics like kanamycin and paromomycin (nptll), hygromycin B (aph IV), spectinomycin (aadA), U.S. Patent Publication 2009/0138985A1 and gentamycin (aac3 and aacC4) or tolerance to herbicides like glyphosate (for example, 5-enolpyruvylshikimate-3-phosphate synthase (EPSPS), U.S. Pat. Nos. 5,627,061; 5,633,435; 6,040,497; 5,094,945), sulfonyl herbicides (for example, acetohydroxyacid synthase or acetolactate synthase conferring tolerance to acetolactate synthase inhibitors such as sulfonylurea, imidazolinone, triazolopyrimidine, pyrimidyloxybenzoates and phthalide (U.S. Pat. Nos. 6,225,105; 5,767,366; 4,761,373; 5,633,437; 6,613,963; 5,013,659; 5,141,870; 5,378,824; 5,605,011)), bialaphos or phosphinothricin or derivatives (e. g., phosphinothricin acetyltransferase (bar) tolerance to phosphinothricin or glufosinate (U.S. Pat. Nos. 5,646,024; 5,561,236; 5,276,268; 5,637,489; 5,273,894); dicamba (dicamba monooxygenase, Patent Application Publications US2003/0115626A1), or sethoxydim (modified acetyl-coenzyme A carboxylase for conferring tolerance to cyclohexanedione), and aryloxyphenoxypropionate (haloxyfop, U.S. Pat. No. 6,414,222).

Transformation vectors of this disclosure can contain one or more “expression cassettes”, each comprising a native or non-native plant promoter operably linked to a polynucleotide sequence of interest, which is operably linked to a 3′ UTR sequence and termination signal, for expression in an appropriate host cell. It also typically comprises sequences required for proper translation of the polynucleotide or transgene. As used herein, the term “transgene” refers to a polynucleotide molecule artificially incorporated into a host cell's genome. Such a transgene can be heterologous to the host cell. The term “transgenic plant” refers to a plant comprising such a transgene. The coding region usually codes for a protein of interest but can also code for a functional RNA of interest, for example an antisense RNA, a non-translated RNA, in the sense or antisense direction, a miRNA, a noncoding RNA, or a synthetic RNA used in either suppression or over expression of target gene sequences. The expression cassette comprising the nucleotide sequence of interest can be chimeric, meaning that at least one of its components is heterologous with respect to at least one of its other components. As used herein the term “chimeric” refers to a DNA molecule that is created from two or more genetically diverse sources, for example a first molecule from one gene or organism and a second molecule from another gene or organism.

Recombinant DNA constructs in this disclosure generally include a 3′ element that typically contains a polyadenylation signal and site. Known 3′ elements include those from Agrobacterium tumefaciens genes such as nos 3′, tml 3, tmr 3′, tms 3′, ocs 3′, tr7 3′, for example disclosed in U.S. Pat. No. 6,090,627; 3′ elements from plant genes such as wheat (Trilicum aesevitum) heat shock protein 17 (Hsp17 3′), a wheat ubiquitin gene, a wheat fructose-1,6-biphosphatase gene, a rice glutelin gene, a rice lactate dehydrogenase gene and a rice beta-tubulin gene, all of which are disclosed in U.S. Patent Application Publication 2002/0192813 A1; and the pea (Pisum sativum) ribulose biphosphate carboxylase gene (rbs 3′), and 3′ elements from the genes within the host plant.

Transgenic plants can comprise a stack of one or more polynucleotides disclosed herein resulting in the production of multiple polypeptide sequences. Transgenic plants comprising stacks of polynucleotides can be obtained by either or both of traditional breeding methods or through genetic engineering methods. These methods include, but are not limited to, crossing individual transgenic lines each comprising a polynucleotide of interest, transforming a transgenic plant comprising a first gene disclosed herein with a second gene, and co-transformation of genes into a single plant cell. Co-transformation of genes can be carried out using single transformation vectors comprising multiple genes or genes carried separately on multiple vectors.

As an alternative to traditional transformation methods, a DNA sequence, such as a transgene, expression cassette(s), etc., may be inserted or integrated into a specific site or locus within the genome of a plant or plant cell via site-directed integration. Recombinant DNA construct(s) and molecule(s) of this disclosure may thus include a donor template sequence comprising at least one transgene, expression cassette, or other DNA sequence for insertion into the genome of the plant or plant cell. Such donor template for site-directed integration may further include one or two homology arms flanking an insertion sequence (i.e., the sequence, transgene, cassette, etc., to be inserted into the plant genome). The recombinant DNA construct(s) of this disclosure may further comprise an expression cassette(s) encoding a site-specific nuclease and/or any associated protein(s) to carry out site-directed integration, or a site-specific nuclease and/or associated protein(s) may be provided separately. A nuclease expressing cassette(s) may be present in the same molecule or vector as the donor template (in cis) or on a separate molecule or vector (in trans).

Any site or locus within the genome of a plant may potentially be chosen for site-directed integration of a transgene, construct or transcribable DNA sequence provided herein. Several methods for site-directed integration are known in the art involving different proteins (or complexes of proteins and/or guide RNA) that cut the genomic DNA to produce a double strand break (DSB) or nick at a desired genomic site or locus. Briefly as understood in the art, during the process of repairing the DSB or nick introduced by the nuclease enzyme, the donor template DNA may become integrated into the genome at or near the site of the DSB or nick. The presence of the homology arm(s) in the donor template may promote the adoption and targeting of the insertion sequence into the plant genome during the repair process through homologous recombination, although an insertion event may also occur through non-homologous end joining (NHEJ). Examples of site-specific nucleases that may be used include zinc-finger nucleases, engineered or native meganucleases, TALE-endonucleases, and RNA-guided endonucleases (e.g., Cas9 or Cpf1). For methods using RNA-guided site-specific nucleases (e.g., Cas9 or Cpf1), the recombinant DNA construct(s) will also comprise a sequence encoding one or more guide RNAs to direct the nuclease to the desired site within the plant genome.

As used herein, the term “homology arm” refers to a polynucleotide sequence that has at least 80%, at least 85%, at least 90%, at least 91%, at least 92%, at least 93%, at least 94%, at least 95%, at least 96%, at least 97%, at least 98%, at least 99% or 100% sequence identity to a target sequence in a plant or plant cell that is being transformed. A homology arm can comprise at least 15, at least 20, at least 25, at least 30, at least 40, at least 50, at least 100, at least 250, at least 500, or at least 1000 nucleotides.

As an alternative to suppression, a target gene may instead be the target of mutagenesis or genome editing to result in loss of function of the target gene. Plant mutagenesis techniques (excluding genome editing) may include chemical mutagenesis (i.e., treatment with a chemical mutagen, such as an azide, hydroxylamine, nitrous acid, acridine, nucleotide base analog, or alkylating agent—e.g., EMS (ethylmethane sulfonate), MNU (N-methyl-N-nitrosourea), etc.), physical mutagenesis (e.g., gamma rays, X-rays, UV, ion beam, other forms of radiation, etc.), and insertional mutagenesis (e.g., transposon or T-DNA insertion). Plants or various plant parts, plant tissues or plant cells may be subjected to mutagenesis. Treated plants may be reproduced to collect seeds or produce a progeny plant, and treated plant parts, plant tissues or plant cells may be developed or regenerated into plants or other plant tissues. Mutations generated with chemical or physical mutagenesis techniques may include a frameshift, missense or nonsense mutation leading to loss of function or expression of a targeted gene. Plants that have been subjected to mutagenesis or genome editing may be screened and selected based on an observable trait or phenotype (e.g., any trait or phenotype described herein).

One method for mutagenesis of a gene is called “TILLING” (for targeting induced local lesions in genomes), in which mutations are created in a plant cell or tissue, preferably in the seed, reproductive tissue or germline of a plant, for example, using a mutagen, such as an EMS treatment. The resulting plants are grown and self-fertilized, and the progeny are used to prepare DNA samples. PCR amplification and sequencing of a nucleic acid sequence of a target gene may be used to identify whether a mutated plant has a mutation in the target gene. Plants having mutations in the target gene may then be tested for an altered trait, such as reduced plant height. Alternatively, mutagenized plants may be tested for an altered trait, such as reduced plant height, and then PCR amplification and sequencing of a nucleic acid sequence of a target gene may be used to determine whether a plant having the altered trait also has a mutation in the target gene. See, e.g., Colbert et al., 2001, Plant Physiol 126:480-484; and McCallum et al., 2000, Nature Biotechnology 18:455-457. TILLING can be used to identify mutations that alter the expression a gene or the activity of proteins encoded by a gene, which may be used to introduce and select for a targeted mutation in a target gene of a plant.

Mutations may also be introduced into a target gene through genome editing techniques through the introduction of a double strand break (DSB) or nick in the genome of a plant. According to this approach, mutations, such as deletions, insertions, inversions and/or substitutions may be introduced at a desired target site at or near (e.g., within) a target gene via imperfect repair of the DSB or nick to produce a knock-out or knock-down of the target gene. Such mutations may be generated by imperfect repair of the targeted locus even without the use of a donor template molecule. A “knock-out” of a target gene may be achieved by inducing a DSB or nick at or near the endogenous locus of the target gene to result in non-expression of the gene or expression from the target gene of a non-functional protein, whereas a “knock-down” of a target gene may be achieved in a similar manner by inducing a DSB or nick at or near the endogenous locus of the target gene at a site that does not affect the coding sequence of the target gene in a manner that would eliminate the function and/or expression of its encoded protein. For example, the site of the DSB or nick within the endogenous locus may be in the upstream or 5′ region of the target gene (e.g., a promoter and/or enhancer sequence) to affect or reduce its level of expression. Similarly, targeted knock-out or knock-down mutations of a target gene may be generated with a donor template molecule to direct a particular or desired mutation at or near the target site via repair of the DSB or nick. The donor template molecule may comprise a homologous sequence with or without an insertion sequence and comprising one or more mutations, such as one or more deletions, insertions, inversions and/or substitutions, relative to the targeted genomic sequence at or near the site of the DSB or nick. For example, targeted knock-out mutations of a target gene may be achieved by deleting or inverting at least a portion of the gene or by introducing a frame shift or premature stop codon into the coding sequence of the gene. A deletion of a portion of a target gene may also be introduced by generating DSBs or nicks at two target sites and causing a deletion of the intervening target region flanked by the target sites.

A site-specific nuclease provided herein may be selected from the group consisting of a zinc-finger nuclease (ZFN), a meganuclease, an RNA-guided endonuclease, a TALE-endonuclease (TALEN), a recombinase, a transposase, or any combination thereof. See, e.g., Khandagale, K. et al., “Genome editing for targeted improvement in plants,” Plant Biotechnol Rep 10: 327-343 (2016); and Gaj, T. et al., “ZFN, TALEN and CRISPR/Cas-based methods for genome engineering,” Trends Biotechnol. 31(7): 397-405 (2013), the contents and disclosures of which are incorporated herein by reference. A recombinase may be a serine recombinase attached to a DNA recognition motif, a tyrosine recombinase attached to a DNA recognition motif or other recombinase enzyme known in the art. A recombinase or transposase may be a DNA transposase or recombinase attached to a DNA binding domain. A tyrosine recombinase attached to a DNA recognition motif may be selected from the group consisting of a Cre recombinase, a Flp recombinase, and a Tnp1 recombinase. According to some embodiments, a Cre recombinase or a Gin recombinase provided herein is tethered to a zinc-finger DNA binding domain. In another embodiment, a serine recombinase attached to a DNA recognition motif provided herein is selected from the group consisting of a PhiC31 integrase, an R4 integrase, and a TP-901 integrase. In another embodiment, a DNA transposase attached to a DNA binding domain provided herein is selected from the group consisting of a TALE-piggyBac and TALE-Mutator. According to embodiments of the present disclosure, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, and homologs or modified versions thereof, Argonaute (non-limiting examples of Argonaute proteins include Thermus thermophilus Argonaute (TtAgo), Pyrococcus furiosus Argonaute (PfAgo), Natronobacterium gregoryi Argonaute (NgAgo) and homologs or modified versions thereof. According to some embodiments, an RNA-guided endonuclease may be a Cas9 or Cpf1 enzyme.

For RNA-guided endonucleases, a guide RNA (gRNA) molecule is further provided to direct the endonuclease to a target site in the genome of the plant via base-pairing or hybridization to cause a DSB or nick at or near the target site. The gRNA may be transformed or introduced into a plant cell or tissue (perhaps along with a nuclease, or nuclease-encoding DNA molecule, construct or vector) as a gRNA molecule, or as a recombinant DNA molecule, construct or vector comprising a transcribable DNA sequence encoding the guide RNA operably linked to a plant-expressible promoter. As understood in the art, a “guide RNA” may comprise, for example, a CRISPR RNA (crRNA), a single-chain guide RNA (sgRNA), or any other RNA molecule that may guide or direct an endonuclease to a specific target site in the genome. A “single-chain guide RNA” (or “sgRNA”) is a RNA molecule comprising a crRNA covalently linked a tracrRNA by a linker sequence, which may be expressed as a single RNA transcript or molecule. The guide RNA comprises a guide or targeting sequence that is identical or complementary to a target site within the plant genome, such as at or near a target gene. A protospacer-adjacent motif (PAM) may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu, X. et al., “Target specificity of the CRISPR-Cas9 system,” Quant Biol. 2(2): 59-70 (2014), the content and disclosure of which is incorporated herein by reference. The genomic PAM sequence on the sense (+) strand adjacent to the target site (relative to the targeting sequence of the guide RNA) may comprise 5′-NGG-3′. However, the corresponding sequence of the guide RNA (i.e., immediately downstream (3′) to the targeting sequence of the guide RNA) may generally not be complementary to the genomic PAM sequence. The guide RNA may typically be a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site at or near (e.g., within) a target gene.

As mentioned above, a target gene for genome editing may be any of the genes proposed herein for suppression, including the following genes in corn or maize: a calcineurin B-like (CBL) interacting protein kinase 8 (Zm.CIPK8), a sorbitol dehydrogenase (Zm.SDH), a cytokinin dehydrogenase 4b or cytokinin oxidase 4b (Zm.CKX4b), or a cytokinin dehydrogenase 10 or cytokinin oxidase 10 (Zm.CKX10) gene; and the following genes in soybean: a homeobox transcription factor 1 (Gm.HB1), a branched 1 (Gm.BRC1) gene, or a fruitful c (Gm.FULc) gene.

For genome editing at or near (e.g., within) the calcineurin B-like (CBL interacting protein kinase 8 (Zm.CIPK8) gene in corn with an RNA-guided endonuclease, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of SEQ ID NO: 141 or a sequence complementary thereto (e.g., 12, 13, 14, 15, 16, 17, 18, 19, 20, 21, 22, 23, 24, 25 or more consecutive nucleotides of SEQ ID NO: 141 or a sequence complementary thereto). As used herein, the term “consecutive” in reference to a polynucleotide or protein sequence means without deletions or gaps in the sequence.

For knockdown (and possibly knockout) mutations of the calcineurin B-like (CBL) interacting protein kinase 8 (Zm.CIPK8) gene in corn through genome editing, an RNA-guided endonuclease may be targeted to an upstream or downstream sequence, such as a promoter and/or enhancer sequence, or an intron, 5′UTR, and/or 3′UTR sequence of the calcineurin B-like (CBL) interacting protein kinase 8 (Zm.CIPK8) gene in corn to mutate one or more promoter and/or regulatory sequences of the Zm.CIPK8 gene to affect or reduce its level of expression. For knockdown (and possibly knockout) of the Zm.CIPK8 gene in corn, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 1-2000 of SEQ ID NO: 141, the nucleotide sequence range 2181-4340, 4404-4568, 4641-6821, 6930-7016, 7092-7168, 7223-7640, 7767-7892, 7983-8462, 8586-8732, 8853-13119, 13237-13340, 13398-13488, or 13564-13756 of SEQ ID NO: 141, or the nucleotide sequence range 13853-14852 of SEQ ID NO: 141, or a sequence complementary thereto (e.g., 10, 11, 12, 13, 14, 15, 16, 17, 18, 19, 20, 21, 22, 23, 24, 25 or more consecutive nucleotides within the nucleotide sequence range 1-2000, 2181-4340, 4404-4568, 4641-6821, 6930-7016, 7092-7168, 7223-7640, 7767-7892, 7983-8462, 8586-8732, 8853-13119, 13237-13340, 13398-13488, 13564-13756, or 13853-14852 of SEQ ID NO: 141, or a sequence complementary thereto), although alternative splicing and different exon/intron boundaries may occur.

For knockout (and possibly knockdown) mutations of the calcineurin B-like (CBL) interacting protein kinase 8 (Zm.CIPK8) gene in corn through genome editing, an RNA-guided endonuclease may be targeted to a coding and/or intron sequence of the calcineurin B-like (CBL) interacting protein kinase 8 (Zm.CIPK8) gene in corn to potentially eliminate expression and/or activity of the Zm.CIPK8 gene and/or its encoded protein. However, a knockout of the Zm.CIPK8 gene expression may also be achieved in some cases by targeting the upstream and/or 5′UTR sequence(s) of the Zm.CIPK8 gene, or other sequences at or near the genomic locus of the Zm.CIPK8 gene. Thus, a knockout of the Zm.CIPK8 gene expression may be achieved by targeting a genomic sequence at or near the site or locus of the targeted the Zm.CIPK8 gene including an upstream or downstream sequence, such as a promoter and/or enhancer sequence, or an intron, 5′UTR, and/or 3′UTR sequence, of the Zm.CIPK8 gene, as described above for knockdown of the Zm.CIPK8 gene.

For knockout (and possibly knockdown) of the calcineurin B-like (CBL) interacting protein kinase 8 (Zm.CIPK8) gene in corn, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 2001-13852 of SEQ ID NO: 141 or the nucleotide sequence range 2001-2180, 4341-4403, 4569-4640, 6822-6929, 7017-7091, 7169-7222, 7641-7766, 7893-7982, 8463-8585, 8733-8852, 13120-13236, 13341-13397, 13489-13563, or 13757-13852 of SEQ ID NO: 141, or a sequence complementary thereto (e.g., 10, 11, 12, 13, 14, 15, 16, 17, 18, 19, 20, 21, 22, 23, 24, 25 or more consecutive nucleotides within the nucleotide sequence range 2001-13852, 2001-2180, 4341-4403, 4569-4640, 6822-6929, 7017-7091, 7169-7222, 7641-7766, 7893-7982, 8463-8585, 8733-8852, 13120-13236, 13341-13397, 13489-13563, and/or 13757-13852 of SEQ ID NO: 141, or a sequence complementary thereto), although alternative splicing and different exon/intron boundaries may occur.

Several site-specific nucleases, such as recombinases, zinc finger nucleases (ZFNs), meganucleases, and TALENs, are not RNA-guided and instead rely on their protein structure to determine their target site for causing the DSB or nick, or they are fused, tethered or attached to a DNA-binding protein domain or motif. The protein structure of the site-specific nuclease (or the fused/attached/tethered DNA binding domain) may target the site-specific nuclease to the target site (e.g., a target site at or near (e.g., within) the genomic locus of a target gene). According to some embodiments, a non-RNA-guided site-specific nuclease, such as a recombinase, zinc finger nuclease (ZFN), meganuclease, or TALEN, may be designed, engineered and constructed according to known methods to target and bind to a target site at or near the genomic locus of the calcineurin B-like (CBL) interacting protein kinase 8 (Zm.CIPK8) gene in corn, to create a DSB or nick at such genomic locus to knockout or knockdown expression of the Zm.CIPK8 gene via repair of the DSB or nick. For example, an engineered site-specific nuclease, such as a recombinase, zinc finger nuclease (ZFN), meganuclease, or TALEN, may be designed to target and bind to a target site within the genome of a plant corresponding to a sequence within SEQ ID NO: 141, or its complementary sequence, to create a DSB or nick at the genomic locus for the Zm.CIPK8 gene, which may then lead to the creation of a mutation or insertion of a sequence at or near the site of the DSB or nick, through cellular repair mechanisms, which may be further guided by a donor molecule or template.

For genome editing at or near (e.g., within) the sorbitol dehydrogenase (Zm.SDH) gene in corn with an RNA-guided endonuclease, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of SEQ ID NO: 142 or a sequence complementary thereto (e.g., 12, 13, 14, 15, 16, 17, 18, 19, 20, 21, 22, 23, 24, 25 or more consecutive nucleotides of SEQ ID NO: 142 or a sequence complementary thereto).

For knockdown (and possibly knockout) mutations of the sorbitol dehydrogenase (Zm. SDH) gene in corn through genome editing, an RNA-guided endonuclease may be targeted to an upstream or downstream sequence, such as a promoter and/or enhancer sequence, or an intron, 5′UTR, and/or 3′UTR sequence of the sorbitol dehydrogenase (Zm.SDH) gene in corn to mutate one or more promoter and/or regulatory sequences of the Zm.SDH gene to affect or reduce its level of expression. For knockdown (and possibly knockout) of the Zm.SDH gene in corn, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 1-2000 of SEQ ID NO: 142, the nucleotide sequence range 2125-3504 or 3573-3669 of SEQ ID NO: 142, or the nucleotide sequence range of SEQ ID NO: 142, or a sequence complementary thereto (e.g., 10, 11, 12, 13, 14, 15, 16, 17, 18, 19, 20, 21, 22, 23, 24, 25 or more consecutive nucleotides within the nucleotide sequence range 1-2000, 2125-3504 or 3573-3669 of SEQ ID NO: 142, or a sequence complementary thereto), although alternative splicing and different exon/intron boundaries may occur.

For knockout (and possibly knockdown) mutations of the sorbitol dehydrogenase (Zm. SDH) gene in corn through genome editing, an RNA-guided endonuclease may be targeted to a coding and/or intron sequence of the sorbitol dehydrogenase (Zm.SDH) gene in corn to potentially eliminate expression and/or activity of the Zm.SDH gene and/or its encoded protein. However, a knockout of the Zm.SDH gene expression may also be achieved in some cases by targeting the upstream and/or 5′UTR sequence(s) of the Zm.SDH gene, or other sequences at or near the genomic locus of the Zm. SDH gene. Thus, a knockout of the Zm. SDH gene expression may be achieved by targeting a genomic sequence at or near the site or locus of the targeted the Zm.SDH gene including an upstream or downstream sequence, such as a promoter and/or enhancer sequence, or an intron, 5′UTR, and/or 3′UTR sequence, of the Zm.SDH gene, as described above for knockdown of the Zm.SDH gene.

For knockout (and possibly knockdown) of the sorbitol dehydrogenase (Zm. SDH) gene in corn, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 2001-4578 of SEQ ID NO: 142, the nucleotide sequence range 2001-2124, 3505-3572, or 3670-4578 of SEQ ID NO: 142, or a sequence complementary thereto (e.g., 10, 11, 12, 13, 14, 15, 16, 17, 18, 19, 20, 21, 22, 23, 24, 25 or more consecutive nucleotides within the nucleotide sequence range 2001-4578, 2001-2124, 3505-3572, or 3670-4578 of SEQ ID NO: 142, or a sequence complementary thereto), although alternative splicing and different exon/intron boundaries may occur.

According to other embodiments, a non-RNA-guided site-specific nuclease, such as a recombinase, zinc finger nuclease (ZFN), meganuclease, or TALEN, may be designed, engineered and constructed according to known methods to target and bind to a target site at or near the genomic locus of the sorbitol dehydrogenase (Zm.SDH) gene in corn, to create a DSB or nick at such genomic locus to knockout or knockdown expression of the Zm.SDH gene via repair of the DSB or nick. For example, an engineered site-specific nuclease, such as a recombinase, zinc finger nuclease (ZFN), meganuclease, or TALEN, may be designed to target and bind to a target site within the genome of a plant corresponding to a sequence within SEQ ID NO: 142, or its complementary sequence, to create a DSB or nick at the genomic locus for the Zm. SDH gene, which may then lead to the creation of a mutation or insertion of a sequence at or near the site of the DSB or nick, through cellular repair mechanisms, which may be further guided by a donor molecule or template.

For genome editing at or near (e.g., within) the cytokinin dehydrogenase/oxidase 4b (Zm.CKX4b) gene in corn with an RNA-guided endonuclease, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of SEQ ID NO: 144 or a sequence complementary thereto (e.g., 12, 13, 14, 15, 16, 17, 18, 19, 20, 21, 22, 23, 24, 25 or more consecutive nucleotides of SEQ ID NO: 144 or a sequence complementary thereto).

For knockdown (and possibly knockout) mutations of the cytokinin dehydrogenase/oxidase 4b (Zm.CKX4b) gene in corn through genome editing, an RNA-guided endonuclease may be targeted to an upstream or downstream sequence, such as a promoter and/or enhancer sequence, or an intron, 5′UTR, and/or 3′UTR sequence of the cytokinin dehydrogenase/oxidase 4b (Zm.CKX4b) gene in corn to mutate one or more promoter and/or regulatory sequences of the Zm.CKX4b gene to affect or reduce its level of expression. For knockdown (and possibly knockout) of the Zm.CKX4b gene in corn, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 1-2000 of SEQ ID NO: 144, the nucleotide sequence range 2608-2770, 2899-3658, 3923-4204, or 4477-5520 of SEQ ID NO: 144, or the nucleotide sequence range 4855-5854 of SEQ ID NO: 144, or a sequence complementary thereto (e.g., 10, 11, 12, 13, 14, 15, 16, 17, 18, 19, 20, 21, 22, 23, 24, 25 or more consecutive nucleotides within the nucleotide sequence range 1-2000, 2608-2770, 2899-3658, 3923-4204, 4477-5520, or 5855-6854 of SEQ ID NO: 144, or a sequence complementary thereto), although alternative splicing and different exon/intron boundaries may occur.

For knockout (and possibly knockdown) mutations of the cytokinin dehydrogenase/oxidase 4b (Zm.CKX4b) gene in corn through genome editing, an RNA-guided endonuclease may be targeted to a coding and/or intron sequence of the cytokinin dehydrogenase/oxidase 4b (Zm.CKX4b) gene in corn to potentially eliminate expression and/or activity of the Zm.CKX4b gene and/or its encoded protein. However, a knockout of the Zm.CKX4b gene expression may also be achieved in some cases by targeting the upstream and/or 5′UTR sequence(s) of the Zm.CKX4b gene, or other sequences at or near the genomic locus of the Zm.CKX4b gene. Thus, a knockout of the Zm.CKX4b gene expression may be achieved by targeting a genomic sequence at or near the site or locus of the targeted the Zm.CKX4b gene including an upstream or downstream sequence, such as a promoter and/or enhancer sequence, or an intron, 5′UTR, and/or 3′UTR sequence, of the Zm.CKX4b gene, as described above for knockdown of the Zm.CKX4b gene.

For knockout (and possibly knockdown) of the cytokinin dehydrogenase/oxidase 4b (Zm.CKX4b) gene in corn, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 2001-5854 of SEQ ID NO: 144 or the nucleotide sequence range 2001-2607, 2771-2898, 3659-3922, 4205-4476, or 5521-5854 of SEQ ID NO: 144, or a sequence complementary thereto (e.g., 10, 11, 12, 13, 14, 15, 16, 17, 18, 19, 20, 21, 22, 23, 24, 25 or more consecutive nucleotides within the nucleotide sequence range 2001-5854, 2001-2607, 2771-2898, 3659-3922, 4205-4476, or 5521-5854 of SEQ ID NO: 144, or a sequence complementary thereto), although alternative splicing and different exon/intron boundaries may occur.

According to other embodiments, a non-RNA-guided site-specific nuclease, such as a recombinase, zinc finger nuclease (ZFN), meganuclease, or TALEN, may be designed, engineered and constructed according to known methods to target and bind to a target site at or near the genomic locus of the cytokinin dehydrogenase/oxidase 4b (Zm.CKX4b) gene in corn, to create a DSB or nick at such genomic locus to knockout or knockdown expression of the Zm.CKX4b gene via repair of the DSB or nick. For example, an engineered site-specific nuclease, such as a recombinase, zinc finger nuclease (ZFN), meganuclease, or TALEN, may be designed to target and bind to a target site within the genome of a plant corresponding to a sequence within SEQ ID NO: 144, or its complementary sequence, to create a DSB or nick at the genomic locus for the Zm.CKX4b gene, which may then lead to the creation of a mutation or insertion of a sequence at or near the site of the DSB or nick, through cellular repair mechanisms, which may be further guided by a donor molecule or template.

For genome editing at or near (e.g., within) the cytokinin dehydrogenase/oxidase 10 (Zm.CKX10) gene in corn with an RNA-guided endonuclease, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of SEQ ID NO: 145 or a sequence complementary thereto (e.g., 12, 13, 14, 15, 16, 17, 18, 19, 20, 21, 22, 23, 24, 25 or more consecutive nucleotides of SEQ ID NO: 145 or a sequence complementary thereto).

For knockdown (and possibly knockout) mutations of the cytokinin dehydrogenase/oxidase 10 (Zm.CKX10) gene in corn through genome editing, an RNA-guided endonuclease may be targeted to an upstream or downstream sequence, such as a promoter and/or enhancer sequence, or an intron, 5′UTR, and/or 3′UTR sequence of the cytokinin dehydrogenase/oxidase 10 (Zm.CKX10) gene in corn to mutate one or more promoter and/or regulatory sequences of the Zm.CKX10 gene to affect or reduce its level of expression. For knockdown (and possibly knockout) of the Zm.CKX10 gene in corn, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 1-2000 of SEQ ID NO: 145, the nucleotide sequence range 2694-2778, 3070-3742, or 4015-4453 of SEQ ID NO: 145, or the nucleotide sequence range 4776-5775 of SEQ ID NO: 145, or a sequence complementary thereto (e.g., 10, 11, 12, 13, 14, 15, 16, 17, 18, 19, 20, 21, 22, 23, 24, 25 or more consecutive nucleotides within the nucleotide sequence range 1-2000, 2694-2778, 3070-3742, 4015-4453, or 4776-5775 of SEQ ID NO: 145, or a sequence complementary thereto), although alternative splicing and different exon/intron boundaries may occur.

For knockout (and possibly knockdown) mutations of the cytokinin dehydrogenase/oxidase 10 (Zm.CKX10) gene in corn through genome editing, an RNA-guided endonuclease may be targeted to a coding and/or intron sequence of the cytokinin dehydrogenase/oxidase 10 (Zm.CKX10) gene in corn to potentially eliminate expression and/or activity of the Zm.CKX10 gene and/or its encoded protein. However, a knockout of the Zm.CKX10 gene expression may also be achieved in some cases by targeting the upstream and/or 5′UTR sequence(s) of the Zm.CKX10 gene, or other sequences at or near the genomic locus of the Zm.CKX10 gene. Thus, a knockout of the Zm.CKX10 gene expression may be achieved by targeting a genomic sequence at or near the site or locus of the targeted the Zm.CKX10 gene including an upstream or downstream sequence, such as a promoter and/or enhancer sequence, or an intron, 5′UTR, and/or 3′UTR sequence, of the Zm.CKX10 gene, as described above for knockdown of the Zm.CKX10 gene.

For knockout (and possibly knockdown) of the cytokinin dehydrogenase/oxidase 10 (Zm.CKX10) gene in corn, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 2001-4775 of SEQ ID NO: 145 or the nucleotide sequence range 2001-2693, 2779-3069, 3743-4014, or 4454-4775 of SEQ ID NO: 145, or a sequence complementary thereto (e.g., 10, 11, 12, 13, 14, 15, 16, 17, 18, 19, 20, 21, 22, 23, 24, 25 or more consecutive nucleotides within the nucleotide sequence range 2001-4775, 2001-2693, 2779-3069, 3743-4014, or 4454-4775 of SEQ ID NO: 145, or a sequence complementary thereto), although alternative splicing and different exon/intron boundaries may occur.

According to other embodiments, a non-RNA-guided site-specific nuclease, such as a recombinase, zinc finger nuclease (ZFN), meganuclease, or TALEN, may be designed, engineered and constructed according to known methods to target and bind to a target site at or near the genomic locus of the cytokinin dehydrogenase/oxidase 10 (Zm.CKX10) gene in corn, to create a DSB or nick at such genomic locus to knockout or knockdown expression of the Zm.CKX10 gene via repair of the DSB or nick. For example, an engineered site-specific nuclease, such as a recombinase, zinc finger nuclease (ZFN), meganuclease, or TALEN, may be designed to target and bind to a target site within the genome of a plant corresponding to a sequence within SEQ ID NO: 145, or its complementary sequence, to create a DSB or nick at the genomic locus for the Zm.CKX10 gene, which may then lead to the creation of a mutation or insertion of a sequence at or near the site of the DSB or nick, through cellular repair mechanisms, which may be further guided by a donor molecule or template.

For genome editing at or near (e.g., within) the homeobox transcription factor 1 (Gm.HB1) gene in soybean with an RNA-guided endonuclease, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of SEQ ID NO: 143 or a sequence complementary thereto (e.g., 12, 13, 14, 15, 16, 17, 18, 19, 20, 21, 22, 23, 24, 25 or more consecutive nucleotides of SEQ ID NO: 143 or a sequence complementary thereto).

For knockdown (and possibly knockout) mutations of the homeobox transcription factor 1 (Gm.HB1) gene in soybean through genome editing, an RNA-guided endonuclease may be targeted to an upstream or downstream sequence, such as a promoter and/or enhancer sequence, or an intron, 5′UTR, and/or 3′UTR sequence of the homeobox transcription factor 1 (Gm.HB1) gene in soybean to mutate one or more promoter and/or regulatory sequences of the Gm.HB1 gene to affect or reduce its level of expression. For knockdown (and possibly knockout) of the Gm.HB1 gene in soybean, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 1-2000 of SEQ ID NO: 143, the nucleotide sequence range 2373-2584 of SEQ ID NO: 143, or the nucleotide sequence range 2951-3950 of SEQ ID NO: 143, or a sequence complementary thereto (e.g., 10, 11, 12, 13, 14, 15, 16, 17, 18, 19, 20, 21, 22, 23, 24, 25 or more consecutive nucleotides within the nucleotide sequence range 1-2000, 2373-2584, or 2951-3950 of SEQ ID NO: 143, or a sequence complementary thereto), although alternative splicing and different exon/intron boundaries may occur.

For knockout (and possibly knockdown) mutations of the homeobox transcription factor 1 (Gm.HB1) gene in soybean through genome editing, an RNA-guided endonuclease may be targeted to a coding and/or intron sequence of the homeobox transcription factor 1 (Gm.HB1) gene in soybean to potentially eliminate expression and/or activity of the Gm.HB1 gene and/or its encoded protein. However, a knockout of the Gm.HB1 gene expression may also be achieved in some cases by targeting the upstream and/or 5′UTR sequence(s) of the Gm.HB1 gene, or other sequences at or near the genomic locus of the Gm.HB1 gene. Thus, a knockout of the Gm.HB1 gene expression may be achieved by targeting a genomic sequence at or near the site or locus of the targeted the Gm.HB1 gene including an upstream or downstream sequence, such as a promoter and/or enhancer sequence, or an intron, 5′UTR, and/or 3′UTR sequence, of the Gm.HB1 gene, as described above for knockdown of the Gm.HB1 gene.

For knockout (and possibly knockdown) of the homeobox transcription factor 1 (Gm.HB1) gene in soybean, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 2001-2950 of SEQ ID NO: 143 or the nucleotide sequence range 2001-2372 or 2585-2950 of SEQ ID NO: 143, or a sequence complementary thereto (e.g., 10, 11, 12, 13, 14, 15, 16, 17, 18, 19, 20, 21, 22, 23, 24, 25 or more consecutive nucleotides within the nucleotide sequence range 2001-2950, 2001-2372 or 2585-2950 of SEQ ID NO: 143, or a sequence complementary thereto), although alternative splicing and different exon/intron boundaries may occur.

According to other embodiments, a non-RNA-guided site-specific nuclease, such as a recombinase, zinc finger nuclease (ZFN), meganuclease, or TALEN, may be designed, engineered and constructed according to known methods to target and bind to a target site at or near the genomic locus of the homeobox transcription factor 1 (Gm.HB1) gene in soybean, to create a DSB or nick at such genomic locus to knockout or knockdown expression of the Gm.HB1 gene via repair of the DSB or nick. For example, an engineered site-specific nuclease, such as a recombinase, zinc finger nuclease (ZFN), meganuclease, or TALEN, may be designed to target and bind to a target site within the genome of a plant corresponding to a sequence within SEQ ID NO: 143, or its complementary sequence, to create a DSB or nick at the genomic locus for the Gm.HB1 gene, which may then lead to the creation of a mutation or insertion of a sequence at or near the site of the DSB or nick, through cellular repair mechanisms, which may be further guided by a donor molecule or template.

For genome editing at or near (e.g., within) the branched 1 or BRC1 (Gm.BRC1) gene in soybean with an RNA-guided endonuclease, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of SEQ ID NO: 146 or a sequence complementary thereto (e.g., 12, 13, 14, 15, 16, 17, 18, 19, 20, 21, 22, 23, 24, 25 or more consecutive nucleotides of SEQ ID NO: 146 or a sequence complementary thereto).

For knockdown (and possibly knockout) mutations of the BRC1 (Gm.BRC1) gene in soybean through genome editing, an RNA-guided endonuclease may be targeted to an upstream or downstream sequence, such as a promoter and/or enhancer sequence, or an intron, 5′UTR, and/or 3′UTR sequence of the BRC1 (Gm.BRC1) gene in soybean to mutate one or more promoter and/or regulatory sequences of the Gm.BRC1 gene to affect or reduce its level of expression. For knockdown (and possibly knockout) of the Gm.BRC1 gene in soybean, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 1-2000 of SEQ ID NO: 146, the nucleotide sequence range 3111-3731 of SEQ ID NO: 146, or the nucleotide sequence range 3780-4779 of SEQ ID NO: 146, or a sequence complementary thereto (e.g., 10, 11, 12, 13, 14, 15, 16, 17, 18, 19, 20, 21, 22, 23, 24, 25 or more consecutive nucleotides within the nucleotide sequence range 1-2000, 3111-3731, or 3780-4779 of SEQ ID NO: 146, or a sequence complementary thereto), although alternative splicing and different exon/intron boundaries may occur.

For knockout (and possibly knockdown) mutations of the BRC1 (Gm.BRC1) gene in soybean through genome editing, an RNA-guided endonuclease may be targeted to a coding and/or intron sequence of the BRC1 (Gm.BRC1) gene in soybean to potentially eliminate expression and/or activity of the Gm.BRC1 gene and/or its encoded protein. However, a knockout of the Gm.BRC1 gene expression may also be achieved in some cases by targeting the upstream and/or 5′UTR sequence(s) of the Gm.BRC1 gene, or other sequences at or near the genomic locus of the Gm.BRC1 gene. Thus, a knockout of the Gm.BRC1 gene expression may be achieved by targeting a genomic sequence at or near the site or locus of the targeted the Gm.BRC1 gene including an upstream or downstream sequence, such as a promoter and/or enhancer sequence, or an intron, 5′UTR, and/or 3′UTR sequence, of the Gm.BRC1 gene, as described above for knockdown of the Gm.BRC1 gene.

For knockout (and possibly knockdown) of the BRC1 (Gm.BRC1) gene in soybean, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 2001-3779 of SEQ ID NO: 146 or the nucleotide sequence range 2001-3110 or 3732-3779 of SEQ ID NO: 146, or a sequence complementary thereto (e.g., 10, 11, 12, 13, 14, 15, 16, 17, 18, 19, 20, 21, 22, 23, 24, 25 or more consecutive nucleotides within the nucleotide sequence range 2001-3779, 2001-3110 or 3732-3779 of SEQ ID NO: 146, or a sequence complementary thereto), although alternative splicing and different exon/intron boundaries may occur.

According to other embodiments, a non-RNA-guided site-specific nuclease, such as a recombinase, zinc finger nuclease (ZFN), meganuclease, or TALEN, may be designed, engineered and constructed according to known methods to target and bind to a target site at or near the genomic locus of the BRC1 (Gm.BRC1) gene in soybean, to create a DSB or nick at such genomic locus to knockout or knockdown expression of the Gm.BRC1 gene via repair of the DSB or nick. For example, an engineered site-specific nuclease, such as a recombinase, zinc finger nuclease (ZFN), meganuclease, or TALEN, may be designed to target and bind to a target site within the genome of a plant corresponding to a sequence within SEQ ID NO: 146, or its complementary sequence, to create a DSB or nick at the genomic locus for the Gm.BRC1 gene, which may then lead to the creation of a mutation or insertion of a sequence at or near the site of the DSB or nick, through cellular repair mechanisms, which may be further guided by a donor molecule or template.

For genome editing at or near (e.g., within) the fruitful c or FULc (Gm.FULc) gene in soybean with an RNA-guided endonuclease, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of SEQ ID NO: 147 or a sequence complementary thereto (e.g., 12, 13, 14, 15, 16, 17, 18, 19, 20, 21, 22, 23, 24, 25 or more consecutive nucleotides of SEQ ID NO: 147 or a sequence complementary thereto).

For knockdown (and possibly knockout) mutations of the FULc (Gm.FULc) gene in soybean through genome editing, an RNA-guided endonuclease may be targeted to an upstream or downstream sequence, such as a promoter and/or enhancer sequence, or an intron, 5′UTR, and/or 3′UTR sequence of the FULc (Gm.FULc) gene in soybean to mutate one or more promoter and/or regulatory sequences of the Gm.FULc gene to affect or reduce its level of expression. For knockdown (and possibly knockout) of the Gm.FULc gene in soybean, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 1-2000 of SEQ ID NO: 147, the nucleotide sequence range 2186-11058, 11135-11339, 11405-12030, 12131-12300, 12343-12868, 12908-13012, 13153-13665 of SEQ ID NO: 147, or the nucleotide sequence range 13766-14765 of SEQ ID NO: 147, or a sequence complementary thereto (e.g., 10, 11, 12, 13, 14, 15, 16, 17, 18, 19, 20, 21, 22, 23, 24, 25 or more consecutive nucleotides within the nucleotide sequence range 1-2000, 2186-11058, 11135-11339, 11405-12030, 12131-12300, 12343-12868, 12908-13012, 13153-13665, or 13766-14765 of SEQ ID NO: 147, or a sequence complementary thereto), although alternative splicing and different exon/intron boundaries may occur.

For knockout (and possibly knockdown) mutations of the FULc (Gm.FULc) gene in soybean through genome editing, an RNA-guided endonuclease may be targeted to a coding and/or intron sequence of the FULc (Gm.FULc) gene in soybean to potentially eliminate expression and/or activity of the Gm.FULc gene and/or its encoded protein. However, a knockout of the Gm.FULc gene expression may also be achieved in some cases by targeting the upstream and/or 5′UTR sequence(s) of the Gm.FULc gene, or other sequences at or near the genomic locus of the Gm.FULc gene. Thus, a knockout of the Gm.FULc gene expression may be achieved by targeting a genomic sequence at or near the site or locus of the targeted the Gm.FULc gene including an upstream or downstream sequence, such as a promoter and/or enhancer sequence, or an intron, 5′UTR, and/or 3′UTR sequence, of the Gm.FULc gene, as described above for knockdown of the Gm.FULc gene.

For knockout (and possibly knockdown) of the FULc (Gm.FULc) gene in soybean, a guide RNA may be used comprising a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides within the nucleotide sequence range 2001-13765 of SEQ ID NO: 147 or the nucleotide sequence range 2001-2185, 11059-11134, 11340-11404, 12031-12130, 12301-12342, 12869-12907, 13013-13152, or 13666-13765 of SEQ ID NO: 147, or a sequence complementary thereto (e.g., 10, 11, 12, 13, 14, 15, 16, 17, 18, 19, 20, 21, 22, 23, 24, 25 or more consecutive nucleotides within the nucleotide sequence range 2001-13765, 2001-2185, 11059-11134, 11340-11404, 12031-12130, 12301-12342, 12869-12907, 13013-13152, or 13666-13765 of SEQ ID NO: 147, or a sequence complementary thereto), although alternative splicing and different exon/intron boundaries may occur.

According to other embodiments, a non-RNA-guided site-specific nuclease, such as a recombinase, zinc finger nuclease (ZFN), meganuclease, or TALEN, may be designed, engineered and constructed according to known methods to target and bind to a target site at or near the genomic locus of the FULc (Gm.FULc) gene in soybean, to create a DSB or nick at such genomic locus to knockout or knockdown expression of the Gm.FULc gene via repair of the DSB or nick. For example, an engineered site-specific nuclease, such as a recombinase, zinc finger nuclease (ZFN), meganuclease, or TALEN, may be designed to target and bind to a target site within the genome of a plant corresponding to a sequence within SEQ ID NO: 147, or its complementary sequence, to create a DSB or nick at the genomic locus for the Gm.FULc gene, which may then lead to the creation of a mutation or insertion of a sequence at or near the site of the DSB or nick, through cellular repair mechanisms, which may be further guided by a donor molecule or template.

According to some embodiments, recombinant DNA constructs and vectors are provided comprising a polynucleotide sequence encoding a site-specific nuclease, such as a zinc-finger nuclease (ZFN), a meganuclease, an RNA-guided endonuclease, a TALE-endonuclease (TALEN), a recombinase, or a transposase, wherein the coding sequence is operably linked to a plant expressible promoter. For RNA-guided endonucleases, recombinant DNA constructs and vectors are further provided comprising a polynucleotide sequence encoding a guide RNA, wherein the guide RNA comprises a guide sequence of sufficient length having a percent identity or complementarity to a target site within the genome of a plant, such as at or near a target gene. According to some embodiments, a polynucleotide sequence of a recombinant DNA construct and vector that encodes a site-specific nuclease or a guide RNA may be operably linked to a plant expressible promoter, such as an inducible promoter, a constitutive promoter, a tissue-specific promoter, etc.

In an aspect, the present disclosure provides a modified corn (maize) or soybean plant, or plant part thereof, or a modified corn or soybean plant tissue or plant cell, comprising a mutant allele(s) of the target gene (i.e., one or more mutation(s) and/or genome edit(s) at or near (e.g., within) the target gene. The modified corn (maize) or soybean plant, or plant part thereof, or a modified corn or soybean plant tissue or plant cell, may be homozygous, heterozygous, heteroallelic (or biallelic) for the mutation(s) and/or edit(s) at or near the genomic locus of the target gene and/or the allele(s) of the target gene. Each such mutation or edit may be a nonsense mutation, missense mutation, frameshift mutation, or splice-site mutation. In an aspect, a mutation or edit may be in a region of the target gene selected from the group consisting of a promoter, enhancer, 5′ UTR, first exon, first intron, second exon, second intron, third exon, 3′ UTR, or terminator. In an aspect, a mutation at or near a target gene (or a mutant or mutant allele of the target gene) may comprise a silent mutation which does not change the encoded amino acid sequence of the target gene, but may affect mRNA transcript expression, mRNA or protein stability or protein translation efficiency, or otherwise contribute to reduced enzyme activity, relative to a corresponding wild type allele of the target gene. In a further aspect, a mutation of a target gene (or a mutant or mutant allele of the target gene) can comprise a mutation or edit at or around the TATA box or other promoter element(s) that affect gene transcription. In an aspect, a mutation in, or an allele of, a target gene in a modified corn or soybean plant may be a recessive, dominant or semi-dominant mutation or allele.

According to some embodiments, a recombinant DNA construct or vector may comprise a first polynucleotide sequence encoding a site-specific nuclease and a second polynucleotide sequence encoding a guide RNA that may be introduced into a plant cell together via plant transformation techniques. Alternatively, two recombinant DNA constructs or vectors may be provided including a first recombinant DNA construct or vector and a second DNA construct or vector that may be introduced into a plant cell together or sequentially via plant transformation techniques, wherein the first recombinant DNA construct or vector comprises a polynucleotide sequence encoding a site-specific nuclease and the second recombinant DNA construct or vector comprises a polynucleotide sequence encoding a guide RNA. According to some embodiments, a recombinant DNA construct or vector comprising a polynucleotide sequence encoding a site-specific nuclease may be introduced via plant transformation techniques into a plant cell that already comprises (or is transformed with) a recombinant DNA construct or vector comprising a polynucleotide sequence encoding a guide RNA. Alternatively, a recombinant DNA construct or vector comprising a polynucleotide sequence encoding a guide RNA may be introduced via plant transformation techniques into a plant cell that already comprises (or is transformed with) a recombinant DNA construct or vector comprising a polynucleotide sequence encoding a site-specific nuclease. According to yet further embodiments, a first plant comprising (or transformed with) a recombinant DNA construct or vector comprising a polynucleotide sequence encoding a site-specific nuclease may be crossed with a second plant comprising (or transformed with) a recombinant DNA construct or vector comprising a polynucleotide sequence encoding a guide RNA. Such recombinant DNA constructs or vectors may be transiently transformed into a plant cell or stably transformed or integrated into the genome of a plant cell.

In an aspect, vectors comprising polynucleotides encoding a site-specific nuclease, and optionally one or more gRNAs are provided or introduced into a plant cell by transformation methods known in the art (e.g., without being limiting, particle bombardment, PEG-mediated protoplast transfection or Agrobacterium-mediated transformation). In an aspect, vectors comprising polynucleotides encoding a Cas9 nuclease, and optionally one or more gRNAs are provided to a plant cell by transformation methods known in the art (e.g., without being limiting, particle bombardment, PEG-mediated protoplast transfection or Agrobacterium-mediated transformation). In another aspect, vectors comprising polynucleotides encoding a Cpf1 and, optionally one or more crRNAs are provided to a cell by transformation methods known in the art (e.g., without being limiting, viral transfection, particle bombardment, PEG-mediated protoplast transfection or Agrobacterium-mediated transformation).

In an aspect, a targeted genome editing technique described herein may comprise the use of a recombinase. In some embodiments, a tyrosine recombinase attached, etc., to a DNA recognition domain or motif may be selected from the group consisting of a Cre recombinase, a Flp recombinase, and a Tnp1 recombinase. In an aspect, a Cre recombinase or a Gin recombinase provided herein may be tethered to a zinc-finger DNA binding domain. The Flp-FRT site-directed recombination system may come from the 2μ plasmid from the baker's yeast Saccharomyces cerevisiae. In this system, Flp recombinase (flippase) may recombine sequences between flippase recognition target (FRT) sites. FRT sites comprise 34 nucleotides. Flp may bind to the “arms” of the FRT sites (one arm is in reverse orientation) and cleaves the FRT site at either end of an intervening nucleic acid sequence. After cleavage, Flp may recombine nucleic acid sequences between two FRT sites. Cre-lox is a site-directed recombination system derived from the bacteriophage P1 that is similar to the Flp-FRT recombination system. Cre-lox can be used to invert a nucleic acid sequence, delete a nucleic acid sequence, or translocate a nucleic acid sequence. In this system, Cre recombinase may recombine a pair of lox nucleic acid sequences. Lox sites comprise 34 nucleotides, with the first and last 13 nucleotides (arms) being palindromic. During recombination, Cre recombinase protein binds to two lox sites on different nucleic acids and cleaves at the lox sites. The cleaved nucleic acids are spliced together (reciprocally translocated) and recombination is complete. In another aspect, a lox site provided herein is a loxP, lox 2272, loxN, lox 511, lox 5171, lox71, lox66, M2, M3, M7, or M11 site.

ZFNs are synthetic proteins consisting of an engineered zinc finger DNA-binding domain fused to a cleavage domain (or a cleavage half-domain), which may be derived from a restriction endonuclease (e.g., Fold). The DNA binding domain may be canonical (C2H2) or non-canonical (e.g., C3H or C4). The DNA-binding domain can comprise one or more zinc fingers (e.g., 2, 3, 4, 5, 6, 7, 8, 9 or more zinc fingers) depending on the target site. Multiple zinc fingers in a DNA-binding domain may be separated by linker sequence(s). ZFNs can be designed to cleave almost any stretch of double-stranded DNA by modification of the zinc finger DNA-binding domain. ZFNs form dimers from monomers composed of a non-specific DNA cleavage domain (e.g., derived from the FokI nuclease) fused to a DNA-binding domain comprising a zinc finger array engineered to bind a target site DNA sequence. The DNA-binding domain of a ZFN may typically be composed of 3-4 (or more) zinc-fingers. The amino acids at positions −1, +2, +3, and +6 relative to the start of the zinc finger α-helix, which contribute to site-specific binding to the target site, can be changed and customized to fit specific target sequences. The other amino acids may form a consensus backbone to generate ZFNs with different sequence specificities. Methods and rules for designing ZFNs for targeting and binding to specific target sequences are known in the art. See, e.g., US Patent App. Nos. 2005/0064474, 2009/0117617, and 2012/0142062, the contents and disclosures of which are incorporated herein by reference. The FokI nuclease domain may require dimerization to cleave DNA and therefore two ZFNs with their C-terminal regions are needed to bind opposite DNA strands of the cleavage site (separated by 5-7 bp). The ZFN monomer can cut the target site if the two-ZF-binding sites are palindromic. A ZFN, as used herein, is broad and includes a monomeric ZFN that can cleave double stranded DNA without assistance from another ZFN. The term ZFN may also be used to refer to one or both members of a pair of ZFNs that are engineered to work together to cleave DNA at the same site.

Without being limited by any scientific theory, because the DNA-binding specificities of zinc finger domains can be re-engineered using one of various methods, customized ZFNs can theoretically be constructed to target nearly any target sequence (e.g., at or near a target gene in a plant genome). Publicly available methods for engineering zinc finger domains include Context-dependent Assembly (CoDA), Oligomerized Pool Engineering (OPEN), and Modular Assembly. In an aspect, a method and/or composition provided herein comprises one or more, two or more, three or more, four or more, or five or more ZFNs. In another aspect, a ZFN provided herein is capable of generating a targeted DSB or nick. In an aspect, vectors comprising polynucleotides encoding one or more, two or more, three or more, four or more, or five or more ZFNs are provided to a cell by transformation methods known in the art (e.g., without being limiting, viral transfection, particle bombardment, PEG-mediated protoplast transfection, or Agrobacterium-mediated transformation). The ZFNs may be introduced as ZFN proteins, as polynucleotides encoding ZFN proteins, and/or as combinations of proteins and protein-encoding polynucleotides.

Meganucleases, which are commonly identified in microbes, such as the LAGLIDADG family of homing endonucleases, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). According to some embodiments, a meganuclease may comprise a scaffold or base enzyme selected from the group consisting of I-CreI, I-CeuI, I-MsoI, I-SceI, AniI, and I-DmoI. The engineering of meganucleases can be more challenging than ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity. Thus, a meganuclease may be selected or engineered to bind to a genomic target sequence in a plant, such as at or near the genomic locus of a target gene. In an aspect, a method and/or composition provided herein comprises one or more, two or more, three or more, four or more, or five or more meganucleases. In another aspect, a meganuclease provided herein is capable of generating a targeted DSB. In an aspect, vectors comprising polynucleotides encoding one or more, two or more, three or more, four or more, or five or more meganucleases are provided to a cell by transformation methods known in the art (e.g., without being limiting, viral transfection, particle bombardment, PEG-mediated protoplast transfection or Agrobacterium-mediated transformation).

TALENs are artificial restriction enzymes generated by fusing the transcription activator-like effector (TALE) DNA binding domain to a nuclease domain (e.g., FokI). When each member of a TALEN pair binds to the DNA sites flanking a target site, the FokI monomers dimerize and cause a double-stranded DNA break at the target site. Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity.

TALENs are artificial restriction enzymes generated by fusing the transcription activator-like effector (TALE) DNA binding domain to a nuclease domain. In some aspects, the nuclease is selected from a group consisting of PvuII, MutH, TevI, FokI, AlwI, MlyI, SbfI, SdaI, StsI, CleDORF, Clo051, and Pept071. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN is also refers to one or both members of a pair of TALENs that work together to cleave DNA at the same site.

Transcription activator-like effectors (TALEs) can be engineered to bind practically any DNA sequence, such as at or near the genomic locus of a target gene in a plant. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

The relationship between amino acid sequence and DNA recognition of the TALE binding domain allows for designable proteins. Software programs such as DNA Works can be used to design TALE constructs. Other methods of designing TALE constructs are known to those of skill in the art. See Doyle et al., Nucleic Acids Research (2012) 40: W117-122.; Cermak et al., Nucleic Acids Research (2011). 39:e82; and tale-nt.cac.cornell.edu/about. In an aspect, a method and/or composition provided herein comprises one or more, two or more, three or more, four or more, or five or more TALENs. In another aspect, a TALEN provided herein is capable of generating a targeted DSB. In an aspect, vectors comprising polynucleotides encoding one or more, two or more, three or more, four or more, or five or more TALENs are provided to a cell by transformation methods known in the art (e.g., without being limiting, viral transfection, particle bombardment, PEG-mediated protoplast transfection or Agrobacterium-mediated transformation). See, e.g., US Patent App. Nos. 2011/0145940, 2011/0301073, and 2013/0117869, the contents and disclosures of which are incorporated herein by reference.

As used herein, a “targeted genome editing technique” refers to any method, protocol, or technique that allows the precise and/or targeted editing of a specific location in a genome of a plant (i.e., the editing is largely or completely non-random) using a site-specific nuclease, such as a meganuclease, a zinc-finger nuclease (ZFN), an RNA-guided endonuclease (e.g., the CRISPR/Cas9 system), a TALE-endonuclease (TALEN), a recombinase, or a transposase. As used herein, “editing” or “genome editing” refers to generating a targeted mutation, deletion, inversion or substitution of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides of an endogenous plant genome nucleic acid sequence. As used herein, “editing” or “genome editing” also encompasses the targeted insertion or site-directed integration of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 750, at least 1000, at least 1500, at least 2000, at least 2500, at least 3000, at least 4000, at least 5000, at least 10,000, or at least 25,000 nucleotides into the endogenous genome of a plant. An “edit” or “genomic edit” in the singular refers to one such targeted mutation, deletion, inversion, substitution or insertion, whereas “edits” or “genomic edits” refers to two or more targeted mutation(s), deletion(s), inversion(s), substitution(s) and/or insertion(s), with each “edit” being introduced via a targeted genome editing technique.

For site-specific nucleases that are not RNA-guided, such as a zinc-finger nuclease (ZFN), a meganuclease, a TALE-endonuclease (TALEN), a recombinase, and/or a transposase, the genomic target specificity for editing is determined by its protein structure, particularly its DNA binding domain. Such site-specific nucleases may be chosen, designed or engineered to bind and cut a desired target site at or near any of the target genes within the genome of a corn (maize) or soybean plant. Similar to transformation with a suppression construct, a corn or soybean plant transformed with a particular guide RNA, or a recombinant DNA molecule, vector or construct encoding a guide RNA, should preferably be the species in which the targeted genomic sequence exists, or a closely related species, strain, germplasm, line, etc., such that the guide RNA is able to recognize and bind to the desired target cut site.

Transgenic or modified plants comprising or derived from plant cells that are transformed with a recombinant DNA of this disclosure can be further enhanced with stacked traits, for example, a crop plant having an enhanced trait resulting from expression of DNA disclosed herein in combination with herbicide and/or pest resistance traits. For example, genes or alleles of the current disclosure can be stacked with other traits of agronomic interest, such as a trait providing herbicide resistance, or insect resistance, such as using a gene from Bacillus thuringensis to provide resistance against lepidopteran, coleopteran, homopteran, hemipteran, and other insects, or improved quality traits such as improved nutritional value. Herbicides for which transgenic plant tolerance has been demonstrated and the method of the present disclosure can be applied include, but are not limited to, glyphosate, dicamba, glufosinate, sulfonylurea, bromoxynil, norflurazon, 2,4-D (2,4-dichlorophenoxy) acetic acid, aryloxyphenoxy propionates, p-hydroxyphenyl pyruvate dioxygenase inhibitors (HPPD), and protoporphyrinogen oxidase inhibitors (PPO) herbicides. Polynucleotide molecules encoding proteins involved in herbicide tolerance known in the art and include, but are not limited to, a polynucleotide molecule encoding 5-enolpyruvylshikimate-3-phosphate synthase (EPSPS) disclosed in U.S. Pat. Nos. 5,094,945; 5,627,061; 5,633,435 and 6,040,497 for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in U.S. Patent No. Application Publication 2003/0083480 A1 also for imparting glyphosate tolerance; dicamba monooxygenase disclosed in U.S. Patent Application Publication 2003/0135879 A1 for imparting dicamba tolerance; a polynucleotide molecule encoding bromoxynil nitrilase (Bxn) disclosed in U.S. Pat. No. 4,810,648 for imparting bromoxynil tolerance; a polynucleotide molecule encoding phytoene desaturase (crtI) described in Misawa et al, (1993) Plant J. 4:833-840 and in Misawa et al, (1994) Plant J. 6:481-489 for norflurazon tolerance; a polynucleotide molecule encoding acetohydroxyacid synthase (AHAS, aka ALS) described in Sathasiivan et al. (1990) Nucl. Acids Res. 18:2188-2193 for imparting tolerance to sulfonylurea herbicides; polynucleotide molecules known as bar genes disclosed in DeBlock, et al. (1987) EMBO J. 6:2513-2519 for imparting glufosinate and bialaphos tolerance; polynucleotide molecules disclosed in U.S. Patent Application Publication 2003/010609 A1 for imparting N-amino methyl phosphonic acid tolerance; polynucleotide molecules disclosed in U.S. Pat. No. 6,107,549 for imparting pyridine herbicide resistance; molecules and methods for imparting tolerance to multiple herbicides such as glyphosate, atrazine, ALS inhibitors, isoxoflutole and glufosinate herbicides are disclosed in U.S. Pat. No. 6,376,754 and U.S. Patent Application Publication 2002/0112260. Molecules and methods for imparting insect/nematode/virus resistance are disclosed in U.S. Pat. Nos. 5,250,515; 5,880,275; 6,506,599; 5,986,175 and U.S. Patent Application Publication 2003/0150017 A1.

### Plant Cell Transformation Methods

Numerous methods for transforming a plant cell with a recombinant DNA, and/or introducing a recombinant DNA into chromosomes and plastids of a plant cell, are known in the art that may be used in methods of producing a transgenic or mutated plant cell and plant. Two effective methods for transformation are Agrobacterium-mediated transformation and microprojectile bombardment-mediated transformation. Microprojectile bombardment methods are illustrated, for example, in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn); U.S. Pat. No. 6,153,812 (wheat) and U.S. Pat. No. 6,365,807 (rice). Agrobacterium-mediated transformation methods are described, for example, in U.S. Pat. No. 5,159,135 (cotton); U.S. Pat. No. 5,824,877 (soybean); U.S. Pat. No. 5,463,174 (canola); U.S. Pat. No. 5,591,616 (corn); U.S. Pat. No. 5,846,797 (cotton); U.S. Pat. No. 8,044,260 (cotton); U.S. Pat. No. 6,384,301 (soybean), U.S. Pat. No. 7,026,528 (wheat) and U.S. Pat. No. 6,329,571 (rice), U.S. Patent Application Publication No. 2004/0087030 A1 (cotton), and U.S. Patent Application Publication No. 2001/0042257 A1 (sugar beet), all of which are incorporated herein by reference in their entirety. Transformation of plant material is practiced in tissue culture on nutrient media, for example a mixture of nutrients that allow cells to grow in vitro. Recipient cell targets include, but are not limited to, meristem cells, shoot tips, hypocotyls, calli, immature or mature embryos, and gametic cells such as microspores, pollen, sperm and egg cells. Callus can be initiated from tissue sources including, but not limited to, immature or mature embryos, hypocotyls, seedling apical meristems, microspores and the like. Cells containing a transgenic nucleus are grown into transgenic plants.

As introduced above, another method for transforming plant cells and chromosomes in a plant cell is via insertion of a DNA sequence using a recombinant DNA donor template at a pre-determined site of the genome by methods of site-directed integration. Site-directed integration may be accomplished by any method known in the art, for example, by use of zinc-finger nucleases, engineered or native meganucleases, TALE-endonucleases, or an RNA-guided endonuclease (for example Cas9 or Cpf1). The recombinant DNA construct may be inserted at the pre-determined site by homologous recombination (HR) or by non-homologous end joining (NHEJ). In addition to insertion of a recombinant DNA construct into a plant chromosome at a pre-determined site, genome editing can be achieved through oligonucleotide-directed mutagenesis (ODM) (Oh and May, 2001; U.S. Pat. No. 8,268,622) or by introduction of a double-strand break (DSB) or nick with a site specific nuclease, followed by NHEJ or repair. The repair of the DSB or nick may be used to introduce insertions or deletions at the site of the DSB or nick, and these mutations may result in the introduction of frame-shifts, amino acid substitutions, and/or an early termination codon of protein translation or alteration of a regulatory sequence of a gene. Genome editing may be achieved with or without a donor template molecule.

In addition to direct transformation or editing of a plant material with a recombinant DNA construct, a modified or transgenic plant can be prepared by crossing a first plant comprising a recombinant DNA, edit or mutation with a second plant lacking the recombinant DNA, edit or mutation. For example, a recombinant DNA, edit or mutation can be introduced into a first plant line that may be amenable to transformation, which can be crossed with a second plant line to introgress the recombinant DNA, edit or mutation into the second plant line. A modified or transgenic plant with a recombinant DNA, edit or mutation providing an enhanced trait, for example, enhanced yield or other yield component trait, can be crossed with a modified or transgenic plant line having another recombinant DNA, edit or mutation that confers another trait, for example herbicide resistance or pest resistance, to produce progeny plants having recombinant DNA sequences, edits or mutations that confer both traits. The progeny of these crosses may segregate, such that some of the plants will carry the recombinant DNA, edit or mutation for both parental traits and some will carry the recombinant DNA, edit or mutation for one of the parental traits; and such plants can be identified by one or both of the parental traits and/or markers associated with one or both of the parental traits or the the recombinant DNA, edit or mutation. For example, marker identification may be performed by analysis or detection of the recombinant DNA, edit or mutation, or in the case where a selectable marker is linked to the recombinant DNA, by application of a selection agent, such as a herbicide for use with a herbicide tolerance marker, or by selection for the enhanced trait or using any molecular technique. Progeny plants carrying DNA for both parental traits can be crossed back into one of the parent lines multiple times, for example 6 to 8 generations, to produce a progeny plant with substantially the same genotype as the original transgenic parental line, but for the recombinant DNA, edit or mutation of the other modified or transgenic parental line.

For transformation, DNA is typically introduced into only a small percentage of target plant cells in any one transformation experiment. Marker genes are used to provide an efficient system for identification of those cells that are stably transformed by receiving and integrating a recombinant DNA construct into their genomes. Preferred marker genes provide selective markers which confer resistance to a selective agent, such as an antibiotic or an herbicide. Any of the herbicides to which plants of this disclosure can be resistant is an agent for selective markers. Potentially transformed cells are exposed to the selective agent. In the population of surviving cells are those cells where, generally, the resistance-conferring gene is integrated and expressed at sufficient levels to permit cell survival. Cells can be tested further to confirm stable integration of the exogenous DNA. Commonly used selective marker genes include those conferring resistance to antibiotics such as kanamycin and paromomycin (nptll), hygromycin B (aph IV), spectinomycin (aadA) and gentamycin (aac3 and aacC4) or resistance to herbicides such as glufosinate (bar or pat), dicamba (DMO) and glyphosate (aroA or EPSPS). Examples of such selectable markers are illustrated in U.S. Pat. Nos. 5,550,318; 5,633,435; 5,780,708; 6,118,047 and 8,030,544. Markers which provide an ability to visually screen transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Plant cells that survive exposure to a selective agent, or plant cells that have been scored positive in a screening assay, may be cultured in vitro to develop or regenerate plantlets. Developing plantlets regenerated from transformed plant cells can be transferred to plant growth mix, and hardened off, for example, in an environmentally controlled chamber at about 85% relative humidity, 600 ppm CO₂, and 25-250 microEinsteins m⁻²s⁻¹ of light, prior to transfer to a greenhouse or growth chamber for maturation. Plants may be regenerated from about 6 weeks to 10 months after a transformant is identified, depending on the initial tissue, and plant species. Plants can be pollinated using conventional plant breeding methods known to those of skill in the art to produce seeds, for example cross-pollination and self-pollination are commonly used with transgenic corn and other plants. The regenerated transformed plant or its progeny seed or plants can be tested for expression of the recombinant DNA and selected for the presence of an altered phenotype or an enhanced agronomic trait.

### Modified and Transgenic Plants and Seeds

Modified or transgenic plants derived from modified or transgenic plant cells having a mutation, edit or transgene of this disclosure are grown to generate modified or transgenic plants having an altered phenotype or an enhanced trait as compared to a control plant, and produce modified or transgenic seed and haploid pollen of this disclosure. Such plants with enhanced traits are identified by selection of modified or transformed plants or progeny seed for the enhanced trait. For efficiency, a selection method is designed to evaluate multiple modified or transgenic plants (events) comprising the recombinant DNA, for example multiple plants from 2 to 20 or more transgenic events. Modified or transgenic plants grown from modified or transgenic seeds provided herein demonstrate improved agronomic traits that contribute to increased yield or other traits that provide increased plant value, including, for example, improved seed quality. Of particular interest are plants having increased water use efficiency or drought tolerance, enhanced high temperature or cold tolerance, increased yield, and increased nitrogen use efficiency.

Table 1 provides a list of sequences of protein-encoding genes as recombinant DNA for production of transgenic plants with enhanced traits. The elements of Table 1 are described by reference to: “NUC SEQ ID NO.” which identifies a DNA sequence; “PEP SEQ ID NO.” which identifies an amino acid sequence; “Gene ID” which refers to an identifier for the gene; and “Gene Name and Description” which is a common name and functional description of the gene.

TABLE 1
Sequences for Protein-Coding Genes

| NUC | PEP | | |
|---|---|---|---|
| SEQ ID | SEQ ID | | |
| NO. | NO. | Gene ID | Gene Name and Description |
| 1 | 32 | TX6-01 | Arabidopsis pescadillo-related transcription coactivator (AT5G14520) |
| 2 | 33 | TX6-02 | Arabidopsis ATP/GTP-binding protein (K10A8_120) |
| 3 | 34 | TX6-03 | corn FLC-like 3 gene |
| 4 | 35 | TX6-04 | Arabidopsis gibberellin 20-oxidase gene (At.GA20ox) |
| 5 | 36 | TX6-05 | rice cryptochrome 1a gene |
| 6 | 37 | TX6-06 | synechocystis fructose-1,6-bisphosphatase F-II |
| 7 | 38 | TX6-07 | corn gibberellin 20 oxidase 2 gene (Zm.GA20ox2) |
| 8 | 39 | TX6-08 | corn GA20oxgene (Zm.GA20ox) |
| 9 | 40 | TX6-09 | Arabidopsis galactose-binding lectin family protein |
| 10 | 41 | TX6-10 | corn gibberellin 20 oxidase 1 gene (Zm.GA20ox1A) |
| 11 | 42 | TX6-11 | corn amino acid permease (Zm.LHT1) |
| 12 | 43 | TX6-12 | Arabidopsis class V heat shock protien (ATHSP15.4) |
| 13 | 44 | TX6-13 | corn gene (ZmG395-2d94) |
| 14 | 45 | TX6-14 | Arabidopsis putative ribulose-5-phosphate-3-epimerase |
| 15 | 46 | TX6-15 | Saccharomyces cerevisiae GDH1 gene (Sc.GDH1) |
| 16 | 47 | TX6-16 | corn histidine rich protein |
| 17 | 48 | TX6-17 | Eutrema halophilum N1682_10 kDa PsbR subunit of photosystem II |
| 18 | 49 | TX6-18 | Sorghum Dehydration-responsive element-binding protein 2B |
| | | | (Sb.Dreb2b) |
| 19 | 50 | TX6-19 | Arabidopsis phytochrome-associated protein 2 PAP2 gene (At.PAP2) |
| 20 | 51 | TX6-20 | Eutrema halophilum N1624 universal stress protein family protein |
| 21 | 52 | TX6-21 | Arabidopsis fumarate hydratase |
| 22 | 53 | TX6-22 | corn MADS-domain transcription factor (Zmm19/ZmMADS19) |
| 23 | 54 | TX6-23 | soybean gene (Gm_W82_CR08.G217520) |
| 24 | 55 | TX6-24 | Arabidopsis starch synthase III |
| 25 | 56 | TX6-25 | rice Arginase |
| 26 | 57 | TX6-26 | Medicago HB1 gene (Mt.HB1) |
| 27 | 58 | TX6-27 | corn AtL1B gene (Zm.AtL1B) |
| 28 | 59 | TX6-28 | corn gene of unknown function (Zm_B73_CR09.G1925990) |
| 29 | 60 | TX6-29 | soybean gene (Gm_W82_CR01.G204980.1 CsID) |
| 30 | 61 | TX6-30 | barley FD2 gene (Hv.FD2) |
| 31 | 62 | TX6-31 | soybean TFL-like PhosphatidylEthanolamine-Binding Protein (PEBP) |
| | | | gene (Glyma16g32080, GmBFT) |

Table 2 provides a list of sequences for suppression of target protein-coding genes, as recombinant DNA for production of transgenic plants with enhanced traits. The elements of Table 2 are described by reference to:

“Target NUC SEQ ID NO.” which identifies a nucleotide coding sequence of the suppression target gene.

“Target PEP SEQ ID NO.” which identifies an amino acid sequence of the suppression target gene.

“Target Gene ID” which is an identifier of the suppression target gene.

“Engineered miRNA precursor SEQ ID NO.” which identifies a nucleotide sequence of the miRNA construct.

“miRNA targeting sequence SEQ ID NO.” which identifies a nucleotide sequence of the miRNA targeting sequence.

“Target Gene Name and Description” which is a common name and functional description of the suppression target gene.

TABLE 2
Sequences for Gene Suppression

| | | | | miRNA | |
|---|---|---|---|---|---|
| | | | Engineered | targeting | |
| Target NUC | Target PEP | Target | miRNA precursor | sequence | Target Gene Name |
| SEQ ID NO. | SEQ ID NO. | Gene ID | SEQ ID NO. | SEQ ID NO. | and Description |
| 63 | 70 | TX6-32T | 77 | 84 | corn Calcineurin |
| | | | | | B-like (CBL) - |
| | | | | | interacting protein |
| | | | | | kinase 8 gene |
| | | | | | homolog |
| | | | | | (Zm.CIPK8) |
| 64 | 71 | TX6-33T | 78 | 85 | corn sorbitol |
| | | | | | dehydrogenase |
| | | | | | gene (Zm.SDH) |
| 65 | 72 | TX6-34T | 79 | 86 | soybean |
| | | | | | HOMEOBOX |
| | | | | | transcription factor |
| | | | | | 1 gene (Gm.HB1) |
| 66 | 73 | TX6-35T | 80 | 87 | corn CKX4b gene |
| | | | | | (Zm.CKX4b, |
| | | | | | Zm_B73_CR08.G2 |
| | | | | | 196890.2) |
| 67 | 74 | TX6-36T | 81 | 88 | corn cytokinin |
| | | | | | dehydrogenase 10 |
| | | | | | (Zm.CKX10) |
| 68 | 75 | TX6-37T | 82 | 89 | soybean BRC1 |
| | | | | | gene (Gm.BRC1) |
| 69 | 76 | TX6-38T | 83 | 90 | soybean FULc |
| | | | | | gene (Gm.FULc) |

As an alternative to suppressing a target gene, the same target gene could instead be targeted for mutagenesis or genome editing to create mutations that reduce or eliminate its expression and/or the activity of a protein encoded by the target gene. Table 3 below provides genomic DNA sequences in corn or soybean encompassing the genomic locus for each target gene in Table 2. These genomic sequences can be used to design a guide RNA or engineer a site-specific nuclease to target and create a double strand break or nick at a target site in the genome of a corn or soy plant at or near the target gene, which may be repaired (with or without a donor template) to create a mutation (substitution, deletion, inversion, insertion, etc.) at or near the genomic target site to reduce or eliminate the expression and/or activity of the target gene.

TABLE 3
Target Gene Sequences for Genome Editing

| Target Gene | | Upstream | | Coding | Downstream |
|---|---|---|---|---|---|
| Name and Target | Genomic | Sequence of | Target Gene | Sequence | Sequence of |
| Gene ID | SEQ ID NO. | Target Gene | Sequence | (Exons) | Target Gene |
| corn Calcineurin | 141 | 1-2000 | 2001-13852 | 2001-2180, | 13853-14852 |
| B-like (CBL) - | | | | 4341-4403, | |
| interacting | | | | 4569-4640, | |
| protein kinase 8 | | | | 6822-6929, | |
| gene homolog | | | | 7017-7091, | |
| (Zm.CIPK8) | | | | 7169-7222, | |
| (TX6-32T) | | | | 7641-7766, | |
| | | | | 7893-7982, | |
| | | | | 8463-8585, | |
| | | | | 8733-8852, | |
| | | | | 13120-13236, | |
| | | | | 13341-13397, | |
| | | | | 13489-13563, | |
| | | | | 13757-13852 | |
| corn sorbitol | 142 | 1-2000 | 2001-4578 | 2001-2124, | 4579-5578 |
| dehydrogenase | | | | 3505-3572, | |
| gene (Zm.SDH) | | | | 3670-4578 | |
| (TX6-33T) | | | | | |
| soybean | 143 | 1-2000 | 2001-2950 | 2001-2372, | 2951-3950 |
| HOMEOBOX | | | | 2585-2950 | |
| transcription | | | | | |
| factor 1 gene | | | | | |
| (Gm.HB1) | | | | | |
| (TX6-34T) | | | | | |
| corn CKX4b gene | 144 | 1-2000 | 2001-5854 | 2001-2607, | 5855-6854 |
| (Zm.CKX4b) | | | | 2771-2898, | |
| (TX6-35T) | | | | 3659-3922, | |
| | | | | 4205-4476, | |
| | | | | 5521-5854 | |
| corn cytokinin | 145 | 1-2000 | 2001-4775 | 2001-2693, | 4776-5775 |
| dehydrogenase | | | | 27793069, | |
| 10 (Zm.CKX10) | | | | 3743-4014, | |
| (TX6-36T) | | | | 4454-4775 | |
| soybean BRC1 | 146 | 1-2000 | 2001-3779 | 2001-3110, | 3780-4779 |
| gene (Gm.BRC1) | | | | 3732-3779 | |
| (TX6-37T) | | | | | |
| soybean FULc | 147 | 1-2000 | 2001-13765 | 2001-2185, | 13766-14765 |
| gene (Gm.FULc) | | | | 11059-11134, | |
| (TX6-38T) | | | | 11340-11404, | |
| | | | | 12031-12130, | |
| | | | | 12301-12342, | |
| | | | | 12869-12907, | |
| | | | | 13013-13152, | |
| | | | | 13666-13765 | |

Table 4 provides a list of constructs with specific expression pattern, for expression or suppression of protein-coding genes, as recombinant DNA for production of transgenic plants with enhanced traits. The elements of Table 4 are described by reference to:

“Construct ID” which identifies a construct with a particular expression pattern by a promoter operably linked to a polynucleotide sequence either expressing or suppressing a protein-coding gene.

“Gene ID” which identifies either an expressed or suppressed gene from Table 1 or Table 2.

“Specific Expression Pattern” which describes the expected expression pattern or promoter type.

TABLE 4
Constructs for Gene expression and suppression

| Construct ID | Gene ID | Specific Expression Pattern |
|---|---|---|
| TX6-01 | TX6-01 | Root Preferred |
| TX6-02 | TX6-02 | Root Preferred |
| TX6-03 | TX6-03 | Root Preferred |
| TX6-04 | TX6-04 | Seed Preferred |
| TX6-05 | TX6-05 | Constitutive |
| TX6-06 | TX6-06 | Constitutive |
| TX6-07 | TX6-07 | Endosperm Preferred |
| TX6-08c1 | TX6-08 | Seed Preferred |
| TX6-08c2 | TX6-08 | Meristem Preferred |
| TX6-08c3 | TX6-08 | Root Preferred |
| TX6-09 | TX6-09 | Constitutive |
| TX6-10 | TX6-10 | Endosperm Preferred |
| TX6-11 | TX6-11 | Seed Preferred |
| TX6-12 | TX6-12 | Constitutive |
| TX6-13 | TX6-13 | Constitutive |
| TX6-14 | TX6-14 | Leaf Bundle Sheath Preferred |
| TX6-15 | TX6-15 | Seed Preferred |
| TX6-16 | TX6-16 | Constitutive |
| TX6-17 | TX6-17 | Constitutive |
| TX6-18 | TX6-18 | Constitutive |
| TX6-19 | TX6-19 | Constitutive |
| TX6-20 | TX6-20 | Seed, Root, Leaf Preferred |
| TX6-21 | TX6-21 | Above Ground Preferred |
| TX6-22 | TX6-22 | Root Preferred |
| TX6-23 | TX6-23 | Constitutive |
| TX6-24c1 | TX6-24 | Seed Preferred |
| TX6-24c2 | TX6-24 | Leaf Mesophyll Preferred |
| TX6-24c3 | TX6-24 | Endosperm Preferred |
| TX6-25 | TX6-25 | Seed, Root, Leaf Preferred |
| TX6-26 | TX6-26 | Constitutive |
| TX6-27 | TX6-27 | Constitutive |
| TX6-28 | TX6-28 | Leaf Preferred |
| TX6-29 | TX6-29 | Root Preferred |
| TX6-30 | TX6-30 | Constitutive |
| TX6-31 | TX6-31 | Meristem Preferred |
| TX6-32T | TX6-32T | Constitutive |
| TX6-33T | TX6-33T | Endosperm Preferred |
| TX6-34T | TX6-34T | Constitutive |
| TX6-35T | TX6-35T | Seed Preferred |
| TX6-36T | TX6-36T | Seed Preferred |
| TX6-37T | TX6-37T | Constitutive |
| TX6-38T | TX6-38T | Constitutive |

Table 5 provides a list of polynucleotide sequences of promoters with specific expression patterns. To convey the specific expression patterns, choices of promoters are not limited to those listed in Table 5.

TABLE 5
Promoter sequences and expression patterns

| Nucleotide SEQ ID NO. | Promoter Expression Pattern |
|---|---|
| 95 | Root Preferred |
| 96 | Seed Preferred |
| 97 | Endosperm Preferred |
| 98 | Meristem Preferred |
| 99 | Leaf Bundle Sheath Preferred |
| 100 | Above Ground Preferred |
| 101 | Leaf Mesophyll Preferred |
| 102 | Leaf Preferred |
| 103 | Endosperm Preferred |

### Selecting and Testing Transgenic Plants for Enhanced Traits

Within a population of transgenic plants each developed or regenerated from a plant cell with a recombinant DNA, many plants that survive to fertile transgenic plants that produce seeds and progeny plants will not exhibit an enhanced agronomic trait. Selection from the population may be necessary to identify one or more transgenic plants with an enhanced trait. Further evaluation with vigorous testing may be important for understanding the contributing components to a trait, supporting trait advancement decisions and generating mode of action hypotheses. Transgenic plants having enhanced traits can be selected and tested from populations of plants developed, regenerated or derived from plant cells transformed as described herein by evaluating the plants in a variety of assays to detect an enhanced trait, for example, increased water use efficiency or drought tolerance, enhanced high temperature or cold tolerance, increased yield or yield components, desirable architecture, optimum life cycle, increased nitrogen use efficiency, enhanced seed composition such as enhanced seed protein and enhanced seed oil.

These assays can take many forms including, but not limited to, direct screening for the trait in a greenhouse or field trial or by screening for a surrogate trait. Such analyses can be directed to detecting changes in the chemical composition, biomass, yield components, physiological property, root architecture, morphology, or life cycle of the plant. Changes in chemical compositions such as nutritional composition of grain can be detected by analysis of the seed composition and content of protein, free amino acids, oils, free fatty acids, starch or tocopherols. Changes in chemical compositions can also be detected by analysis of contents in leaves, such as chlorophyll or carotenoid contents. Changes in biomass characteristics can be evaluated on greenhouse or field grown plants and can include plant height, stem diameter, root and shoot dry weights, canopy size; and, for corn plants, ear length and diameter. Changes in yield components can be measured by total number of kernels per unit area and its individual weight. Changes in physiological properties can be identified by evaluating responses to stress conditions, for example assays using imposed stress conditions such as water deficit, nitrogen deficiency, cold growing conditions, pathogen or insect attack or light deficiency, or increased plant density. Changes in root architecture can be evaluated by root length and branch number. Changes in morphology can be measured by visual observation of tendency of a transformed plant to appear to be a normal plant as compared to changes toward bushy, taller, thicker, narrower leaves, striped leaves, knotted trait, chlorosis, albino, anthocyanin production, or altered tassels, ears or roots. Changes in morphology can also be measured with morphometric analysis based on shape parameters, using dimensional measurement such as ear diameter, ear length, kernel row number, internode length, plant height, or stem volume. Changes in life cycle can be measured by macro or microscopic morphological changes partitioned into developmental stages, such as days to pollen shed, days to silking, leaf extension rate. Other selection and testing properties include days to pollen shed, days to silking, leaf extension rate, chlorophyll content, leaf temperature, stand, seedling vigor, internode length, plant height, leaf number, leaf area, tillering, brace roots, stay green or delayed senescence, stalk lodging, root lodging, plant health, bareness/prolificacy, green snap, and pest resistance. In addition, phenotypic characteristics of harvested grain can be evaluated, including number of kernels per row on the ear, number of rows of kernels on the ear, kernel abortion, kernel weight, kernel size, kernel density and physical grain quality.

Assays for screening for a desired trait are readily designed by those practicing in the art. The following illustrates screening assays for corn traits using hybrid corn plants. The assays can be adapted for screening other plants such as canola, wheat, cotton and soybean either as hybrids or inbreds.

Transgenic corn plants having increased nitrogen use efficiency can be identified by screening transgenic plants in the field under the same and sufficient amount of nitrogen supply as compared to control plants, where such plants provide higher yield as compared to control plants. Transgenic corn plants having increased nitrogen use efficiency can also be identified by screening transgenic plants in the field under reduced amount of nitrogen supply as compared to control plants, where such plants provide the same or similar yield as compared to control plants.

Transgenic corn plants having increased yield can be identified by screening using progenies of the transgenic plants over multiple locations for several years with plants grown under optimal production management practices and maximum weed and pest control or standard agronomic practices (SAP). Selection methods can be applied in multiple and diverse geographic locations, for example up to 16 or more locations, over one or more planting seasons, for example at least two planting seasons, to statistically distinguish yield improvement from natural environmental effects.

Transgenic corn plants having increased water use efficiency or drought tolerance can be identified by screening plants in an assay where water is withheld for a period to induce stress followed by watering to revive the plants. For example, a selection process imposes 3 drought/re-water cycles on plants over a total period of 15 days after an initial stress free growth period of 11 days. Each cycle consists of 5 days, with no water being applied for the first four days and a water quenching on the 5th day of the cycle. The primary phenotypes analyzed by the selection method may be changes in plant growth rate as determined by height and biomass during a vegetative drought treatment.

Although the plant cells and methods of this disclosure can be applied to any plant cell, plant, seed or pollen, for example, any fruit, vegetable, grass, tree or ornamental plant, the various aspects of the disclosure are applied to corn, soybean, cotton, canola, rice, barley, oat, wheat, turf grass, alfalfa, sugar beet, sunflower, quinoa and sugar cane plants.

### Examples

### Example 1. Corn Transformation

This example illustrates transformation methods to produce a transgenic corn plant cell, seed, and plant having altered phenotypes as shown in Tables 6-8, and enhanced traits, increased water use efficiency, increased nitrogen use efficiency, and increased yield and altered traits and phenology as shown in Tables 10-15.

For Agrobacterium-mediated transformation of corn embryo cells, ears from corn plants were harvested and surface-sterilized by spraying or soaking the ears in ethanol, followed by air drying. Embryos were isolated from individual kernels of surface-sterilized ears. After excision, maize embryos were inoculated with Agrobacterium cells containing plasmid DNA with the gene of interest cassette and a plant selectable marker cassette, and then co-cultured with Agrobacterium for several days. Co-cultured embryos were transferred to various selection and regeneration media, and transformed R0 plants were recovered 6 to 8 weeks after initiation of selection, which were transplanted into potting soil. Regenerated R0 plants were selfed, and R1 and subsequent progeny generations were obtained.

The above process can be repeated to produce multiple events of transgenic corn plants from cells that were transformed with recombinant DNA having the constructs identified in Table 3. Progeny transgenic plants and seeds of the transformed plants were screened for the presence and single copy of the inserted gene, and for various altered or enhanced traits and phenotypes, such as increased water use efficiency, increased yield, and increased nitrogen use efficiency as shown in Tables 6-8 and 10-15. From each group of multiple events of transgenic plants with a specific recombinant DNA from Table 3, the event(s) that showed increased yield, increased water use efficiency, increased nitrogen use efficiency, and altered phenotypes and traits were identified.

### Example 2. Soybean Transformation

This example illustrates plant transformation in producing a transgenic soybean plant cell, seed, and plant having an altered phenotype or an enhanced trait, such as increased water use efficiency, drought tolerance and increased yield as shown in Table 14.

For Agrobacterium mediated transformation, soybean seeds were imbibed overnight and the meristem explants excised. Soybean explants were mixed with induced Agrobacterium cells containing plasmid DNA with the gene of interest cassette and a plant selectable marker cassette no later than 14 hours from the time of initiation of seed imbibition, and wounded using sonication. Following wounding, explants were placed in co-culture for 2-5 days at which point they were transferred to selection media to allow selection and growth of transgenic shoots. Resistant shoots were harvested in approximately 6-8 weeks and placed into selective rooting media for 2-3 weeks. Shoots producing roots were transferred to the greenhouse and potted in soil. Shoots that remained healthy on selection, but did not produce roots were transferred to non-selective rooting media for an additional two weeks. Roots from any shoots that produced roots off selection were tested for expression of the plant selectable marker before they were transferred to the greenhouse and potted in soil.

The above process can be repeated to produce multiple events of transgenic soybean plants from cells that were transformed with recombinant DNA having the constructs identified in Table 3. Progeny transgenic plants and seed of the transformed plants were screened for the presence and single copy of the inserted gene, and tested for various altered or enhanced phenotypes and traits as shown in Tables 7-9 and 11-16.

### Example 3. Identification of Altered Phenotypes in Automated Greenhouse

This example illustrates screening and identification of transgenic corn plants for altered phenotypes in an automated greenhouse (AGH). The apparatus and the methods for automated phenotypic screening of plants are disclosed, for example, in U.S. Patent Publication No. 2011/0135161, which is incorporated herein by reference in its entirety.

Corn plants were tested in three screens in the AGH under different conditions including non-stress, nitrogen deficit, and water deficit stress conditions. All screens began with non-stress conditions during days 0-5 germination phase, after which the plants were grown for 22 days under the screen-specific conditions shown in Table 6.

TABLE 6
Description of the three AGH screens for corn plants

| | | Germination | Screen specific |
|---|---|---|---|
| Screen | Description | phase (5 days) | phase (22 days) |
| Non-stress | well watered | 55% VWC | 55% VWC |
| | sufficient nitrogen | water | 8 mM nitrogen |
| Water deficit | limited watered | 55% VWC | 30% VWC |
| | sufficient nitrogen | water | 8 mM nitrogen |
| Nitrogen deficit | well watered | 55% VWC | 55% VWC |
| | low nitrogen | water | 2 mM nitrogen |

Water deficit is defined as a specific Volumetric Water Content (VWC) that is lower than the VWC of a non-stressed plant. For example, a non-stressed plant might be maintained at 55% VWC, and the VWC for a water-deficit assay might be defined around 30% VWC. Data were collected using visible light and hyperspectral imaging as well as direct measurement of pot weight and amount of water and nutrient applied to individual plants on a daily basis.

Nitrogen deficit is defined (in part) as a specific mM concentration of nitrogen that is lower than the nitrogen concentration of a non-stressed plant. For example, a non-stressed plant might be maintained at 8 mM nitrogen, while the nitrogen concentration applied in a nitrogen-deficit assay might be maintained at a concentration of 2 mM.

Up to ten parameters were measured for each screen. The visible light color imaging based measurements are: biomass, canopy area, and plant height. Biomass (Bmass) is defined as the estimated shoot fresh weight (g) of the plant obtained from images acquired from multiple angles of view. Canopy Area (Cnop) is defined as leaf area as seen in a top-down image (mm²). Plant Height (PlntH) refers to the distance from the top of the pot to the highest point of the plant derived from a side image (mm). Anthocyanin score and area, chlorophyll score and concentration, and water content score are hyperspectral imaging-based parameters. Anthocyanin Score (AntS) is an estimate of anthocyanin in the leaf canopy obtained from a top-down hyperspectral image. Anthocyanin Area (AntA) is an estimate of anthocyanin in the stem obtained from a side-view hyperspectral image. Chlorophyll Score (ClrpS) and Chlorophyll Concentration (ClrpC) are both measurements of chlorophyll in the leaf canopy obtained from a top-down hyperspectral image, where Chlorophyll Score measures in relative units, and Chlorophyll Concentration is measured in parts per million (ppm) units. Water Content Score (WtrCt) is a measurement of water in the leaf canopy obtained from a top-down hyperspectral image. Water Use Efficiency (WUE) is derived from the grams of plant biomass per liter of water added. Water Applied (WtrAp) is a direct measurement of water added to a pot (pot with no hole) during the course of an experiment to maintain a stable soil water content.

These physiological screen runs were set up so that tested transgenic lines were compared to a control line. The collected data were analyzed against the control using % delta and certain p-value cutoff. Tables 7, 8 and 9 are summaries of transgenic corn plants comprising the disclosed recombinant DNA constructs with altered phenotypes under non stress, nitrogen deficit, and water deficit conditions, respectively. “ConstructID” refers to the construct identifier as defined in Table 4.

The test results are represented by three numbers: the first number before letter “p” denotes number of events with an increase in the tested parameter at p<0.1; the second number before letter “n” denotes number of events with a decrease in the tested parameter at p<0.1; the third number before letter “t” denotes total number of transgenic events tested for a given parameter in a specific screen. The increase or decrease is measured in comparison to non-transgenic control plants. A designation of “-” indicates that it has not been tested. For example, 2p1n5t indicates that 5 transgenic plant events were screened, of which 2 events showed an increase, and 1 showed a decrease of the measured parameter.

TABLE 7
Summary of transgenic plants with altered phenotypes in AGH non-stress screens

| Construct | | | | | | | | | | |
|---|---|---|---|---|---|---|---|---|---|---|
| ID | AntS | Bmass | Cnop | ClrpS | PlntH | WtrAp | WtrCt | WUE | ClrpC | AntA |
| TX6-05 | 0p3n5t | 0p5n5t | 0p4n5t | 4p0n5t | 0p5n5t | 0p5n5t | 0p0n5t | 0p5n5t | — | — |
| TX6-07 | 2p0n3t | 1p0n3t | 0p1n3t | — | 0p1n3t | 0p1n3t | — | 1p1n3t | 0p0n3t | — |
| TX6-08c1 | 0p0n5t | 0p2n5t | 0p2n5t | — | 0p3n5t | 0p1n5t | — | 0p2n5t | 0p1n5t | — |
| TX6-08c3 | 0p0n5t | 0p1n5t | 1p1n5t | — | 1p0n5t | 0p0n5t | — | 0p1n5t | 0p0n5t | 2p0n5t |
| TX6-09 | 0p0n5t | 1p1n5t | 0p1n5t | — | 0p0n5t | 0p2n5t | — | 0p1n5t | 1p1n5t | — |
| TX6-10 | 0p0n5t | 0p2n5t | 0p1n5t | — | 0p1n5t | 0p3n5t | — | 0p3n5t | 0p0n5t | — |
| TX6-11 | 0p1n5t | 0p0n5t | 0p0n5t | — | 1p1n5t | 0p0n5t | — | 1p0n5t | 0p0n5t | 1p0n5t |
| TX6-12 | 0p0n5t | 0p0n5t | 0p0n5t | — | 0p2n5t | 0p0n5t | — | 0p0n5t | 1p0n5t | 0p0n5t |
| TX6-15 | 3p0n5t | 0p3n5t | 0p5n5t | — | 0p4n5t | 0p3n5t | — | 0p3n5t | 0p0n5t | 2p0n5t |
| TX6-24c2 | 0p0n10t | 1p2n10t | 3p2n10t | — | 0p0n10t | 3p0n10t | — | 1p1n10t | 0p0n10t | 0p0n10t |

TABLE 8
Summary of transgenic plants with altered phenotypes in AGH nitrogen-deficit screens

| Construct | | | | | | | | | | |
|---|---|---|---|---|---|---|---|---|---|---|
| ID | AntA | AntS | Bmass | Cnop | ClrpC | PlntH | WtrAp | WUE | ClrpS | WtrCt |
| TX6-01 | 2p0n5t | 0p0n5t | 0p0n5t | 0p0n5t | 0p0n5t | 0p0n5t | 1p0n5t | 0p0n5t | — | — |
| TX6-02 | 0p0n5t | 0p0n5t | 0p2n5t | 0p1n5t | 1p0n5t | 0p0n5t | 0p0n5t | 0p2n5t | — | — |
| TX6-03 | 0p1n5t | 0p1n5t | 0p1n5t | 0p1n5t | 0p0n5t | 0p0n5t | 0p0n5t | 0p1n5t | — | — |
| TX6-05 | — | 0p4n5t | 0p4n5t | 0p4n5t | — | 0p4n5t | 0p4n5t | 0p4n5t | 4p0n5t | 2p0n5t |
| TX6-06 | 0p0n5t | — | 3p0n5t | 1p1n5t | — | 1p0n5t | 3p0n5t | 3p0n5t | — | — |
| TX6-07 | — | 0p1n3t | 0p2n3t | 0p2n3t | 0p0n3t | 0p1n3t | 0p2n3t | 0p1n3t | — | — |
| TX6-08c1 | — | 0p1n5t | 0p0n5t | 0p0n5t | 1p1n5t | 1p1n5t | 0p1n5t | 1p0n5t | — | — |
| TX6-08c3 | 4p0n5t | 0p0n5t | 2p0n5t | 3p0n5t | 0p2n5t | 4p0n5t | 3p0n5t | 2p0n5t | — | — |
| TX6-09 | — | 1p0n5t | 0p1n5t | 0p2n5t | 0p2n5t | 0p0n5t | 0p2n5t | 0p1n5t | — | — |
| TX6-10 | 0p0n5t | 0p2n10t | 4p1n10t | 2p1n10t | 4p0n10t | 4p0n10t | 4p2n10t | 4p0n10t | — | — |
| TX6-11 | 0p2n5t | 0p0n5t | 0p1n5t | 0p2n5t | 0p0n5t | 0p0n5t | 2p0n5t | 0p1n5t | — | — |
| TX6-12 | 1p0n5t | 1p0n5t | 0p3n5t | 0p4n5t | 0p1n5t | 0p2n5t | 0p1n5t | 0p3n5t | — | — |
| TX6-13 | 0p1n5t | — | 2p0n5t | 0p0n5t | — | 1p0n5t | 0p0n5t | 3p0n5t | — | — |
| TX6-15 | 0p1n5t | 0p0n5t | 0p0n5t | 0p1n5t | 1p0n5t | 0p4n5t | 0p0n5t | 0p1n5t | — | — |
| TX6-16 | 0p0n5t | 0p0n5t | 0p3n5t | 1p1n5t | 0p2n5t | 0p1n5t | 0p1n5t | 0p3n5t | — | — |
| TX6-18 | 0p2n5t | 0p0n5t | 5p0n5t | 4p0n5t | 3p0n5t | 4p0n5t | 1p0n5t | 5p0n5t | — | — |
| TX6-19 | 0p1n5t | 0p0n5t | 4p0n5t | 5p0n5t | 1p0n5t | 1p0n5t | 0p0n5t | 5p0n5t | — | — |
| TX6-20 | 1p0n5t | 0p0n5t | 0p4n5t | 0p2n5t | 0p1n5t | 0p3n5t | 0p2n5t | 0p4n5t | — | — |
| TX6-25 | 2p0n5t | 0p0n5t | 0p1n5t | 0p0n5t | 0p1n5t | 0p0n5t | 0p0n5t | 0p1n5t | — | — |
| TX6-27 | 1p1n8t | 0p1n8t | 1p1n8t | 3p1n8t | 1p0n8t | 0p1n8t | 3p1n8t | 1p1n8t | — | — |
| TX6-32T | 0p1n5t | 1p0n5t | 0p3n5t | 0p0n5t | 0p2n5t | 0p5n5t | 0p5n5t | 0p0n5t | — | — |
| TX6-33T | 0p1n10t | Ip0n10t | 5p0n10t | 6p1n10t | 2p0n10t | 2p1n10t | 6p0n10t | 5p0n10t | — | — |

TABLE 9
Summary of transgenic plants with altered phenotypes in AGH water-deficit screens

| Construct | | | | | | | | | | |
|---|---|---|---|---|---|---|---|---|---|---|
| ID | AntA | AntS | Bmass | Cnop | ClrpC | PlntH | WtrAp | WUE | ClrpS | WtrCt |
| TX6-03 | 0p0n5t | 0p0n5t | 0p1n5t | 0p2n5t | 0p0n5t | 0p0n5t | 0p1n5t | 0p2n5t | — | — |
| TX6-05 | — | Ip2n5t | 0p3n5t | 0p4n5t | — | 0p4n5t | 2p1n5t | 0p4n5t | 4p0n5t | 1p0n5t |
| TX6-06 | 5p0n5t | 0p1n5t | 0p5n5t | 0p5n5t | 0p3n5t | 0p4n5t | 0p5n5t | 0p1n5t | — | — |
| TX6-07 | — | 0p0n3t | 0p0n3t | 0p2n3t | 0p0n3t | 0p1n3t | 0p2n3t | 0p0n3t | — | — |
| TX6-08c1 | — | 0p0n5t | 0p3n5t | 0p3n5t | 0p0n5t | Ip3n5t | 0p3n5t | 0p1n5t | — | — |
| TX6-08c3 | 0p0n5t | 1p0n5t | 2p0n5t | 0p0n5t | 0p1n5t | 4p0n5t | 1p0n5t | 1p0n5t | — | — |
| TX6-09 | — | 0p1n5t | 3p0n5t | 4p0n5t | 0p0n5t | 1p0n5t | 5p0n5t | 0p0n5t | — | — |
| TX6-10 | — | 0p1n5t | 1p0n5t | 0p0n5t | 1p0n5t | 0p1n5t | 1p0n5t | 0p0n5t | — | — |
| TX6-11 | 0p3n5t | 1p0n5t | 0p2n5t | 0p1n5t | 0p1n5t | 0p1n5t | 1p1n5t | 0p2n5t | — | — |
| TX6-12 | 0p0n5t | 1p0n5t | 0p0n5t | 0p0n5t | 0p0n5t | 0p1n5t | 0p1n5t | 0p0n5t | — | — |
| TX6-13 | 1p0n5t | 2p0n5t | 0p2n5t | 0p1n5t | 0p0n5t | 0p1n5t | 0p4n5t | 0p0n5t | — | — |
| TX6-15 | 0p0n5t | 1p0n5t | 0p1n5t | 0p0n5t | 0p1n5t | 0p4n5t | 0p3n5t | 0p0n5t | — | — |
| TX6-16 | 0p0n5t | 1p1n5t | 0p1n5t | 0p0n5t | 0p0n5t | 0p1n5t | 0p0n5t | 0p1n5t | — | — |
| TX6-18 | 0p2n5t | 1p0n5t | 3p0n5t | 1p0n5t | 0p0n5t | 2p0n5t | 0p0n5t | 3p0n5t | — | — |
| TX6-19 | 0p4n5t | 1p0n5t | 4p0n5t | 4p0n5t | 0p0n5t | 0p0n5t | 0p0n5t | 4p0n5t | — | — |
| TX6-22 | 0p0n5t | 0p0n5t | 0p0n5t | 1p0n5t | 0p0n5t | 0p0n5t | 0p0n5t | 0p0n5t | — | — |
| TX6-27 | 4p0n8t | 1p0n8t | 0p6n8t | 0p3n8t | 0p3n8t | 0p1n8t | 0p7n8t | 0p3n8t | — | — |
| TX6-32T | 2p0n5t | 1p0n5t | 0p2n5t | 0p2n5t | 0p1n5t | 0p2n5t | 0p2n5t | 0p2n5t | — | — |
| TX6-33T | 0p2n10t | 0p0n10t | 2p1n10t | 2p1n10t | 1p1n10t | 3p1n10t | 2p5n10t | 4p0n10t | — | — |

### Example 4. Evaluation of Transgenic Plants for Trait Characteristics

Trait assays were conducted to evaluate trait characteristics and phenotypic changes in transgenic plants as compared to non-transgenic controls. Corn and soybean plants were grown in field and greenhouse conditions. Up to 18 parameters were measured for corn in phenology, morphometrics, biomass, and yield component studies at certain plant developmental stages. For root assays, soybean plants were grown in the greenhouse in transparent nutrient medium to allow the root system to be imaged and analyzed.

Corn developmental stages are defined by the following development criteria:

Developed leaf: leaf with a visible leaf collar;

V-Stages: Number of developed leaves on a corn plant corresponds to the plant's vegetative growth stage—i.e., a V6 stage corn plant has 6 developed (fully unfolded) leaves;

R1 (Silking): Plants defined as R1 must have one or more silks extending outside the husk leaves. Determining the reproductive stage of the crop plant at R1 or later is based solely on the development of the primary ear;

R3 (Milk): Typically occurs 18-22 days after silking depending on temperature and relative maturity. Kernels are usually yellow in color and the fluid inside each kernel is milky white;

R6 (Physiological maturity): Typically occurs 55-65 days after silking (depending on temperature and relative maturity group of the germplasm being observed). Kernels have reached their maximum dry matter accumulation at this point, and kernel moisture is approximately 35%.

Soybean developmental stages are defined by criteria as following:

Fully developed trifoliate leaf node: A leaf is considered completely developed when the leaf at the node immediately above it has unrolled sufficiently so the two edges of each leaflet are no longer touching. At the terminal node on the main stem, the leaf is considered completely developed when the leaflets are flat and similar in appearance to older leaves on the plant;

VC: Cotyledons and Unifoliolates are fully expanded;

R1: Beginning of flowering—i.e., one open flower at any node on the main stem.

Table 10 describes the trait assays. TraitRefID is the reference ID of each trait assay. Trait Assay Name is the descriptive name of the assay. The Description provides what the assay measures, and how the measurement is conducted. Direction For Positive Call indicates whether an increase or decrease in the measurement quantity corresponds to a “positive” call in the assay results.

TABLE 10
Description of Trait Assays

| | | | Direction For |
|---|---|---|---|
| TraitRefID | Trait Assay Name | Description | Positive Call |
| HINDXR6 | Harvest Index at R6 | Ratio of grain weight to total plant weight at | increase |
| | | harvest. Weights are determined on a dry | |
| | | weight basis. | |
| DBMSR6 | Dry Biomass by Seed | Ratio of grain weight to total plant weight at R6 | increase |
| | at R6 | stage. Weights are determined on a dry weight | |
| | | basis. | |
| AGDWR6 | Total Dry Biomass | Total aboveground oven-dried biomass at R6. | increase |
| | at R6 | Plants are cut at ground level, oven-dried at 70 | |
| | | deg. C. to a constant weight, and weighed. | |
| DFL50 | Days from Planting to | Days from Planting to 50% Flowering | neutral |
| | 50% Flowering | | |
| PDPPR8 | Number of Pods per | Total pods per soybean plant. Quotient of count | increase |
| | Plant at R8 | of pods from plants in a defined linear distance | |
| | | (20″) on a plot row divided by number of plants. | |
| PDNODER8 | Pods per Node at R8 | Total pods per flowering node on a soybean | increase |
| | | plant. Quotient from count of pods on plants in a | |
| | | defined linear distance (20″) on a plot row | |
| | | divided by count of nodes on those plants. | |
| ARDR2 | Average Root Diameter | Estimated average diameter of all root classes of | increase |
| | at R2 | root at R2 stage, using WinRHIZO (TM) image | |
| | | analysis system software. | |
| RBNR2 | Root branch number | Number of root branches per plant determined | increase |
| | at R2 | by automated analysis of digitized root images | |
| | | from field root digs. | |
| DOV12 | Days from Planting | number of days from the date of planting to the | decrease |
| | to V12 | date when 50% of the plants in a plot reaches | |
| | | V12 stage. | |
| EAR6 | Ear Area at R6 | plot average of size of area of a ear from a 2- | increase |
| | | dimentional view. The measurement is done | |
| | | through imaging of ear, including kernels and | |
| | | void. Typically 10 representative ears are | |
| | | measured per plot. Measurement is taken at R6 | |
| | | stage. | |
| EDR6 | Ear Diameter at R6 | plot average of the ear diameter. It measures | increase |
| | | maximal “wide” axis over the ear on the largest | |
| | | section of the ear. Measurement is taken at R6 | |
| | | stage. | |
| EDWR1 | Ear Dry Weight at R6 | plot average of the ear dry weight of a plant. | increase |
| | | Measurement is taken at R6 stage. | |
| ELR6 | Ear Length at R6 | plot average of the length of ear. It measures | increase |
| | | from tip of ear in a straight line to the base at the | |
| | | ear node. Measurement is taken at R6 stage. | |
| ETVR6 | Ear Tip Void | plot average of area percentage of void at the | decrease |
| | Percentage at R6 | top 30% area of a ear, from a 2-dimentional | |
| | | view. The measurement is done through imaging | |
| | | of ear, including kernels and void. Typically 10 | |
| | | representative ears are measured per plot. | |
| | | Measurement is taken at R6 stage. | |
| EVR6 | Ear Void Percentage | plot average of area percentage of void on a ear, | decrease |
| | at R6 | from a 2-dimentional view. The measurement is | |
| | | done through imaging of ear, including kernels | |
| | | and void. Typically 10 representative ears are | |
| | | measured per plot. Measurement is taken at R6 | |
| | | stage. | |
| KPER6 | Kernels per Ear | plot average of the number of kernels per ear. It | increase |
| | at R6 | is calculated as (total kernel weight/(Single | |
| | | Kernel Weight * total ear count), where total | |
| | | kernel weight and total ear count are measured | |
| | | from ear samples from an area between 0.19 to | |
| | | 10 square meters, and Single Kernel Weight | |
| | | (SKWTR6) is described below. Measurement is | |
| | | taken at R6 stage. | |
| KRLR6 | Kernels per Row | (also known as rank number) the plot average of | increase |
| | Longitudinally at R6 | the number of kernels per row longitudinally. It is | |
| | | calculated as the ratio of (total kernel count per | |
| | | ear)/(kernel row number). Measurement is | |
| | | taken at R6 stage. | |
| KRNR6 | Kernel Row Number | plot average of the number of rows of kernels on | increase |
| | at R6 | an ear, by counting around the circumference of | |
| | | the ear. Measurement is taken at R6 stage. | |
| LFTNR3 | Leaf Tip Number at | plot average of the number of leaves per plant, | increase |
| | R3 | by counting the number of leaf tips. | |
| | | Measurement is taken at R3 stage. | |
| P50DR1 | Days to 50% Pollen | number of days from the date of planting to the | decrease |
| | Shedding | date when 50% of the plants in a plot reaches | |
| | | Pollen Shed stage. | |
| PHTR3 | Plant Height at R3 | plot average of plant height. It measures from | decrease |
| | | soil line to base of highest collared leaf. | |
| | | Measurement is taken at R3 stage. | |
| PLTHGR | Plant Height Growth | plot average of growth rate of a plant from V6 to | increase |
| | Rate from V6 to V12 | V12 stage. It is calculated as (Plant Height | |
| | | measured at V12 - Plant Height measured at | |
| | | V6)/Days between measurements. | |
| RBPN | Root Branch Point | number of root branch tip points of a plant. The | increase |
| | Number at VC or V2 | measurement is done through imaging of the | |
| | | root system of a plant grown in a transparent | |
| | | Gelzan(TM) gum gel nutrient medium to VC | |
| | | stage for soybean, or to V2 stage for corn. The | |
| | | root system image is skeletonized for the root | |
| | | length measurement. Up to 40 images are taken | |
| | | at various angles around the root vertical axis | |
| | | and measurement is averaged over the images. | |
| | | Gelzan is a trademark of CP Kelco U.S., Inc. | |
| RTL | Root Total Length at | cumulative length of roots of a plant, as if the | increase |
| | VC or V2 | roots were all lined up in a row. The | |
| | | measurement is done through imaging of the | |
| | | root system of a plant grown in a transparent | |
| | | Gelzan(TM) gum gel nutrient medium to VC | |
| | | stage for soybean, or to V2 stage for corn. The | |
| | | root system image is skeletonized for the root | |
| | | length measurement. Up to 40 images are taken | |
| | | at various angles around the root vertical axis | |
| | | and measurement is averaged over the images. | |
| | | Gelzan is a trademark of CP Kelco U.S., Inc. | |
| S50DR1 | Days to 50% Visible | number of days from the date of planting to the | decrease |
| | Silk | date when 50% of the plants in a plot reaches | |
| | | visible Silking (R1) stage. | |
| SKWTR6 | Single Kernel Weight | plot average of weight per kernel. It is calculated | increase |
| | at R6 | as the ratio of (sample kernel weight adjusted to | |
| | | 15.5% moisture)/(sample kernel number). The | |
| | | sample kernel number ranges from 350 to 850. | |
| | | Measurement is taken at R6 stage. | |
| STDIR3 | Stalk Diameter at R3 | plot average of the stalk diameter of a plant. It | increase |
| | | measures maximal “long” axis in the middle of | |
| | | the internode above first visible node. | |
| | | Measurement is taken at R3 stage. | |
| EDWPPR6 | Ear Dry Weight Per | plot average of the ear dry weight of a plant. | increase |
| | Plant at R6 | Measurement is taken at R6 stage. | |

These trait assays were set up so that the tested transgenic lines were compared to a control line. The collected data were analyzed against the control, and positives were assigned if there was a p-value of 0.2 or less. Tables 11-14 are summaries of transgenic plants comprising the disclosed recombinant DNA constructs for corn phenology and morphometrics assays, corn yield/trait component assays, soybean phenology and morphometrics, and yield/trait component assays, and corn and soybean root assays, respectively.

The test results are represented by three numbers: the first number before letter “p” denotes number of tests of events with a “positive” change as defined in Table 10; the second number before letter “n” denotes number of tests of events with a “negative” change which is in the opposite direction of “positive” as defined in Table 10; the third number before letter “t” denotes total number of tests of transgenic events for a specific assay for a given gene. The “positive” or “negative” change is measured in comparison to non-transgenic control plants. A designation “-” indicates that it has not been tested. For example, 2pin5t indicates that 5 transgenic plant events were tested, of which 2 events showed a “positive” change and 1 showed a “negative” change of the measured parameter. The assay is indicated with its TraitRefID as in Table 10.

TABLE 11
Summary of assay results for corn phenology and morphometric trait assays

| Construct | | | | | | | |
|---|---|---|---|---|---|---|---|
| ID | DOV12 | KRLR6 | KRNR6 | LFTNR3 | P50DR1 | S50DR1 | STDIR3 |
| TX6-03 | — | — | — | 1p0n4t | 1p0n4t | 0p0n4t | — |
| TX6-04 | — | 2p0n8t | 0p6n10t | 0p0n1t | 3p2n8t | 0p2n8t | — |
| TX6-05 | — | — | — | 2p0n4t | 2p0n2t | 2p0n2t | 0p4n4t |
| TX6-07 | — | 0p0n4t | 0p1n4t | — | — | — | — |
| TX6-08c1 | 0p1n3t | 1p1n7t | 1p2n10t | 0p0n4t | 1p1n10t | 2p3n10t | — |
| TX6-08c2 | — | 2p6n18t | 2p1n18t | — | 4p4n14t | 0p5n16t | — |
| TX6-10 | — | 0p0n4t | 0p0n4t | — | — | — | — |
| TX6-11 | — | 0p0n4t | 2p0n4t | — | — | — | — |
| TX6-12 | — | 0p1n4t | 0p3n4t | — | — | — | — |
| TX6-13 | 0p1n4t | — | — | — | 0p1n4t | 0p2n4t | — |
| TX6-15 | — | 2p3n16t | 2p5n16t | — | 1p0n12t | 2p4n16t | — |
| TX6-16 | 0p1n4t | 2p1n8t | 0p2n8t | — | Ip3n12t | 2p4n12t | — |
| TX6-18 | — | 1p0n4t | 1p0n4t | — | — | — | — |
| TX6-19 | — | 0p0n8t | 2p4n8t | — | 4p0n8t | 3p0n8t | — |
| TX6-22 | — | 1p1n6t | 0p0n6t | — | 0p0n6t | 1p0n6t | — |
| TX6-25 | — | 1p1n4t | 0p2n4t | — | — | — | — |
| TX6-27 | — | 3p4n13t | 0p1n13t | — | 3p3n10t | 1p3n10t | — |
| TX6-28 | — | 2p0n8t | Ip2n8t | — | 2p0n4t | 1p0n4t | — |
| TX6-30 | — | 0p/6n/6t | 1p/0n/6t | 1p/0n/4t | 0p/9n/10t | 1p/9n/10t | — |
| TX6-32T | — | 0p1n4t | 0p0n4t | — | 1p0n4t | 2p1n7t | — |
| TX6-33T | — | 4p0n8t | 0p1n8t | — | 0p0n4t | 0p0n4t | — |
| TX6-35T | — | 2p0n4t | 0p1n4t | — | — | — | — |
| TX6-36T | — | 0p0n4t | 1p0n4t | — | — | — | — |

TABLE 12
Summary of results for corn trait component assays

| Construct | | | | | | | | | |
|---|---|---|---|---|---|---|---|---|---|
| ID | AGDWR6 | EAR6 | EDR6 | EDWPPR6 | ELR6 | EVR6 | HINDXR6 | KPER6 | SKWTR6 |
| TX6-02 | 1p0n4t | 2p0n4t | 1p1n4t | 2p1n4t | 2p0n4t | 1p0n4t | 1p1n4t | 2p0n4t | 0p2n4t |
| TX6-03 | — | 2p0n4t | 1p0n4t | — | 1p0n4t | 1p1n4t | — | 2p0n4t | 0p1n4t |
| TX6-04 | — | 3p0n10t | 2p3n10t | — | 4p0n10t | 0p0n6t | — | 1p3n10t | 4p0n10t |
| TX6-06 | 0p0n7t | 2p0n7t | 1p1n7t | 1p0n7t | 2p0n7t | 0p0n4t | 2p1n7t | 1p0n7t | 2p1n7t |
| TX6-07 | — | 0p0n4t | 0p1n4t | — | 0p0n4t | — | — | 0p0n4t | 0p2n4t |
| TX6-08c1 | 0p0n4t | 1p4n12t | 1p2n12t | 0p3n7t | 1p2n12t | 3p0n8t | 1p0n7t | 2p3n12t | Ip3n12t |
| TX6-08c2 | 1p0n2t | 2p9n20t | 2p5n20t | 0p0n2t | 2p10n20t | — | 0p0n2t | 2p6n20t | 2p4n20t |
| TX6-10 | — | 0p0n4t | 0p2n4t | — | 1p0n4t | — | — | 0p0n4t | 0p1n4t |
| TX6-11 | — | 0p1n4t | 0p1n4t | — | 1p1n4t | — | — | 1p0n4t | 0p0n4t |
| TX6-12 | — | 1p1n4t | 0p3n4t | — | 1p1n4t | — | — | 0p2n4t | 1p0n4t |
| TX6-14 | 1p0n4t | 2p0n4t | 0p0n4t | 1p0n4t | 2p0n4t | 0p0n4t | 0p3n4t | 0p0n4t | 0p0n4t |
| TX6-15 | 1p0n4t | 6p3n20t | 1p2n20t | 1p0n4t | 5p3n20t | — | 0p1n4t | 2p4n20t | 1p1n20t |
| TX6-16 | — | 1p0n8t | 1p0n8t | — | 2p0n8t | — | — | 1p1n8t | 1p1n8t |
| TX6-18 | — | 0p2n4t | 0p3n4t | — | 0p2n4t | — | — | 2p0n4t | 0p4n4t |
| TX6-19 | 0p1n4t | 2p1n12t | 1p7n12t | 0p2n4t | 4p2n12t | — | 0p1n4t | 1p7n12t | 6p0n12t |
| TX6-20 | 4p0n4t | 3p0n4t | 1p0n4t | 0p0n4t | 4p0n4t | 0p0n4t | 0p0n4t | 4p0n4t | 0p3n4t |
| TX6-22 | 1p0n3t | 3p0n9t | 2p0n9t | 1p0n3t | 4p0n9t | — | 0p0n3t | 2p1n9t | 4p1n9t |
| TX6-24c1 | 0p1n4t | 0p1n4t | 0p1n4t | 0p1n4t | 0p0n4t | 1p1n4t | 2p1n4t | 0p1n4t | 1p0n4t |
| TX6-24c2 | 0p0n4t | 0p0n4t | 0p0n4t | 0p0n4t | 0p0n4t | 1p0n4t | 0p0n4t | 3p0n4t | 0p1n4t |
| TX6-24c3 | 0p1n4t | 0p1n4t | 0p1n4t | 0p0n4t | 0p0n4t | 0p1n4t | 3p0n4t | 0p3n4t | 3p0n4t |
| TX6-25 | 1p1n2t | 1p3n6t | 1p3n6t | 0p1n2t | 2p1n6t | — | 0p2n2t | 1p2n6t | 3p0n6t |
| TX6-27 | 0p1n3t | 4p3n16t | 5p1n16t | 0p0n3t | 3p6n16t | — | 0p1n3t | 3p5n16t | 3p0n16t |
| TX6-28 | 0p2n4t | 2p1n12t | 1p2n12t | 1p2n4t | 3p1n12t | — | 0p1n4t | 4p2n12t | 3p1n12t |
| TX6-30 | — | — | — | — | — | — | — | 0p/8n/10t | 1p/9n/10t |
| TX6-32T | 0p0n3t | 1p1n7t | 3p2n7t | 0p0n3t | 1p1n7t | — | 0p0n3t | 1p1n7t | 1p1n7t |
| TX6-33T | 0p0n3t | 3p0n11t | 1p2n11t | 0p0n3t | 3p0n11t | — | 0p2n3t | 2p0n11t | 0p2n11t |
| TX6-35T | — | 0p0n4t | 0p0n4t | — | 2p0n4t | — | — | 0p0n4t | 0p0n4t |
| TX6-36T | 1p0n2t | 0p0n6t | 0p0n6t | 0p0n2t | 3p0n6t | — | 0p0n2t | 0p1n6t | 3p0n6t |

TABLE 13
Summary of results for soybean phenology, morphometries and trait component assays

| Construct | | | | | | | |
|---|---|---|---|---|---|---|---|
| ID | AGDWR6 | ARDR2 | DBMSR6 | DFL50 | HINDXR6 | PDNODER8 | PDPPR8 |
| TX6-17 | 4p0n8t | — | — | 0p0n6t | — | — | — |
| TX6-21 | 0p0n8t | — | 0p0n4t | — | 2p1n4t | — | — |
| TX6-23 | — | — | — | — | — | 2p2n8t | 0p2n8t |
| TX6-26 | — | 0p1n8t | — | — | — | — | — |
| TX6-29 | — | 1p0n8t | — | — | — | — | — |
| TX6-31 | — | — | — | — | — | 0p8n8t | 0p2n8t |
| TX6-34T | — | 0p1n8t | — | — | — | — | — |
| TX6-37T | — | — | — | — | — | 0p2n8t | 0p4n8t |
| TX6-38T | — | — | — | — | — | 0p8n8t | 0p6n8t |

TABLE 14
Summary of assay results for corn and soybean root assays

| Crop | Construct ID | RBPN | RTL | RBNR2 |
|---|---|---|---|---|
| corn | TX6-04 | — | — | 0p1n1t |
| corn | TX6-22 | 0p0n4t | 0p0n4t | — |
| soybean | TX6-26 | 3p0n4t | 3p0n4t | 2p1n8t |
| soybean | TX6-29 | 2p0n4t | 2p0n4t | 0p3n8t |
| soybean | TX6-34T | 2p0n4t | 3p0n4t | 1p0n8t |

### Example 5. Phenotypic Evaluation of Transgenic Plants in Field Trials for Increased Nitrogen Use Efficiency, Increased Water Use Efficiency, and Increased Yield

Corn field trials were conducted to identify genes that can improve nitrogen use efficiency (NUE) under nitrogen limiting conditions leading to increased yield performance as compared to non transgenic controls. For the Nitrogen field trial results shown in Table 15, each field was planted under nitrogen limiting condition (60 lbs/acre), and corn ear weight or yield was compared to non-transgenic control plants.

Corn field trials were conducted to identify genes that can improve water use efficiency (WUE) under water limiting conditions leading to increased yield performance as compared to non transgenic controls. Results of the water use efficiency trials conducted under managed water limiting conditions are shown in Table 15, and the corn ear weight or yield was compared to non-transgenic control plants.

Corn and soybean field trials were conducted to identify genes that can improve broad-acre yield (BAY) under standard agronomic practice. Results of the broad-acre yield trials conducted under standard agronomic practice are shown in Table 15, and the corn or soybean yield was compared to non-transgenic control plants.

Table 15 provides a list of genes that produce transgenic plants having increased nitrogen use efficiency (NUE), increased water use efficiency (WUE), and/or increased broad-acre yield (BAY) as compared to a control plant. Polynucleotide sequences in constructs with at least one event showing significant yield or ear weight increase across multiple locations at p<0.2 are included. The genes were expressed with constitutive promoters unless noted otherwise under the “Specific Expression Pattern” column. A promoter of a specific expression pattern was chosen over a constitutive promoter, based on the understanding of the gene function, or based on the observed lack of significant yield increase when the gene was expressed with constitutive promoter. The elements of Table 15 are described as follows: “Crop” refers to the crop in trial, which is either corn or soybean; “Condition” refers to the type of field trial, which is BAY for broad acre yield trial under standard agronomic practice (SAP), WUE for water use efficiency trial, and NUE for nitrogen use efficiency trial; “Construct ID” refers to the construct identifier as defined in Table 4; “Gene ID” refers to the gene identifier as defined in Table 1; “Yield results” refers to the recombinant DNA in a construct with at least one event showing significant yield increase at p<0.2 across locations. The first number refers to the number of tests of events with significant yield or ear weight increase, whereas the second number refers to the total number of tests of events for each recombinant DNA in the construct. Typically 4 to 8 distinct events per construct are tested.

TABLE 15
Recombinant DNA with protein-coding genes for increased nitrogen use
efficiency, increased water use efficiency and increased yield

| Crop | Condition | Construct ID | Gene ID | Yield results |
|---|---|---|---|---|
| Corn | BAY | TX6-03 | TX6-03 | 0/8 |
| Corn | BAY | TX6-04 | TX6-04 | 9/39 |
| Corn | BAY | TX6-05 | TX6-05 | 1/16 |
| Corn | BAY | TX6-06 | TX6-06 | 0/7 |
| Corn | BAY | TX6-07 | TX6-07 | 2/22 |
| Corn | NUE | TX6-07 | TX6-07 | 4/10 |
| Corn | WUE | TX6-07 | TX6-07 | 0/5 |
| Corn | BAY | TX6-08c1 | TX6-08 | 0/8 |
| Corn | BAY | TX6-08c3 | TX6-08 | 2/22 |
| Corn | BAY | TX6-09 | TX6-09 | 5/29 |
| Corn | NUE | TX6-09 | TX6-09 | 1/11 |
| Corn | WUE | TX6-09 | TX6-09 | 0/6 |
| Corn | BAY | TX6-10 | TX6-10 | 4/23 |
| Corn | NUE | TX6-10 | TX6-10 | 1/11 |
| Corn | WUE | TX6-10 | TX6-10 | 1/6 |
| Corn | BAY | TX6-11 | TX6-11 | 7/35 |
| Corn | BAY | TX6-12 | TX6-12 | 6/23 |
| Corn | BAY | TX6-13 | TX6-13 | 0/7 |
| Corn | BAY | TX6-15 | TX6-15 | 1/18 |
| Corn | BAY | TX6-16 | TX6-16 | 0/8 |
| Corn | BAY | TX6-18 | TX6-18 | 0/8 |
| Corn | BAY | TX6-19 | TX6-19 | 0/8 |
| Corn | BAY | TX6-27 | TX6-27 | 0/8 |

Table 16 provides a list of suppression target genes and miRNA construct elements provided as recombinant DNA for production of transgenic corn or soybean plants with increased nitrogen use efficiency, increased water use efficiency and increased yield. The elements of Table 16 are described by reference to:

“Crop” which refers to the crop in trial, which is either corn or soy;

“Condition” which refers to the type of field trial, which is BAY for broad acre yield trial under standard agronomic practice, WUE for water use efficiency trial, and NUE for nitrogen use efficiency trial;

“Construct ID” refers to the construct identifier as defined in Table 4

“Target Gene ID” which refers to the suppression target gene identifier as defined in Table 2;

“Engineered miRNA precursor SEQ ID NO.” which identifies a nucleotide sequence of the miRNA construct;

“Yield results” which refers to the recombinant DNA in a construct with at least one event showing significant yield increase at p<0.2 across locations. The first number refers to the number of events with significant yield or ear weight increase, whereas the second number refers to the total number of events tested for each sequence in the construct.

TABLE 16
miRNA Recombinant DNA constructs suppressing targeted
genes for increased nitrogen use efficiency, increased
water use efficiency and increased yield

| | | | | Engineered | |
|---|---|---|---|---|---|
| | | | Target | miRNA precursor | Yield |
| Crop | Condition | Construct ID | Gene ID | SEQ ID NO. | Results |
| Corn | BAY | TX6-32T | TX6-32T | 77 | 1/8 |
| Corn | BAY | TX6-33T | TX6-33T | 78 | 3/8 |

### Example 6. Homolog Identification

This example illustrates the identification of homologs of proteins encoded by the DNA sequences identified in Table 1, which were used to provide transgenic seed and plants having enhanced agronomic traits. From the sequences of the homolog proteins, corresponding homologous DNA sequences can be identified for preparing additional transgenic seeds and plants with enhanced agronomic traits.

An “All Protein Database” was constructed of known protein sequences using a proprietary sequence database and the National Center for Biotechnology Information (NCBI) non-redundant amino acid database (nr.aa). For each organism from which a polynucleotide sequence provided herein was obtained, an “Organism Protein Database” was constructed of known protein sequences of the organism; it is a subset of the All Protein Database based on the NCBI taxonomy ID for the organism.

The All Protein Database was queried using amino acid sequences provided in Table 1 using NCBI “blastp” program with E-value cutoff of 1e-8. Up to 1000 top hits were kept, and separated by organism names. For each organism other than that of the query sequence, a list was kept for hits from the query organism itself with a more significant E-value than the best hit of the organism. The list contains likely duplicated genes of the polynucleotides provided herein, and is referred to as the Core List. Another list was kept for all the hits from each organism, sorted by E-value, and referred to as the Hit List.

The Organism Protein Database was queried using polypeptide sequences provided in Table 1 using NCBI “blastp” program with E-value cutoff of 1e-4. Up to 1000 top hits were kept. A BLAST searchable database was constructed based on these hits, and is referred to as “SubDB”. SubDB is queried with each sequence in the Hit List using NCBI “blastp” program with E-value cutoff of 1e-8. The hit with the best E-value was compared with the Core List from the corresponding organism. The hit is deemed a likely ortholog if it belongs to the Core List, otherwise it is deemed not a likely ortholog and there is no further search of sequences in the Hit List for the same organism. Homologs with at least 95% identity over 95% of the length of the polypeptide sequences provided in Table 1 are reported below in Tables 17 and 18.

Table 17 provides a list of homolog genes, the elements of which are described as follows: “PEP SEQ ID NO.” identifies an amino acid sequence. “Homolog ID” refers to an alphanumeric identifier, the numeric part of which is the NCBI Genbank GI number; and “Gene Name and Description” is a common name and functional description of the gene. Table 18 describes the correspondence between the protein-coding genes in Table 1, suppression target genes in Table 2, and their homologs, and the level of protein sequence alignment between the gene and its homolog.

TABLE 17
Homologous gene information

| PEP SEQ | | |
|---|---|---|
| ID NO. | Homolog ID | Gene Name and Description |
| 104 | gi_9791187 | gi\|9791187\|gb\|AAC39314.2\| gibberellin 20-oxidase [Arabidopsis thaliana] |
| 105 | gi_169786744 | gi\|169786764\|gb\|ACA79920.1\| DRE-binding protein 2 [Sorghum bicolor] |
| 106 | gi_160558713 | gi\|169786768\|gb\|ACA79922.1\| DRE-binding protein 2 [Sorghum bicolor] |
| 107 | gi_29372750 | gi\|116175318\|emb\|CAH64526.1\| putative MADS-domain transcription factor |
| | | [Zea mays] |
| 108 | gi_15231742 | gi\|91806578\|gb\|ABE66016.1\| galactose-binding lectin family protein [Arabidopsis thaliana] |
| 109 | gi_34582315 | gi\|48686495\|emb\|CAF29498.1\| NADP-specific glutamate dehydrogenase 1 |
| | | [Saccharomyces uvarum] |
| 110 | gi_78560967 | gi\|78560967\|gb\|ABB46391.1\| soluble starch synthase III [Arabidopsis thaliana] |
| 111 | gi_223943985 | gi\|223943985\|gb\|ACN26076.1\| unknown [Zea mays] |
| 112 | gi_9791186 | gi\|9791186\|gb\|AAC39313.2\| gibberellin 20-oxidase [Arabidopsis thaliana] |
| 113 | gi_1581592 | gi\|1581592\|prf\|\|2116434A gibberellin 20-oxidase |
| 114 | gi_171592 | gi\|171592\|gb\|AAB03898.1\| glutamate dehydrogenase [Saccharomyces cerevisiae] |
| 115 | gi_194703858 | gi\|194703858\|gb\|ACF86013.1\| unknown [Zea mays] |
| 116 | gi_62320340 | gi\|62320340\|dbj\|BAD94705.1\| gibberellin 20-oxidase - Arabidopsis thaliana |
| 117 | gi_169786752 | gi\|169786752\|gb\|ACA79914.1\| DRE-binding protein 2 [Sorghum bicolor] |
| 118 | gi_169786762 | gi\|169786762\|gb\|ACA79919.1\| DRE-binding protein 2 [Sorghum bicolor] |
| 119 | gi_1346871 | gi\|967968\|gb\|AAA74957.1\| photosystem II 10 kDa polypeptide [Brassica rapa |
| | | subsp. campestris] |
| 120 | gi_162458757 | gi\|110333721\|gb\|ABG67710.1\| gibberellin 20-oxidase [Zea mays] |
| 121 | gi_169786748 | gi\|169786748\|gb\|ACA79912.1\| DRE-binding protein 2 [Sorghum bicolor] |
| 122 | gi_116831297 | gi\|116831297\|gb\|ABK28602.1\| unknown [Arabidopsis thaliana] |
| 123 | gi_226492274 | gi\|195627904\|gb\|ACG35782.1\| gibberellin 20 oxidase 2 [Zea mays] |
| 124 | gi_15221083 | gi\|156891690\|gb\|ABU96740.1\| chloroplast starch synthase III [Arabidopsis thaliana] |
| 125 | gi_218191029 | gi\|222623102\|gb\|EEE57234.1\| hypothetical protein OsJ_07222 [Oryza sativa Japonica Group] |
| 126 | gi_226495313 | gi\|195614004\|gb\|ACG28832.1\| hypothetical protein [Zea mays] |
| 127 | gi_116310891 | gi\|218194206\|gb\|EEC76633.1\| hypothetical protein OsI_14570 [Oryza sativa Indica Group] |
| 128 | gi_21554001 | gi\|21554001\|gb\|AAM63082.1\| putative phosphatidic acid phosphatase [Arabidopsis thaliana] |
| 129 | gi_169786766 | gi\|169786766\|gb\|ACA79921.1\| DRE-binding protein 2 [Sorghum bicolor] |
| 130 | gi_242056287 | gi\|241929264\|gb\|EES02409.1\| hypothetical protein SORBIDRAFT_03g004980 |
| | | [Sorghum bicolor] |
| 131 | gi_169786756 | gi\|169786756\|gb\|ACA79916.1\| DRE-binding protein 2 [Sorghum bicolor] |
| 132 | gi_171594 | gi\|224706\|prf\|\|11111238A dehydrogenase, NADP specific Glu |
| 133 | gi_48686487 | gi\|48686491\|emb\|CAF29085.1\| glutamate dehydrogenase 1 enzyme [Saccharomyces pastorianus] |
| 134 | gi_115446841 | gi\|113536731\|dbj\|BAF09114.1\| Os02g0573200 [Oryza sativa Japonica Group] |
| 135 | gi_1109695 | gi\|1109695\|emb\|CAA58293.1\| gibberellin 20-oxidase [Arabidopsis thaliana] |
| 136 | gi_194699642 | gi\|195644016\|gb\|ACG41476.1\| gibberellin 20 oxidase 1 [Zea mays] |
| 137 | gi_121483553 | gi\|121483553\|gb\|ABM54168.1\| PSII 10 Kd peptide [Brassica juncea] |
| 138 | gi_297817704 | gi\|297322573\|gb\|EFH52994.1\| ATPAP1 [Arabidopsis lyrata subsp. lyrata] |
| 139 | gi_293335691 | gi\|224030825\|gb\|ACN34488.1\| unknown [Zea mays] |
| 140 | gi_226492052 | gi\|195636538\|gb\|ACG37737.1\| RING-H2 finger protein ATL1R [Zea mays] |

TABLE 18
Correspondence of Genes and Homologs

| | | Percent | Percent | |
|---|---|---|---|---|
| | | Gene | Homolog | Percent |
| Gene ID | Homolog ID | Coverage | Coverage | Identity |
| TX6-04 | gi_1109695 | 100 | 100 | 99 |
| TX6-04 | gi_9791186 | 100 | 100 | 99 |
| TX6-04 | gi_62320340 | 100 | 100 | 99 |
| TX6-04 | gi_1581592 | 100 | 100 | 99 |
| TX6-04 | gi_9791187 | 100 | 100 | 97 |
| TX6-05 | gi_115446841 | 100 | 99 | 100 |
| TX6-05 | gi_218191029 | 100 | 100 | 98 |
| TX6-07 | gi_226492274 | 100 | 100 | 98 |
| TX6-07 | gi_194703858 | 100 | 100 | 98 |
| TX6-09 | gi_116831297 | 100 | 99 | 98 |
| TX6-09 | gi_15231742 | 100 | 100 | 98 |
| TX6-10 | gi_194699642 | 100 | 100 | 98 |
| TX6-10 | gi_162458757 | 100 | 100 | 98 |
| TX6-15 | gi_171592 | 100 | 100 | 99 |
| TX6-15 | gi_171594 | 100 | 100 | 98 |
| TX6-15 | gi_48686487 | 100 | 100 | 95 |
| TX6-15 | gi_34582315 | 100 | 100 | 95 |
| TX6-16 | gi_226495313 | 100 | 100 | 98 |
| TX6-17 | gi_1346871 | 100 | 100 | 96 |
| TX6-17 | gi_121483553 | 100 | 100 | 95 |
| TX6-18 | gi_242056287 | 100 | 100 | 98 |
| TX6-18 | gi_160558713 | 100 | 100 | 98 |
| TX6-18 | gi_169786756 | 100 | 100 | 97 |
| TX6-18 | gi_169786752 | 100 | 100 | 97 |
| TX6-18 | gi_169786744 | 100 | 100 | 97 |
| TX6-18 | gi_169786748 | 100 | 100 | 97 |
| TX6-18 | gi_169786762 | 100 | 100 | 97 |
| TX6-18 | gi_169786766 | 100 | 100 | 97 |
| TX6-19 | gi_21554001 | 100 | 92 | 99 |
| TX6-19 | gi_297817704 | 100 | 92 | 97 |
| TX6-22 | gi_29372750 | 100 | 100 | 99 |
| TX6-22 | gi_223943985 | 100 | 100 | 99 |
| TX6-24 | gi_15221083 | 98 | 100 | 100 |
| TX6-24 | gi_78560967 | 98 | 100 | 99 |
| TX6-25 | gi_116310891 | 100 | 100 | 99 |
| TX6-27 | gi_226492052 | 100 | 100 | 95 |
| TX6-28 | gi_293335691 | 100 | 100 | 99 |

### Example 7. Use of Suppression Methods to Suppress Expression of Target Genes

This example illustrates monocot and dicot plant transformation with recombinant DNA constructs that are useful for stable integration into plant chromosomes in the nuclei of plant cells to provide transgenic plants having enhanced traits by suppression of the expression of target genes.

Various recombinant DNA constructs for use in suppressing the expression of a target gene in transgenic plants are constructed based on the nucleotide sequence of the gene encoding the protein that has an amino acid sequence selected from the group consisting of SEQ ID NOs: 70-76, where the DNA constructs are designed to express (a) a miRNA that targets the gene for suppression, (b) an RNA that is a messenger RNA for a target protein and has a synthetic miRNA targeting sequence that results in down modulation of the target protein, (c) an RNA that forms a dsRNA and that is processed into siRNAs that effect down regulation of the target protein, (d) a ssRNA that forms a transacting siRNA which results in the production of siRNAs that effect down regulation of the target protein.

Each of the various types of recombinant DNA constructs is used in transformation of a corn cell using the vector and method of Examples 1 and 2 to produce multiple events of transgenic corn cell. Such events are regenerated into transgenic corn plants and are screened to confirm the presence of the recombinant DNA and its expression of RNA for suppression of the target protein. The population of transgenic plants from multiple transgenic events are also screened to identify the transgenic plants that exhibit altered phenotype or enhanced trait.

### Example 8. Use of Site-Directed Integration to Introduce Transgenes or Modulate Expression of Endogenous Genes in Plants

As introduced above, a DNA sequence comprising a transgene(s), expression cassette(s), etc., such as one or more coding sequences of genes identified in Tables 1, 2 and 17, or homologs thereof, may be inserted or integrated into a specific site or locus within the genome of a plant or plant cell via site-directed integration. Recombinant DNA constructs and molecules of this disclosure may thus include a donor template having an insertion sequence comprising at least one transgene, expression cassette, or other DNA sequence for insertion into the genome of the plant or plant cell. Such donor template for site-directed integration may further include one or two homology arms flanking the insertion sequence to promote insertion of the insertion sequence at the desired site or locus. Any site or locus within the genome of a plant may be chosen for site-directed integration of the insertion sequence. Several methods for site-directed integration are known in the art involving different proteins (or complexes of proteins and/or guide RNA) that cut the genomic DNA to produce a double strand break (DSB) or nick at a desired genomic site or locus. Examples of site-specific nucleases that may be used include zinc-finger nucleases, engineered or native meganucleases, TALE-endonucleases, and RNA-guided endonucleases (e.g., Cas9 or Cpf1). For methods using RNA-guided site-specific nucleases (e.g., Cas9 or Cpf1), the recombinant DNA construct(s) will also comprise a sequence encoding one or more guide RNAs to direct the nuclease to the desired site within the plant genome. The recombinant DNA molecules or constructs of this disclosure may further comprise an expression cassette(s) encoding a site-specific nuclease, a guide RNA, and/or any associated protein(s) to carry out the desired site-directed integration event.

The endogenous genomic loci of a plant or plant cell corresponding to the genes identified in Tables 1 and 17, or a homolog thereof, may be selected for site-specific insertion of a recombinant DNA molecule or sequence capable of modulating expression of the corresponding endogenous genes. As described above, the recombinant DNA molecule or sequence serves as a donor template for integration of an insertion sequence into the plant genome. The donor template may also have one or two homology arms flanking the insertion sequence to promote the targeted insertion event. Although a transgene, expression cassette, or other DNA sequence may be inserted into a desired locus or site of the plant genome via site-directed integration, a donor template may instead be used to replace, insert, or modify a 5′ untranslated region (UTR), upstream sequence, promoter, enhancer, intron, 3′ UTR and/or terminator region of an endogenous gene, or any portion thereof, to modulate the expression level of the endogenous gene. Another method for modifying expression of an endogenous gene is by genome editing of an endogenous gene locus. For example, a targeted genome editing event may be made to disrupt or abolish a regulatory binding site for a transcriptional repressor of an endogenous gene to increase or modify expression of the endogenous gene.

For genome editing or site-specific integration of an insertion sequence of a donor template, a double-strand break (DSB) or nick is made in the selected genomic locus. The DSB or nick may be made with a site-specific nuclease, for example a zinc-finger nuclease, an engineered or native meganuclease, a TALE-endonuclease, or an RNA-guided endonuclease (for example Cas9 or Cpf1). In the presence of a donor template, the DSB or nick may be repaired by homologous recombination between the homology arms of the donor template and the plant genome, resulting in site-directed integration of the insertion sequence to make a targeted genomic modification or insertion at the site of the DSB or nick. For genes or suppression elements shown herein to cause or produce a desired phenotype or trait in a plant, an expression construct or transgene comprising the coding sequence of the gene or suppression element operably linked to a plant expressible promoter may be inserted at a desired or selected site within the genome of the plant via site-directed integration as discussed above. Alternatively, the sequence of a corresponding endogenous gene, such as within a regulatory region of the endogenous gene, may be modified via genome editing or site-directed integration to augment or alter the expression level of the endogenous gene, such as by adding a promoter or intron sequence, or by modifying or replacing a 5′ UTR sequence, promoter, enhancer, transcription factor or repressor binding site, intron, 3′ UTR sequence, and/or terminator region, or any portion thereof, of the endogenous gene.

Following transformation of a plant cell with a recombinant molecule(s) or construct(s), the resulting events are screened for site-directed insertion of the donor template insertion sequence or genome modification. Plants containing these confirmed edits, events or insertions may then be tested for modulation or suppression of an endogenous gene, expression of an integrated transgene, and/or modification of yield traits or other phenotypes.

1. A recombinant DNA construct comprising:
a) a polynucleotide sequence with at least 90%, at least 91%, at least 92%, at least 93%, at least 94%, at least 95%, at least 96%, at least 97%, at least 98%, at least 99%, or 100% identity to a sequence selected from the group consisting of SEQ ID NOs: 1-31;
b) a polynucleotide sequence that encodes a polypeptide comprising an amino acid sequence with at least 90%, at least 91%, at least 92%, at least 93%, at least 94%, at least 95%, at least 96%, at least 97%, at least 98%, at least 99%, or 100% identity to a sequence selected from the group consisting of SEQ ID NOs: 32-62 and 104-140;
c) a polynucleotide sequence that encodes a RNA molecule for suppressing the expression of an endogenous gene, wherein the endogenous gene encodes a mRNA molecule comprising a polynucleotide sequence with at least 90%, at least 91%, at least 92%, at least 93%, at least 94%, at least 95%, at least 96%, at least 97%, at least 98%, at least 99%, or 100% identity to a sequence selected from the group consisting of SEQ ID NOs: 63-69; or
d) a polynucleotide sequence that encodes a RNA molecule for suppressing the expression of an endogenous gene, wherein the endogenous gene encodes a protein comprising an amino acid sequence with at least 90%, at least 91%, at least 92%, at least 93%, at least 94%, at least 95%, at least 96%, at least 97%, at least 98%, at least 99%, or 100% identity to a sequence selected from the group consisting of SEQ ID NOs: 70-76.

2. The recombinant DNA construct of claim 1, wherein the polynucleotide sequence encodes a RNA molecule for suppressing the expression of an endogenous gene, and wherein the RNA molecule comprises a polynucleotide sequence that is at least 80%, at least 85%, at least 90%, at least 91%, at least 92%, at least 93%, at least 94%, at least 95%, at least 96%, at least 97%, at least 98%, at least 99%, or 100% complementary to at least 15, at least 16, at least 17, at least 18, at least 19, at least 20, at least 21, at least 22, at least 23, at least 24, at least 25, at least 26, or at least 27 consecutive nucleotides of a sequence selected from the group consisting of SEQ ID NOs: 63-69.

3. The recombinant DNA construct of claim 1, wherein the polynucleotide sequence encodes a RNA molecule for suppressing the expression of an endogenous gene, and wherein the RNA molecule comprises a polynucleotide sequence that is at least 80%, at least 85%, at least 90%, at least 91%, at least 92%, at least 93%, at least 94%, at least 95%, at least 96%, at least 97%, at least 98%, at least 99%, or 100% complementary to at least 15, at least 16, at least 17, at least 18, at least 19, at least 20, at least 21, at least 22, at least 23, at least 24, at least 25, at least 26, or at least 27 consecutive nucleotides of a mRNA sequence encoding a protein with an amino acid sequence that is at least 90%, at least 91%, at least 92%, at least 93%, at least 94%, at least 95%, at least 96%, at least 97%, at least 98%, at least 99%, or 100% identical to a sequence selected from the group consisting of SEQ ID NOs: 70-76.

4. The recombinant DNA construct of claim 1, wherein the polynucleotide sequence encodes a RNA molecule for suppressing the expression of an endogenous gene, and wherein the RNA molecule comprises a polynucleotide sequence that is at least 80%, at least 85%, at least 90%, at least 91%, at least 92%, at least 93%, at least 94%, at least 95%, at least 96%, at least 97%, at least 98%, at least 99%, or 100% identical to a sequence selected from the group consisting of SEQ ID NOs: 84-90.

5. The recombinant DNA construct of claim 1, further comprising a heterologous promoter functional in a plant cell and operably linked to the polynucleotide sequence.

6. A vector or plasmid comprising the recombinant DNA construct of claim 1.

7. A plant comprising the recombinant DNA construct of claim 1.

8. The plant of claim 7, wherein the plant is a field crop.

9. The plant of claim 8, wherein the field crop plant is selected from the group consisting of corn, soybean, cotton, canola, rice, barley, oat, wheat, turf grass, alfalfa, sugar beet, sunflower, quinoa and sugarcane.

10. The plant of claim 7, wherein the plant has an altered phenotype or an enhanced trait as compared to a control plant.

11. The plant of claim 10, wherein the enhanced trait is selected from the group consisting of: decreased days from planting to maturity, increased stalk size, increased number of leaves, increased plant height growth rate in vegetative stage, increased ear size, increased ear dry weight per plant, increased number of kernels per ear, increased weight per kernel, increased number of kernels per plant, decreased ear void, extended grain fill period, reduced plant height, increased number of root branches, increased total root length, increased yield, increased nitrogen use efficiency, and increased water use efficiency as compared to a control plant.

12. The plant of claim 10, wherein the altered phenotype is selected from the group consisting of plant height, biomass, canopy area, anthocyanin content, chlorophyll content, water applied, water content, and water use efficiency.

13. A plant part or propagule comprising the recombinant DNA construct of claim 1, wherein the plant part or propagule is selected from the group consisting of cells, pollen, ovule, flower, embryo, leaf, root, stem, shoot, meristem, grain and seed.

14. A method for altering a phenotype, enhancing a trait, increasing yield, increasing nitrogen use efficiency, or increasing water use efficiency in a plant comprising producing a transgenic plant comprising a recombinant DNA construct of claim 1.

15. The method of claim 14, wherein the recombinant DNA construct further comprises a heterologous promoter functional in a plant cell and operably linked to the polynucleotide sequence of the recombinant DNA construct.

16. The method of claim 14, wherein the transgenic plant is produced by transforming a plant cell or tissue with the recombinant DNA construct, and regenerating or developing the transgenic plant from the plant cell or tissue comprising the recombinant DNA construct.

17. The method of claim 14, further comprising:
producing a progeny plant comprising the recombinant DNA construct by crossing the transgenic plant with:
a) itself;
b) a second plant from the same plant line;
c) a wild type plant; or
d) a second plant from a different plant line,
to produce a seed, growing the seed to produce a progeny plant; and
selecting a progeny plant with increased yield, increased nitrogen use efficiency, or increased water use efficiency as compared to a control plant.

18. The method of claim 14, wherein the transgenic plant is produced by site-directed integration of the recombinant DNA construct into the genome of a plant cell or tissue using a donor template comprising the recombinant DNA construct, and regenerating or developing the transgenic plant from the plant cell or tissue comprising the recombinant DNA construct.

19. A plant produced by the method of claim 14.

20.-86. (canceled)
